(19) United States
(12) Patent Application Publication
(10) Pub. No.: US 2020/0224136 A1
(43) Pub. Date: Jul. 16, 2020

(54) INTESTINE-CHIP: DIFFERENTIAL GENE EXPRESSION MODEL

(71) Applicant: EMULATE, INC., Boston, MS (US)

(72) Inventors: Magdalena Kasendra, Boston, MA (US); Athanasia Apostolou, Brookline, MA (US); Carolina Lucchesi, Westwood, MA (US)

(21) Appl. No.: 16/742,497

(22) Filed: Jan. 14, 2020

Related U.S. Application Data
(60) Provisional application No. 62/792,212, filed on Jan. 14, 2019.

(51) Int. Cl. C12M 3/00; B01L 3/00; C12M 3/06; G01N 33/50; C12N 5/071

(52) U.S. Cl. CPC C12M 21/08; B01L 3/502715; C12M 23/16; C12N 2503/04; C12N 5/069; B01L 2300/0819; G01N 33/5082

(57) ABSTRACT

The present invention relates to fluidic systems for use in providing biomarkers for human Intestine On-Chip. More specifically, in some embodiments, a microfluidic chip containing intestinal epithelial cells co-cultured with intestinal endothelial cells in the presence of stretch and flow are used for identifying differentially expressed genes as biomarkers, e.g. for specific types of drug testing for use in treating gastrointestinal disorders or diseases related to intestinal function.

### CROSS-REFERENCE TO RELATED APPLICATIONS

This application claims benefit under 35 U. S. C. § 119(e) of the U.S. provisional application No. 62/792,212 filed 14 Jan. 2019, the contents of which are incorporated herein by reference in its entirety.

### FIELD OF THE DISCLOSURE

The present invention relates to fluidic systems for use in providing biomarkers for human Intestine On-Chip. More specifically, in some embodiments, a microfluidic chip containing intestinal epithelial cells co-cultured with intestinal endothelial cells in the presence of flow and (optionally) stretch are used for identifying differentially expressed genes as biomarkers, e.g. for specific types of drug testing for use in treating gastrointestinal disorders or diseases related to intestinal function. In addition, agents are tested on cells of a barrier (e.g. intestinal barrier) that degrade function (e.g. increase permeability) and restore function (e.g. reduce permeability).

### BACKGROUND

In search for new medicines, low oral bioavailability and poor pharmacokinetics are the common problems faced by pharmaceutical industry leading to approximately 10% of drug withdrawal¹⁻³. Human proximal small intestine represents a primary site of drug absorption and first-pass metabolism after oral administration. Indeed, it expresses a broad spectrum of drug transport (DTs) and drug metabolism enzymes (DMEs) shown to play cooperative role in detoxifying and excreting xenobiotics⁴⁻⁷. Cumulative data have demonstrated that intestinal cytochrome P450 (CYP3A4)-mediated metabolism can eliminate a large portion of some orally administered drugs before they reach systemic circulation, while leaving the passage of others unimpeded. Drugs that are subject to high intestinal metabolism not only suffer from low bioavailability, but they are also more likely to be susceptible to drug-drug interactions (DDI) with other P450 substrate or inducers drugs and show large inter-individual variations in pharmacokinetic profiles⁸⁻¹⁰.

Currently pursued extensive in vivo evaluation in preclinical species is costly and time consuming, but moreover it can give rise to misleading results and unreliable predictions because of intrinsic differences between humans and other species^{11,12}. In vitro assays offer a more efficient and cost-effective means to assess the potential oral bioavailability of large number of drug candidates. However, these assays so far utilize mostly Caco-2 monolayers, which are derived from a human colon adenocarcinoma cell line. Despite the widespread use and acceptability of these cellular model in the pharmaceutical industry, it possesses several shortcomings (e.g. distinct from in vivo expression of drug transport or metabolism enzymes) that precludes from the direct translation of the findings to humans¹³⁻¹⁵.

Additionally, intestinal cell monolayers lack organ-specific microarchitecture, physiological extracellular matrix microenvironment and mechanical forces to capture the complex and dynamic nature of the in vivo tissue development and function, including oral absorption, metabolism and inflammatory response¹⁶.

Recently, protocols to generate three-dimensional intestinal organoids (or enteroids) from human biopsy specimens were introduced as an attractive alternative for the in vitro modeling of human intestinal tissue and drug-development studies¹⁶⁻¹⁹. However, to our knowledge, the number of reports describing the pharmacokinetic properties of human intestinal organoids or their application into drug screening or pharmacology and toxicology evaluations is very limited²⁰⁻²⁴. These could be due to the substantial technical challenges associated with the use of this technology such as impaired access to lumen, which is crucial for assessing intestinal permeability or drug absorption, presence of relatively rigid ECM that could limit drug penetration, as well as heterogeneity of organoids in terms of viability, size and shape, which may impede phenotypic screening and lead to inconsistent results²⁵.

Therefore, there is a need for accurate human-relevant preclinical models able to evaluate oral absorption and metabolism of drug candidates before moving forward with drug development.

### SUMMARY OF THE INVENTION

The present invention relates to fluidic systems for use in providing biomarkers for human Intestine On-Chip. More specifically, in some embodiments, a microfluidic chip containing intestinal epithelial cells co-cultured with intestinal endothelial cells in the presence of flow and (optionally) are used for identifying differentially expressed genes as biomarkers, e.g. for specific types of drug testing for use in treating gastrointestinal disorders or diseases related to intestinal function. In addition, agents are tested on cells of a barrier (e.g. intestinal barrier) that degrade function (e.g. increase permeability) and restore function (e.g. reduce permeability).

Cellular permeability barriers in organs, tissues and vasculature maintain a physical barrier while providing an interface balancing communications. Such communications include, but are not limited to, controlling substances crossing from one side to the other side of the barrier, e.g., by preventing or allowing diffusion of substances between cells, inhibiting cellular migration through the cell layer and providing active transport of certain molecules from one side to the other side of the barrier, etc. These physical barriers include or are provided by either epithelial cell layers, endothelial cell layers or a combination of both epithelial cell and endothelial cell layers.

Moreover, the permeability of these physical barriers is disrupted, e.g. by inducing a leaky barrier, during pathophysiology associated with numerous types of disorders and diseases. Thus, treatments are needed for reversing a diseased/leaky barrier phenotype to normal, or at least reverse the disease barrier phenotype towards normal in a manner that may in part alleviate at least one disease associated symptom. Further, methods of screening potential treatments are needed to avoid adverse drug-drug interactions (DDIs). Alternatively, methods of screening potential treatments are needed to identify synergistic drug-drug interactions.

In vivo and in vitro methods using animal models show species specific nature of drug transporters and enzymes, particularly when compared to available human models. Therefore, human based in vitro testing systems are used, including but not limited to using organoids alone in static cultures, and cell lines, such as Caco-2 cell lines for testing drugs associated with intestinal barrier function.

Therefore, the invention provides in vitro methods of using microfluidic devices for testing substances that may be used for restoring a disrupted leaky barrier to a more normal barrier phenotype. For providing such methods, in some embodiments, a first substance may be used for inducing a leaky barrier, e.g. an injury inducing substance. In some embodiments, the injury caused by the injury inducing substance is known to be reversible. In some embodiments, methods are provided for determining whether the injury is reversible (or to what degree it is reversible) using at least one known barrier rescue substance (or a candidate test substance screened to identify whether it is a rescue substance).

A substance for inducing a leaky barrier is not limited to a molecular substance such that in some embodiments, the use of diseased cells or cells derived from a diseased patient may be used for providing the actual leaky barrier of epithelial and/or endothelia cells. In some embodiments, the use of diseased cells or cells derived from a diseased patient may be used for providing a cell barrier more susceptible to a first substance for inducing a leaky barrier, such that the first level of permeability is higher than a comparable level of permeability

In further embodiments, such diseased patient derived cells may be contacted with a first substance. Regardless of how cells comprising a leaky barrier are provided, in some embodiments, a leaky barrier phenotype is treated with a second substance for reversing, i.e. for decreasing permeability, as a rescue substance. However, it is not intended that only complete reversal is sought or observed; partial reversal is also sought and observed.

Thus, in one embodiment, the invention provides a method for identifying a reversible injury substance, comprising, a) providing; i) a microfluidic device having an inlet and an outlet fluidically connected by a microchannel, wherein said microchannel comprises a cell monolayer having a first level of permeability; ii) a test injury inducing substance in fluid for increasing permeability of said barrier, and b) flowing said test injury inducing substance fluid through said inlet into said microchannel for contacting said cells so as to create a treated barrier under conditions for allowing increases in said first level of permeability to a second level of permeability.

In one embodiment, the invention provides a method for identifying a reversible injury substance, comprising, a) providing; i) a microfluidic device comprising a cell barrier having a first level of permeability; ii) a test injury inducing substance in a fluid for increasing permeability of said barrier, and b) flowing said fluid test injury inducing substance through said microfluidic device for contacting said cells so as to create a treated barrier under conditions for allowing increases in said first level of permeability to a second level of permeability.

In one embodiment, the invention provides a method for treating a cell barrier (or testing agents on cells that make up a cellular barrier), comprising, a) providing; i) a microfluidic device comprising one or more layers of cells comprising a barrier having a first level of permeability; ii) a first substance capable of increasing the permeability of said barrier to a second level, and iii) a second substance capable of decreasing the permeability of said barrier (or a second substance that is a candidate substance for screening to determine to what degree it is capable of decreasing the permeability of the cellular barrier); b) contacting said barrier with said first substance so as to create a treated barrier; and c) contacting said treated barrier with said second substance. In one embodiment, said barrier is an intestinal barrier, i.e. made up of cells found in the intestine (whether from the colon, small intestine, or other region ask discussed herein). It is not meant to limit the number of levels of permeability. For example, in one embodiment, said second substance is capable of decreasing said permeability to a third level that is higher than said first level and lower than said second level. In other words, said second substance improves said permeability barrier but does not completely restore it to said first level. As another example, in one embodiment, said method further includes a third level of permeability whereas said third level is located in between said first level and said second level. In one embodiment, said second substance is capable of decreasing said permeability to a fourth level that is lower than said first level. In other words, in some embodiments, said second substance is capable of improving said permeability level over said first permeability level. In one embodiment, said method further comprises, prior to step c), determining whether said first substance increases said permeability of said barrier. In one embodiment, said method further comprises, after step c), determining whether said second substance decreases said permeability of said treated barrier. In one embodiment, said second level is up to 2-fold greater, more typically up to 5 fold barrier, and in some cases up to 50-fold greater than said first level. In one embodiment, said second substance counteracts up to 20%, more typically up to 50%, and in some cases up to 90% (or even 100%) of said increase in said permeability caused by said first substance. In one embodiment, said microfluidic device comprises an inlet and an outlet that are fluidically connected by a microchannel, wherein said microchannel comprises said one or more layers of cells perfused by fluid. In one embodiment, said contacting of step b) is performed by introducing said first substance into said inlet. In one embodiment, said second substance is a zonulin receptor antagonist. In one embodiment, said second substance blocks zonulin receptors. In one embodiment, said second substance is N-(2-bromophenyl)-9-methyl-9-azabicyclo[3.3.1] nonan-3-amine (AT1001/larazotide acetate). In one embodiment, said second substance is larazotide acetate. In one embodiment, said cell layer is selected from the group consisting of epithelial cells and endothelial cells. In one embodiment, said cells are selected from the group consisting of primary cells, biopsy derived cells, induced pluripotent (iPS) cells, organoid-derived cells and cell lines. In one embodiment, said cells are selected from the group consisting of healthy cells, disease cells, cells derived from patients having a disease, cells derived from a patient suspected of developing a disease, and cells derived from a patient identified as having a disease susceptibility. In one embodiment, said cells are selected from the group consisting of cells derived from a disease affected tissue of a patient and cells derived from an area of tissue next to a disease affected tissue of a patient. In one embodiment, said cells are selected from the group consisting of cells having at least one known gene sequence, cells having at least one known gene mutation, cells having at least one known genetic allele, and cells having at least one gene that is genetically engineered. In one embodiment, said cells are derived from patients having at least one disease symptom selected from the group consisting of an inflammatory bowel disease (IBD), celiac disease, Crohn's disease (CD), and ulcerative colitis (UC). In one embodiment, said cells are derived from patients having at least one disease symptom selected from the group consisting of neurodegenerative disorders, neuro-inflammatory disorders, and X-linked adrenoleukodystrophy (X-ALD). In one embodiment, said cells are derived from patients having at least one disease symptom selected from the group consisting of diabetes and chronic kidney disease (CKD). In one embodiment, said cells are derived from patients having at least one disease symptom selected from the group consisting of alcoholic liver disease (ALD) and non-alcoholic fatty liver disease (NAFLD). In one embodiment, said microfluidic device comprises at least a first cell layer and a second cell layer. In one embodiment, said first cell layer comprises epithelial cells and said second cell layer comprises endothelial cells. In one embodiment, said epithelial cells are selected from the group consisting of organoid-derived epithelial cells. In one embodiment, said organoid-derived epithelial cells are human. In one embodiment, said epithelial cells are selected from the group consisting of intestine-derived cells, organoid-derived intestine epithelial cells, organoid-derived duodenal epithelial cells, organoid-derived ileal epithelial cells, and organoid-derived colonic epithelial cells. In one embodiment, said epithelial cells are human organoid-derived colonic epithelial cells. In one embodiment, said endothelial cells are selected from the group consisting of microvascular endothelial cells (MECs) and umbilical vein endothelial cells (HUVECs). In one embodiment, said endothelial cells are selected from the group consisting of intestine-derived endothelial cells and intestinal microvascular endothelial cells (IMEC). In one embodiment, said endothelial cells are selected from the group consisting of brain microvascular endothelial cells (BMECs). In one embodiment, said endothelial cells are selected from the group consisting of renal glomerular endothelial cells (GEC). In one embodiment, said endothelial cells are human. In one embodiment, said endothelial cells are human organoid-derived endothelial cells. In one embodiment, said endothelial cells are rat. In one embodiment, said endothelial cells are human biopsy derived endothelial cells. In one embodiment, said endothelial cells are a human cell line. In one embodiment, said first substance comprises a cytokine. In one embodiment, said first substance is selected from the group consisting of a live microbe; a live bacterium; a bacterial substance; Lipopolysaccharides (LPS); and endotoxins. In one embodiment, said first substance is selected from the group consisting of IFN-γ, INF-α, IL-1β, IL-4, IL-6, IL-12, IL-17, IL-22, IL-23, and IL-26. In one embodiment, said first substance is IFN-γ. In one embodiment, said first substance is a population of white blood cells comprising neutrophils (PMNs). In one embodiment, said barrier of step a) i) has tight junctions. In one embodiment, said first substance opens at least a portion of said tight junctions. In one embodiment, said first substance is a six-mer synthetic peptide H-FCIGRL-OH of a Zonula occludens toxin (AT1002). In one embodiment, said level of permeability comprises molecule permeability, dye permeability, transepithelial electrical resistance, transendothelial electrical resistance, expression of permeability related proteins and visual observation of permeability related proteins. In one embodiment, said level of permeability is measured by a molecule diffusion assay. In one embodiment, said level of permeability is measured by a dye diffusion assay. In one embodiment, said level of permeability is electrically measured. In one embodiment, said level of permeability is visually observed. In one embodiment, said barrier is on a membrane. In one embodiment, said barrier is under stretch.

The present invention provides a method for growing colon cells on a fluidic device, comprising: a) providing; i) a fluidic device comprising at least one membrane separating at least two chambers, said device (or mechanism) capable of stretching the membrane; ii) epithelial cells; and iii) endothelial cells; b) seeding said device with said cells under condition such that said epithelial cells are on a first side of said membrane and said endothelial cells are on a second side of said membrane; c) initiating a first period of membrane stretching starting on any one of days 1-3; and d) increasing the amount of membrane stretching on any of days 2-4. In one embodiment, said epithelial cells are intestinal epithelial cells. In one embodiment, epithelial cells are human colonoid derived epithelial cells. In one embodiment, endothelial cells are intestinal endothelial cells. In one embodiment, endothelial cells are colonic HIMECs. In one embodiment, membrane stretching of said first period is 2%, 0.15 Hz cyclic stretching. In one embodiment, said membrane stretching is initiated on day 3. In one embodiment, membrane stretching is increased on day 4. In one embodiment, membrane stretching is increased to 10%, 0.15 Hz cyclic stretching. In one embodiment, said method further comprises, prior to step c), introducing culture media into said device at a flow rate. In one embodiment, said flow rate is 60 ul/hr. In one embodiment, said two chambers comprise a first and second microchannel.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Exemplary embodiments are illustrated in referenced figures. It is intended that the embodiments and figures disclosed herein are to be considered illustrative rather than restrictive.

FIG. 1A shows exemplary illustrations of a perspective view of a microfluidic device with microfluidic channels in accordance with an embodiment (left) with a mirror image CAD illustration (right).

FIG. 1B shows exemplary illustrations of an exploded view of a device in accordance with an embodiment, showing a microfluidic channel in a top piece and a microfluidic channel in a bottom piece, separated by a membrane.

FIG. 1C illustrates an exemplary schematic representation for one embodiment of a microfluidic intestine-on-chip: 1. Epithelial Channel (blue); 2. Human Intestinal Epithelial Cells, e.g. Caco2, duodenal, ileum, colon, etc., as enteroids-derived, primary intestinal cells, cancer cells, etc.; 3. Vacuum Channel; 4, a flexible, porous, ECM-coated PDMS membrane; 5. Human Intestinal Endothelial Cells e.g., HIMEC, iHIMEC, etc.; and 6. Vascular Channel (pink). Designing and engineering the microenvironment allows us to recreate a “home away from home” for the cells within microfluidic Organ-Chips, including: Extracellular matrix and cell interactions; cell shape and cytoarchitecture; tissue-tissue interactions; mechanical forces as part of a dynamic system; flow may include test substances, drugs, cytokines, resident immune cells or circulating immune cells, etc.

FIG. 1D shows one embodiment of a microfluidic intestine-on-chip. In one embodiment a chip is comprised of at least two micro-channels (blue and pink channels) separated by a porous flexible-membrane. The material is functionalized with extracellular matrix and different types of cells are seeded into the two different channels. In one embodiment, of an Intestine On-Chip model, human endothelial cells are seeded in the bottom compartment (pink) and human epithelial cells in the top compartment (blue) to emulate the basic functioning unit of at least a portion of the intestine. Vacuum pressure can be applied to the side channels (gray) to mechanically stretch the membrane. Fluids can be continuously pumped through the channels to mimic shear forces, bring in nutrients, bring in bacteria, bring in test compounds and antibiotics, flush away wastes and provide effluent fluids for sampling.

FIG. 1E-G shows exemplary immunofluorescent micrographs of immunostained embodiments of intestine on-chip derived from exemplary cell sources: including in some embodiments, incorporating patient-derived lamina propria immune cells on-Chip.

FIG. 1E Caco-2 (BBE) stained with Phalloidin—Actin (green), MUC2-Mucin 2 (purple), and DAP1-stained nuclei colored blue.

FIG. 1F shows exemplary primary Enteroids-derived intestine showing Muc2 stained —Goblet Cells (pink), Lysozyme—Paneth Cells (green), and DAP1 stained nuclei colored blue.

FIG. 1G shows exemplary iPSC organoids-derived intestine showing ZO-1—Tight Junction (green), E-cadherin (blue), Cdx2 stained nuclei colored red.

FIG. 1H shows exemplary incorporation of Lamina Propria Derived Immune Cells on the Intestine-Chip (schematic left). Upper panel epithelium, middle panel showing immune cells and lower panel endothelium. Tight junctions (red), F-actin (green), immune cells (pink), DAP1 stained nuclei colored blue.

FIG. 2A-B shows an exemplary schematic of an open top microfluidic chip.

FIG. 2A illustrates an exemplary perspective view of a microfluidic open top device with microfluidic channels in accordance with an embodiment.

FIG. 2B illustrates an exemplary exploded view of an open top device in accordance with an embodiment, showing a microfluidic culture channel in a top piece and a microfluidic channel in a bottom piece, separated by a membrane.

FIG. 3A-B shows an exemplary time course and extent of villus differentiation in the Intestine Chip. Representative phase contrast images of duodenal organoid-derived epithelial cells cultured on chip under continuous flow and peristalsis-like motions. (FIG. 3A) Comparison of the same field of view (center of the channel) imaged at 1, 4, 8 and 12 days of culture demonstrates that the villi formed de novo within these cultures. (FIG. 3B) Views of three different regions beginning (top), middle (center) and end (bottom) of the culture channel confirming that villi-like structures form at high density along the entire length of the channel.

FIG. 4 shows an exemplary genome-wide hierarchical clustering of the Intestine Chip versus other intestinal culture models. Hierarchical clustering analysis of genome-wide transcriptome profiles of Intestine Chip, Organoid, Caco-2 Gut Chip or Caco-2 Transwell cultured in static condition compared with normal human small intestinal tissues (Duodenum, Jejunum, and Ileum; microarray data from a published GEO database). The dendrogram was generated based on the averages calculated across all replicates, and all branches in the cluster have the approximately unbiased (AU) P value larger than 95. The y-axis next to the dendrogram represents the metric for maximum distance between samples. Corresponding pseudocolored GEDI maps analyzing profiles of 650 metagenes between samples described above.

FIG. 5A-D shows exemplary micrographs of duodenal organoids (enteroids) and a chart showing comparative permeability between three different tissue donars. FIG. 5A shows exemplary bright field images of human duodenal organoids (top) and human microvascular endothelial cells (bottom) acquired before their seeding into an epithelial and endothelial channel of one embodiment of an Organ-on-Chip platform, respectively.

FIG. 5B shows exemplary scanning electron micrograph showing complex intestinal epithelial tissue architecture achieved by one embodiment of duodenal epithelium grown for 8 days on the chip (top) in the presence of constant flow of media (30 μl and cyclic membrane deformations (10% strain, 0.2 Hz). High magnification of the apical epithelial cell surface shows densely packed intestinal microvilli (bottom).. See FIG. 6A-D demonstrating the effect of mechanical forces on the cytoarchitecture of epithelial cells and the formation of intestinal microvilli.

FIG. 5C shows an exemplary composite tile scan fluorescence image (top) showing a fully confluent monolayer of organoids-derived intestinal epithelial cells (magenta, ZO-1 staining) lining the lumen of one embodiment of an engineered Duodenum Intestine-Chip and interfacing with microvascular endothelium (green, VE-cadherin staining) seeded in the adjacent vascular channel. Lower panels show higher magnification views of epithelial tight junctions (bottom left) stained against ZO-1 (magenta) and endothelial adherence junctions visualized by VE-cadherin (green) staining. Cell nuclei are shown in grey. Scale bar, 1000 μm (top) 100 μm (bottom).

FIG. 5D shows exemplary apparent permeability values in one embodiment of Duodenum-Chips, each of which were established from organoids of 3 different donors, grown in the presence of flow and stretch (30 μA, 10% 0.2 Hz) for up to 10 days. Papp values were calculated from the diffusion of 3 kDa Dextran from the luminal to the vascular channel. Data represent 3 independent experiments, each using different donors of biopsy-derived organoids, i.e. 3 different chips/donors; Error bars indicate S.E.M.

FIG. 6A-D shows exemplary flow-induced increase of primary intestinal epithelial cell height and microvilli formation in one embodiment of duodenal organoid-derived epithelial cells.

FIG. 6A shows an exemplary representative confocal images of x-y (top) and x-z (bottom) optical sections of duodenal organoids-derived epithelial cells cultured under static (Static) or fluid flow (30 μl h⁻¹; Flow) or flow and stretch (30 μl h⁻¹; 10% strain, 0.2 Hz; Flow+Stretch) conditions and stained for apical marker villin (green) and basolateral protein E-cadherin (magenta). Nuclei we counterstained with DAP1 (grey). Scale bar, 50 μm.

FIG. 6B shows an exemplary quantitative analysis of the average cell height measured from Z-stack images as the distance between apical marker villin (green, 6A) and PDMS membrane (dotted line, 6A). The data represent the mean±s.e.m; One-way ANOVA, ****p<0.0001.

FIG. 6C shows an exemplary scanning electron microscopy surface images of duodenal enteroid(organoid)-derived epithelium cultured for 72 hours under static or flow+/− stretch conditions. Cells seeded in the top channel of Organ-on-Chip, e.g., Intestine-Chip, platform were grown with (Flow) or without (Static) medium perfusion (30 μl h⁻¹) in both channels and 10% of mechanical stretch (0.2 Hz) (Flow+Stretch). Images were captured at the center area of the chamber. Scale bar, 5 μm.

FIG. 6D shows an exemplary quantification of microvilli. Density of microvilli per μm² was measured from the SEM images (100 μm2, 20 FOV) as described in the Methods. Data represent the mean±±s.e.m; One-way ANOVA, ****p<0.0001.

FIG. 7A-C shows exemplary multi-lineage differentiation and barrier function in one embodiment of a duodenal-chip mimicking those characteristics found in native human intestine.

FIG. 7A shows an exemplary comparison of the relative gene expression levels of the intestinal cell types specific markers, including mucin 2 (MUC2) for goblet cells, alkaline phosphatase (ALPI) for absorptive enterocytes, chromogranin A (CHGA) for enteroendocrine cells, lysozyme (LYZ) for Paneth cells. Leucine-rich repeat-containing G-protein coupled receptor 5 (LGR5) was also used for stem cells and proliferation marker (KI67). Staining was across Intestine-Chips established from 3 individual organoids donors at different time of their fluidic culture (day 2, 4, 6, 8, 10) and RNA was isolated directly from the duodenal tissue of other 3 different donors (In vivo). In each graph, values represent average gene expression±s.e.m (error bars) from three independent experiments, each using different donors of biopsy-derived organoids and at least three different chips per time point. Values are shown relative to duodenal tissue expressed as 1. EPCAM expression was used as normalizing control. One-way ANOVA, ****p<0.0001, ***p<0.001, **p<0.01, *p<0.05, ns p>0.05.

FIG. 7B shows an exemplary representative confocal fluorescent micrographs in one embodiment of in Duodenum Intestine-Chip demonstrating the presence of major intestinal cell types (green) at in vivo relevant ratios at day 8 of fluidic culture, including goblet cells stained with anti-protein antibodies, anti-Muc2 (green); enteroendocrine cells visualized with anti-chromogranin A (green), absorptive enterocytes stained with anti-villin (green) and Paneth cells labeled with anti-lysozyme (green). Cell-cell borders were stained with anti-E-cadherin and are shown in magenta. Scale bar, 10 μm. Exemplary in vivo ratios, see, Karam S M. Front Biosci 1999, 4:D286-298.

FIG. 7C shows exemplary quantification of different epithelial intestinal cell types present in one embodiment of in Duodenum Intestine-Chip at day 8 of fluidic culture and identified by immunostaining, as described in FIG. 7B. Cell ratios as percentage of different cell types based on 10 different fields of view (10 FOV) counted in three individual chips (each from a different donor) per staining. DAP1 staining was used to evaluate the total cell number. Duodenum values, representative cell ratios observed in the histological sections and are based on the literature (Karam 1999).

FIG. 8 Shows exemplary relative average gene expression of intestinal drug transporters in one embodiment of a Duodenum Intestine-Chip compared to Caco-2 Intestine-Chips and in vivo duodenum tissue where RNA was isolated directly from the duodenal tissue of three independent individuals (In vivo Duodenum), n=3. Three donor-specific Duodenum Intestine-Chips and organoids. Comparison of the relative average gene expression levels of drug efflux (MDR1, BCRP, MRP2, MRP3) and uptake (PEPT1, OATP2B1, OCT1, SLC40A1) transporters in Caco-2 Intestine-Chip, Duodenum Intestine-Chip, both assessed on day 8 of culture, and RNA isolated directly from the duodenal tissue (Duodenum). The results show that Duodenum Intestine-Chip expresses drug transport proteins at the levels close to human duodenal tissue. Note, the expression of OATP2B1 and OCT1 in Caco-2 Intestine-Chip were significantly higher than in human duodenum while the difference between Duodenum Intestine-Chip and adult duodenum is not significant. Each value represents average gene expression±s.e.m (error bars) from three independent experiments, each involving Duodenum Intestine-Chip established from a tissue of three different donors (three chips/donor), RNA tissue from three independent biological specimens, and Caco-2 Intestine-Chip (three chips). Values are shown relative to the duodenal tissue expressed as 1. Two-way ANOVA, ****p<0.0001, *** p<0.001, **p<0.01. EPCAM expression was used as normalizing control.

FIG. 9A-C Shows exemplary correct localization and function of efflux pump MDR1 (P-gp) expression in one embodiment of a Duodenum Intestine-Chip. FIG. 9A shows an exemplary representative confocal immunofluorescence micrographs (left) and graphs (right) showing localization and fluorescent signal distribution, respectively, of efflux transporters MDR1 (green), and villin (magenta) across vertical cross-sections of the differentiated epithelium in Duodenum-Chip. Merge (shown in white). Comparison of the line profiles reveals apical localization of the efflux transporter protein MDR1 (green) co-localize precisely with luminal cell surface marker villin (magenta) in a vertical cross section of monolayer (top) in the cells which are closely attached to membrane (monolayer) at day 4 as well as in cells later formed lining 3D villi-like structures (bottom) at day 8. Fluorescent signal representing cell nuclei is visualized in cyan. Scale bar, 10 μm. Exemplary line plots (right) corresponding to confocal images (left) showing the distribution of fluorescent intensities for 3 different channels: MDR1 (green), villin (magenta) and nuclei (cyan) along the basal-apical axis of enterocytes forming a monolayer or villi-like structures in Duodenum Intestine-Chip. The fluorescent intensity was analyzed in 3D reconstructed confocal images of Duodenum Intestine-Chip and plotted as average across 20 different z-stacks. Distribution of MDR1 and villin shows significant overlap. See also FIG. 10A-B showing luminal localization of additional efflux (BCRP) and uptake (PEPT1) transporters in Duodenum Intestine-Chip.

FIG. 9B shows an exemplary activity of efflux pump proteins in DMSO (carrier) (left) and an inhibitor (vinblastine) right) in Duodenum Intestine-Chip.

FIG. 9C shows an exemplary activity of efflux pump proteins in DMSO (carrier) (left) and an inhibitor (vinblastine) right) in Caco-2-Chip and Duodenum Intestine-Chip. The intracellular accumulation of the fluorescent substrate of MDR1, Rhodamine 123, was significantly increased in response to the MDR1 inhibitor vinblastine (black bars) in comparison to vehicle (DMSO) control (grey bars) in both systems Caco2 and organoids-derived Duodenum-Chips. Data are presented as mean±s.e.m (error bars) of at least three independent experiments involving chips generated from organoids of at least three individual donors or Caco-2 cell line, assessed eight days post-seeding. Two-way ANOVA, **p<0.01, *p<0.05.

FIG. 10A-B shows exemplary Luminal localization of efflux (BCRP) and uptake (PEPT1) transporters in Duodenum-Chip. Representative cross-sectional confocal images of epithelial tissue grown inside of Duodenum-chip (left) showing apical localization of efflux BCRP (FIG. 10A; green) and uptake PEPT1 drug transporters (FIG. 10B; green) that co-localize with luminal cell surface marker villin (magenta) at the time of confluent monolayer formation (day 4 of culture) as well as within successively formed villi-like structures (at day 8). Line plots representing distribution of the fluorescence signal across epithelial cell z-axis revealed close overlap of green (transporters; BRCP and PEPT1) and magenta (apical cell marker; villin) signals confirming co-distribution of these proteins on the luminal cell surface. Cell nuclei are visualized in cyan. The fluorescent intensity for each channel was analyzed in 3D reconstructed confocal images of Duodenum Intestine-Chip and plotted as average across 26 different z-stacks (for BCRP) and 21 different z-stacks (for PEPT1). Scale bar, 10 μm. Data from 3 independent experiments, each using different donors of biopsy-derived organoids and Caco-2 Intestine-Chips, n=3.

FIG. 11A-D shows exemplary CYP3A4 expression levels and drug-mediated induction in a Duodenum-Chip and Caco-2 based Intestine-Chip system.

FIG. 11A shows a exemplary comparative average gene expression levels of CYP3A4 s.e.m (error bars) in Intestine-Chips established from a Caco-2 cell line and Duodenum-Chip developed from organoids of at least 3 individual donors at day 8 and adult duodenal tissue (Duodenum). Values are shown relative to in vivo tissue expressed as 1. One-way ANOVA, ****p<0.0001, **p<0.01. Epcam expression was used as normalizing control.

FIG. 11B shows exemplary protein analysis of CYP3A4 in Caco-2 cells and organoids-derived epithelium grown inside of Organ-on-Chip platform using western blotting. Assessed at day 8.

FIG. 11C shows exemplary CYP3A4 induction potency in Duodenum-Chips (organoids) and Intestine-Chips (Caco-2) treated with solvent (DMSO), 20 μM rifampicin (RIF) or 100 nM 1,25-dihidroxyvitamin D3 (VD3) treated at day 6 for 48 h at day 8. The gene expression levels (top) of CYP3A4 were examined by real-time PCR analysis. On the y-axis, the gene expression levels in the DMSO-treated chips were taken as 1.0. Data are represented as means±s.e.m. Two-way ANOVA, ****p<0.0001 (compared with DMSO-treated cells). The corresponding CYP3A4 protein expression levels (bottom) were measured at day 8 by western blotting analysis. Data shown is from 3 independent experiments, each using different donors of biopsy-derived organoids and Caco-2 Intestine-Chips, n=3.

FIG. 11D shows exemplary gene expression analysis of a Drug Metabolism Enzyme, e.g. CYP3A4, and nuclear factors, e.g. PXR and VDR in organoids and Caco-2 cell-derived chips examined by real-time RT-PCR analysis at day 8 and compared to their expression in adult duodenal tissue (Duodenum). On the y-axis, the gene expression levels in adult tissue were taken as 1.0. All data are represented as means±s.e.m. Two-way ANOVA, ***p<0.001, **p<0.01, *p<0.05. RIF—20 uM rifampicin. VD3-100 nM 1,25-dihidroxyvitamin. Relative average gene expression data from Caco-2 Intestine-Chips, three donor-specific Duodenum Intestine-Chips and RNA isolated directly from the duodenal tissue of three independent individuals (In vivo Duodenum), n=3. CYP3A4 enzyme activity was also determined by monitoring the formation of 6β-hydroxytestosterone in the medium of Duodenum Intestine-Chip and Caco-2 Intestine-Chip, as measured by LC-MS. For induction studies, 20 μM RIF or 100 nM VD3 was added 48 h before measurement. Data are expressed as mean±s.e.m of three independent experiments each involving Duodenum Intestine-Chip established from organoid-derived cells of a different donor and Caco-2 Intestine-Chip. At least three different chips were used per condition. Two-way ANOVA, ****p<0.0001.

FIG. 12 shows exemplary CYP3A4 Enzyme Activity Across Multiple Donors in The Duodenum Intestine-Chip. Data from 3 independent experiments, each using different donors of biopsy-derived organoids, n=3. In vivo referenced from: Obach R. S. et all. Drug Metabolism and Disposition. 2001, 29 (3) 347-352 (human small intestinal microsomal preparations).

FIG. 13A-D Shows exemplary On-Chip growth promoting higher transcriptomic similarity of human primary intestinal epithelium to adult duodenal tissue than to 3D organoid culture.

FIG. 13A shows an exemplary Venn diagram generated using the genes found from the differential gene analyses Duodenum-Chip vs Organoids (cyan circle) and Adult Duodenum vs Organoids (yellow circle). The intersection (green) contains 305 genes that are commonly differentiated in Duodenum-Chip and Adult Duodenum from the organoids. Sample sizes were as follows: Duodenum-Chip, n=3; Organoids, n=3; Adult duodenum, n=2. All samples were biologically independent (each derived from a different donor). Samples from the same 3 donors were used for the establishment of Duodenum-Chip and Organoids cultures.

FIG. 13B shows an exemplary REViGO Scatterplot plot presenting the results of the GO analysis of 305 commonly differentially expressed genes. Each circle corresponds to a specific GO term and the circle size is proportional to the number of genes included in each of the enriched GO terms. The color of a circle indicates the significance of the specific GO term enrichment. Example, red highly enriched, blue least enriched, shows the cluster representatives (i.e. terms remaining after the redundancy reduction) in a two dimensional space derived by applying multidimensional scaling to a matrix of the GO terms' semantic similarities.

FIG. 13C shows exemplary results of the KEGG pathway analysis of exemplary Duodenal Functional Pathways using the 305 differentially expressed genes. Results showed seven significantly enriched (FDR adjusted p-value <0.05) pathways related to absorption, metabolism, digestion and chemical carcinogenesis. The length of the bars indicates the fold-enrichment of the corresponding pathways. Presented pathways are significantly enriched (FDR corrected p-value <0.05).

FIG. 13D exemplary curated heatmaps showing the expression intensity (log₂(FPKM)) of the genes contained in the corresponding KEGG pathways. The provided results clearly indicate the significant gene expression differences of the Organoids compared to Duodenum-Chip (Duo Chip) and Adult Duodenum (Adult Duo). Sample sizes were as follows: Duodenum-Chip, n=3; Organoids. n=3; In vivo. Adult duodenum, n=2. All samples were biologically independent (derived from a different donor). Intestinal crypts derived from the same three independent donors were used for the establishment of Duodenum Intestine-Chip and organoid cultures. Both chips and organoids were cultured in parallel as described in 13A). Samples from the same 3 individual donors were used for the establishment of Duodenum-Chip and Organoids cultures. See also FIG. 14A-B showing the results of DGE analysis followed by functional enrichment performed between Organoids or Duodenum Intestine-Chip and Adult Duodenum.

FIG. 14A-B shows an exemplary differentially expressed genes and enriched Gene Ontology categories in Organoids (FIG. 14A) or Duodenum-Chip (FIG. 14B) in respect to Adult Duodenum.

FIG. 14A shows an exemplary Volcano plot with a functional enrichment analysis (Table 3) of differentially expressed (DE) genes between Organoids and Adult Duodenum. Organoids Vs. Adult Duodenum showed 1437 Differentially Expressed Genes. The red dots represent genes that are significantly (adjusted p-value<0.05) up- or down-regulated. The black dots correspond to the non-differentially expressed genes. The vertical lines correspond to 2.0-fold up and down and the horizontal line has been drawn at the level of the selected cutoff adjusted p-value (adjusted p-value<0.05). Sample sizes were as follows: Duodenum Organoids, n=3. Adult Duodenum, n=2. All samples were biologically independent (derived from a different donor). Samples from the same 3 donors were used for the establishment of Duodenum-Chip and Organoids cultures. Organoids were cultured in the presence of expansion media for 6 days, followed by 2 days of differentiation media. Analysis was performed in samples collected 8 days post-seeding. Functional enrichment analysis demonstrated over (+) and under (−) represented biological processes in the GO categories concerning digestion, extracellular matrix organization, angiogenesis, cell adhesion, tissue development, cell response to drugs and toxic substances, while nucleic acid metabolic process and RNA processing were under represented. GO, Gene Ontology.

FIG. 14B shows an exemplary differential gene expression and functional enrichment analysis between Duodenum-Chip and adult human tissue demonstrating up- and down-regulated genes (volcano plot) and annotated to them biological processes (Table 3S). Primary Duodenum Intestine-Chip Vs. Adult Duodenum showed 1032 Differentially Expressed Genes. Differential genes were involving but not limited to protein synthesis and targeting as well as cell cycle and cell proliferation. Red dots: significant genes (adjusted p-value<0.05). Black dots: non-differentially expressed genes. Sample sizes were as follows: Duodenum-Chip, n=3; Adult Duodenum Human tissue, n=2. Sample sizes were as follows: Duodenum Intestine-Chip, n 3; Adult Duodenum, n=2. All samples were biologically independent (derived from a different donor). Duodenum Intestine-Chips were grown in the presence of expansion media for 6 days, followed by 2 days of differentiation media. Analysis was performed in samples collected 8 days post-seeding.

FIG. 15 shows exemplary Goblet Cell Function in Duodenum Intestine-Chip. Muc 2 (ng/ml). Data from 3 independent experiments, each using different donors of biopsy-derived organoids and Caco-2 Intestine-Chips. n=3.

FIG. 16 shows exemplary Functional Characteristics in Duodenum Intestine-Chip. Sucrase activity (U g protein). Intestine-Chip (Enteroids derived) Intestine-Chip Caco2 Intestine-Chip. 30 mM Mannitol (Day 12); 30 mM Sucrose (Day 4); 30 mM Sucrose (Day 12); 30 mM Mannitol (Day 4). Data from 3 independent experiments, each using different donors of biopsy-derived organoids and Caco-2 Intestine-Chips. n=3.

FIG. 17 shows exemplary gene expression compared by fold induction over untreated duplicate duodenal chips. CYP450 related CYP genes; and MDR1 efflux pump (P-gp), and UGT1A1 (i.e. UDP Glucuronosyltransferase Family 1 Member A1; Bilirubin-Specific UDPGT Isozyme; etc.) UDP-glucuronosyltransferase refers to enzymes of the glucuronidation pathway that transforms small lipophilic molecules, such as steroids, bilirubin, hormones, and drugs, into water-soluble, excretable metabolites. UDP-glucuronosyltransferases may be involved in the elimination of potentially toxic xenobiotics and endogenous compounds. Vehicle (0.1% DMSO, 48 h). Rifampicin (20 uM, 48 h). ⋅ Data from 3 independent experiments, each using different donors of biopsy-derived organoids. n=3.

FIG. 18 shows exemplary blunting of villi-like structures and appearance of apoptotic cells in Duodenum Intestine-Chips at concentrations of 1 mM and higher. Epithelium (upper panels); Endothelium (lower panels). Left to right: Vehicle 0.1% DMSO; Indomethacin 0.5 mM; Indomethacin 0.75 mM; Indomethacin 1 mM; Indomethacin 1.5 mM. ⋅ Data from 1 experiment, a single donor of biopsy-derived organoids. n=4.

FIG. 19 shows exemplary Indomethacin disruption of cell-cell junction integrity and barrier function in one embodiment of a Duodenum Intestine-Chip. Vehicle 0.1% DMSO (upper panels); Indomethacin 1.5 mM (lower panels). Epithelium (left panels); endothelium (right panels). VE-cadherin (pink); ZO-1 (green). ⋅ Data from 1 experiments, a single donor of biopsy-derived organoids. ⋅ n=4.

FIG. 20A-B shows exemplary induction of injury related biomarkers, e.g. LDH, ROS and I-FABP. Duodenal organoids vs. Intestine-Chip. Safety Assessment Applications: Organoids vs Intestine-Chip.

FIG. 20A Intestine-Chip induction of injury related biomarkers, e.g. LDH, ROS and I-FABP in relation to loss of barrier function (Papp). ⋅ Data from 1 experiment, a single donor of biopsy-derived organoids. ⋅ n=4.

FIG. 20B Duodenal organoids induction of injury related biomarkers, e.g. LDH, ROS and I-FABP. ⋅ Data from 1 experiments, a single donor of biopsy-derived organoids. ⋅ n=4. Left to right: Vehicle 0.1% DMSO; Indomethacin 0.5 mM; Indomethacin 0.75 mM; Indomethacin 1 mM; and Indomethacin 1.5 mM.

FIG. 21 Abnormal morphology (lamellar shape) of mitochondria in the presence of indomethacin is observed in Primary Duodenum Intestine-Chips. Left to right: Vehicle 0.1% DMSO; Indomethacin 0.5 mM; Indomethacin 0.75 mM; Indomethacin 1 mM; and Indomethacin 1.5 mM. Left to right: Vehicle 0.1% DMSO; Indomethacin 1.5 mM. Ileum-Chip.

FIG. 22 shows an exemplary schematic timeline for Induction of Inflammation in Ileum-Chip with a chart showing that vascular exposure to TNF 100 U/ml (approximately 2 ng/ml) does not affect intestinal barrier function.

FIG. 23 shows exemplary TNF-alpha induced increased membrane expression of I-CAM1 and MadCAM1 in intestinal microvascular Endothelium. Shows exemplary Inflammation-Induced Expression of I-CAM1 and MadCAM1 in ileum-Chip. Shows one embodiment of an exemplary timeline for providing an inflammatory Ileum-Chip by induction with TNF-alpha. TNF-alpha induces increased membrane expression of I-CAM1 and MadCAM1 in intestinal microvascular endothelium. Ileum; I-CAM1; MadCAM1. Shows an exemplary barrier function of an Ileum-Chip: untreated and treated with TNF-alpha.

FIG. 24 shows an exemplary Modeling of Lymphocytes Chemoattraction in Ileum-Chip: CCL25. Untreated TNF-alpha: Untreated; treated TNF-alpha. Predominantly basolateral secretion of TECK was observed in Ileum-Chip that increases in the context of TNF-induced inflammation. Schematic.

FIG. 25 shows an exemplary Modeling of Lymphocytes Chemoattraction in ileum-Chip—CXC chemokines. During inflammation CXC chemokines such as CXCL8 (IL-8), CXCL10 (IP-10) and CXCL11 (I-TAC) are produced at the site of inflammation and contributes to the recruitment of immune cell. TNF-alpha stimulation induces a significant increase in the vascular release of IL-8, IP-10 and 1-TAC in ileum-Chip. Ileum-Chip exhibits polarized production of 1-TAC that results in a basal-to-apical chemokine gradient, previously observed in bronchial epithelium, that could mediate lymphocytes directional migration.

FIG. 26 shows an exemplary Modeling of Lymphocytes Chemoattraction in ileum-Chip—CCL20. Ileum-Chip exposure to TNF-alpha results in the vascular release of MIP-3 alpha. Surprisingly high secretion of MIP-3 alpha into the epithelial channel of Ileum-Chip. Untreated; treated TNF-alpha. n=1, 4 chips/condition.

FIG. 27 shows an exemplary Presence of Epithelium Influences Levels of Chemokines Production in Ileum-Chip. CXCL8 (IL-8), CXCL10 (IP-10), MIP-3 alpha and CXCL11 (1-TAC). Ileum-Chip. Microvasculature alone.

FIG. 28 shows an exemplary Ileum-Chip possessing L-cells at physiologically relevant ratios.

FIG. 29 shows one embodiment of an exemplary timeline for providing L-cells in Ileum-Chip.

FIG. 30 shows an exemplary L-cells Present in Ileum Intestine-Chips that Are Biologically Active. Predominantly basolateral release of GLP-1 was detected in Ileum-Chip. Luminal secretion and intracellular GLP-1 content remain constant, vascular release seemed to increase with stretch. n=1, 3 chips/condition.

FIG. 31 shows an exemplary Identification of Tuft Cells in one embodiment of a Colon-Chip System.

FIG. 32 shows an exemplary Enrichment of Tuft Cell Population by IL-13 Treatment in one embodiment of a Colon-Chip.

FIG. 33 shows an exemplary results that IL-13 Does Not Affect Establishment Of Barrier Function one embodiment of a Colon-Chip.

FIG. 34 shows an exemplary Propagation of Suspension Enteroids (duodenum, jejunum, ileum, colon)/Colonoids.

FIG. 35 shows an exemplary timeline for providing one embodiment of an ileal-Chip Using Heal Enteroids and storing in Biobank. Cluster of Ileal enteroids embedded in Matrigel. Image representing 1 well of 24-well plate in which organoids are grown embedded in ECM gel and overlaid with IntestiCult™ media. Human Intestinal Microvascular Endothelial Cells (HIMEC) from small intestine. Image representing endothelial cells grown in flask filled with EGM2-MV media.

FIG. 36 shows an exemplary timeline for providing one embodiment of an ileal-chip. Representative images of epithelium on-chip from Day 0-Day 12; Cell attachment: Formation of confluent monolayer; Morphogenesis of villi-like structures.

FIG. 37 shows an exemplary 3D Tissue Architecture: Formation of Intestinal Villi-like Structures: Ileum. Morphogenesis of villi-like structures in ileum-Chip is achieved across entire length of the epithelial channel. Representative images from day 8 of growth are shown.

FIG. 38 shows an exemplary presence of endothelial cells rescues loss of intestinal barrier function: Ileum. Presence of HIMEC in vascular channel improves maintenance of intestinal barrier functions. Representative images from day 14 of growth are shown.

FIG. 39 shows an exemplary Comparison Between ileum-Chip and 3D Ileal Organoids. Heal enteroids and ileum-Chip has been grown for up to 4, 8 and 12 days in the presence of Intesticult™ media and compared using imaging and gene expression analysis.

FIG. 40 shows one embodiment of an exemplary Experimental Timeline: Ileum. Ileum-Chip and Ileal enteroids were analyzed at day 4, 8 and 12 of post-seeding.

FIG. 41 shows an exemplary Cell Types Detected in 3D Ileal Organoids. Presence of major intestinal cell types (except for tuft cells) has been detected in 3D ileal enteroids. Representative images from day 8 of growth are shown.

FIG. 42 shows an exemplary Presence of Major Intestinal Cell Types Detected in Ileum-Chip. Maturation of major intestinal cell types (except for Tuft cells) was confirmed by confocal imaging. Representative images from day 8 of growth are shown.

FIG. 43 shows an exemplary On-Chip Culture Improves Maturation of Intestinal Cells: Ilium. On-Chip culture resulted in significantly increased expression (in comparison to 3D enteroids cultures) of markers specific for absorptive enterocytes, goblet cells and enteroendocrine cells.

FIG. 44 shows an exemplary On-Chip Culture Increases Expression of Enteroendocrine Cells Specific Markers: Ileum. Ileum-Chip showed improved maturation of enteroendocrine cell population, including L-cells and enterochromaffin cells in comparison to 3D enteroids

FIG. 45 shows an exemplary 3D Enteroids: Highly Proliferative Cultures Rich in Paneth and Tuft Cells: Ileum. Higher expression of lysozyme and Trpm5, markers specific for Paneth and Tuft cells, respectively, in enteroids cultures. Decreased levels of stem cell marker LGR5 on-Chip in comparison to 3D enteroids

FIG. 46 shows an exemplary successful barrier formation across different donors. Ileum-Chips established from the organoids of 3 individual donors reached similar levels of intestinal impermeability to 3 kDa Dextran.

FIG. 47 shows an exemplary time-dependent increase in differentiation of enterocytes and goblet cells. Increased expression of markers specific for absorptive enterocytes and goblet cells in cells grown on-Chip across 3 different donors.

FIG. 48 shows an exemplary Successful Differentiation of Enteroendocrine Cells in Ileum-Chips. Markers specific for different subpopulations of enteroendocrine have been detected in Ileum-Chips established from the enteroids of 3 different donors.

FIG. 49 shows an exemplary Increased Differentiation Balanced by Decrease in Proliferation and Sternness. Time-dependent decreased in the population of Paneth cells and cycling Lgr5+ stem cells observed across different donors.

FIG. 50 shows an exemplary Expression of Tuft Cells Specific Marker in Ileum-Chips. TRPM5 expression showed donor-specific fluctuations. CHAT and DCLK1 were not detected. Expression of CHAT and DCLK1 markers were undetectable.

FIG. 51 shows an exemplary Physiological Ratios of Major Intestinal Cell Types in embodiments of an Ileum-Chip. Most of the major intestinal cell types are present in embodiments of an Ileum-Chip at the physiologically-relevant ratios. Quantification performed across 3 donor samples at day 8 of fluidic culture.

Colon

FIG. 52 shows one embodiment of an exemplary timeline for establishment of a Colon-Chip using human colonoids and comparison between a Colon-Chip and 3D Colonic Organoids. A Colon-Chip platform was successfully established using human colonoids (left) with human colonic microvascular endothelial cells (cHIMECs) right. Colon-Chip: 3D Tissue Morphology. Confocal microscopy micrographs showing colonic epithelium forming some distinct morphological features—folds and pouches—in the Colon-Chip. Colonoid-Chip, colonoids, and ileum-Chip and Ileal enteroids were analyzed at day 5, 8 and 10 of post-seeding.

FIG. 53A-D shows one embodiment of an exemplary timeline for an exemplary Colon-Chip Using Human Colonoids. FIG. 43A cross section of the Colon Intestine-Chip, 1: Epithelial Channel, 2: Human Colonoids derived Epithelial Monolayer, 3: Vacuum Chamber, 4: Porous PDMS Membrane, 5: colonic: endothelial cells, e.g. cHIMECs, 6: Vascular Channel. FIG. 53B The automated instrument perfusion manifold. FIG. 53C Representative contrast phase microscopy images of each cell type morphology, on a conventional plate culture (left) and right after their introduction in the Colon Intestine-Chip (right). FIG. 53D Experimental timeline.

Stretching vs non-stretching. The establishment of a tight epithelial barrier in the Colon Intestine-Chip over time was confirmed across three healthy donors. The presence of endothelium accelerated the establishment of the epithelial barrier, as assessed by the apparent permeability of 3 kDa Dextran Cascade Blue.

FIG. 54 Shows exemplary presence of colonic endothelial cells decreases the time required for epithelial barrier formation in Colon-Chips. Colonic endothelial cells decrease the time required for epithelial barrier formation in Colon-Chips. Surprisingly, combining endothelial cells with stretching results in a synergistic effect of quick and long lasting barrier function over time for at least 3 individual intestinal tissue donors tested.

FIG. 55 Shows exemplary Increased Differentiation of Epithelial Cell Types. Shows an exemplary Comparison Between Colon-Chip and 3D Colonic Organoids. Colon-Chip and Colon enteroids were analyzed at day 5, 8 and 10 of post-seeding. Similarly to ileum, Colon-chip culture has showed an increased level of differentiation of absorptive enterocytes, Goblet cells and enteroendocrine cells in comparison to colonoids.

FIG. 56 Shows an exemplary Similar Levels of Tuft Cells Maturation in Colon-Chips and Colonoids. Similar levels of Trpm5 expression, marker specific for Tuft cells observed in Colon-Chip and colonoids. Decreased levels of stem cell marker LGR5 on-Chip in comparison to 3D colonoids.

FIG. 57 Shows an exemplary presence of major intestinal cell types detected in one embodiment of Colon-Chip. Representative images from day 8 of growth are shown. Absorptive Enterocytes stained with villin (pink), nuclei shown in white. Goblet cells stained with Muc2 (pink), nuclei shown in white. EEC cells stained with ChgA (pink), nuclei shown in white. EEC cells stained with ChgA (pink), L-cells stained with Gcg (green), nuclei shown in white.

FIG. 58 Shows an exemplary quantification (lower charts within panels) of the major intestinal cell types (shown stained in immunofluorescent micrographs as insert-upper right within panels) in one embodiment of Colon-Chip compared to colon tissue biopsies. In vivo referenced tissue from: Karam S M, et al. Front Biosci 1999, 4:D286-298; Lund M L, et al. Molecular Metabolism. 2018; 11:70-83; Petersen N, et al. The Journal of Clinical Investigation. 2015; 125(1):379-385. Quantification was performed on Day 8 of Donor 1 culture on the Colon-Chip. Absorptive Enterocytes stained with villin (pink), nuclei shown in white. Goblet cells stained with Muc2 (pink), nuclei shown in white. EEC cells stained with ChgA (pink), nuclei shown in white. EEC cells stained with ChgA (pink), L-cells stained with Gcg (green), nuclei shown in white.

FIG. 59A-D Shows an exemplary epithelial barrier establishment in one embodiment of Colon-Chips. Shows an exemplary Presence of Endothelial Cells Accelerates Barrier Formation in Colon-Chip. Colonic endothelial cells decrease the time required for epithelial barrier formation in Colon-Chips. [FIG. 49A] Representative contrast phase microscopy images of the epithelial monolayer morphology, on Day 1 (left) and Day 10 (right) of the fluidic culture [FIG. 49B] Colon-Chips established from 3 donors reached similar levels of intestinal barrier function. Apparent Permeability of 3 kDa Dextran across three different donors over 10-days in culture, Mean±SD [FIG. 49C] Representative fluorescence image depicting F-actin staining in the colonic epithelial monolayer. N=3 chips/donor.

FIG. 60 Shows an exemplary time-dependent epithelial maturation in one embodiment of Colon-Chip. Expression of mature intestinal cell-type specific markers in Colon-Chips confirms increased differentiation during the on-Chip culture across 3 independent donors.

FIG. 61 Shows an exemplary sternness and proliferation in one embodiment of Colon-Chips. Concomitant with epithelial differentiation, expression of sternness and proliferative cell-types decreases over time.

FIG. 62 Shows an exemplary differentiation of enteroendocrine cell subtypes in one embodiment of Colon-Chips. Expression of enteroendocrine cells-specific markers reveals donor-dependent variability.

FIG. 63 Shows an exemplary confirmation of tuft cell differentiation in one embodiment of Colon-Chips. CHAT and DCLK1 were not detected. TRPM5 expression in Colon-Chip showed donor-dependent fluctuations while expression alternate markers (CHAT and DCLK1) were undetectable.

FIG. 64A-1 shows an exemplary differentiation state in one embodiment in one embodiment of Colon-Chips. [FIG. 64A-D-G] Representative immunofluorescence images of Villin, Mucin 2 (Muc2) and Chromogranin A (ChgA) staining depicting the populations of absorptive enterocytes, Goblet and enteroendocrine cells respectively, on day 8 of the fluidic culture [FIG. 64B-E-H] Abundance of each epithelial cell type, expressed as a percentage over the total number of nuclei. Quantification was performed, in 5 different fields of view per chip. [FIG. 64A-C] Assessment of Absorptive Enterocytes. Absorptive enterocytes at human tissue relevant abundance were identified in the Colon-Chip by immunostaining and qRT-PCR. [FIG. 64D-F] Assessment of Goblet Cells. Goblet cells were identified in the Colon-Chip by detection of mucin 2. Their prevalence varied across different donors. [FIG. 64G-1] Assessment of Enteroendocrine Cells. Presence of enteroendocrine cells population in the Colon-Chip was confirmed by immunostaining and qRT-PCR.

One experiment, n=3 chips/donor, Mean±SD, One-way ANOVA, Tukey's test, *: p<0.05, **: p<0.01 [Fig. C—F-I] Identification of each epithelial cell type by qPCR for the Alkaline Phosphatase (Alk1), Mucin 2 (Muc2) and Chromogranin A (ChgA) genes respectively. 1-4 experiments, n=2-10 chips/donor, Mean±SD.

FIG. 65 shows an exemplary differentiation in one embodiment of Colon-Chip: Assessment of Tuft Cells. Presence of Tuft cells was confirmed in the Colon-Chip by immunofluorescent staining for Trpm5 and ChAT.

Shows an exemplary Quantification of Tuft Cell Population in Colon-Chips. Confirmation that Trpm5+/ChAT+ Tuft cells are present in the Colon-Chip at physiological levels. Scale Bar: 50 um. Similar levels of Trpm5 expression, marker specific for Tuft cells observed in Colon-Chip and colonoids.

FIG. 66 shows an exemplary differentiation in one embodiment of a Colon-Chip: Assessment of L Cells. Subpopulation of Gcg-positive EEC, namely L-cells, was identified in the Colon-Chip using immunostaining and qRT-PCR.

FIG. 67 shows an exemplary differentiation in one embodiment of a Colon-Chip: Assessment of Intestinal Stem Cells. Time-dependent decrease in the population of proliferating cells, such as Lg5-positive stem cells, in Colon-Chips. Decreased levels of stem cell marker LGR5 on-Chip in comparison to 3D colonoids.

Proteomic Analysis of Colon-Chip. Goal: To evaluate the role of intestinal microvasculature and stretching on the maturation and function of human colonic epithelium.

FIG. 68 shows exemplary illustrations of comparative growth conditions (upper panels) and one exemplary embodiment of an experimental workflow and timeline over one year. e=endothelium: s=stretch. Exemplary time points in lower panels, open circles left to right: June; June; September; October-November. Barrier function data shown significant differences on barrier function with the introduction of endothelial cells. However, the data does not express relevant differences between stretch and non-stretch. Gene expression shown differences in the presence of endothelium and/or stretching.

Exemplary RNAseq Analysis in One Embodiment of a Colon-Chip.

We use Spearman correlation to measure the similarity of the expression rankings for the 166 colon Atlas genes (signature) in CIEC (CIEC refers to colon intestine epithelial cells isolated from human tissue) vs Colonoids (static cultures on plates/tranwells) and embodiments of colon Chips.

The higher the Spearman correlation coefficient the higher the similarity of Colonoids and Chip samples to the CIEC tissue (maximum possible value is +1). Average CIEC-to-Chip Spearman correlations are considerably higher than CIEC-to-Colonoids, both at Day 5 (D5) and Day 8 (D8) and across all four chip conditions. The CIEC-Chips similarity is considerably higher than the CIEC-Colonoids similarity.

FIG. 69 Shows an exemplary colon-chip Spearman Correlation Analysis (based on 166 Atlas genes) as an assessment of the Intestine-Chip “Intestine-ness”. Exemplary Experimental Conditions Tested: Donor 1 at two different experimental time points (day 5 and day 8).

FIG. 70 Shows an exemplary colon-chip_{Boxplot} Spearman Correlation Analysis (based on 166 Atlas genes) as an assessment of the Intestine-Chip “Intestine-ness”. Exemplary Experimental Conditions Tested: Donor 1 at two different experimental time points (day 5 and day 8).

FIG. 71 Shows an exemplary colon-chip_{Boxplot} Spearman Correlation Analysis (based on 79 Atlas genes). Exemplary Experimental Conditions Tested: Donor 1 at two different experimental time points (day 5 and day 8).

FIG. 72 Shows an exemplary colon-chip PCA Analysis (1) from the 166 Atlas genes.

FIG. 73 Shows an exemplary colon-chip PCA Analysis (2) from the 166 Atlas genes comparing presence or absence of endothelium (+e and −e, respectively), under conditions of stretch (+s) or no stretch (−s).

FIG. 74 Shows an exemplary colon-chip Differential Gene Expression (DGE) Analysis as a volcano plot of 1800 DE (Differentially Expressed) genes. Using the batch corrected dataset, we applied Differential Gene Expression Analyses between: colon tissue as colonic Intestinal Epithelial Cells (cIECs, public data) and colonoids, CT vs cIECs, respectively.

FIG. 75 shows an exemplary Differential Expression in the Colon Intestine-Chip Analysis as a volcano plot. Colon Intestine-Chip gene expression was found to be closer to colon tissue (CT) with a difference of 134 DEGs, in comparison to colonoids in static cultures (e.g. −e and −s) with a difference of 527 DEGs. Volcano plot of the Differential Gene Expression Analysis between cIECS and colonic epithelial cells on day 5 in the Colon Intestine-Chip under four different culture conditions, with and without endothelial cells plus with and without stretching. Differential Gene Expression Analysis Upper panels (CT vs Day 5-endo), left to right: CT vs s (stretch); CT vs −e/−s. Differential Gene Expression Analysis; Lower panels (CT vs Day 5+endo), left to right: CT vs +e/−s and CT vs+e/+s. Adjusted p-value 0.05, log 2FoldChange|≥2. Stretch appears to decrease the number of DE genes indicating a closer physiology to colon tissue.

FIG. 76 Shows an exemplary schematic of exemplary experimental conditions and timeline for Proteomic Analysis of Colon-Chips. Experimental Workflow and Timeline. Conditions were tested in Donor 1 at one experimental time point (day 8).

FIG. 77 Shows an exemplary Colon-Chips (+/−endothelium) vs 3D colonoids: Gene Ontology Analysis. No Endothelium; No Stretch (−e, −s). No Stretch (+e/−s).

FIG. 78 Shows an exemplary Colon-Chips (+1-endothelium) vs 3D colonoids: KEGG Pathway Analysis. −e/−s vs colonoids/+e/−s vs. colonoids.

FIG. 79 Shows an exemplary Colon-Chips (+/−stretch) vs 3D colonoids: Gene Ontology Analysis. +e/−s vs colonoids/+e/+s vs. colonoids.

FIG. 80 Shows an exemplary Colon-Chips (+/−stretch) vs 3D colonoids: Gene Ontology Analysis. −e/−s vs/+e/−s vs. colonoids.

FIG. 81 Shows an exemplary Colon-Chips (+/−stretch) vs 3D colonoids: KEGG Pathway Analysis.

FIG. 82 Shows an exemplary Testing of GPR35 agonists in Colon-Chip. Aim, Timeline, Readouts.

FIG. 83 Shows an exemplary GPR35 Treatment Effect on Colon-Chip Morphology and Barrier Function. No significant changes in Colon-Chips morphology and permeability were observed in response to GPR35 and/or TNFα treatment.

FIG. 84 Shows an exemplary GPR35 Treatment Effect on the Number of Goblet Cells Colon-Chip (Donor 1). Treatment of Colon-Chips (Donor 1) with active agonist of GPR35 results in a significant enrichment of Goblet cell population.

FIG. 85 Shows an exemplary GPR35 Treatment Effect on the Number of Goblet Cells Colon-Chip (Donor 2). Treatment of Colon-Chips (Donor 2) with active agonist of GPR35 led to slight but not significant increase in the Goblet cell population.

FIG. 86 Shows an exemplary GPR35 stimulation in Caco-2 Intestine-Chip. Aim, Timeline, Readouts. Shows an exemplary Testing of GPR35 agonists in Colon-Chip Experimental Repeat Timeline.

FIG. 87 shows an exemplary schematic of a background adsorption analysis.

FIG. 88 shows an exemplary Characterization of Time-Dependent Compound Loss.

FIG. 89 shows an exemplary STEP 2: Standard 72 hour absorption studies run for PDMS (glass vials). A: K=10; D==10−8 cm 2/s. B: K=150; D=10−8 cm 2/s.

FIG. 90 shows an exemplary STEP 3: 2D Chip model run to determine cellular exposure concentrations. Flow rate for Compound B exposure—150 μL/hr flow rate recommended for experiments, in some preferred embodiments.

FIG. 91 shows an exemplary STEP 4: Comparison of 2D Chip model and experimental results. % recovered in perfusion manifold outlet. % compound A recovered in culture module. % compound B recovered on culture module.

FIG. 92 shows an image of an exemplary organoid fragment seeding density. A minimum 6 hours is typical for the organoid fragments to fully attach to one embodiment of a membrane (208) surface.

FIG. 93 shows an exemplary time line for embodiments of a rescue assay; first inducing barrier injury (e.g. reducing apparent permeability) then testing a substance for improving (e.g. increasing apparent permeability).

FIG. 94 shows an exemplary concentration dependent response of an organoid-derived colon chip to AT1002 measured by Apparent Permeability using 3 KDa Dextran Cascade Blue (P^{App} (cm/s)×10⁻⁷).

FIG. 95 shows exemplary efficacy of Larazotide Acetate to restore/rescue up to 100% of barrier integrity in Colon Intestine-Chip exposed to AT1002 after 24 hours. Larazotide acetate at 20 mM. N=5. Apparent Permeability was measured using 3 KDa Dextran Cascade Blue.

FIG. 96 shows an exemplary time line for embodiments of a cytokine substance rescue assay; first inducing barrier injury using a cytokine (e.g. IFN-γ) then testing a substance for improving (e.g. increasing apparent permeability), e.g. Larazotide acetate (12.5 mM).

FIG. 97 shows an exemplary concentration independent response of an organoid-derived colon chip to a substance, e.g. cytokine, such as IFN-gamma, 10, 25 and 50 ng/ml. Apparent Permeability to 3 KDa Dextran Cascade Blue (P^{App} (cm/s)×10⁻⁷). N=5.

FIG. 98 shows an exemplary restoration by Larazotide acetate (12.5 mM) of barrier permeability disrupted by a cytokine (e.g. IFN-γ). Statistically significance between 0 and 24 hours after disruption (injury). Apparent Permeability: 3 KDa Dextran Texas Red (P^{App} (cm/s)×10⁻⁷). N=5.

### DEFINITIONS

As used herein, “damage” or “injure” or “injury” in general refers to a cell or tissue, barrier or organ having an altered function indicative of a compromised and/or disease condition, e.g. reduction of barrier function (e.g. increased permeability), loss of villi-crypt architecture, increase in secretion of inflammatory cytokines, increased in cell division, etc.

Non-limiting examples of damage include damage by any one or combination of exposure to a substance such as microbes, an immune cell; a chemical; a cytokine, etc. resulting in damage, such as a loss of barrier function, cellular abnormalities, etc. Damage may also be considered resulting from a genetic alteration, such an allele or somatic mutation(s), resulting in increasing susceptibility to damage or associated with a loss of function, e.g., barrier function.

Damage may be induced to a cell layer on chip by exposure to one injury inducing substance, or more than one as mixture of injury inducing substances or sequential exposure.

As used herein, a “microfluidic device” includes Organ-on-Chip devices, including but not limited to intestine-chips, small intestine-chips, duodenum intestine-chips, jejunum intestine-chips and ileum intestine-chips, large intestine, colon intestine, etc., brain chips, kidney chips, airway-chips, respiratory chips, liver chips, etc.

The term “microfluidic” as used herein relates to components where moving fluid is constrained in or directed through one or more channels wherein one or more dimensions are 1 mm or smaller (microscale). Microfluidic channels may be larger than microscale in one or more directions, though the channel(s) will be on the microscale in at least one direction. In some instances the geometry of a microfluidic channel may be configured to control the fluid flow rate through the channel (e.g. increase channel height to reduce shear). Microfluidic channels can be formed of various geometries to facilitate a wide range of flow rates through the channels.

“Channels” are pathways (whether straight, curved, single, multiple, in a network, etc.) through a medium (e.g., silicon) that allow for movement of liquids and gasses. Channels thus can connect other components, i.e., keep components “in communication” and more particularly, “in fluidic communication” and still more particularly, “in liquid communication.” Such components include, but are not limited to, liquid-intake ports and gas vents. Microchannels are channels with dimensions less than 1 millimeter and greater than 1 micron.

As used herein, the phrases “connected to,” “coupled to,” “in contact with” and “in communication with” refer to any form of interaction between two or more entities, including mechanical, electrical, magnetic, electromagnetic, fluidic, and thermal interaction. For example, in one embodiment, channels in a microfluidic device are in fluidic communication with cells and (optionally) a fluid reservoir. Two components may be coupled to each other even though they are not in direct contact with each other. For example, two components may be coupled to each other through an intermediate component (e.g. tubing or other conduit).

As used herein, the term “biopsy” refers to a sample of the tissue that is removed from a body.

As used herein, “Caco-2” or “Caco2” refer to a human epithlial intestinal cell line demonstrating a well-differentiated brush border on the apical surface with tight junctions between cells. Although this cell line was originally derived from a large intestine (colon) carcinoma, also called an epithelial colorectal adenocarcinoma, this cell line can express typical small-intestinal microvillus hydrolases and nutrient transporters, see. Meunier, et al., “The human intestinal epithelial cell line Caco-2; pharmacological and pharmacokinetic applications.” Cell Biol Toxicol. 11(3-4): 187-94, 1995, abstract. Examples of Caco-2 cell lines include but are not limited to CRL-2102, American Type Culture Collection (Rockville, Md.); a BBE subclone of Caco-2 cells; etc.

### DESCRIPTION OF INVENTION

The present invention relates to fluidic systems for use in providing biomarkers for human Intestine On-Chip. In another embodiment, of an Intestine On-Chip, neurons may be included for providing an innervated intestine, e.g. sensory neurons. In another embodiment, of an Intestine On-Chip, resident immune cells are incorporated. In some embodiments, additional cells such as immune cells may be added for providing environmental and host factors implicated in GI Pathogenesis. More specifically, in some embodiments, a microfluidic chip containing intestinal epithelial cells co-cultured with intestinal endothelial cells in the presence of stretch and flow are used for identifying differentially expressed genes as biomarkers, e.g. for specific types of drug testing for use in treating gastrointestinal disorders or diseases related to intestinal function.

It is not intended to limit the type of Intestine On-Chip, indeed, intestine cells may be derived from any one or more of intestinal tissues including but not limited to: small intestinal areas comprising Duodenum, Jejunum, and Ileum, where the duodenum may be divided into at least four parts; superior, descending, inferior and ascending; Jejunum comprising the part of the small intestine between the duodenum and ileum; an Ileum which follows the jejunum and ends at the ileocecal junction, where the terminal ileum communicates with the cecum of the large intestine through the ileocecal valve; large intestine connected to the small intestine at one end and the anus at the other, comprising at least four parts: cecum, colon, rectum, and anal canal. Partly digested and digested food moves from the small intestine through the cecum into the colon, where water and some nutrients and electrolytes are removed. The remaining material, solid waste called stool, moves through the colon, is stored in the rectum, and leaves the body through the anal canal and anus

In some embodiments, readouts include genome-wide transcriptome profiles of embodiments of Intestine Chips, including but not limited to Organoid-derived Intestine Chips, Caco-2 Gut Chips or Transwell Caco-2 cells cultured in static conditions compared with embodiments of intestine Chips.

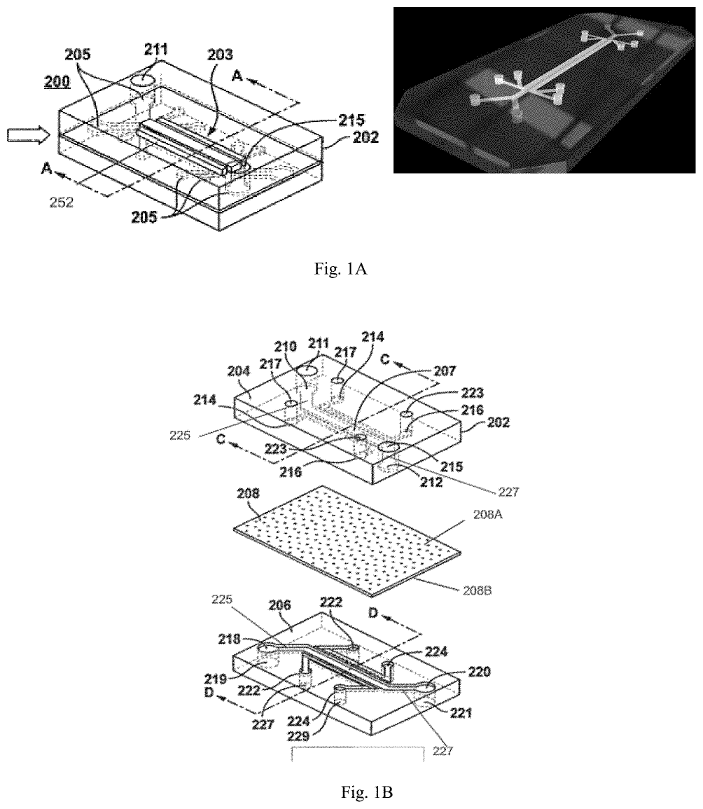

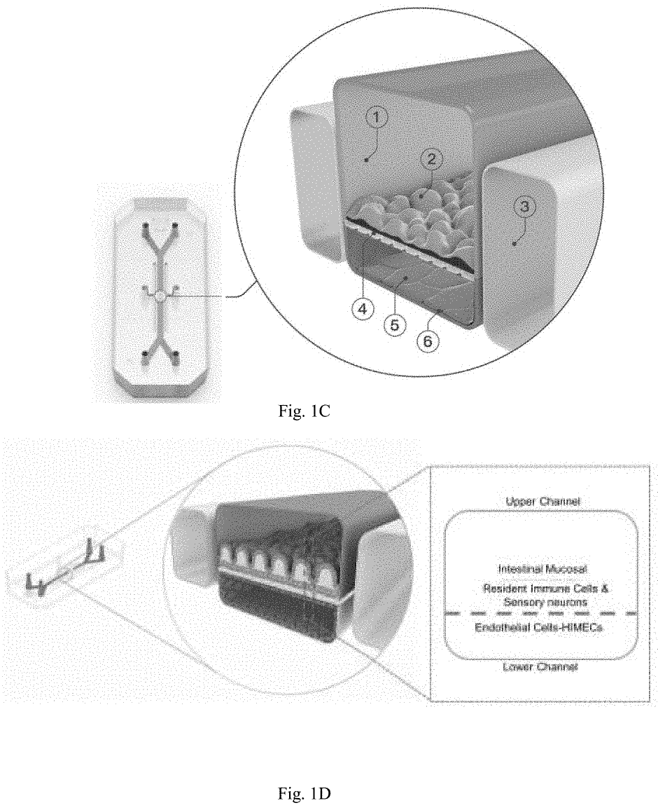

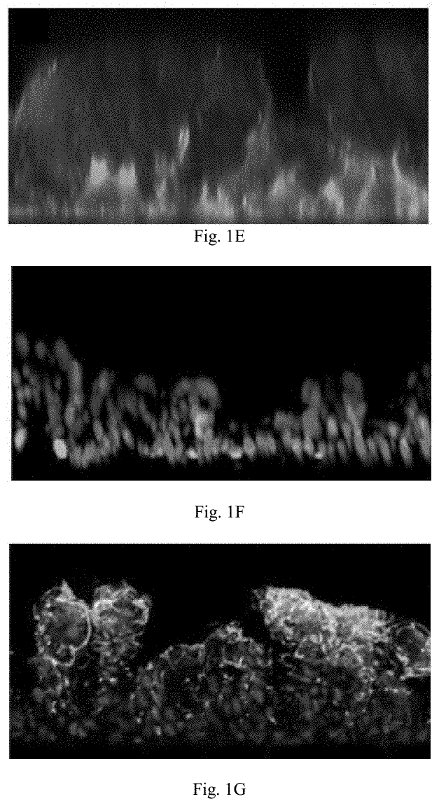

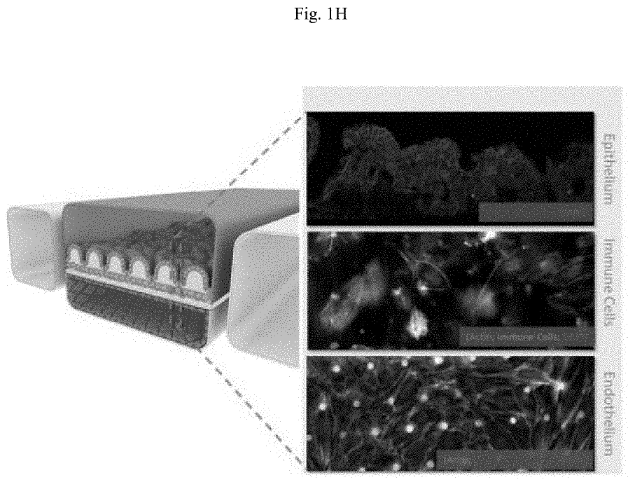

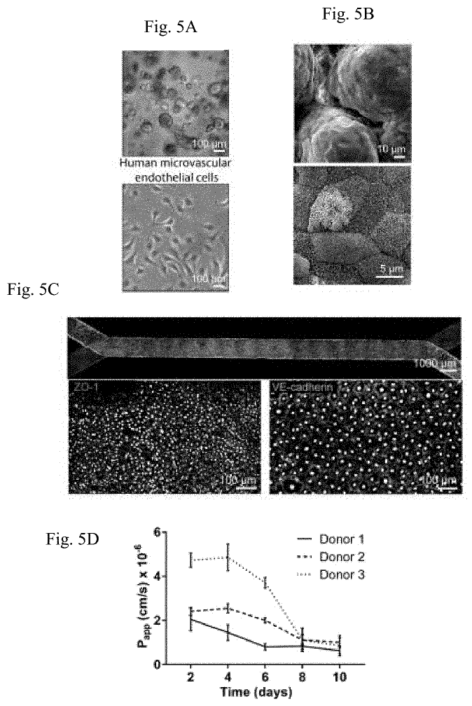

In brief, we established organoid cultures (FIG. 5A; top) from crypts isolated from endoscopic biopsies of at least three different healthy adult individuals, the organoids were then dissociated into fragments and seeded on the top of the ECM-coated porous flexible polydimethylsiloxane (PDMS) membrane of the chips (FIG. 1C; 2: indicates the epithelial tissue). Primary human intestinal microvascular endothelial cells (HIMECs, Cell Biologics), derived from the human small intestine (FIG. 5A: bottom), were used to populate the other surface of the PDMS membrane in the vascular channel (FIG. 1C; 5: indicates the endothelial cells).

A. Flow and (Optional) Stretch.

The Duodenum Intestine-Chip was perfused continuously through the luminal and vascular compartment with fresh cell culture medium. Once the epithelial monolayers reached confluency they were subjected to the optional step of cyclic mechanical strain (10% strain, 0.2 Hz) in order to emulate physiologically relevant forces of intestinal peristalsis. In some preferred embodiments of human Intestine On-Chip, the presence of stretch and flow is desired for use. A cyclic stretching regimen has an effect on F-actin staining in FIG. 1H.

FIG. 1H shows exemplary incorporation of Lamina Propria Derived Immune Cells on the Intestine-Chip (schematic left). Upper panel epithelium, middle panel showing immune cells and lower panel endothelium. Tight junctions (red), F-actin (green), immune cells (pink), DAP1 stained nuclei colored blue.

In preferred embodiments, we start stretch at 2% and then graduate up to 10%. In some embodiments, applying 10% stretch right away (without first starting at 2%) seems to damage the epithelial cells; in contrast, having an “acclimation period” with 2% stretch resulted in better epithelial morphology. In some embodiments, 2% starts on day 1 (instead of day 3) and 10% starts on day 2 (instead of day 4).

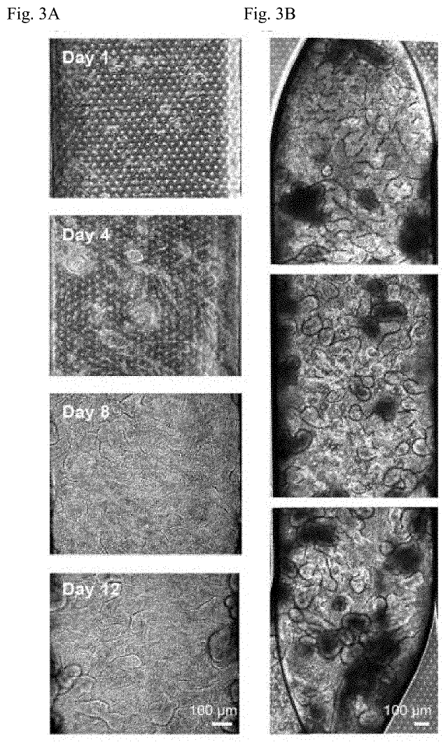

FIG. 3A-B shows an exemplary time course and extent of villus differentiation in the Intestine Chip. Representative phase contrast images of duodenal organoid-derived epithelial cells cultured on chip under continuous flow and peristalsis-like motions. (FIG. 3A) Comparison of the same field of view (center of the channel) imaged at 1, 4, 8 and 12 days of culture demonstrates that the villi formed de novo within these cultures. (FIG. 3B) Views of three different regions beginning (top), middle (center) and end (bottom) of the culture channel confirming that villi-like structures form at high density along the entire length of the channel.

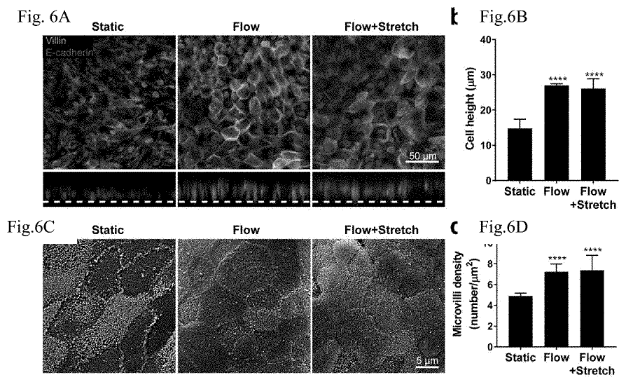

In some embodiments of intestine chips, e.g. Duodenum (Intestine) Chip, a dynamic microenvironment and mechanical forces improves columnar morphology, cell height, and microvilli formation, See FIG. 6A-6D. Flow and Stretch Effects Cytoarchitecture of Duodenum Intestine-Chip. We assessed the effect of applied mechanical stimulation (in the form of fluid flow and stretch) on the phenotypic characteristics of the human primary intestinal cells grown on the chip. To this end, we used multiple endpoints including immunofluorescent staining for apical (villin) and basolateral (E-cadherin) cell surface markers and scanning electron microscopy (SEM) for the identification of apical microvilli. Exposure of the Duodenum Intestine-Chip to flow for 72 hours resulted in accelerated polarization of the epithelial cells and formation of apical microvilli, what is in line with our previous findings reported for the Caco-2 cells (Kim, Huh et al. 2012). We observed that culture of primary intestinal epithelial cells in the chips maintained under static conditions resulted in the formation of a monolayer of flat (14.8±2.6 μm) squamous cells with poorly defined cell-cell junctions (e.g., FIG. 6A) and sparsely distributed microvilli (e.g., FIG. 6C). In contrast, cells cultured under flow (30 μl/hr) with or without concomitant application of cyclic stretch (10%, 0.2 Hz), exhibited a well-polarized and cobblestone-like morphology with increased cell height (27.0±1.3 μm), strongly delineated junctions, and densely packed microvilli. In line with our previous observations, application of the constant flow (shear stress) was important for promoting maturation of a well-polarized epithelium, while short-term application of cyclic strain did not show any additional effect.

Moreover, prolonged cell exposure to flow and cyclic strain resulted in the spontaneous development (around day 6 of culture) of epithelial undulations (“villi-like structures”) extending into the lumen of the epithelial channel and covered by continuous brush border (FIG. 6C). Immunofluorescence confocal analysis confirmed the establishment of confluent epithelial and endothelial monolayers across the entire length of the chip (FIG. 6D), with well-defined epithelial tight junctions, as demonstrated by ZO-1 protein staining and endothelial adherent junctions visualized using antibodies against VE-cadherin (Dawson, Dyer et al.). These culture conditions resulted in a time-dependent improvement of intestinal permeability as indicated by the low permeability coefficient (Papp) of fluorescently labeled dextran recorded in the Duodenum Intestine-Chip generated from organoid-derived cells of 3 different individuals (FIG. 5D).

Thus, barrier Function in embodiments of Duodenum Intestine-Chip demonstrated in vivo relevant tight junction protein expression and localization forming a strong intestinal barrier which is reproducible across multiple donors.

Overall, this data indicates that the human adult Duodenum Intestine-Chip supports the formation of a functional barrier with in vivo relevant cytoarchitecture, cell-cell interactions, and permeability parameters.

B. Cells.

Establishment of normal human organoids in suspension. Exemplary establishment of 3 normal colonic organoids and 3 normal small intestine (ileal) organoids in suspension are capable of being thawed and regrown in culture. Established and optimized protocols for propagation and bio-banking of suspension ileal and colonic organoids. Successfully created a mini-biobank of 3 normal human small intestinal organoids (enteroids) and 4 normal human colonic organoids (colonoids) for the purpose of this project.

In some preferred embodiments of human Intestine On-Chip, HIMECs are used, as shown herein. HIMECs (intestine-specific) have advantages over using generalize endothelial cells (e.g. HUVECs), as one example, as these HIMECs express certain desired receptors, such as involved with transport for drug testing. As one example described herein, colonic HIMECs are used for colon chips. In some preferred embodiments, a human (dermal) microvascular endothelial cell line (HMEC) is used.

C. Seeding Methods and Media.

In some embodiments, in the duodenum Chip, we introduce the epithelial on day 0 of culture and introduce the endothelial on day 8 (at the same time as introducing the aforementioned differentiation medium; i.e. endothelial are introduced in the “differentiation period”). For the colon, we seed the endothelial together with the epithelial cells for better barrier function and a full monolayer of epithelial earlier in culture when endothelial cells were present than when not.

In some embodiments, organoid cells from static cultures of biopsy cells are seeded into chips before endothelial cells, followed by a two step increase in stretching percentage. As one example, D0 seeding organoid cells in Complete Organoid Growth Medium into the upper channel of a two channel chip.

Day 2 seed HIMECs into a static culture, e.g. flask. Day 3 adding Organoid Growth Medium (maintenance media), e.g. IntestiCult™ Organoid Growth Medium (Human), without Y-27632 and CHIR99021, to organoid cells growing in chips by replacing the Complete Growth Medium. Day 6: seed HIMECs from static cultures in Complete HIMEC Culture Medium (comprising Endothelial Cell Basal Medium PromoCell; Endothelial Cell Growth Medium PromoCell; MV2 Supplement Pack and Primocin™) into the channel opposite of the epithelial cells. Set stretch to 2%, 0.20 Hz. Day 7: Set stretch to 10%, 0.20 Hz. Remove samples as desired.

In some embodiments, in the duodenum Chip, we use two types of media: expansion and differentiation media (these relate to the two media types that are used for organoids). In the colon and ileum Chips, we use the expansion medium for unexpectedly good cell differentiation.

Complete Organoid Growth Medium comprises IntestiCult™ Stemcell Technologies (e.g. by mixing IntestiCult™ OGM Component A with IntestiCult™ OGM Component B (1:1), a non-toxic antimicrobial agent for primary cells (e.g. 100 μg/mL Primocin, e.g. commercial source InvivoGen), with 10μM Y-27632 as a selective inhibitor of ROCK1 and ROCK2 used to enhance survival of cells, and 5 CHIR99021, a small molecule inhibitor glycogen synthase kinase 3 β (GSK-3β).

D. Characterization of Normal Human Organoids On-Chip, Cell Types.

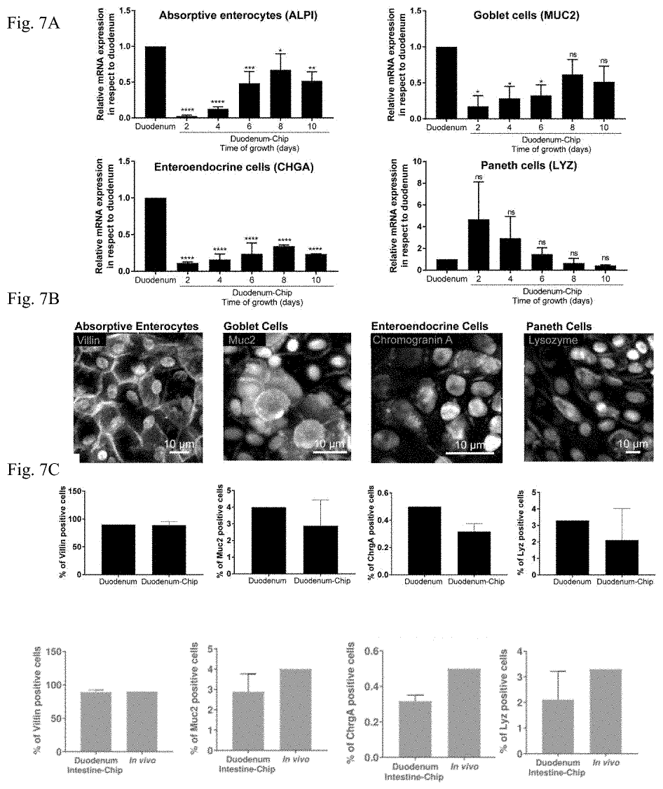

To confirm differentiation of the organoid-derived cells within the chip to the specific intestinal cell lineages as found in vivo, we assessed average mRNA gene expression levels of cell type specific markers in the Duodenum Intestine-Chips established from the cells of 3 different donors including: alkaline phosphatase (ALPI) for absorptive enterocytes, mucin 2 (MUC2) for goblet cells, chromogranin A (CHGA) for enteroendocrine cells, and lysozyme (LYZ) for Paneth cells. In addition, we compared the expression levels of these genes in a chip and in freshly isolated adult duodenal tissue (Duodenum). As shown in FIG. 7A, expression of most of the markers tested, with the exception of lysozyme, increased over time in culture. Notably, on day 8 of chip culture the mRNA expression of alkaline phosphatase and mucin 2 reached levels similar to those detected in RNA isolated directly from the human duodenal tissue. Lysozyme expression showed the opposite trend (declined expression over time in culture) suggesting a reduction in Paneth cell population, which is in line with the observed increased differentiation of the villus epithelium and decreased sternness. In addition, immunostaining followed by quantitative image analysis confirmed the presence of major differentiated intestinal cell types and revealed that the relative abundance of these cell types within the chips is similar to their ratios observed in in vivo tissue (FIG. 7B). Indeed, the ratios of absorptive enterocytes, goblet cells, enteroendocrine and Paneth cells observed in the chips on day 8 of fluidic culture, were close to those reported following histopathological evaluation of sections from normal human duodenum (FIG. 7C) (Karam 1999). Taken together, these results demonstrate the successful establishment of the adult Duodenum Intestine-Chip that closely recreates the barrier function and multilineage differentiation of human intestinal tissue.

We will confirm that human organoids in suspension and on chips express examples of known types of epithelial cell lineages including enterocytes, L cells, Tuft cells, Paneth cells, LGR5 cells, and goblet cells. We will assess intestinal permeability to small fluorescence molecule-dextran. We will confirm expression of MUC2 by antibody staining.

We will compare poly(A) RNA-seq profiles of suspension organoids, gut on chip organoids, human mucosal biopsies, and human epithelial cells isolated from human biopsies.

Summary of Progress:

Established ileum-Chip and Colon-Chip platforms and confirmed: formation of intact barrier function, presence of major intestinal cell types (absorptive enterocytes, goblet cells, stem cells, enteroendocrine cells, L cells, enterochromaffin cells, Tuft cells and Paneth cells) by immunofluorescence and qPCR.

Compared these platforms with organoids in suspension at the level of imaging and gene expression analysis S Confirmed reproducibility of Ileum-Chip and Colon-Chip across 3 different donors.

Performed RNAseq analysis of Colon-Chip in the presence or absence of cyclic stretch and microvascular endothelium—data analysis is on-going. Assess functionality of multiple cell types of Organoids-on-Chip compared to suspension organoid.

Exemplary embodiment: 1. Epithelial Channel; 2. Human Intestine Epithelial Cells; 3. Vacuum Channel; 4. Membrane; 5. Human Intestine Endothelial Cells (or other types of endothelial cells) and 6. Vascular Channel.

Some embodiments of duodenal intestine-chips comprise intestinal cells derived from tissue obtained from human adults. Some embodiments of duodenal intestine-chips comprise intestinal cells derived from tissue obtained from human youths, children and teens. Some embodiments of duodenal intestine-chips comprise intestinal cells derived from tissue obtained from human infants. Unless otherwise designated, experimental data shown herein are from chips comprising intestinal epithelial cells derived from adult humans, e.g. for providing organoids and enteroids, i.e. an “adult” intestinal chip platform.

II. Intestine-on-Chip.

Induction of intestinal drug metabolizing enzymes can complicate the development of new drugs, owing to the potential to cause drug-drug interactions (DDIs) leading to changes in pharmacokinetics, safety and efficacy. The development of a human-relevant model of the adult intestine that accurately predicts CYP450 induction could help address this challenge as species differences preclude extrapolation from animals.

As described herein, embodiments of Intestine-Chip (cultured) with organoids demonstrated superior human-relevant intestine function than organoids alone. In some embodiments, embodiments of Intestine-Chip (cultured) with organoids demonstrated superior human-relevant intestine function than shown in animal models. More specifically, embodiments of Duodenum Intestine-Chip, Ileium Intestine-Chip, and Colon Intestine-Chip, etc., enables robust platform for preclinical drug assessment of drug transport, metabolism and drug-drug interactions in the intestine for predicting actual in vivo intestinal responses. Furthermore, as demonstrated herein, superior functionality of Intestine-Chip (e.g., duodenum and colon) cultured with organoids were shown compared to organoids cultured alone (e.g. static cultures). Moreover, Intestine-Chips were successfully established from individual biopsies showing potential for personalized medicine applications.

Here, we combined organoids and Organs-on-Chips technology to create a human Duodenum Intestine-Chip that emulates intestinal tissue architecture and functions, that are relevant for the study of drug transport, metabolism, and DDI. Duodenum Intestine-Chip demonstrates the polarized cell architecture, intestinal barrier function, presence of specialized cell subpopulations, and in vivo-relevant expression, localization, and function of major intestinal drug transporters. Notably, in comparison to Caco-2, it displays improved CYP3A4 expression and induction capability. This model could enable improved in vitro to in vivo extrapolation for better predictions of human pharmacokinetics and risk of DDIs.

In particular, as demonstrated herein, embodiments of Intestine-Chip produced a nearly identical global transcriptomic profile compared to human intestine duodenum tissue, whereas the signature from the organoids alone had significant differences from the human intestine tissue. The authors further showed that this transcriptional likeness also resulted in significantly more accurate physiologic function of the Intestine-Chip compared to organoids alone. These results demonstrate the potential of the Intestine-Chip as a robust platform to accurately recreate the human intestine tissues to enable highly predictive and human-relevant preclinical drug assessment, including drug transport, metabolism and drug-drug interactions.

More specifically, as demonstrated herein, embodiments of Intestine-Chip seeded from organoids derived from endoscopic biopsies of healthy adult human donors and primary human intestinal microvascular endothelial cells derived from human small intestine. The Intestine-Chip recreated the barrier function and multilineage differentiation of adult human intestinal tissue. Global gene expression was assessed by RNA-sequencing analysis and showed that the profile of the Intestine-Chip and freshly isolated human adult duodenum tissue were remarkably similar; in contrast, the organoids alone from the same donor showed significant differences in RNA-sequencing analysis. Additionally, the biology for drug transporters and drug metabolizing enzymes of the intestine remains intact in the Duodenum Intestine-chip, which is not effectively modeled with current animal testing due to the species specific nature of these drug transporters and enzymes. These findings show a path forward to using more robust technology to better predict human pharmacokinetics and drug-dug interaction.

Exemplary Applications of Methods Described Herein.

Low bioavailability and pharmacokinetics caused by drug-drug interactions of orally administered drugs represents a significant challenge in the modern drug development. High affinity of certain drugs for cellular transporters combined with the extensive activity of metabolic enzymes present in the human intestine are the main factors limiting drug bioavailability (Dietrich, Geier et al. 2003, Thummel 2007, Shugarts and Benet 2009, Peters, Jones et al. 2016). After decades of research, the hepatic drug clearance is well-understood and relatively well predicted by pre-clinical models, while the accurate prediction of the first-pass extraction of xenobiotics in human intestinal epithelium still remains elusive. This is due to a number of confounding factors that affect oral drug absorption including the properties of the compound (solubility, permeability), physiology of the intestinal tract (transit time, blood flow), and patient phenotype (including age, gender, polymorphism in drug metabolizing enzymes, disease states) (Pang 2003). Species differences in the isoforms, regional abundances, differences in substrate specificity of drug metabolism enzymes (Martignoni, Groothuis et al. 2006, Paine, Hart et al. 2006, Komura and Iwaki 2011) and transporters (Tucker, Milne et al. 2012, Groer, Bruck et al. 2013), and mechanism regulating transcriptional activation (LeCluyse 2001, Mackowiak, Hodge et al. 2018), precludes accurate extrapolation of the data from animal models to the clinic. In mice, for example, there are 34 cytochrome P450 (CYPs) in the major gene families involved in drug metabolism, i.e., the CYP1A, CYP2C, CYP2D, and CYP3A gene clusters, while in humans, there are only eight (Nelson, Zeldin et al. 2004).

Interestingly three human enzymes, CYP2C9, CYP2D6, and CYP3A4, account for approximately 75% of all reactions, with CYP3A4 being the single most abundant human CYP450 accounting for approximately 45% of phase 1 drug metabolism in humans (Guengerich 2008). In addition, the expression levels of many of the major human CYP450 enzymes and drug transporter (which determine levels and variability in drug exposure) are controlled by multiple transcription factors, primarily the xenosensors: constitutive androstane receptor (CAR), pregnane×receptor (PXR), and aryl hydrocarbon receptor (AhR). These nuclear receptors also exhibit marked species differences in their activation by drugs and exogenous chemicals (Mackowiak, Hodge et al. 2018). For example, rifampicin and SR12813 are potent agonists for human PXR (hPXR) but not for mouse PXR (mPXR), whereas the potent mPXR agonist 5-pregnen-3β-ol-20-one-16α-carbonitrile (PCN) is a poor agonist for hPXR (Kliewer, Moore et al. 1998). On the other hand, 6-(4-chlorophenyl)imidazo[2,1-b][1,3]thiazole-5-carbaldehyde-O-(3,4-dichlorobenzyl)oxime (CITCO) is a strong agonist for human CAR (hCAR) but not mouse CAR (mCAR) (Maglich, Parks et al. 2003), while 1,4-bis-[2-(3,5-dichloropyridyloxy)]benzene,3,3′.5.5′-tetrachloro-1,4-bis(pyridyloxy)benzene (TCPOBOP) is more selective for mCAR than hCAR. Such species differences together with the complex interplay between drug metabolizing enzymes and drug transporters in the intestine and liver, as well as the overlap of substrate and inhibitor specificity (Shi and Li 2014), make it difficult to predict human pharmacokinetics at the preclinical stage of drug development.

Numerous in vitro models have been developed and applied routinely for characterization and prediction of absorption, distribution, metabolism, and excretion (ADME) of potential drug candidates in humans. Among these is a Caco-2 monolayer culture on a transwell insert, which is one of the most widely used models across the pharmaceutical industry as an in vitro representation of the human small intestine.

However, inherent limitations, such as lack of in vivo relevant three-dimensional cytoarchitecture, lack of appropriate ratio of cell populations, altered expression profiles of drug transporters and drug metabolizing enzymes, especially CYP450s, and aberrant CYP450 induction response, challenge the use of these model for predicting human responses in the clinic (Sun, Chow et al. 2008).

A promising alternative to conventional cell monolayer systems emerged with the establishment of the protocols for a generation of three-dimensional intestinal organoids (or enteroids) from human biopsy specimens (Sato, Vries et al. 2009, Sato, Stange et al. 2011, Eglen and Randle 2015, Liu, Huang et al. 2016). Using these methods, organoids derived from all regions of the intestinal tract can be established (Sato, Stange et al. 2011, Wang, Yamamoto et al. 2015) and applied into different areas of research including organ development, disease modeling, and regenerative medicine (Fatehullah, Tan et al. 2016). However, the characterization of the pharmacokinetic properties of this system, as well as the validation for its use in drug discovery and development, is still very limited (Dekkers, Wiegerinck et al. 2013, van, de Wetering, Francies et al. 2015, Zhang, Zhao et al. 2017, Zhao, Zeng et al. 2017, Vlachogiannis, Hedayat et al. 2018). This could be due to the substantial technical challenges associated with the use of oragnoid technology for ADME applications. The 3D-spherical architecture of the organoid restricts access to its lumen, which is crucial for assessing intestinal permeability or drug absorption. Indeed, exposure of the apical cell surface of intestinal organoid to compounds requires the use of time-consuming and labor-intensive procedures, such as microinjection. The presence of relatively thick gel of extracellular matrix (Matrigel™) surrounding organoids, might limit drug penetration.

While heterogeneity of organoids in terms of their size, shape, and viability, can also impede studies in ADME and robust results (Fatehullah, Tan et al. 2016). In addition, none of these models have fully recapitulated critical aspects of an organ microenvironment such as the presence of microvasculature, mechanical forces of fluid flow (shear stress) and peristalsis, all of which contribute to the complex and dynamic nature of in vivo tissue function (Gayer and Basson 2009). For these reasons, there is a need for new systems for predicting human ADME and determining risk for drug-drug interactions mediated by intestinal CYP450s and drug transporters in the clinic.

Findings from the published functional testing mainly aimed at protein expression, demonstrated that the Duodenum Intestine-Chip more accurately recapitulated several aspects of the human intestine, compared to organoids or Caco-2 models, including: Expression of drug transporters. Expression levels of several transporters for efflux (MDR1, CVRP, MRP2 and MRP3) and uptake (PepT1, OATP2B1, OCT1 and SLC40A1) were similar for the Duodenum Intestine-chip and freshly isolated duodenal tissue, but not the previously described Caco-2 Intestine-Chip which showed significant variation in expression of OATPB1 and OCT1.

Localization and function of drug transporters. In vivo relevant localization of the luminal efflux pumps, MDR1 and BCRP, and the uptake pump PEPT1 co-distributed together with villin, a marker specific for apical cell membrane, at the intestinal cell brush border in Duodenum Intestine-Chip. The MDR1 activity was confirmed by measuring the intracellular accumulation of rhodamine 123 in the presence and absence of specific MDR1 inhibitor, vinblastine, across Duodenum Intestine-Chips.

Drug-mediated CYP3A4 expression and induction potential. The Duodenum Intestine-Chip expressed CYP3A4 at the much higher gene (approximately 6000-times higher, p<0.0001) and protein level compared to the Caco-2 Intestine-chip, reaching the expression similar to the one observed in the adult human duodenum. Expected CYP3A4 induction was observed in the Duodenum Intestine-Chip when exposed to rifamycin and Vitamin D3, two prototypical CYP3A4 inducers. The Caco-2 Intestine-chip showed induction when exposed to Vitamin D3 but not rifamycin.

Study results were derived from testing of Duodenum Intestine-Chips established from individual donor of biopsy-derived organoids. The ability to establish Duodenum Intestine-Chips composed of the cells isolated from individual patients opens the possibility of personalized testing to assess interindividual differences in drug disposition and responses, studies of the effect of genetic polymorphisms on pharmacokinetics and pharmacodynamics as well as decoupling of the effect of various factors such as age, sex, disease state and diet on metabolism, clearance and bioavailability of drugs.

We recently developed a human Duodenum Intestine-Chip that combines healthy intestinal organoids with our Organs-on-Chips technology (Kasendra, Tovaglieri et al. 2018) to overcome the existing limitations of current systems. Here, we demonstrated the Duodenum Intestine-Chip provides a model closer to the human tissue, compared to organoids, as supported by the comparison of RNA seq expression profiles, that could provide a human-relevant platform for the studies of drug transport, drug metabolism, and drug-drug interactions. Presence of the mechanical forces, which we apply in this system in order to recapitulate the blood flow and shear stress, showed to improve the formation of polarized cytoarchitecture and the appearance of intestinal microvilli on the apical cell surface. In addition, Duodenum Intestine-Chip supported successful maturation of all major intestinal cell types in the physiologically relevant ratios and demonstrated low paracellular permeability.

Expression of Drug Uptake and Efflux Transporters Duodenum Intestine-Chip Demonstrated Expression of Major Intestinal Drug Transporters, with Average Expression of OATP2B1 and OCT1 Closer to In Vivo than Observed in Caco-2 Intestine-Chips.

Relative average gene expression levels of drug efflux (MDR1, BCRP, MRP2, MRP3) and uptake (PepT1, OATP2B1, OCT1, SLC40A1) transporters in Caco-2 based Intestine-Chips (Intestine-Chip) or 3 donor-specific Duodenum-Chips and RNA isolated directly from the duodenal tissue of 3 different individuals (Duodenum) reveals that Duodenum-Chips, similarly to the previous Caco-2 based system, express drug transport proteins at levels close to in vivo tissue. Note, that the expression of OATP2B1 and OCT1 in Caco-2 was significantly higher than in vivo while in organoids-derived system shown no significant difference in respect to Duodenum. Each value represents average gene expression±s.e.m (error bars) from three independent experiments, involving Duodenum-Chips generated from organoids of three individual donors (3 chips/donor), Caco-2 cells-based Intestine-Chips (3 chips) and RNA tissue of three different subjects, and are shown relative to the in vivo tissue expressed as 1.

Demonstrated expression of major intestinal drug transporters, with average expression of OATP2B1 and OCT1 closer to in vivo than observed in Caco-2 Intestine-Chips.

For its use in pharmacokinetic studies, embodiments of Duodenum Intestine-Chip showed closer to in vivo expression of drug uptake and efflux transporters, as compared to Intestine-Chip developed with Caco-2 cells. Additionally, we were able to show that the Duodenum Intestine-Chip exhibits the correct luminal localization and functional activity of MDR1 (P-gp), and supports high expression of the cytochrome CYP450 (CYP) 3A4-close to the levels observed in the human duodenal tissue.

Exposure of the Duodenum Intestine-Chip to known CYP450 inducers in humans, such as rifampicin and 1,25-dihydroxyvitamin D3, resulted in significantly increased CYP3A4 mRNA, protein levels, and drug metabolism activity. Our results indicate that the organoid-derived intestinal cells can be combined with Organs-on-Chips technology to provide a robust and human-relevant system for preclinical assessment of CYP450-mediated metabolism, activity of drug transporters, and the potential risk of drug-drug interactions.

III. Small-Intestine-on-Chip: Duodenum.

We previously developed a human primary Intestine-Chip, referred at the time as the “Small Intestine-on-a-Chip”, which combined the use of intestinal organoids isolated from pediatric donors and Organ-Chips (Kasendra, Tovaglieri et al. 2018) to recapitulate critical features of intestinal morphology and associated functions. Here we sought to establish a Duodenum Intestine-Chip from adult organoid-derived cells to serve as a platform in the preclinical assessment of drug transport and metabolism. Thus, in one embodiment, we combined duodenal organoids and organ-on-chip technologies to develop an adult Duodenum-Chip platform emulating intestinal tissue architecture and function relevant for the studies of drug transport and metabolism.

An adult Duodenum-Chip recapitulates intestinal barrier function, multi-lineage differentiation and close to in vivo expression of major intestinal drug uptake and efflux transporters. In addition, it demonstrates correct localization and function of intestinal P-glycoprotein (P-gp)—drug efflux pump. Duodenum-Chip, in comparison to previous Caco-2 cells-based model, possess an improved CYP3A4 expression and induction potential enabling its application into the studies of drug metabolism in humans.

Intestinal drug metabolizing enzyme induction complicates the development of new drugs owing to altered efficacy of concomitant treatment, reduction in the bioavailability of drugs, and potential disruption of the balance between toxification and detoxification. Generation of faithful human in vitro model of adult proximal small intestine able to accurately predict drug-mediated CYP3A4 induction could facilitate preclinical drug development.

Here we report the establishment of one embodiment of a Duodenum-Chip platform, that combines the use intestinal organoids isolated from healthy adult individuals and organ-on-chip platform, for accurate modeling of drug transport and drug mediated CYP3A4 induction. A Duodenum-Chip demonstrates successful maturation of major intestinal cell types, low paracellular permeability and improved (in comparison to Caco-2 cells-based system) expression of drug uptake and efflux transporters. It exhibits correct luminal localization and efflux function of MDR1/P-gp, as well as closer to in vivo expression of the drug-metabolizing enzyme cytochrome P450 (CYP) 3A4. Human P-glycoprotein is encoded by the multidrug resistance 1 (MDR1) gene, also referred to as ATP-binding cassette sub-family B member 1 (ABCB1). Surprisingly, Duodenum-Chip exposure to different drug treatments, such as rifampicin and 1,25-dihydroxyvitamin D3, known P450 inducers in humans, resulted in significant increases in mRNA and protein levels of CYP3A4. Our results indicate that human organoids-derived Duodenum-Chip can serve as a useful in vitro experimental system in drug development studies.

A. Development of a Primary Human Small Intestine-On-A-Chip Using Biopsy-Derived Organoids.

To recapitulate the human adult duodenal tissue-on-chip we adopted an approach established previously for the development of first human primary Intestine-Chip (Small Intestine-on-a-chip) that combined the use of intestinal organoids derived from children with Organ-on-Chip fabrication protocols²⁶. Given the fact, that clinical pharmacology studies are typically conducted in healthy adults and it is well known that pharmacokinetics in children is very different than in adults with respect to drug absorption, distribution, metabolism, and elimination²⁷ we decided to use tissue biopsies derived from adult healthy donors as a starting material for this study, in contrast to the previous experiments based on the use of pediatric specimens.

In brief, after successful generation of three different cultures of 3D organoids (FIG. 5A; top) established from the endoscopic biopsies of healthy individuals, these organoids were dissociated into the fragments and seeded on the top of the ECM-coated porous flexible PDMS membrane in the upper ‘epithelial’ channel of Organ-on-Chip platform (FIG. 1C; epithelial channel is marked with 1, epithelial tissue formed from organoid's fragments and PDMS membrane are labeled with 2 and 4, respectively).

While primary human intestinal microvascular endothelial cells (HIMECs) isolated from the human small intestine (FIG. 5A; bottom) were used to populate the bottom surface of the same membrane in the lower ‘microvascular’ channel (FIG. 1C; endothelial tissue is marked with 5, vascular channel is labeled with 6). Similarly, to our previous observations from the Caco-2 based Gut-on-a-Chip²⁸, exposure of the organoids-derived intestinal epithelium to flow for 72 hours showed to accelerate cell polarization, in addition to the improvement of the formation of apical microvilli (e.g., FIG. 6D). Immunofluorescence microscopic analysis of the distribution of the apical (villin) and basolateral (E-cadherin) cell surface markers and SEM of the luminal cell surface revealed that cells grown under static conditions in Organ-on-Chip platform formed very flat (14.8±2.6 μm) and squamous monolayers with weakly defined cell-cell junctions (e.g., FIG. 6A) and sparsely localized microvilli (e.g., FIG. 6D).

In contrast, cells grown in the presence of fluid flow (30 μLh⁻¹) with or without concomitant cyclic strain (10%, 0.2 Hz) exhibited more polarized cobblestone morphology with increased cell height (27.0±1.3 μm), strongly delineated junctions and densely packed microvilli. In line with the previous Caco-2 findings, flow or shear stress were determinants of this response as cyclic strain did not produce any significant additive effect.

Longer time of the cells exposure (4-6 days) to flow and cyclic strain resulted in the spontaneous development of intestinal folds (“villi-like structures”) extending into the lumen of epithelial microchannel and covered by continuous brush border (FIGS. 6B and 6D).

Immunofluorescence confocal analysis revealed establishment of confluent epithelial and endothelial monolayers across the entire length of the chip (FIG. 5D) with well-defined cell-cell junctions: epithelial tight junctions stained against ZO-1 protein (magenta) and endothelial adherens junctions visualized using antibody against VE-cadherin (green). Surprisingly, these culture conditions resulted in a time-dependent improvement of intestinal permeability as indicated by the decreased permeability coefficient (Papp) of fluorescently labeled dextran recorded for the Duodenum-Chips generated from the tissue of 3 different individuals (FIG. 5E), suggesting that this model forms a functional barrier.

B. Modeling of the Human Adult Duodenal Tissue-On-Chip: Gene Expression.

To confirm efficient differentiation of enterocytes we assessed the gene expression levels of cell type specific markers indicative for the presence of mature enterocytes, including alkaline phosphatase (ALPI) for absorptive enterocytes, mucin 2 (MUC2) for goblet cells, chromogranin A (CHGA) for enteroendocrine cells, lysozyme (LYZ) for Paneth cells, across 3 donor-specific Duodenum-Chips (each established from the tissue of a different individual) and compared it to the freshly isolated adult duodenal tissue (Duodenum). Most of these genes showed to increase in cells grown inside of the Organ-on-Chip platform starting from day 2 up to day 8 of fluidic culture (FIG. 6A), with the exception for the lysozyme (Paneth cell marker) which showed the opposite trend, suggesting increased differentiation of the villus epithelium. Notably, some of these markers such as alkaline phosphatase and mucin 2 showed (around day 8) to reach similar levels to the ones observed in RNA isolated directly from the human duodenum. Identification and quantification of all major differentiated intestinal cell types using confocal microscopy revealed their presence at physiologically relevant ratios (FIG. 6C), close to the ones reported for the histopathological sections of human duodenum (FIG. 6D)²⁹.

Taken together, these results demonstrate the successful establishment of adult Duodenum-Chip platform that closely resemble the architecture, barrier function and multilineage differentiation of adult human small intestine.

C. Differential Gene Expression: Transcriptomic Comparison of the Duodenum Intestine-Chip Versus Organoids.

To further verify whether the Duodenum Intestine-Chip faithfully recapitulates human adult duodenum tissue and to better understand how much it differs from the organoids used for its establishment, we performed RNA-seq analysis (FIG. 3). We compared global RNA expression data obtained from: i) duodenum organoids (Organoids; n=3) cultured for 8 days in a conventional plastic-adherent Matrigel™ drop overlaid with growth medium; ii) Duodenum Intestine-Chip established using cells derived from the above organoids (Duodenum Intestine-Chip; n-3) and grown for 8 days in the presence of constant flow and stretch; iii) human adult duodenum tissue (Adult Duodenum; n=2; full-thickness samples) (Supplementary File 1). The same experimental conditions i.e. maintenance in expansion media for 6 days, followed by 2 days in differentiation media were used for both organoids and chips.

A. On-Chip Growth, Under Flow and Stretch, Promotes Higher Transcriptomic Similarity of Primary Intestinal Epithelium to Adult Duodenum than in 3D Organoid Cultures.

To more clearly demonstrate the effect of dynamic culture conditions on the development and maturation of human primary duodenal epithelium, we conducted RNA-seq analysis of tissues from one embodiment of mechanically active Duodenum Intestine-Chips and compared it to global gene expression data obtained for 3D duodenal organoids and to existing RNA-seq datasets for human adult duodenal tissue (Table 1). 3D duodenal organoids were cultured in conventional plastic-adherent Matrigel drop overlaid with growth medium.

We examined the differential gene expression in 3D organoids compared to a dataset published for human adult tissue. We annotated 13,735 genes in the genome and performed differential gene expression analysis (DGE). For the DGE analysis, we used the “limma” R package which has an excellent performance even at low sample numbers (Ritchie, Phipson et al. 2015, Baccarella, Williams et al. 2018). For all the differentially expressed gene analyses, we used the “Iimma” R package and applied the widely thresholds: adjusted p-value<0.05 and |log₂FoldChange|>2 in order to select the differentially expressed gene³⁰. These strict thresholds allowed for a significant increase in the statistical power of the results.

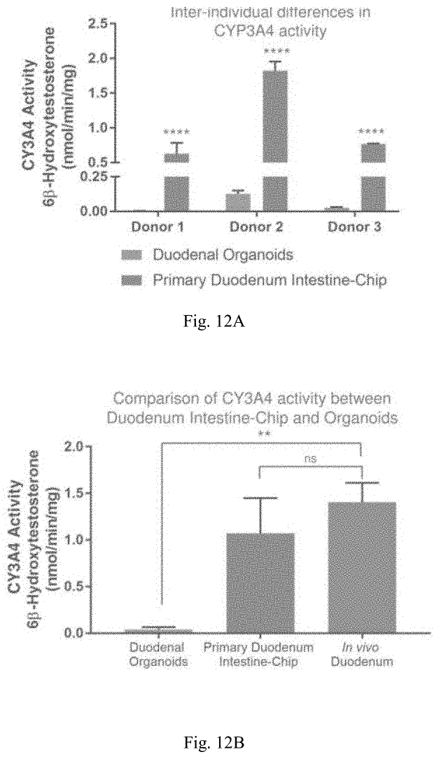

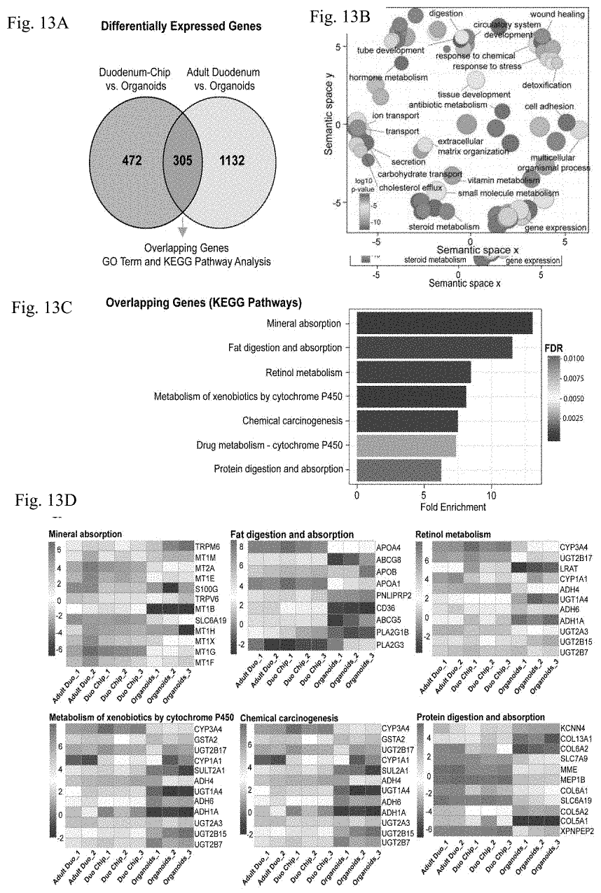

Out of 13,735 genes annotated in the genome, 1437 were significantly differentially regulated among these samples: 562 and 875 genes were respectively up- or down-regulated, respectively. FIG. 12A.

Next a functional enrichment analysis was performed using the PANTHER Classification System to highlight biological processes—significantly enriched gene ontology (GO) terms—within these gene sets (Ashburner, Ball et al. 2000, Mi, Muruganujan et al. 2013, The Gene Ontology 2017). The majority of differentially expressed genes belonged to the pathways related to digestion, extracellular matrix organization, angiogenesis, cell adhesion, tissue development, cell response to drugs, toxic and xenobiotics substances (FIG. 12B). This comparison allowed us to identify genes responsible for global transcriptomic differences between organoid technology and native human tissue and highlight biological functions which could be affected by the observed differences.

Interestingly, among the identified pathways numerous GO terms have been previously associated with the prolonged absence of mechanoluminal stimulation in human small intestine that might occur in patients subjected to the surgical treatment of acute and chronic intestinal insults³⁴. These include but are not limited to: over-represented pathways, such as digestion (GO:0007586), localization (GO:0051179), small molecule metabolic process (GO:0044281), response to wounding (GO:0009611), cellular response to chemical stimulus (GO:0070887), system development (GO:0048731), blood vessel morphogenesis (GO:0048514) and biological regulation (GO:0065007) and under-represented pathways, such as cellular macromolecule metabolic process (GO:0044260), nucleic acid metabolic process (GO:0090304), RNA metabolic process (GO:0016070) and RNA processing (GO:0006396). This implies that application of mechanical forces to the culture of human primary intestinal epithelial cells might be necessary for the accurate in vitro modeling of the healthy human gut.

We applied a similar analysis performed for Duodenum-Chip versus Adult Duodenum that resulted in 1023 differentially expressed genes that were significantly up-(382 genes) or downregulated (641 genes), suggesting a presence of higher degree of similarity in global transcriptional activity between duodenal epithelium grown on-Chip and human in vivo tissue. Thus, the significantly smaller number of differentially expressed genes identified with this analysis, as compared to the DGE performed between organoids and adult duodenum tissue, suggests that combining organoids with Organs-on-Chips provides significant advantages to the emulation of the human duodenal tissue (FIG. 12A).

Functional enrichment analysis highlighted biological processes, such as protein synthesis and targeting, cell cycle and cell proliferation, underlying the above differences in gene expression profiles.

Analysis of Gene Expression in Exemplary Intestinal Pathways.

In order to determine which genes are responsible for the closer similarity of the Duodenum Intestine-Chip, compared to organoids, to human duodenal tissue, we carried out additional DGE analysis.

Gene Ontology (GO) term enrichment is a technique for interpreting sets of genes making use of the Gene Ontology system of classification, in which genes are assigned to a set of predefined bins depending on their functional characteristics. Gene Ontology (GO) knowledgebase is the world's largest source of information on the functions of genes. This knowledge is both human-readable and machine-readable, and is a foundation for computational analysis of large-scale molecular biology and genetics experiments in biomedical research. Gene Ontology enRIchment anaLysis and visuaLkAtion tool.

This time, we assessed the differences between Duodenum Intestine-Chip and organoids, and examined these differences relative to those that exist when comparing the adult human duodenal tissue and organoids (Duodenum Intestine-Chip versus organoids and human adult duodenum versus organoids). We discovered pathways enriched in Duodenum Intestine-Chips but not organoids are associated with drug metabolism, nutrients absorption, and digestion suggesting a transcriptomic profile that is more in vivo-relevant in the chip.

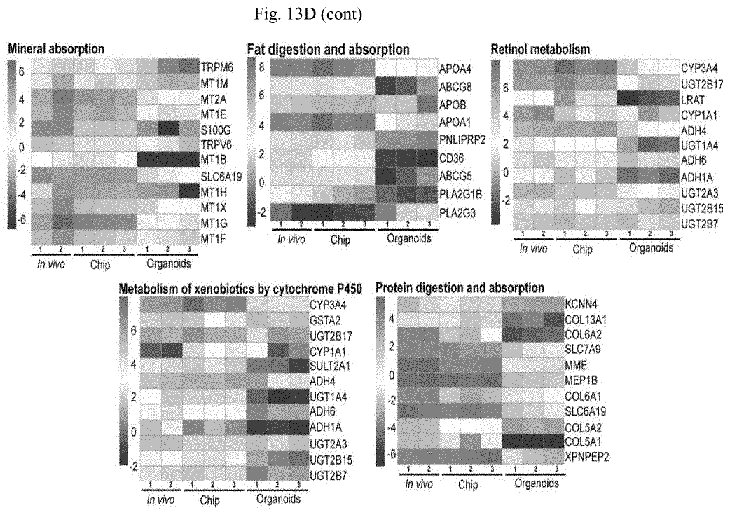

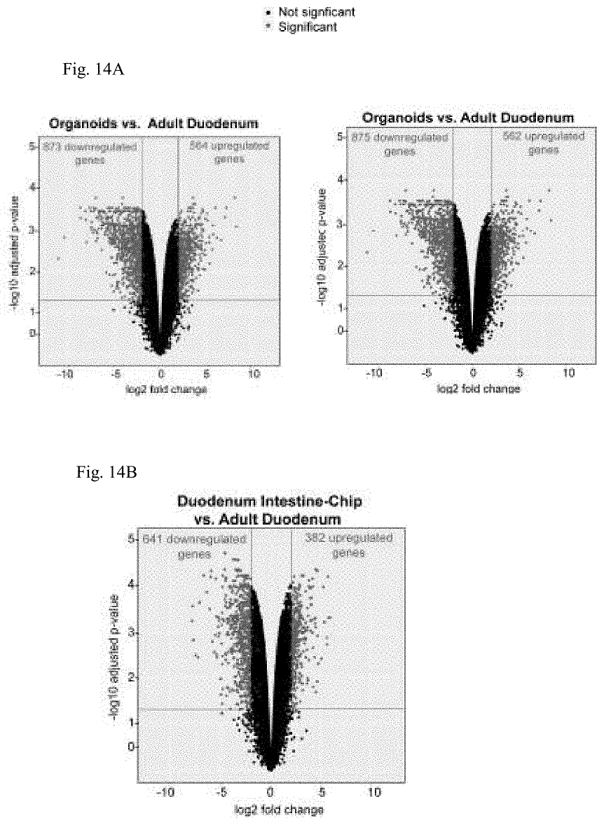

Differential gene expression analysis was carried out to identify genes that are up- or down-regulated in Duodenum Intestine-Chip compared to organoids (blue circle) and adult duodenum compared to organoids (yellow circle), FIG. 13A. We identified genes that were significantly up- and downregulated in the Duodenum Intestine-Chip relative to the organoid culture and identified the proportion of these genes that were also significantly different in adult duodenal tissue relative to organoids. The gene lists were then compared to determine how many genes overlap between those two comparisons, and the results are shown as a Venn diagram, FIG. 13A. We found 305 genes which were common for Duodenum Intestine-Chip and adult duodenum but different from organoids, representing 39.25% of differentially expressed genes in Duodenum Intestine-Chip versus organoids comparison, and 21.22% of differentially expressed genes in human adult duodenal tissue versus organoids comparison. 305 genes were identified as common and responsible for the closer resemblance of Duodenum Intestine-Chip to human adult duodenum than organoids from which chips were derived. Intestinal crypts derived from the same three independent donors were used for the establishment of Duodenum Intestine-Chip and organoid cultures. Both chips and organoids were cultured in parallel, in the presence of expansion media for 6 days, followed by 2 days of differentiation media. Experiment was terminated and samples were processed for analyses 8 days post-seeding.

To further explore overlapping genes, we used the Gene Ontology and Kyoto Encyclopedia of Genes and Genomes (KEGG) Pathway Analysis to examine biological processes and pathways that are enriched in this gene list (Kanehisa and Goto 2000, Thomas, Campbell et al. 2003). In addition, we used the well-established Reduce and Visualize Gene Ontology (REVIGO) tool to reduce redundancies by clustering semantically related GO terms and outputting them as a scatter plot for visualization (Eden, Lipson et al. 2007, Supek, Bosnjak et al. 2011).

The list of overlapping genes was subjected to GO analysis to identify enriched biological processes (GO terms). The results are shown as REVIGO scatterplots in which similar GO terms are grouped in arbitrary two-dimensional space based on semantic similarity. Each circle corresponds to a specific GO term and circle sizes are proportional to the number of genes included in each of the enriched GO terms. Finally, the color of a circle indicates the significance of the specific GO term enrichment. GO terms enriched in the overlapping gene set demonstrate that Duodenum Intestine-Chip is more similar to human duodenum with respect to important biological functions of the intestine, including digestion, transport and metabolism, FIG. 13B.

As a result, the list of 305 overlapping genes was associated with 117 significant GO terms, which were reduced to 74 unique GO terms in REVIGO, FIG. 13B. Biological processes enriched in this gene set were associated with intestinal functions such as digestion and transport of nutrients and ions, metabolism, detoxification, as well as tissue and tube development (FIG. 13B).

Merely for descriptive information, GO terms may be large and highly redundant, and thus difficult to interpret. REVIGO refers to a Web server (http://revigo.irb.hr/) that summarizes long, unintelligible lists of GO terms by finding a representative subset of the terms using a simple clustering algorithm that relies on semantic similarity measures. Furthermore, REVIGO visualizes this non-redundant GO term set in multiple ways to assist in interpretation: multidimensional scaling and graph-based visualizations accurately render the subdivisions and the semantic relationships in the data, while treemaps and tag clouds are also offered as alternative views. REVIGO (reduce and visualize gene ontology) takes long lists of Gene Ontology terms and summarizes them by removing redundant GO terms. Supek F, Bos̆njak M, S̆kunca N, S̆muc T. “REVIGO summarizes and visualizes long lists of Gene Ontology terms” PLoS ONE 2011.

Curated heatmaps were generated to examine particular genes that belong to the enriched KEGG pathways and to show the expression levels (log e (FPKM)) of these genes across different samples. Genes belonging to five different pathways, including: “mineral absorption”, “fat digestion and absorption”, “retinol metabolism”, “metabolism of xenobiotics by cytochrome P450” and “protein digestion and absorption”, are shown, FIG. 13C. KEGG Pathway analysis identified a total of 7 significantly enriched pathways (threshold FDR adjusted p-value <0.05) among the overlapping genes. The corresponding fold enrichments of these pathways are shown in FIG. 13C.

For the identification of the enriched pathways we used the FDR adjusted p-value (instead of the p-value) which significantly increased the statistical power of our findings. Many of these pathways were linked to drug metabolism, such as—cytochrome CYP450 (associated with 10 genes), metabolism of xenobiotic by cytochrome CYP450 (associated with 12 genes), and other pathways related to nutrients absorption and digestion, such as mineral absorption (associated with 12 genes), fat digestion and absorption (associated with 9 genes), protein digestion and absorption (associated with 11 genes) (FIG. 13C).

To more closely examine gene signatures that belong to these pathways, we used log (FPKM) values generated from the RNA-seq dataset, and generated heatmaps for differentially expressed genes that fall under the 7 significantly enriched pathways (FIG. 3D). A large number of genes involved in the nutrient absorption, digestion, as well as, metabolism of xenobiotics were highly expressed in Duodenum Intestine-Chip (Chip) and adult duodenum tissue (Adult Duo), but expressed at the low level in organoids. The expression levels of genes associated with “chemical carcinogenesis” (CYP3A4, GSTA2, UGT2B17, CYP1A1, SULT2A1, ADH4, UGT1A4, ADH6, ADH1A, UGT2A3, UGT2B15, UGT2B7) and “drug metabolism—cytochrome 450” CYP3A4, GSTA2, UGT2B17, ADH4, UGT1A4, ADH6, ADH1A, UGT2A3, UGT2B15, UGT2B7) were included in the heatmap representing “metabolism of xenobiotics by cytochrome P450” as they showed to overlap in between 3 different pathways, FIG. 1 3D. See also FIG. 14A-B showing the results of DGE analysis followed by functional enrichment performed between Organoids or Duodenum Intestine-Chip and Adult Duodenum.

To further assess the differences between Duodenum-Chip and 3D organoids and to examine these differences relative to those that exist when comparing adult human duodenum and organoids, we carried out additional differential gene expression analysis (Duodenum-Chip versus 3D organoids and Adult Duodenum versus 3D organoids). We identified genes significantly up- and down-regulated in duodenal cells grown on-Chip relative to organoids culture and identified the proportion of these genes that are also significantly different in adult duodenal tissue relative to organoids.

We found that 305 genes commonly differentiate Duodenum-Chip and adult duodenum from organoids, representing 39.25% of differentially expressed genes in Duodenum-Chip versus organoids comparison and 21.22% of differentially expressed genes in adult duodenum versus organoids comparison (FIG. 13A).

To further explore common genes, we used the Gene Ontology and Kyoto Encyclopedia of Genes and Genomes (KEGG) Pathway Analysis to examine biological processes and pathways that are enriched in the overlapping gene list^{35,36}. In addition, we used REVIGO tool (Reduce and Visualize Gene Ontology) to reduce redundancies by clustering semantically related GO terms and outputting them as a scatter plot for visualization^{37,38}. As a result, the list of 305 overlapping genes was associated with 117 significant GO terms, which were reduced to 74 unique GO terms in REVIGO.

Notably, biological processes enriched in this gene set were associated with intestinal functions such as digestion and transport of nutrients and ions, metabolism, detoxification as well as tissue and tube development (FIG. 13B). In addition, KEGG Pathway analysis identified the total of 7 significantly enriched pathways (threshold FDR p-value <0.05) among the overlapping genes (FIG. 13C). The corresponding fold enrichments of these pathways are shown in FIG. 13C.

Surprisingly, many of these pathways were link to drug metabolism, such as drug metabolism—cytochrome P450 (associated with 10 genes), metabolism of xenobiotic by cytochrome P 450 (associated with 12 genes) and other pathways related to nutrients absorption and digestion, such as mineral absorption (associated with 12 genes), fat digestion and absorption (associated with 9 genes), protein digestion and absorption (associated with 11 genes) (FIG. 13C).

To more closely examine gene signatures that belong to these pathways, we used log(FPKM) values generated from the RNA-seq dataset, and generated heatmaps for differentially expressed genes that fall under the 7 significantly enriched pathways (FIG. 7D). The heatmaps provided in FIG. 7D, clearly indicate the significant difference of Adult Duodenum and Duodenum-Chips versus 3D organoids with respect to these biological functions of the intestine.

Pathways enriched in Duodenum Intestine-Chips but not organoids are associated with drug metabolism, nutrients absorption, and digestion suggesting a transcriptomic profile that is more in vivo-relevant in the chips.

Cumulatively, this data demonstrated an increased similarity in the global gene expression profile between Duodenum Intestine-Chip and human adult duodenal tissue, compared to organoids.

Thus, intestinal transport, metabolism, and tissue development pathways are expressed similarly in Duodenum Intestine-Chips and adult duodenal tissue but differ in organoids.

E. Duodenum-Chip Shows Close to In Vivo Expression of Major Intestinal Drug Transporters and Recapitulates MDR/P-Gp Efflux Activity.

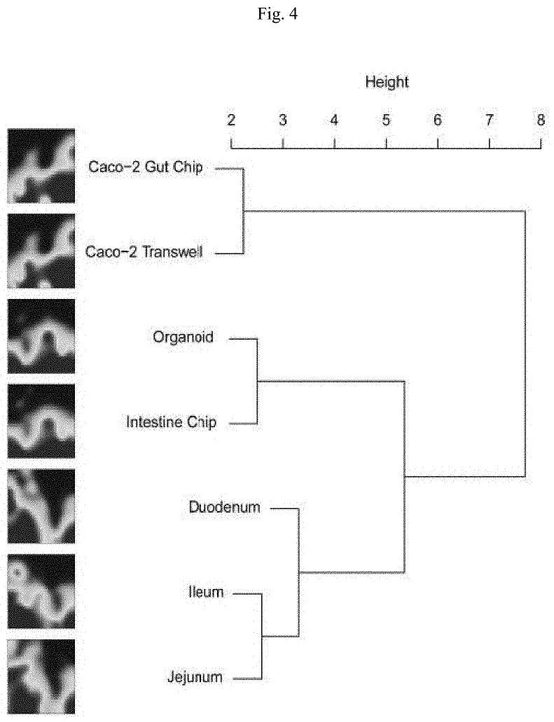

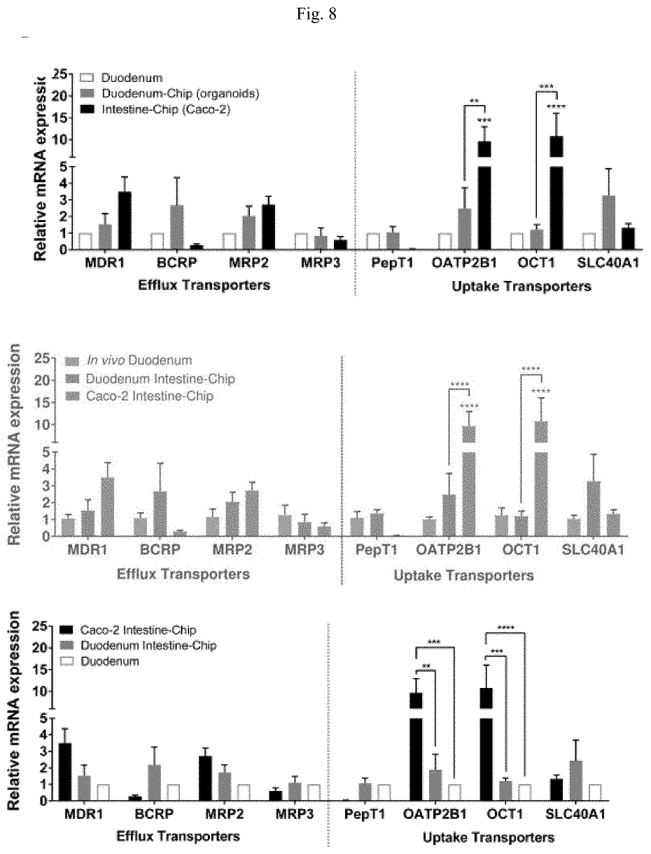

To evaluate the applicability of this system for the studies of pharmacology related questions we assessed the presence and localization of major intestinal drug transporters in Duodenum-Chips established from the tissue of 3 different donors by qRT-PCR and immunofluorescent imaging. The average gene expression levels of efflux (MDR1, BCRP, MRP2, MRP3) and uptake (PepT1, OATP2B1, OCT1, SLC40A1) drug transporters in organoids-derived Duodenum-Chips showed to reached levels similar to in vivo—RNA isolated directly from duodenal tissue (Duodenum) (FIG. 8A) also in the case of genes which were strongly upregulated in the previous Intestine-Chip model, OATP2B1 and OCT1, due to the cancer origin of Caco-2 cells. We demonstrated correct localization of luminal efflux pumps, P-glycoprotein (P-gp) (FIG. 8B) and Breast cancer resistance protein (BCRP) (Supplementary FIG. 3A) as well as uptake Peptide Transporter 1 (PEPT1) (Supplementary FIG. 3B), all of which showed to co-distribute with apical cell marker—villin in Duodenum-Chips at the time of confluent monolayer formation and in successively formed villi-like structures. This co-localization can be noticed in the cross-sectional confocal images of the duodenal epithelium and corresponding to them plots showing the distribution of the fluorescence signal across cell z-axis where an overlap of green (transporters) and magenta (villin) channels results in the presence of white merge areas and spectra overlap between the two channels, respectively. The P-gp activity was confirmed by measuring the intracellular accumulation of rhodamine 123 in the presence and absence of specific inhibitor—vinblastine across Duodenum-Chips established from 3 different donors and compared to the previous Caco-2 based Intestine-Chip model (FIG. 4C). Addition of the inhibitor induced-2-fold increase in intracellular accumulation of rhodamine in both systems (1.84-fold increase in Duodenum-Chip and 2.14-fold increase in Caco-2 cells-based model) confirming the presence of the active P-gp efflux pumps.

F. Duodenum-Chip Model Drug-Mediated CYP3A4 Induction.

Since the induction of drug metabolism enzyme p450 in human intestine, is known to affect pharmacokinetics and bioavailability of various oral drugs we decided to evaluate the capability of our new platform to emulate CYP3A4 drug-induction.

Previously developed Caco-2 cells-based Intestine-Chip showed to possess increased activity of the drug metabolism enzyme p450 in comparison to the conventional static culture of these cells on a Transwell insert (25). Nevertheless, the gene expression level of CYP3A4 measured in this system is significantly lower than in the adult human intestine (FIG. 8A), limiting the use of this platform for pharmaceutical research.

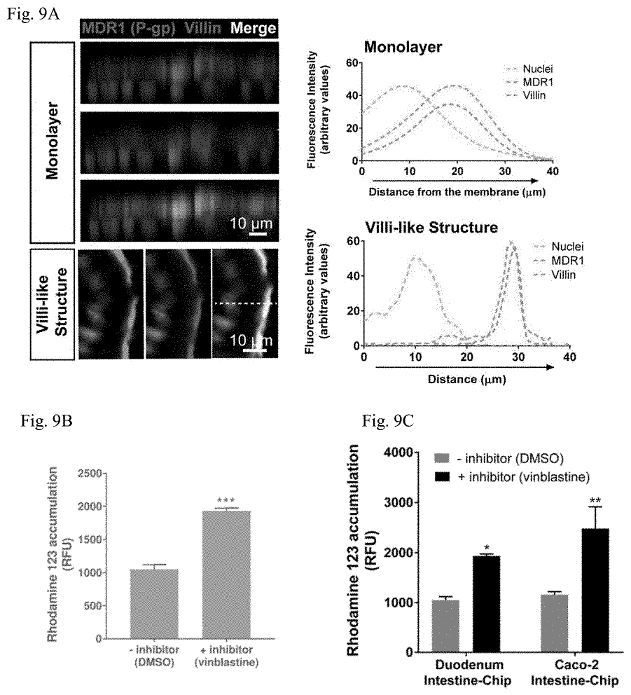

Here, the comparison between Caco-2 based system and our new Duodenum-Chip revealed much higher CYP3A4 gene (6000-times higher, p<0.0001) (FIG. 9A) and protein expression levels (FIG. 9B) in organoids-derived platform than those in the Caco-2 cells. Notably, the CYP3A4 expression in Duodenum-Chip reached the levels similar to the ones observed in the adult human duodenum.

Next, to assess the drug-mediated CYP3A4 induction potency of Duodenum-Chip, we exposed it to rifampicin (RIF) and 1,25-dihidroxyvitamin D3 (VD3), both of which are known as CYP3A4 inducers. Treatment of all 3 donor-specific Duodenum-Chips established from the tissue of different individuals with RIF and VD3 resulted in the average 5.3-fold and 4.1-fold induction of CYP3A4 expression, respectively, relative to that of DMSO-treated controls (FIG. 9C). Consistent with the previous reports³⁹⁻⁴¹, we found that CYP3A4 expression in Intestine-Chip based on the use of Caco-2 cell line was induced only by VD3 treatment (FIG. 9).

Nevertheless 316-fold enhancement of CYP3A4 mRNA level in vitamin D-treated Caco-2 cells, the CYP3A4 protein level assessed by Western Blot analysis was still below the limit of detection (FIG. 9D, bottom). The differences between drug-mediated CYP3A4 induction potency of these two systems could be attributed to differences in gene expression levels of intestinal nuclear receptors, including Pregnane×Receptor (PXR) and vitamin D receptor (VDR). The gene expression levels of PXR and VDR in Duodenum-Chip were significantly higher than those in Caco-2 based Intestine-Chip. The lack of PXR, which is known to be a nuclear receptor of rifampicin, in Caco-2 cells explains why the rifampicin-mediated CYP3A4 induction was successfully confirmed in the Duodenum-Chip, but not in the previous Intestine-Chip platform.

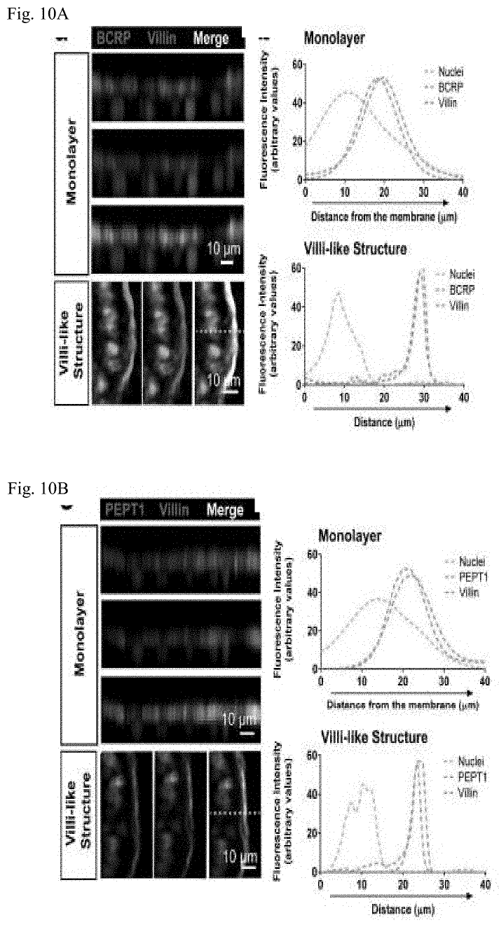

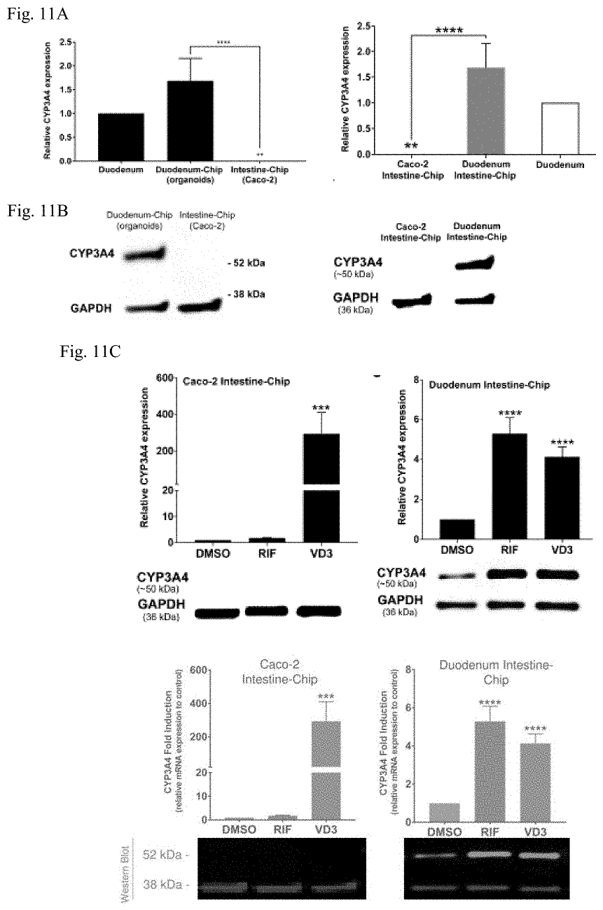

Expression of CYP3A4, PXR, and VDR in Duodenum Intestine-Chip is closer to in vivo versus Caco-2-Chip. Caco-2 cells lack PXR expression limiting utility for drug metabolism, drug transport, and drug-drug interaction studies. See, FIG. 10A-B, for example.

Additionally, our results suggest that the gene expression pattern of intestinal nuclear receptors in organoids-derived chips is more similar to that in the adult small intestine than is the pattern in the Caco-2 cells.

Our findings demonstrate that Duodenum-Chip represent a superior and more appropriate model for drug-mediated induction than the previous Caco-2 cell-based platform and thereby may have a great value for those involved in drug discovery and development.

G. Discussion.

Many in vitro cell cultures and in vivo animal models were developed and successfully applied to characterize and predict the absorption, distribution, metabolism and excretion (ADME) of xenobiotics in humans. Among them, one of the most widely used models is the conventional Caco-2 monolayer culture, that has been and is considered as the ‘gold standard’ in studying intestinal disposition of drugs in vitro. However, these cell cultures suffer several limitations, as they represent one specific region of the intestine, they do not reflect the tissue multicellularity, e.g., lack of appropriate ratio of major cell populations, lack three-dimensional (3D) structure (cytoarchitecture) and exhibit altered expression profiles of drug transporters (DTs) and drug metabolizing enzymes (DMEs), especially cytochromes P450 and aberrant CYP450 induction responses, thus limiting the accuracy of the prediction for drug transport and metabolism in human patients in the clinic. On the other side of the spectrum, the in vivo models retain the proper physiological conditions, exhibit species differences in both drug metabolism and drug transport, as well as substrate specificity for nuclear receptors regulating CYP450s and transporters. Further, their screening capacity is too low and the cost of their use is exceptionally high. Thus, current preclinical models are not able to fully recapitulate the complex nature and function of human intestinal tissue, leading to a limited accuracy and poor predictability in drug development.

In part for reasons described herein, there is a need for new models for predicting human ADME and determining risk for drug-drug interactions mediated by intestinal CYP450s and drug transporters.

In the present study we describe an alternative system, Duodenum-Chip, that represents an engineered intestinal microenvironment able to support multicellular tissue organization and differentiation, physiological polarized expression and function of DTs and drug-mediated induction of DME. Additionally, by combining the use of intestinal organoids, that possess an unlimited expansion capacity, and Human Emulation System, that allows an automated control over the biochemical and physical parameters of chip culture (composition of media, fluid flow, stretch), this model enables generation of patient-specific platforms compatible with the high throughput demands of pharmaceutical industries. We believe that in a close future, Duodenum-Chip could facilitate the assessment of inter-individual differences in human drug responses already at the preclinical stage of drug development process and enable discovery of new therapeutics which are tailored for an individual patient's specific genetic background. In fact, we obtained data showing inter-individual differences in human drug responses supporting the use of a Duodenum-Chip for preclinical drug testing and for use in determining an individual's response to a drug.

H. Materials and Methods.

Human Tissue Collection, Generation and Culture of Organoids

Human duodenal organoids cultures were established from biopsies obtained during endoscopic or surgical procedures utilizing the methods developed by the laboratory of Dr. Hans Clevers (Sato, Stange et al. 2011). De-identified biopsy tissue was obtained from healthy adult subjects provided informed consent at Johns Hopkins University and methods were carried out in accordance with approved guidelines and regulations.

Experimental protocols were approved by the Johns Hopkins University Institutional Review Board (IRB #). Briefly, organoids generated from isolated intestinal crypts were grown embedded in Matrigel (Corning, USA) in the presence of Expansion Medium consisting of Advanced DMEM F12 supplemented with 50% v/v Wnt3A conditioned medium (produced by L-Wnt3A cell line, ATCC CRL-2647), 20% v/v R-spondin-1 conditioned medium (produced by HEK293T cell line stably expressing mouse R-spondin1; kindly provided by Dr Calvin Kuo, Stanford University, Stanford, Calif.), 10% v/v Noggin conditioned medium (produced by HEK293T cell line stably expressing mouse Noggin), 10 mM HEPES, 0.2 mM GlutaMAX, 1×B27 supplement, 1×N2 supplement, 1 mM n-acetyl cysteine, 50 ng/ml human epithelial growth factor, 10 nM human [Leu15]-gastrin, 500 nM A83-01, 10 μM SB202190, 100 μg/ml primocin. EM was replaced every other day and supplemented with 10 μM CHIR99201 and 10 μM Y-27632 during the first 2 days after passaging. Organoids were passaged every 7 days and used for chip seeding between passage number 5 and 30.

Duodenum Intestine-Chip and Human Emulation System.

The design and fabrication of Organ-on-Chip platforms used to develop Duodenum-Chip was based in part on previously described protocols (Huh, Kim et al. 2013). However, it is not intended to limit a microfluidic device to a closed top chip. In some embodiments, a microfluidic device to an open top chip.

In one exemplary embodiment, a microfluidic device (Chip) is made of a transparent, flexible polydimethylsiloxane (PDMS) elastomer (elastomeric polymer). In one exemplary embodiment, a chip contains two parallel, cell culture microchannels (1×1 mm and 1×0.2 mm, epithelial and vascular channel, respectively) separated by a thin (50 μm), porous membrane (7 μm diameter pores with 40 μm spacing) coated with ECM (200 μg/ml collagen IV and 100 μg/ml Matrigel™ at the epithelial side and 200 μg/ml collagen IV and 30 μg/ml fibronectin at the vascular side).

Chips were seeded with intestinal epithelial cells obtained from non-enzymatic dissociation of organoids, as described previously, enzymatic dissociation of organoids, as described previously (Kasendra, Tovaglieri et al. 2018), and incubated overnight before washing with fresh media²⁶.

The next day, in some embodiments, Chips were connected to the Human Emulation System, a semi-automatic system that can hold up to 12 chips and allows for control of gas requirements, flow and stretching of each chip, while eliminating the use of the connecting tubing and a central gas supply, in some embodiments, chips were connected to the culture module instrument (inside the incubator), that can hold up to 12 chips and allows for control of flow and stretching within the chips using pressure driven flow (Vatine, Barrile et al. 2019). Thus, Chips were maintained under constant perfusion of fresh expansion medium at 30 μl/hr through top and bottom channels of chips until day 6. Use of Emulation System allowed a simultaneous and constant perfusion of fresh EM media at 30 μl/hr through top and bottom channels of chips until day 6.

Human Intestinal Microvascular Endothelial Cells (HIMECs; Cell Biologics) were than plated on the lower side of the ECM-coated porous membrane in EGM2-MV medium, which contains human epidermal growth factor, hydrocortisone, vascular endothelial growth factor, human fibroblastic growth factor-B, R3-Insulin-like Growth Factor-1, Ascorbic Acid and 5% fetal bovine serum (Lonza Cat. no. CC-3202) while the media in the epithelial channel was switched to Differentiation Media (DM). Differentiation medium consisted of the same components as those of Expansion Medium (EM) without the addition of Wnt3A, SB202190, as well as 50% reductions in the concentrations of Noggin and R-spondin.

Continuous flow of the DM and EGM2-MV media were applied through the top and bottom channels, respectively. In some embodiments, cyclic, peristalsis-like, deformations of tissue attached to the membrane (10% strain; 0.2 Hz) were initiated after formation of confluent monolayers (˜4 days). Both of the dynamic components of Duodenum-Chip culture, flow and cyclic stretch, were supported by the use of Human Emulation System.

Permeability Assays.

In order to evaluate the establishment and integrity of the intestinal barrier, in some embodiments, dye permeability assays are used to demonstrate permeability levels (alterations/changes). As one example, to evaluate the integrity of the intestinal barrier 3 kDa Cascade Blue dextran (Sigma, D7132) was added to the epithelial compartment of the Duodenum-Chip at 0.1 mg/ml on the day of connecting a microfluidic device to flow, e.g., see exemplary timelines. Effluents of the endothelial compartment were sampled 3 hours after the addition of a dye, up to 24 hours, up to 48 hours, up to 72 hours, or at 48 hour intervals until the end of the assay, in order to determine the concentration of dye that has diffused through the membrane.

The apparent paracellular permeability (Papp) was calculated based on a standard curve and using the following formula:

$$Papp ( \frac{cm}{s} ) = \frac{{C}_{effluent} ( \frac{mg}{ml} ) \times Flow rate ( \frac{ml}{s} )}{{C}_{dosing} ( \frac{mg}{ml} ) \times A ( {cm}^{2} )}$$

Where C_{effluent} (C output) is the concentration of dextran in the effluents of the endothelial compartment, A is the seeded area, and C_{dosing} (C input) is the dosing concentration of dextran spiked into the epithelial compartment.

The establishment of intestinal barrier function in Intestine-Chips, e.g., Duodenum Intestine-Chip, Colon Intestine-Chip, etc., was evaluated in at least three independent experiments, each performed using chips established from a different donor of biopsy-derived organoids. At least three different chips were used per test condition.

It is not intended to limit the direction of the dye diffusion assay, indeed, dye may be added to the endothelial channel then effluent sampled from the epithelial channel.

Morphological Analysis.

Immunofluorescent staining of cells in Organ-on-Chip, e.g. Intestine-Chip, was performed as described previously with some modifications (Kasendra, Tovaglieri et al. 2018). Cells were fixed with 4% formaldehyde or cooled methanol, and when required, were permeabilized, using 0.1% Triton X-100. 5% (v/v) donkey serum solution in PBS was used for blocking. Incubation with primary antibodies directed against ZO-1, VE-cadherin, E-cadherin, villin, mucin 2, lysozyme, chromogranin A, MDR1 (P-gp), BCRP, PEPT1 (see Key Resources Table) Supplementary Table 8) was performed overnight at 4′C. Chips treated with corresponding appropriate Alexa Fluor secondary antibodies (Abcam) were incubated in the dark for 2 hours at room temperature. Cells were then counterstained with nuclear dye DAP1. Images were acquired with an inverted laser-scanning confocal microscope (Zeiss LSM 880 with Airyscan).

Chips processed for SEM samples were fixed in 2.5% glutaraldehyde, treated with 1% osmium tetroxide in 0.1 M sodium cacodylate buffer, dehydrated in a graded series of ethanols and critical point dried (Kasendra, Tovaglieri et al. 2018). Prior to imaging, samples were coated with a thin (10 nm) layer of Pt/Pd using a sputter coater.

Measurement of the Density of Microvilli.

Images of microvilli on the surface of cells were captured with a scanning electron microscope (JSM-5600LV; JEOL). The morphological analysis and quantification of microvilli was performed using ImageJ. Number of intestinal microvilli per μm 2 were calculated after applying two image processing techniques, namely binarization and particle analysis, and Otsu's thresholding method, as described previously (Julio, Merindano et al. 2008).

MDR-1/P-Gp Efflux Pump Activity.

The transporter activity of MDR-1/P-gp was assessed using the MDR1 Efflux Assay Kit (e.g., ECM910, Millipore), as per manufacturer's instructions. Briefly, Duodenum Intestine-Chips and Caco-2 Intestine-Chips were perfused apically with rhodamine 123, a fluorescent transport substrate of MDR1. Intracellular accumulation of dye, was detected by fluorescent imaging (Olympus IX83) and measured in the presence and absence of MDR-1 specific inhibitor vinblastine (22 μM). At least three independent experiments were performed for Caco2 Intestine-Chips and Duodenum-Chips, each using chips established from a different donor of biopsy-derived organoids. At least three different chips were used per condition, images of each were taken at three different fields of view, digitally processed and quantified using Fiji software.

CYP3A4 Induction.

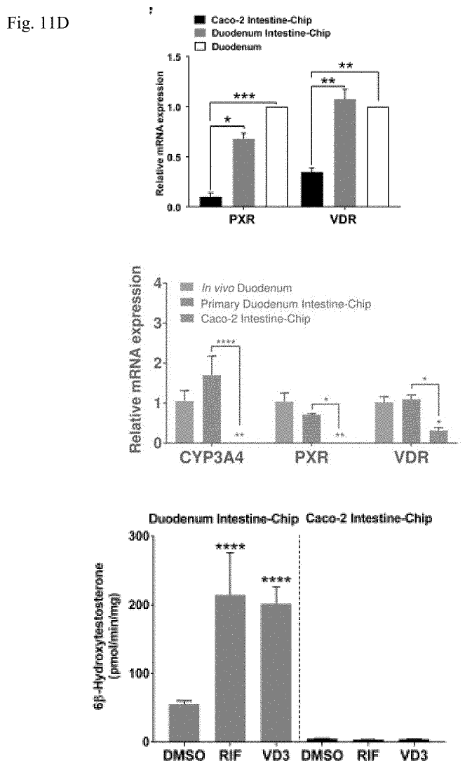

Duodenum-Chips and Caco-2 Intestine-Chips were treated, for 48 hours with 100 nM 1,25-Dihydroxyvitamin D3 (Sigma) or 20 μM rifampicin (Sigma), which are known to induce CYP3A4. Controls were treated with DMSO (final concentration 0.1%). Observed significant increased in mRNA and protein expression after induction with CYP3A4 prototypical inducer rifampicin. For example, see, FIG. 11C. CYP3A4 enzyme activity was also determined using prototypical substrate testosterone (Sigma). Duodenum Intestine-Chips were incubated with 200 μM testosterone for 1 hour under a flow rate of 300 μL/hr. The reaction was stopped using acetonitrile with 0.1% formic acid, and formation of 6β-hydroxytestosterone was measured using LC-MS at In Vitro ADMET Laboratories, Inc. (IVAL). Specific activity of CYP3A4 was determined by dividing the total metabolite formed by the incubation time and normalized to protein contents (pmol/min/mg protein). In order to assess the level of CYP3A4 induction at the gene and protein level cells in the epithelial channel were harvested either for RNA isolation and gene expression analysis or for Western blotting, respectively.

Western Blotting.

Total protein was extracted from Chips using RIPA cell lysis buffer (Pierce) supplemented with protease and phosphatase inhibitors (Sigma) and protein concentration was determined using the bicinchoninic acid method. Equal amounts (15 μg) of protein lysates were heat denatured and separated on a 4-10% Mini-Protean Precast Gel (Bio-Rad), followed by their transfer on o nitrocellulose membrane (Bio-Rad). After blocking with 5% nonfat milk, blots were probed with primary antibodies for CYP3A4 (mouse monoclonal, Santa Cruz Biotechnology) and GAPDH (rabbit polyclonal, Abcam) overnight at 4° C. and IRDye-conjugated secondary antibodies against rabbit and mouse immunoglobulin G (LI-COR) for 1 hour at room temperature. Blots were scanned using an Odyssey Infrared Imaging System (LI-COR) and the protein bands were visualized and quantified using Image Studio software (LI-COR). Gluceraldehyde-3-phosphatase dehydrogenase (GAPDH) was used as a loading control.

Gene Expression Analysis.

Total RNA was isolated from the Chip using PureLink RNA Mini kit (Fisher Scientific) and reverse transcribed to cDNA using SuperScript IV Synthesis System (Thermo Fisher Scientific). qRT-PCR was performed using TaqMan Fast Advanced Master Mix (Applied Biosystems) and TaqMan Gene Expression Assays, Thermo Fisher Scientific) in QuantStudio 5 PCR System (Thermo Fisher Scientific). Relative expression of gene was calculated using 2-ΔΔCt method.

RNA Isolation and Sequencing.

RNA was extracted using TRIzol (e.g. Life Technologies, TRI reagent, Sigma) according to manufacturer's guidelines. Samples were submitted to GENEWIZ South Plainfield, N.J. for next generation sequencing. After quality control and a complementary DNA library creation, all samples were sequenced using HiSeq 4000 or equivalent platform with 2×150 bp paired-end reads per sample.

RNA Sequencing Bioinformatics.

Pre-processing: Raw sequence data (.bcl files) generated from Illumina HiSeq was converted into fastq files and de-multiplexed using Illumina's bcl2fastq 2.17 software. Read quality was assessed using FastQC. Adaptor and low-quality (<15) sequences were removed using Trimmomatic v.0.36. The trimmed reads were mapped to the Homo sapiens reference genome available on ENSEMBL using the STAR aligner v.2.5.2b. The STAR aligner uses a splice aligner that detects splice junctions and incorporates them to help align the entire read sequences. BAM files were generated as a result of this step. Unique gene raw counts were calculated by using feature Counts from the Subread package v.1.5.2. Unique reads that fell within exon regions were counted.

Differential Gene Expression Analysis.

To combine our gene expression dataset with the publicly available adult duodenum gene expression samples (provided as Fragments Per Kilobase of transcript per Million mapped read (FPKM) (Finkbeiner, Hill et al. 2015), we converted the raw counts to FPKMs. Using the log 2 (FPKM) expressions of the combined datasets, we applied DE gene analysis using the R package “limma” (Ritchie, Phipson et al. 2015). For each comparison, the thresholds used to identify the DE genes were set to a) adjusted p-values <0.05 and b) absolute log 2 fold change >2.

GO Term Enrichment Analysis and KEGG Pathway Analysis.

After expression pattern clustering, the transcripts from specific groups were subjected to functional annotation, including GO (The Gene Ontology) functional annotation and KEGG (Kyoto Encyclopedia of Genes and Genomes) pathway annotation. The GO terms and KEGG pathway enrichment was performed using The Database for Annotation, Visualization and Integrated Discovery (DAVID v 6.8, david.abcc.ncifcrf.gov).

Statistical Analysis.

Experiments were performed in triplicates and repeated with Chips established from organoids of minimum three different donors. One-way or Two-way ANOVA was performed to determine statistical significance, as indicated in the figure descriptions (error bars represent standard error of the mean (s.e.m); P values <0.05 and above are considered to be significant).

TABLE 1
RNAseq Datasets Downloaded from Public Databases

| Sample Label | Description | Source | Donor ID | Accession # |
|---|---|---|---|---|
| Adult Duo_1 | Adult Duodenum | EMBL-EBI ArrayExpress | V145 | E-MTAB-1733 |
| | | | | (duodenum_4b) |
| Adult Duo_2 | Adult Duodenum | EMBL-EBI ArrayExpress | V150 | E-MTAB-1733 |
| | | | | (duodenum_4c) |

TABLE S1
List of genes used to generate Duodenal heatmap.

| GENE | Full Name | GO Term |
|---|---|---|
| ADAMTS | ADAM metallopeptidase with | GO0006952_defense_response |
| 13 | thrombospondin type 1 motif 13 | |
| NTHL1 | nth-like DNA glycosylase 1 | GO0006952_defense_response |
| HMGB1 | high mobility group box 1 | GO0006952_defense_response |
| AGER | advanced glycosylation end | GO0006952_defense_response |
| | product-specific receptor | |
| IGLL1 | immunoglobulin lambda like | GO0006952_defense_response |
| | polypeptide 1 | |
| PLA2G1B | phospholipase A2 group IB | GO0006952_defense_response |
| IGFBP4 | insulin like growth factor binding | GO0006952_defense_response |
| | protein 4 | |
| CCL2 | C-C motif chemokine ligand 2 | GO0006952_defense_response |
| ELM01 | engulfment and cell motility 1 | GO0006952_defense_response |
| CTSL | cathepsin L | GO0006952_defense_response |
| S1PR3 | sphingosine-1-phosphate receptor | GO0006952_defense_response |
| | 3 | |
| PM20D1 | peptidase M20 domain containing | GO0006952_defense_response |
| | 1 | |
| CTSB | cathepsin B | GO0006952_defense_response |
| PTX3 | pentraxin 3 | GO0006952_defense_response |
| SERPINE | serpin family E member 1 | GO0006952_defense_response |
| 1 | | |
| BMP6 | bone morphogenetic protein 6 | GO0006952_defense_response |
| JAM3 | junctional adhesion molecule 3 | GO0006952_defense_response |
| ICAM2 | intercellular adhesion molecule 2 | GO0006952_defense_response |
| SLC22A5 | solute carrier family 22 member 5 | GO0015893_drug_transport |
| ATP8B1 | ATPase phospholipid transporting | GO0015893_drug_transport |
| | 8B1 | |
| MFSD10 | major facilitator superfamily | GO0015893_drug_transport |
| | domain containing 10 | |
| SLC22A1 | solute carrier family 22 member 1 | GO0015893_drug_transport |
| ABCB4 | ATP binding cassette subfamily B | GO0015893_drug_transport |
| | member 4 | |
| SLC47A1 | solute carrier family 47 member 1 | GO0015893_drug_transport |
| SLC22A2 | solute carrier family 22 member 2 | GO0015893_drug_transport |
| SLC38A7 | solute carrier family 38 member 7 | GO0015893_drug_transport |
| SLC38A1 | solute carrier family 38 member 1 | GO0015893_drug_transport |
| SLC19A3 | solute carrier family 19 member 3 | GO0015893_drug_transport |
| SLC19A2 | solute carrier family 19 member 2 | GO0015893_drug_transport |
| SLC38A3 | solute carrier family 38 member 3 | GO0015893_drug_transport |
| MUC6 | mucin 6, oligomeric mucus/gel- | GO0022600_digestive_system_process |
| | forming | |
| SLC22A5 | solute carrier family 22 member 5 | GO0022600_digestive_system_process |
| AP0A4 | apolipoprotein A-IV | GO0022600_digestive_system_process |
| CD36 | CD36 molecule | GO0022600_digestive_system_process |
| NR1H3 | nuclear receptor subfamily 1 | GO0022600_digestive_system_process |
| | group H member 3 | |
| AKR1C1 | aldo-keto reductase family 1, | GO0022600_digestive_system_process |
| | member C1 | |
| TAC4 | tachykinin 4 (hemokinin) | GO0022600_digestive_system_process |
| WNK4 | WNK lysine deficient protein | GO0022600_digestive_system_process |
| | kinase 4 | |
| MUC4 | mucin 4, cell surface associated | GO0022600_digestive_system_process |
| AC01 | aconitase 1 | GO0022600_digestive_system_process |
| OPRL1 | opioid related nociceptin receptor | GO0022600_digestive_system_process |
| | 1 | |
| PAWR | pro-apoptotic WT1 regulator | GO0022600_digestive_system_process |
| NKX3-1 | NK3 homeobox 1 | GO0050678_regulation_of_epithelial_cell_proliferation |
| AGER | advanced glycosylation end | GO0050678_regulation_of_epithelial_cell_proliferation |
| | product-specific receptor | |
| MAP2K5 | mitogen-activated protein kinase | GO0050678_regulation_of_epithelial_cell_proliferation |
| | kinase 5 | |
| ITGB3 | integrin subunit beta 3 | GO0050678_regulation_of_epithelial_cell_proliferation |
| SPARC | secreted protein acidic and | GO0050678_regulation_of_epithelial_cell_pro1iferation |
| | cysteine rich | |
| BMP6 | bone morphogenetic protein 6 | GO0050678_regulation_of_epithelial_cell_proliferation |
| CD109 | CD109 molecule | GO0050678_regulation_of_epithelial_cell_proliferation |
| ACVRL1 | activin A receptor like type 1 | GO0050678_regulation_of_epithelial_cell_proliferation |
| ENG | endoglin | GO0050678_regulation_of_epithelial_cell_proliferation |
| KDR | kinase insert domain receptor | GO0050678_regulation_of_epithelial_cell_proliferation |
| CAV1 | caveolln 1 | GO0050678_regulation_of_epithelial_cell_proliferation |
| FLT4 | fms related tyrosine kinase 4 | GO0050678_regulation_of_epithelial_cell_proliferation |
| EGFL7 | EGF like domain multiple 7 | GO0050678_regulation_of_epithelial_cell_proliferation |
| CCL2 | C-C motif chemokine ligand 2 | GO0050678_regulation_of_epithelial_cell_proliferation |
| PLAU | plasminogen activator, urokinase | GO0050678_regulation_of_epithelial_cell_proliferation |
| MYDGF | myeloid-derived growth factor | GO0050678_regulation_of_epithelial_cell_pro1iferation |
| HTR2B | 5-hydroxytryptamine receptor 2B | GO0050678_regulation_of_epithelial_cell_proliferation |
| A4GNT | alpha-1,4-N- | GO0050678_regulation_of_epithelial_cell_proliferation |
| | acetylglucosaminyltransferase | |
| C2 | complement component 2 | GO0007584_response_to_nutrient |
| TTPA | tocopherol (alpha) transfer protein | GO0007584_response_to_nutrient |
| LIPG | lipase G, endothelial type | GO0007584_response_to_nutrient |
| PAWR | pro-apoptotic WT1 regulator | GO0007584_response_to_nutrient |
| OGT | O-linked N-acetylglucosamine | GO0007584_response_to_nutrient |
| | (GlcNAc) transferase | |
| VLDLR | very low density lipoprotein | GO0007584_response_to_nutrient |
| | receptor | |
| SPARC | secreted protein acidic and | GO0007584_response_to_nutrient |
| | cysteine rich | |
| CAV1 | caveolin 1 | GO0007584_response_to_nutrient |
| TXN2 | thioredoxin 2 | GO0007584_response_to_nutrient |
| ALDH1A2 | aldehyde dehydrogenase 1 family | GO0007584_response_to_nutrient |
| | member A2 | |
| MOG | myelin oligodendrocyte | GO0007584_response_to_nutrient |
| | glycoprotein | |
| OXCT1 | 3-oxoacid CoA-transferase 1 | GO0007584_response_to_nutrient |
| PIM1 | Pim-1 proto-oncogene, | GO0007584_response_to_nutrient |
| | serine/threonine kinase | |
| OTC | ornithine carbamoyltransferase | GO0007584_response_to_nutrient |

SUPPLEMENTARY TABLE 2
Biological processes and GO terms of Differentially Expressed
Genes in Duodenal Organoids vs. Adult Duodenum.

| | | Fold | | Corrected |
|---|---|---|---|---|
| Biological Process | GO Term | Enrichmcnt | +/− | P-value |
| digestion | GO:0007586 | 4.06 | + | 4.06E−05 |
| extracellular matrix organization | GO:0030198 | 3.52 | + | 4.74E−14 |
| cell-matrix adhesion | GO:0007160 | 3.04 | + | 4.47E−02 |
| blood vessel morphogenesis | GO:0048514 | 2.60 | + | 2.04E−07 |
| cellular response to inorganic substance | GO:0071241 | 2.50 | + | 2.75E−02 |
| wound healing | GO:0042060 | 2.49 | + | 9.00E−08 |
| cellular response to wounding | GO.0009611 | 2.43 | + | 3.83E−09 |
| cell adhesion | GO:0007155 | 2.22 | + | 8.69E−12 |
| tube morphogenesis | GO:0035295 | 2.02 | + | 5.49E−07 |
| cell migration | GO:0016477 | 2.03 | + | 2.43E−08 |
| response to toxic substance | GO:0009636 | 1.96 | + | 1.59E−02 |
| regulation of response to external stimulus | GO:0032101 | 1.82 | + | 7.77E−04 |
| anatomical structure morphogenesis | GO:0009653 | 1.80 | + | 5.03E−14 |
| tissue development | GO:0009888 | 1.66 | + | 5.09E−07 |
| response to drug | GO:0042493 | 1.66 | + | 2.27E−02 |
| secretion | GO:0046903 | 1.63 | + | 9.93E−03 |
| response to external stimulus | GO:0009605 | 1.53 | + | 6.86E−05 |
| ion transport | GO:0006811 | 1.53 | + | 3.55E−02 |
| system development | GO:0048731 | 1.47 | + | 4.50E−12 |
| localization | GO:0051179 | 1.27 | + | 7.12E−05 |
| RNA metabolic proces | GO:0016070 | 0.53 | − | 4.63E−12 |
| nucleic acid metabolic process | GO:0090304 | 0.52 | − | 2.85E−16 |
| RNA processing | GO:0006396 | 0.21 | − | 2.80E−08 |

TABLE 2
Differentially Expressed Genes in Duodenal Organoids vs. Adult Duodenum, Related to FIG. 3B.

| | Gene | | OrgDuo_1 | OrgDuo_2 | OrgDuo_3 | AdultDuo_1 | AdultDuo_2 | | adj | |
|---|---|---|---|---|---|---|---|---|---|---|
| No | Symbol | Description | FPKM | FPKM | FPKM | FPKM | FPKM | p_value | p_value | logFC |
| 1 | HLA- | major histo- | 0.0512 | 0.0474 | 0.0432 | 218.542 | 255.202 | 2.19E−08 | 0.00167 | −7.05572 |
| | DPA1 | compatibility | | | | | | | | |
| | | complex, class | | | | | | | | |
| | | II, DQ beta | | | | | | | | |
| | | I(HLA-DQB1) | | | | | | | | |
| 2 | EEFIG | EGF containing fibulin | 0.183 | 0.2564 | 0.3228 | 1082.44 | 1317.28 | 3.69E−08 | 0.000167 | −7.04608 |
| | | like extracellular | | | | | | | | |
| | | matrix prptein | | | | | | | | |
| | | I(EFEMP [symbol] ) | | | | | | | | |
| 3 | KLK6 | keratin 12(KRT12) | 41.7733 | 41.8318 | 70.8024 | 0.145806 | 0.128211 | 4.13E−08 | 0.000167 | 8.194658 |
| 4 | TXNDC5 | U2 small nuclear RNA | 1.5036 | 2.0076 | 1.6392 | 243.476 | 249.134 | 5.32E−08 | 0.000167 | −4.08578 |
| | | auxiliary factor | | | | | | | | |
| | | I(U2AF [symbol] ) | | | | | | | | |
| 5 | PLCD3 | pleckstren homology | 58.0248 | 50.866 | 57.5498 | 3.90012 | 3.6906 | 6.29E−08 | 0.000167 | 3.646078 |
| | | domain containing | | | | | | | | |
| | | N [symbol] (PLEKN [symbol] ) | | | | | | | | |
| 6 | CD68 | CD7 molecule(CD7) | 1.1208 | 1.4714 | 1.565 | 221.647 | 218.338 | 1.21E−07 | 0.000235 | −4.18044 |
| 7 | [symbol] L [symbol] E41 | basic helix-loop-helix | 36.3788 | 39.3067 | 32.4307 | 2.62998 | 2.86326 | 1.30E−07 | 0.000235 | 3.644955 |
| | | family member | | | | | | | | |
| | | [symbol] ( [symbol] L [symbol] E41) | | | | | | | | |
| 8 | DCUN1D5 | di [symbol] coidin domain | 13.1604 | 12.2298 | 17.0124 | 1.19277 | 1.10167 | 1.42E−07 | 0.000235 | 3.817746 |
| | | recepter tr [symbol] ine | | | | | | | | |
| | | kinase 2(DDR2) | | | | | | | | |
| 9 | E [symbol] A1 | Extracellular leucine | 3.1824 | 3.9919 | 3.6915 | 188.34 | 243.698 | 2.70E−07 | 0.000291 | −3.28121 |
| | | [symbol] ich [symbol] epeat and | | | | | | | | |
| | | fibronection type III | | | | | | | | |
| | | domain containing | | | | | | | | |
| | | 2(ELFN2) | | | | | | | | |
| 10 | FNDC4 | FosB proto-oncogene, | 10.6333 | 10.0502 | 9.0663 | 1.10926 | 1.10947 | 2.77E−07 | 0.000291 | 3.606099 |
| | | AP [symbol] transcription | | | | | | | | |
| | | factor subunit(FOSB) | | | | | | | | |
| 11 | AQP10 | aquaporin 10(AQP10) | 0.0909 | 0.1717 | 0.1069 | 89.4573 | 64.9797 | 2.93E−07 | 0.000291 | 5.21557 |
| 12 | CD36 | CD3e molecule | 0.1171 | 0.0836 | 0.049 | 89.4558 | 100.847 | 3.08E−07 | 0.000291 | −5.76206 |
| | | associated | | | | | | | | |
| | | protein(CD3EAP) | | | | | | | | |
| 13 | APOA4 | apolipoprotein | 15.1045 | 16.5772 | 20.5891 | 2344.59 | 2206.07 | 3.15E−07 | 0.000291 | 3.95867 |
| | | A4(APOA4) | | | | | | | | |
| 14 | MZB1 | N(alpha)-acetyl- | 0.146 | 0.1809 | 0.2467 | 117.397 | 132.236 | 3.20E−07 | 0.000291 | 5.3072 |
| | | transferase | | | | | | | | |
| | | 38, NatC au [symbol] liary | | | | | | | | |
| | | subunit(NAA38) | | | | | | | | |
| 15 | MYEOV | myosin heavy chain | 30.7906 | 21.9561 | 16.8681 | 0.3014 | 0.387316 | 3.77E−07 | 0.000291 | 6.086302 |
| | | 15(MY [symbol] 5) | | | | | | | | |
| 16 | PLVAP | plexin domain | 0.001 | 0.001 | 0.001 | 123.604 | 53.1941 | 3.79E−07 | 0.000291 | −8.74773 |
| | | containing | | | | | | | | |
| | | 1(PLXDC1) | | | | | | | | |
| 17 | ALPI | alkaline phosphatase, | 2.9125 | 2.6429 | 1.9936 | 271.842 | 200.168 | 4.23E−07 | 0.000291 | 3.70978 |
| | | intestinal(ALPI) | | | | | | | | |
| 18 | TSPAN [symbol] | tetraspanin | 373.2637 | 342.0591 | 402.6593 | 35.245 | 32.1228 | 4.26E−07 | 0.000291 | 3.301212 |
| | | [symbol] (TSPAN [symbol] ) | | | | | | | | |
| 19 | ANKRD22 | ankyrin repeat | 27.2451 | 22.628 | 22.3339 | 2.73728 | 2.15772 | 4.48E− 07 | 0.000291 | 3.409654 |
| | | domain | | | | | | | | |
| | | 22(ANKRD22) | | | | | | | | |
| 20 | KRT7 | keratin 80(KRT80) | 181.1735 | 86.6288 | 116.1648 | 0.502242 | 0.542924 | 4.58E−07 | 0.000291 | 7.295143 |
| 21 | PRSSI2 | protease, [symbol] ine | 24.0727 | 26.9325 | 26.3468 | 3.20939 | 2.44815 | 4.72E−07 | 0.000291 | 1.286194 |
| | | 22(PRSS22) | | | | | | | | |
| 22 | GMFG | G protein subunit | 0.0287 | 0.0368 | 0.0173 | 34.6824 | 28.4595 | 4.90E−07 | 0.000291 | 5.56479 |
| | | alpha [symbol] (GNAO [symbol] ) | | | | | | | | |
| 23 | MATR3 | MCM3AP | 0.4525 | 0.4059 | 0 2877 | 42.1975 | 54.3283 | 5.15E−07 | 0.000291 | 3.8058 |
| | | antisense RNA | | | | | | | | |
| | | [symbol] (MCM3AP [symbol] AS [symbol] ) | | | | | | | | |
| 24 | COL6A2 | collagen type VI alpha | 0.0048 | 0.0076 | 0.01 | 89.1592 | 111.455 | 5.48E−07 | 0.000291 | −7.71204 |
| | | 3 chain(COL6A3) | | | | | | | | |
| 25 | GIMAP7 | GIMAP family P [symbol] oop | 0.001 | 0.001 | 0.001 | 18.4391 | 19 7819 | 5.75E−07 | 0.000291 | 7.30825 |
| | | NTPase domain | | | | | | | | |
| | | containing | | | | | | | | |

[symbol]

indicates data missing or illegible when filed

TABLE 3
Biological processes and GO terms of Differentially Expressed
Genes in Organoids compared to Adult Duodenum. 1437 genes differently
expressed in Organoids compared to Adult Duodenum.

| | | Fold | | Adjusted |
|---|---|---|---|---|
| Biological Process | GO Term | Enrichment | +/− | p-value |
| digestion | GO:0007586 | 4.06 | + | 4.06E−05 |
| extracellular matrix organization | GO:0030198 | 3.52 | + | 4.74E−14 |
| cell-matrix adhesion | GO:0007160 | 3.04 | + | 4.47E−02 |
| blood vessel morphogenesis | GO:0048514 | 2.60 | + | 2.04E−07 |
| cellular response to inorganic | GO:0071241 | 2.50 | + | 2.75E−02 |
| substance | | | | |
| wound healing | GO:0042060 | 2.49 | + | 9.00E−08 |
| cellular response to wounding | GO:0009611 | 2.43 | + | 3.83E−09 |
| cell adhesion | GO:0007155 | 2.22 | + | 8.69E−12 |
| tube morphogenesis | GO:0035295 | 2.02 | + | 5.49E−07 |
| cell migration | GO:0016477 | 2.03 | + | 2.43E−08 |
| response to toxic substance | GO:0009636 | 1.96 | + | 1.59E−02 |
| regulation of response to external | GO:0032101 | 1.82 | + | 7.77E−04 |
| stimulus | | | | |
| anatomical structure morphogenesis | GO:0009653 | 1.80 | + | 5.03E−14 |
| tissue development | GO:0009888 | 1.66 | + | 5.09E−07 |
| response to drug | GO:0042493 | 1.66 | + | 2.27E−02 |
| secretion | GO:0046903 | 1.63 | + | 9.93E−03 |
| response to external stimulus | GO:0009605 | 1.53 | + | 6.86E−05 |
| ion transport | GO:0006811 | 1.53 | + | 3.55E−02 |
| system development | GO:0048731 | 1.47 | + | 4.50E−12 |
| localization | GO:0051179 | 1.27 | + | 7.12E−05 |
| RNA metabolic proces | GO:0016070 | 0.53 | − | 4.63E−12 |
| nucleic acid metabolic process | GO:0090304 | 0.52 | − | 2.85E−16 |
| RNA processing | GO:0006396 | 0.21 | − | 2.80E−08 |

TABLE 3S
Biological processes and GO terms of Differentially Expressed
Genes in Duodenum-chip vs. Adult Duodenum. 1023 genes
differently expressed in Duodenum Intestine-Chip compared
to Adult Duodenum Related to Supplementary FIG. 3A.

| | | Fold | | Adjusted |
|---|---|---|---|---|
| Biological Process | GO Term | Enrichment | +/− | p-value |
| protein targeting to ER | GO:0045047 | 5 59 | + | 7.25E−08 |
| cytoplasmic translation | GO:0002181 | 5 29 | + | 1.76E−03 |
| translational initiation | GO:0006413 | 4.49 | + | 6.85E−07 |
| protein targeting to | GO:0006612 | 3.99 | + | 3.10E−05 |
| membrane | | | | |
| response to interferon- | GO:0034341 | 3.04 | + | 2.07E−02 |
| gamma | | | | |
| mRNA catabolic process | GO:0006402 | 2.81 | + | 3.42E−01 |
| translation | GO:0006412 | 2.30 | + | 2.26E−02 |
| protein localization to | GO:0072657 | 2.29 | + | 4.49E−03 |
| membrane | | | | |
| peptide biosynthetic process | GO:0043043 | 2.21 | + | 4.72E−02 |
| regulation of cell motility | GO:2000145 | 2.13 | + | 4.54E−06 |
| cell migration | GO:0016477 | 2.06 | + | 9.61E−06 |
| locomotion | GO:0040011 | 1.94 | + | 1.11E−06 |
| cell motility | GO:0048870 | 1.93 | + | 7.01E−05 |
| regulation of cell | GO:0042127 | 1.70 | + | 1.23E−04 |
| proliferation | | | | |
| cell surface receptor | GO:0007166 | 1.63 | + | 1.37E−06 |
| signaling pathway | | | | |
| immune response | GO:0006955 | 1.60 | + | 1.59E−03 |
| immune system process | GO:0002376 | 1.58 | + | 1.92E−06 |
| regulation of cell | GO:0010646 | 1.44 | + | 7.69E−05 |
| communication | | | | |
| regulation of signaling | GO:0023051 | 1.43 | + | 1.83E−04 |

Supplementary Table 3.

Differentially Expressed Genes in Duodenum-chip vs. Adult Duodenum.

Supplementary Table 4.

Differentially Expressed Genes in Duodenum-chip vs. Duodenal Organoids, Related to FIG. 5.

Supplementary Table 5.

Differentially Expressed Genes in Adult Duodenum vs. Duodenal Organoids, Related to FIG. 7.

Supplementary Table 6.

Differentially Expressed Genes Common in Duodenum-Chip and Adult Duodenum versus Duodenal Organoids, Related to FIG. 7.

SUPPLEMENTARY TABLE 7
Enriched GO Terms from a List of Differentially Expressed Genes Common in Duodenum-
Chip and Adult Intestine versus Duodenal Organoids, Related to FIG. 7.

| | | | Bonferroni | Eliminated |
|---|---|---|---|---|
| GO term | Description | Enrichment | p-value | in REVIGO |
| GO:0007586 | digestion | 12.45 | 2.86E−11 | 0 |
| GO:0016070 | RNA metabolic process | 0.16 | 0.00305 | 0 |
| GO:0090304 | nucleic acid metabolic process | 0.26 | 0.00249 | 0 |
| GO:0043062 | extracellular structure organization | 5.53 | 1.7E−09 | 0 |
| GO:0065008 | regulation of biological quality | 1.96 | 1.73E−09 | 0 |
| GO:0050896 | response to stimulus | 1.51 | 1.33E−08 | 0 |
| GO:0010273 | detoxification of copper ion | 37.82 | 0.000000404 | 1 |
| GO:1990169 | stress response to copper ion | 37.82 | 0.000000404 | 0 |
| GO:0071280 | cellular response to copper ion | 24.01 | 0.00000114 | 1 |
| GO:0015850 | organic hydroxy compound | 8.22 | 0.00000131 | 0 |
| | transport | | | |
| GO:0061687 | detoxification of inorganic | 35.59 | 0.00000061 | 0 |
| | compound | | | |
| GO:0097501 | stress response to metal ion | 33.61 | 0.000000903 | 0 |
| GO:0032501 | multicellular organismal process | 1.51 | 0.0000237 | 0 |
| GO:0042221 | response to chemical | 1.74 | 0.00000829 | 0 |
| GO:0071294 | cellular response to zinc ion | 25.21 | 0.00000688 | 1 |
| GO:0071276 | cellular response to cadmium ion | 15.92 | 0.000203 | 1 |
| GO:0044281 | small molecule metabolic process | 2.37 | 0.00000125 | 0 |
| GO:0009653 | anatomical structure morphogenesis | 2.15 | 0.0000319 | 1 |
| GO:0015711 | organic anion transport | 4.18 | 0.0000102 | 0 |
| GO:0022600 | digestive system process | 11.52 | 0.000023 | 1 |
| GO:0051239 | regulation of multicellular | 1.85 | 0.000283 | 0 |
| | organismal process | | | |
| GO:0046688 | response to copper ion | 15.28 | 0.0000468 | 1 |
| GO:0042632 | cholesterol homeostasis | 10.34 | 0.0000686 | 0 |
| GO:0030198 | extracellular matrix organization | 4.83 | 0.0000141 | 0 |
| GO:0055092 | sterol homeostasis | 10.21 | 0.000078 | 1 |
| GO:0006820 | anion transport | 3.84 | 0.00000414 | 0 |
| GO:0006882 | cellular zinc ion homeostasis | 18.33 | 0.0000708 | 0 |
| GO:0046686 | response to cadmium ion | 10.34 | 0.00124 | 1 |
| GO:0033344 | cholesterol efflux | 23.38 | 0.0001 | 1 |
| GO:0055069 | zinc ion homeostasis | 16.81 | 0.000135 | 1 |
| GO:0071248 | cellular response to metal ion | 5.34 | 0.00319 | 1 |
| GO:0001525 | angiogenesis | 4.92 | 0.00001 | 1 |
| GO:0010038 | response to metal ion | 3.9 | 0.00228 | 1 |
| GO:0032879 | regulation of localization | 1.94 | 0.000102 | 0 |
| GO:0070887 | cellular response to chemical | 1.85 | 0.00118 | 1 |
| | stimulus | | | |
| GO:0040012 | regulation of locomotion | 2.82 | 0.0000619 | 0 |
| GO:2000145 | regulation of cell motility | 2.91 | 0.0000678 | 1 |
| GO:0010043 | response to zinc ion | 12 | 0.000353 | 0 |
| GO:0009636 | response to toxic substance | 3.83 | 0.0000307 | 1 |
| GO:0009611 | response to wounding | 3.34 | 0.000497 | 0 |
| GO:0051241 | negative regulation of multicellular | 2.53 | 0.000315 | 0 |
| | organismal process | | | |
| GO:0051186 | cofactor metabolic process | 3.52 | 0.000589 | 0 |
| GO:0010035 | response to inorganic substance | 3.34 | 0.00152 | 0 |
| GO:0001944 | vasculature development | 3.57 | 0.000239 | 1 |
| GO:0006766 | vitamin metabolic process | 7.28 | 0.000725 | 0 |
| GO:0006869 | lipid transport | 4.37 | 0.00165 | 0 |
| GO:0048731 | system development | 1.63 | 0.00138 | 1 |
| GO:0071241 | cellular response to inorganic | 4.73 | 0.0131 | 0 |
| | substance | | | |
| GO:0051I79 | localization | 1.51 | 0.00194 | 0 |
| GO:0006950 | response to stress | 1.73 | 0.00162 | 0 |
| GO:0048856 | anatomical structure development | 1.53 | 0.00237 | 0 |
| GO:0072358 | cardiovascular system development | 3.5 | 0.00035 | 0 |
| GO:0035295 | tube development | 2.9 | 0.000191 | 0 |
| GO:0050793 | regulation of developmental process | 1.81 | 0.0148 | 1 |
| GO:0009888 | tissue development | 2.15 | 0.00113 | 1 |
| GO:0030334 | regulation of cell migration | 2.79 | 0.00115 | 1 |
| GO:0010876 | lipid localization | 4.28 | 0.00108 | 0 |
| GO:0008643 | carbohydrate transport | 10.9 | 0.0000403 | 0 |
| GO:0048514 | blood vessel morphogenesis | 4 | 0.000195 | 1 |
| GO:0048878 | chemical homeostasis | 2.51 | 0.00176 | 0 |
| GO:0098754 | detoxification | 7.6 | 0.000459 | 0 |
| GO:0042060 | wound healing | 3.42 | 0.00313 | 0 |
| GO:0051270 | regulation of cellular component | 2.67 | 0.00063 | 1 |
| | movement | | | |
| GO:0006575 | cellular modified amino acid | 5.31 | 0.0034 | 0 |
| | metabolic process | | | |
| GO:0006811 | ion transport | 2.39 | 0.000561 | 0 |
| GO:0001568 | blood vessel development | 3.6 | 0.000372 | 1 |
| GO:0030301 | cholesterol transport | 10.43 | 0.00492 | 1 |
| GO:0006641 | triglyceride metabolic process | 8.96 | 0.0041 | 0 |
| GOO032502 | developmental process | 1.48 | 0.011 | 0 |
| GO:0008202 | steroid metabolic process | 4.55 | 0.00436 | 0 |
| GO:0035239 | tube morphogenesis | 3.14 | 0.000625 | 0 |
| GO:0072359 | circulatory system development | 2.68 | 0.003 | 1 |
| GO:0045926 | negative regulation of growth | 4.38 | 0.00726 | 0 |
| GO:0055088 | lipid homeostasis | 6.35 | 0.00881 | 0 |
| GO:0034371 | chylomicron remodeling | 42.02 | 0.00668 | 1 |
| GO:0006721 | terpenoid metabolic process | 8.17 | 0.000211 | 0 |
| GO:0015918 | sterol transport | 8.9 | 0.0163 | 1 |
| GO:0002576 | platelet degranulation | 6.3 | 0.00951 | 0 |
| GO:1901615 | organic hydroxy compound | 3.56 | 0.00279 | 0 |
| | metabolic process | | | |
| GO:0050892 | intestinal absorption | 15.69 | 0.00837 | 1 |
| GO:0001523 | retinoid metabolic process | 9.06 | 0.000259 | 1 |
| GO:0007275 | multicellular organism development | 1.5 | 0.0384 | 1 |
| GO:0046916 | cellular transition metal ion | 6.72 | 0.015 | 1 |
| | homeostasis | | | |
| GO:0042445 | hormone metabolic process | 5.63 | 0.000652 | 0 |
| GO:0022603 | regulation of anatomical structure | 2.39 | 0.0186 | 1 |
| | morphogenesis | | | |
| GO:0007155 | cell adhesion | 2.5 | 0.0186 | 0 |
| GO:0022610 | biological adhesion | 2.48 | 0.0201 | 0 |
| GO:0016999 | antibiotic metabolic process | 6.07 | 0.0138 | 0 |
| GO:0046903 | secretion | 2.39 | 0.00625 | 0 |
| GO:0016101 | diterpenoid metabolic process | 8.49 | 0.000497 | 1 |
| GO:0051180 | vitamin transport | 12.72 | 0.0285 | 0 |
| GO:0097006 | regulation of plasma lipoprotein | 8.4 | 0.0251 | 0 |
| | particle levels | | | |
| GO:0006639 | acylglycerol metabolic process | 6.86 | 0.0377 | 1 |
| GO:0032101 | regulation of response to external | 2.8 | 0.00405 | 0 |
| | stimulus | | | |
| GO:0016042 | lipid catabolic process | 3.95 | 0.0269 | 0 |
| GO:0006638 | neutral lipid metabolic process | 6.79 | 0.041 | 0 |
| GO:0006810 | transport | 1.55 | 0.0225 | 0 |
| GO:0044241 | lipid digestion | 28.01 | 0.0306 | 0 |
| GO:0034370 | triglyceride-rich lipoprotein particle | 28.01 | 0.0306 | 0 |
| | remodeling | | | |
| GO:0048513 | animal organ development | 1.68 | 0.0385 | 1 |
| GO:0006720 | isoprenoid metabolic process | 6.94 | 0.00122 | 0 |
| GO:0051234 | establishment of localization | 1.52 | 0.0404 | 1 |

SUPPLEMENTARY TABLE 8
Key Resources Table. List of primary antibodies used for immunostaining.

| Antibody for | | | | Dilution | |
|---|---|---|---|---|---|
| Target Protein | | Vendor | | IF = | |
| or compound | Host | Source or reference | Catalog Number | immunofluorescence | |
| BCRP | Mouse | EMD Millipore | MAB4155 | 1:100 | |
| | | | | IF(1:100) | |
| Chromogranin A | Goat | Santa Cruz | sc-1488 | 1:100 | |
| | | Biotechnology | | IF(1:100) | |
| E-Cadherin | Mouse | Abcam | ab1416 | 1:100 | |
| | | | | IF(1:100) | |
| Lysozyme | Rabbit | Dako | A0099 | 1:500 | |
| | | Agilent | | IF(1:500) | |
| MDR-1 (P-gp) | Mouse | Thermo Fisher | MA5-13854 | 1:100 | |
| | | Scientific | | IF(1:100) | |
| Mucin-2 | Mouse | Santa Cruz | sc-7314 | 1:400 | |
| | | Biotechnology | | IF(1:400) | |
| | | Thermo | | | |
| | | Fisher | | | |
| | | Scientific | | | |
| PEPT1 | Mouse | Santa Cruz | sc-373742 | 1:100 | |
| | | Biotechnology | | IF(1:100) | |
| | | Thermo | | | |
| | | Fisher | | | |
| | | Scientific | | | |
| VE-Cadherin | Rabbit | Abcam | ab232880 | 1:400 | |
| | | | | IF(1:400) | |
| Villin | Rabbit | Abcam | ab130751 | 1:100 | |
| | | | | 1F(1:100) | |
| ZO-1 | Mouse | Abcam | ab6135733- | 1:100 | |
| | | Thermo | 9100 RR1D: | IF(1:100) | |
| | | Fisher | AB_2533147 | | |
| | | Scientific | | | |
| Chemical | 3 kDa | Thermo | Cat# D7132 | 0.1 | mg/ml |
| compound, | Dextran, | Fisher | | | |
| drug | Cascade | Scientific | | | |
| | Blue | | | | |
| Chemical | 1α,25- | Sigma | Cat# D1530 | 100 | nM |
| compound, | Dihydroxy- | | | | |
| drug | vitamin D3 | | | | |
| Chemical | Rifampicin | Sigma | Cat# R35O1 | 20 | μM |
| compound, | | | | | |
| drug | | | | | |
| Chemical | Testosterone | Sigma | Cat# T1500 | 200 | μM |
| compound, | | | | | |
| drug | | | | | |

| | | | | |
|---|---|---|---|---|
| Commercial | MDR1 | Millipore | Cat# ECM910 | — |
| assay or kit | Efflux | | | |
| | Assay Kit | | | |
| Software, | Prism | GraphPad | — | — |
| algorithm | | | | |
| Software, | Fiji | — | RRID: | — |
| algorithm | | | SCR_002285 | |

SUPPLEMENTARY TABLE 9
List of human TaqMan Gene Expression Assays used for qRT-PCR.

| Gene | Gene Name | TaqMan Assay ID |
|---|---|---|
| ALPI | Alkaline Phosphatase | Hs00357579_gl |
| BCRP/ABCG2 | ATP binding cassette subfamily G member 2 | Hs01053790_ml |
| CHGA | Chromogranin A | Hs00900375_ml |
| CYP3A4 | cytochrome P450 family 3 subfamily A member 4 | Hs00604506_ml |
| EpCAM | Epithelial Cell Adhesion Molecule | Hs00901885_ml |
| LYZ | Lysozyme | Hs00426232_ml |
| MDR1/ABCB1 | ATP binding cassette subfamily B member 1 | Hs00184500_ml |
| MRP2/ABCC2 | ATP binding cassette subfamily C member 2 | Hs00960489_ml |
| MRP3/ABCC3 | ABCC3 | Hs00978452_ml |
| MUC2 | Mucin 2 | Hs00894025_ml |
| OAT2B1/ | solute earner organic anion transporter family | Hs01030343_ml |
| SLC02B1 | member 2B1 | |
| OCT1/SLC22AI | solute carrier family 22 member 1 | Hs00427552 ml |
| PEPT1/SLC15A1 | solute carrier family 15 member 1 | Hs00192639_ml |
| PXR/NR1I2 | nuclear receptor subfamily I group I member 2 | Hs01114267_ml |
| SLC40A1 | solute carrier family 40 member 1 | Hs00205888_ml |
| VDR | vitamin D (1,25-dihydroxyvitamin D3) receptor | Hs01045843_ml |

The following references are referred to in the Background and Duodenal Sections:
1 Waring, M. J. et al. An analysis of the attrition of drug candidates from four major pharmaceutical companies. Nat Rev Drug Discov 14, 475-486, doi:10.1038/nrd4609 (2015).
2 Frank, R. & Hargreaves, R. Clinical biomarkers in drug discovery and development. Nat Rev Drug Discos, 2, 566-580, doi:10.1038/nrd1130 (2003).
3 Ward, K. W. Optimizing pharmacokinetic properties and attaining candidate selection. In Reducing Drug Attrition. 73-95 (Springer-Verlag Berlin Heidelberg, 2012).
4 Pacifici, G. M. et al. Tissue distribution of drug-metabolizing enzymes in humans. Xenobiotica 18, 849-856, doi:10.3109/00498258809041723 (1988).
5 Kaminsky, L. S. & Zhang, Q. Y. The small intestine as a xenobiotic-metabolizing organ. Drug Metab Dispos 31, 1520-1525, doi:10.11241dmd.31.12.1520 (2003).
6 Benet, L. Z. & Cummins, C. L. The drug efflux-metabolism alliance: biochemical aspects. Adv Drug Delis, Rev 50 Suppl 1, S3-11 (2001).
7 Jeong, E. J., Liu, X., Jia, X., Chen, J. & Hu, M. Coupling of conjugating enzymes and efflux transporters: impact on bioavailability and drug interactions. Curr Drug Metab 6, 455-468 (2005).
8 Cummins, C. L., Mangravite, L. M. & Benet, L. Z. Characterizing the expression of CYP3A4 and efflux transporters (P-gp, MRP1, and MRP2) in CYP3A4-transfected Caco-2 cells after induction with sodium butyrate and the phorbol ester 12-O-tetradecanoylphorbol-13-acetate. Pharm Res 18, 1102-1109 (2001).
9 Glaeser, H. et al. Impact of concentration and rate of intraluminal drug delivery on absorption and gut wall metabolism of verapamil in humans. Clin Pharmacol Ther 76, 230-238, doi:10.1016/j.clpt.2004.04.013 (2004).
10 Lampen, A., Ebert, B., Stumkat, L., Jacob, J. & Seidel, A. Induction of gene expression of xenobiotic metabolism enzymes and ABC-transport proteins by PAH and a reconstituted PAH mixture in human Caco-2 cells. Biochim Biophys Acta 1681, 38-46, doi:10.1016/j.bbaexp.2004.09.010 (2004).
11 Chu, X., Bleasby, K. & Evers, R. Species differences in drug transporters and implications for translating preclinical findings to humans. Expert Opin Drug Metab Toxicol 9, 237-252, doi:10.1517/17425255.2013.741589 (2013).
12 Chanteux, H. et al. Cross-Species Differences in the Preclinical Pharmacokinetics of CT7758, an alpha4beta1/alpha4beta7 Integrin Antagonist. Drug Metab Dispos 43, 1381-1391, doi:10.1124/dmd.115.064436 (2015).
13 Nakamura, T. et al. Real-time quantitative polymerase chain reaction for MDR1, MRP1, MRP2, and CYP3A-mRNA levels in Caco-2 cell lines, human duodenal enterocytes, normal colorectal tissues, and colorectal adenocarcinomas. Drug Metab Dispos 30, 4-6 (2002).
14 Sun, D. et al. Comparison of human duodenum and Caco-2 gene expression profiles for 12,000 gene sequences tags and correlation with permeability of 26 drugs. Pharm Res 19, 1400-1416 (2002).
15 Borlak, J. & Zwadlo, C. Expression of drug-metabolizing enzymes, nuclear transcription factors and ABC transporters in Caco-2 cells. Xenobiotica 33, 927-943, doi:10.1080/00498250310001614286 (2003).
16 Eglen, R. M. & Randle, D. H. Drug Discovery Goes Three-Dimensional: Goodbye to Flat High-Throughput Screening? Assay Drug Dev Technol 13, 262-265, doi:10.1089/adt.2015.647 (2015).
17 Sato, T. et al. Long-term expansion of epithelial organoids from human colon, adenoma, adenocarcinoma, and Barrett's epithelium. Gastroenterology 141, 1762-1772, doi:10.1053/j.gastro.2011.07.050 (2011).
18 Sato, T. et al. Single Lgr5 stem cells build crypt-villus structures in vitro without a mesenchymal niche. Nature 459, 262-265, doi:10.1038/nature07935 (2009).
19 Liu, F. et al. Drug Discovery via Human-Derived Stem Cell Organoids. Front Pharmacol 7, 334, doi:10.3389/fphar.2016.00334 (2016).
20 Zhao, J. et al. A Novel Model of P-Glycoprotein Inhibitor Screening Using Human Small Intestinal Organoids. Basic Clin Pharmacol Toxicol 120, 250-255, doi:10.1111/bcpt.12680 (2017).
21 Dekkers, J. F. et al. A functional CFTR assay using primary cystic fibrosis intestinal organoids. Nat Med 19, 939-945, doi:10.1038/nm.3201 (2013).
22 van de Wetering, M. et al. Prospective derivation of a living organoid biobank of colorectal cancer patients. Cell 161, 933-945, doi:10.1016/j.cell.2015.03.053 (2015).
23 Vlachogiannis, G. et al. Patient-derived organoids model treatment response of metastatic gastrointestinal cancers. Science 359, 920-926, doi:10.1126/science.aao2774 (2018).
24 Zhang, L. et al. A novel biosensor based on intestinal 3D organoids for detecting the function of BCRP. Drug Deliv 24, 1453-1459, doi:10.1080/110717544.2017.1381199 (2017).
25 Fatehullah, A., Tan, S. H. & Barker, N. Organoids as an in vitro model of human development and disease. Nat Cell Biol 18, 246-254, doi:10.1038/ncb3312 (2016).
26 Kasendra, M. et al. Development of a primary human Small Intestine-on-a-Chip using biopsy-derived organoids. Sci Rep 8, 2871, doi:10.1038/s41598-018-21201-7 (2018).
27 Grimsrud, K. N. et al. Special population considerations and regulatory affairs for clinical research. Clin Res Regul Aff 32, 47-56, doi:10.3109/10601333.2015.1001900 (2015).
28 Kim, H. J., Huh, D., Hamilton, G. & Ingber, D. E. Human gut-on-a-chip inhabited by microbial flora that experiences intestinal peristalsis-like motions and flow. Lab Chip 12, 2165-2174, doi:10.1039/c21c40074j (2012).
29 Karam, S. M. Lineage commitment and maturation of epithelial cells in the gut. Front Biosci 4, D286-298 (1999).
30 Ritchie, M. E. et al. limma powers differential expression analyses for RNA-sequencing and microarray studies. Nucleic acids research 43, e47, doi:10.1093/nar/gkv007 (2015).
31 Mi, H., Muruganujan, A., Casagrande, J. T. & Thomas, P. D. Large-scale gene function analysis with the PANTHER classification system. Nat Protoc 8, 1551-1566, doi:10.1038/nprot.2013.092 (2013).
32 Ashburner, M. et al. Gene ontology: tool for the unification of biology. The Gene Ontology Consortium. Nature genetics 25, 25-29, doi:10.1038/75556 (2000).
33 The Gene Ontology, C. Expansion of the Gene Ontology knowledgebase and resources. Nucleic acids research 45, D331-D338, doi:10.1093/nar/gkw1108 (2017).
34 Wieck, M. M. et al. Prolonged Absence of Mechanoluminal Stimulation in Human Intestine Alters the Transcriptome and Intestinal Stem Cell Niche. Cellular and molecular gastroenterology and hepatology 3, 367-388 e361, doi:10.1016/j.jcmgh.2016.12.008 (2017).
35 Thomas, P. D. et al. PANTHER: a library of protein families and subfamilies indexed by function. Genome research 13, 2129-2141, doi:10.1101/gr.772403 (2003).
36 Kanehisa, M. & Goto, S. KEGG: kyoto encyclopedia of genes and genomes. Nucleic acids research 28, 27-30 (2000).
37 Eden, E., Lipson, D., Yogev, S. & Yakhini, Z. Discovering motifs in ranked lists of DNA sequences. PLoS Comput Biol 3, e39, doi:10.1371/journal.pcbi.0030039 (2007).
38 Supek, F., Bosnjak, M., Skunca, N. & Smuc, T. REVIGO summarizes and visualizes long lists of gene ontology terms. PLoS One 6, e21800, doi:10.1371/journal.pone.0021800 (2011).

39 Negoro, R. et al. Modeling of drug-mediated CYP3A4 induction by using human iPS cell-derived enterocyte-like cells. Biochem Biophys Res Commit 472, 631-636, doi:10.1016.j.bbrc.2016.03.012 (2016).
40 Schmiedlin-Ren, P. et al. Expression of enzymatically active CYP3A4 by Caco-2 cells grown on extracellular matrix-coated permeable supports in the presence of 1alpha,25-dihydroxyvitamin D3. Mol Pharmacol 51, 741-754 (1997).
41 Ozawa, T. et al. Generation of enterocyte-like cells from human induced pluripotent stem cells for drug absorption and metabolism studies in human small intestine. Sci Rep 5, 16479, doi:10.1038/srep16479 (2015).
42 Huh, D. et al. Microfabrication of human organs-on-chips. Nat Protoc 8, 2135-2157, doi:10.1038/nprot.2013.137 (2013).
43 Finkbeiner, S. R. et al. Transcriptome-wide Analysis Reveals Hallmarks of Human Intestine Development and Maturation In Vitro and In Vivo. Stem cell reports, doi:10.1016j.stemcr.2015.04.010 (2015).

In this study, we leveraged Organs-on-Chips technology and primary adult duodenal organoids to emulate multicellular complexity, physiological environment, native intestinal tissue architecture and functions and to create an alternative human-relevant model for the assessment of ADME of orally administered drugs. Since clinical pharmacology studies are typically conducted in adult individuals and it is well known that pharmacokinetics in children are very different than in adults with respect to drug absorption, distribution, metabolism, and elimination (Grimsrud, Sherwin et al. 2015), we used organoids isolated from adult healthy donors to develop Duodenum Intestine-Chip. Our new findings, presented here, complement the methodology and characterization of the Intestine-Chip model established in our previous report (Kasendra, Tovaglieri et al. 2018) using children biopsies-derived organoids. We demonstrate that the physiological mimicry of the Duodenum Intestine-Chip is excellent and superior to the organoids in their original form (static, not on-chip). The synergistic use of organoid-derived cells and Organs-on-Chips enables the establishment of intact tissue-tissue interface formed by adult intestinal epithelium and small intestinal microvascular endothelial cells. The application of mechanical stimulation in the form of continuous luminal and vascular flow, showed to exert beneficial effects on the intestinal tissue architecture. Increased epithelial cell height, acquisition of cobblestone-like cell morphology, formation of well-defined cell-cell junctions, and dense intestinal microvilli were attributed to the presence of flow.

We confirmed the development of comparable levels of intestinal barrier function to hydrophilic solute (dextran) across Duodenum Intestine-Chip cultures established from organoid-derived cells of three independent donors. This functional feature of intestinal tissue becomes important when studying drug uptake, efflux, and disposition in polarized organ systems. Although many studies have demonstrated that Caco-2 transwell allow good prediction of transcellular drug absorption, they showed to be unreliable in the assessment of passive diffusion of polar molecules such as hydrophilic drugs and peptides. This is related to much smaller, in respect to small intestinal villi, effective area of the monolayer and its higher tight junctional resistance (Press and Di Grandi 2008, Kumar, Karnati et al. 2010). Organoids have quickly become a model of interest in drug discovery and development applications, yet the use of organoids for the studies of intestinal permeability has been linked with major technical difficulties related to their three-dimensional structure, limited access to lumen, and the need for the use of sophisticated microinjection techniques for the application of the compound at the apical cell surface. On the other hand, Duodenum Intestine-Chip emulates complex intestinal tissue architecture, as evidenced by the presence of three-dimensional villi-like structures on scanning electron micrographs acquired on day 8 of fluidic culture, while allowing a direct exposure of apical cell surface to test compounds. This represents a major technological advantage over Caco-2 transwell and organoids and might provide a valid alternative for the preclinical studies of intestinal drug absorption.

Immunofluorescent Imaging and Gene Expression Analysis Demonstrated that Duodenum Intestine-Chip Possess Specialized Intestinal Cell Subpopulations that are Absent in Tumor-Derived Cell Lines Including Caco-2.

Subpopulations of intestinal cells are present in the Duodenum Intestine-Chip at the physiologically relevant ratios, similar to the ones observed in the native human duodenum. Expression of the markers specific for the intestinal types that naturally reside in the villus compartment—alkaline phosphatase for absorptive enterocytes, mucin 2 for goblet cells, chromogranin A for enteroendocrine cells—showed to increase up to day 8 of Duodenum Intestine-Chip culture. This has been accompanied by the decline in the expression of genes specific for crypt-residing cells-lysozyme for Paneth cells and Lgr5 for stem cells (data not shown) suggesting the acquisition of terminally differentiated cell phenotype by all of the cells present in the chip. This is in line with well-known effect of Wnt3a withdrawal on organoids-derived primary intestinal epithelium (Sato, Vries et al. 2009, Sato, Stange et al. 2011).

Because human intestinal cells do not produce significant amounts of Wnt3a, EGF, or several other growth factors essential for stem cell division and cell proliferation, removal of Wnt3a supplementation leads to a loss of Lgr5-positive cells, decreased cell proliferation, appearance of secretory cell lineages, including goblet and enteroendocrine cells, and the transformation of immature crypt-like enterocytes into differentiated nutrient-absorptive cells. The presence of differentiated cell subpopulations is critical for modelling various aspects of intestinal biology and function, including mucus production, antimicrobial response, host-microbiome interaction, secretion of intestinal hormones, nutrients digestion and absorption. Given that we have shown that the Duodenum Intestine-Chip recapitulates faithfully the physiologically relevant ratios of these cells it could be readily applied beyond ADME applications in development of new approaches and therapeutics that target specific cell populations. For example, it could serve to evaluate specific targeting of Paneth cells, which through a series of genetic studies have been implicated in inflammatory bowel disease (Xavier and Podolsky 2007, Khor, Gardet et al. 2011, Adolph, Tomczak et al. 2013, Liu, Gurram et al. 2016).

We compared global RNA expression profiles, obtained by RNA-seq, of Duodenum intestine-Chip, duodenal organoids and human native duodenal tissue in order to determine which of the two models better reflects their natural counterpart.

Surprisingly, although chips and organoids were established with the cells of the same origin (donors, source of the tissue) their transcriptomic profiles were shown to significantly differ from each other the total number of 472 up- and down-regulated genes were found in this comparison. Moreover, several different analyses demonstrated that the transcriptome of Duodenum Intestine-Chip more closely resembles global gene expression in human adult duodenum than do the organoids, strongly suggesting that Duodenum Intestine-Chip constitute a closer approximation of human in vivo tissue.

The subset of 305 genes were found to be common for Duodenum Intestine-Chip and human tissue but different from organoids, highlighting the beneficial changes in the transcriptional profile of duodenal organoids achieved by combining them with Organs-on-Chips. These genes showed to be associated with biological functions including: digestion, transport of nutrients and ions, extracellular matrix organization, wound healing, metabolism, detoxification, and tissue development.

In addition, several genes involved in drug metabolism, including but not limited to drug metabolism enzymes: CYP3A4, UGT1A4, UGT2A3, UGT2B7, UGT2B15, UGT2B17, were observed to exhibit similar pattern of expression in Duodenum Intestine-Chip and adult duodenal tissue while they differed from organoids, suggesting the improved potential of this system to study biotransformation of xenobiotics and drug-drug interactions.

Intestinal efflux and uptake transporters are key determinants of absorption and subsequent bioavailability of a large number of orally administered drugs. In vivo-like expression of the major intestinal drug transporters, including clinically relevant MDR1, BCRP and PEPT1, was demonstrated in Duodenum Intestine-Chip system.

Additionally, we confirmed their apical localization, which is known to be crucial for the unique gatekeeper function of these proteins in controlling drug access to metabolizing enzymes and excretory pathways (Shugarts and Benet 2009, Estudante, Morais et al. 2013), within the plasma membrane of intestinal epithelial cells grown on chip.

Functional assessment of MDR1 efflux revealed similar level of activity as observed in Caco-2 Intestine-Chip and its successful inhibition by vinblastine. Notably, in comparison to Caco-2 Intestine-Chip the chip system established using duodenal organoid-derived cells showed improved relative mRNA expression levels of organic anion and cation transporters: OATP2B1 and OCT1. These transporters are responsible for the uptake of numerous xenobiotics, including statins, antivirals, antibiotics, and anticancer drugs (Roth, Obaidat et al. 2012). While significantly higher expression of these proteins, in comparison to human duodenal tissue, was observed in Caco-2 model, their levels showed to be closer to in vivo in Duodenum Intestine-Chip.

Our findings suggest that Duodenum Intestine-Chip system could be applied to assess the specific contribution of efflux transporters to drug disposition, to evaluate the active transport of xenobiotics across intestinal barrier, as well as, modelling increased absorption by targeting of specific uptake transporters such as PEPT1 or OATP2B1. Evaluation of the expression, activity and drug-mediated induction of the CYP3A4, a major enzyme involved in human metabolism of xenobiotics, revealed a key advantage of Duodenum Intestine-Chip over Caco-2 models. Significantly higher and much closer to in vivo expression of CYP3A4 along with increased metabolism of testosterone (6β-hydroxylation) were observed in Duodenum Intestine-Chip in comparison to Caco-2 cells-based system. Consistent with the results of others (Negoro, Takayama et al. 2016) CYP3A4 was undetectable at the protein level in Caco-2 cells and remained unchanged upon stimulation with RIF (a PXR agonist). Caco-2 cells lack PXR expression limiting utility for drug metabolism, drug transport, and drug-drug interaction studies.

Additionally, while vitamin D3 stimulation led to a significant increase in the mRNA expression of cytochrome P450 in Caco-2 Intestine-Chip, it showed no effect on the metabolic activity of this enzyme.

In contrast, successful and reproducible modulation of the expression and enzymatic activity of CYP3A4 using agonists specific for PXR and VDR, rifampicin and vitamin D3, respectively, was achieved in Duodenum Intestine-Chips engineered individually from the organoid-derived cells of three independent donors. CYP3A4 induction levels observed were similar to those shown for intestinal slices (van de Kerkhof, de Graaf et al. 2008), suggesting suitability of this model for the studies of drug metabolism that cannot be supported by the previous Caco-2 models. Expression of CYP3A4, PXR, and VDR in Duodenum Intestine-Chip is closer to in vivo versus Caco-2-Chip. Caco-2 cells lack PXR expression limiting utility for drug metabolism, drug transport, and drug-drug interaction studies.

In conclusion, Duodenum Intestine-Chip provides a closer to in vivo model of the human duodenum, compared to organoids or Caco-2 cells, and represents a potential tool for preclinical drug assessment in a more human-relevant model. Moreover, as it is composed of cells isolated from individual patients, it could be personalized as needed, in order to assess interindividual differences in drug disposition and responses, study the effect of genetic polymorphisms on pharmacokinetics and pharmacodynamics, as well as decoupling the effect of various factors such as age, sex, disease state, and diet on metabolism, clearance, and bioavailability of xenobiotics. This system could also help us to better understand the basic biology of human intestinal tissue in healthy and disease states and potentially enable novel therapeutic development as we further our understanding of the mechanisms driving key disease phenotypes.

IV. Small-Intestine-On-Chip: Ileum (Ileum-Chip).

Establishment of Normal Human Organoids in Suspension.

In one embodiment, establishment of at least 3 normal colonic organoids and at least 3 normal small intestine (ileal) organoids in suspension capable of being thawed and regrown in culture. In one embodiment, establishment and optimized protocols for propagation and bio-banking of suspension ileal and colonic organoids. In one embodiment, establishment successfully created a mini-biobank of at least 3 normal human small intestinal organoids (enteroids) and 4 normal human colonic organoids (colonoids). Characterization of normal human organoids on-Chip: established that human organoids in suspension and on Chips express all known types of epithelial cell lineages including enterocytes, L cells, Tuft cells, Paneth cells, LGR5 cells, goblet cells; assessed intestinal permeability to small fluorescence molecule-dextran; assessed expression of MUC2 by antibody staining; compared poly(A) RNA-seq profiles of suspension organoids, gut on chip organoids, human mucosal biopsies, and human epithelial cells isolated from human biopsies.

Summary: Established Ileum-Chip and Colon-Chip platforms and confirmed: formation of intact barrier function, presence of major intestinal cell types (absorptive enterocytes, goblet cells, stem cells, enteroendocrine cells. L cells, enterochromaffin cells, Tuft cells and Paneth cells) by immunofluorescence and qPCR.

Compared these platforms with organoids in suspension at the level of imaging and gene expression analysis. Confirmed reproducibility of Ileum-Chip and Colon-Chip across 3 different donors. Performed RNAseq analysis of Colon-Chip in the presence or absence of cyclic stretch and microvascular endothelium.

A. Induction of Inflammation in Ileum-Chip.

Stimulation: Vascular exposure to TNF-α 100 U/ml (˜2 ng/ml)—that does not affect intestinal barrier function. TNFα induces increased membrane expression of I-CAM1 and MadCAM1 in intestinal microvascula endothelium.

Identification of MadCAM1 and characterization of microvascular response to inflammation in Ileum-Chip. Inflammation-Induced Expression of I-CAM1 and MadCAM1 in Ileum-Chip. Read-outs: mRNA expression of vascular adhesion molecules (I-CAM1, MadCAM1); Immunofluorescent detection of I-CAM1 and MadCAM1; Epithelial and vascular release of inflammatory chemokines using ELISA (Meso Scale Discovery). FIG. 13. FIG. 14.

Modeling of Lymphocytes Chemoattraction in Ileum-Chip: CCL25.

Ileum: In Homeostasis.

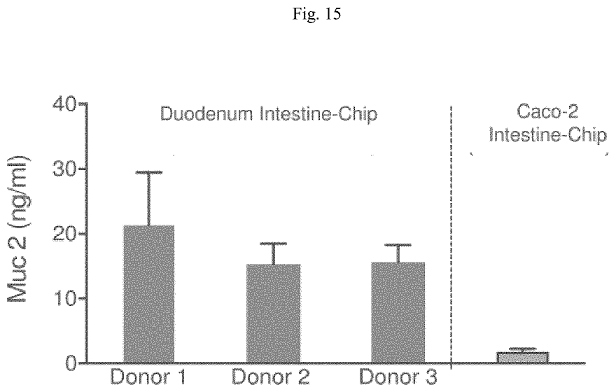

Localization of T and B cells to the small intestine is mediated by integrin a4beta7 (which binds the vascular addressin MadCAM1) and CCR9. CCR9 interacts with the chemokine CCL25 (TECK) produced by small intestinal epithelium. There is increasing evidence that CCL25 can be induced by inflammation and that this chemokine-receptor pair can promote inflammation in particular contexts. n=1,4 chips/condition. Predominantly basolateral secretion of TECK was observed in Ileum-Chip, that increases in the context of TNF-α-induced inflammation. FIG. 15.

Modeling of Lymphocytes Chemoattraction in Ileum-Chip—CXC chemokines.

Ileum: During Inflammation.

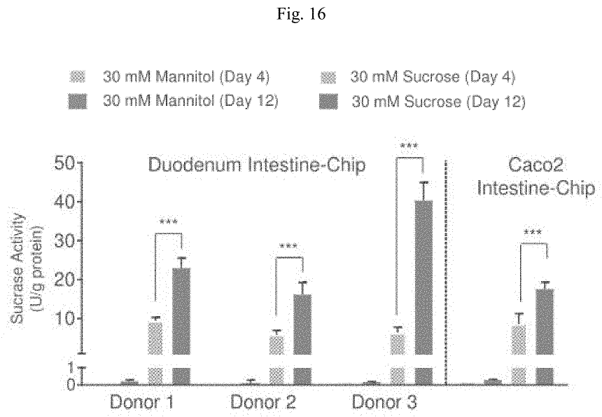

CXC chemokines such as CXCL8 (IL-8), CXCL10 (IP-10) and CXCL11 (1-TAC) are produced at the site of inflammation and contributes to the recruitment of immune cell. FIG. 16.

Modeling of Lymphocytes Chemoattraction in Ileum-Chip—CCL20.

Ileum: During Inflammation CCR6 and its Ligand CCL20 (MIP-3 Alpha) Contribute to the Localization of T Cells to the Inflamed Intestines (but not in the Absence of Inflammation). CCL20 Expression can be Induced by Inflammation.

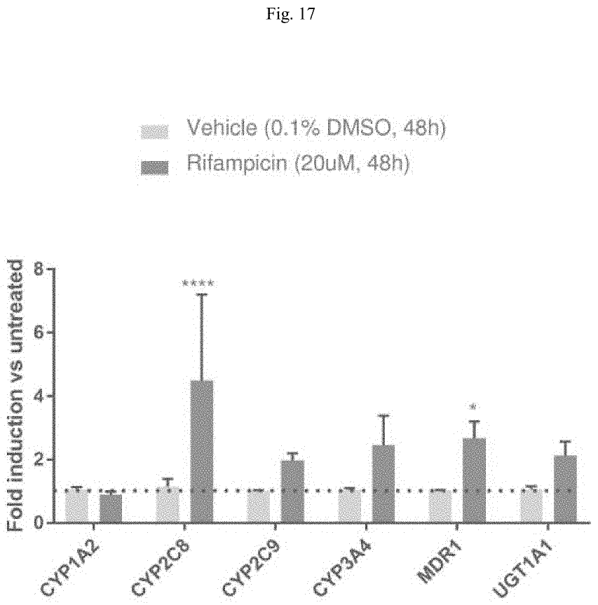

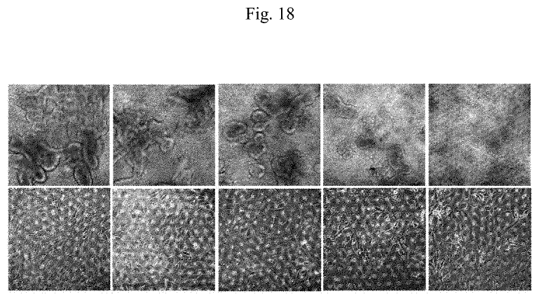

Ileum-Chip exposure to TNF-α results in the vascular release of Macrophage inflammatory protein-3 alpha (MIP-3 alpha). Surprisingly high secretion of MIP-3 alpha into the epithelial channel of Ileum-Chip. FIG. 17.

TABLE 4
Comparison Between Ileum-Chip and Microvasculature alone.

| | | Ileum-Chip | | Microvasculature alone | |
|---|---|---|---|---|---|
| | | | TNF-α | | TNF-α |
| | Chemokine | Untreated | (100 U/ml) | Untreated | (100 U/ml) |
| Apical | CCL25 (TECK) | 16.42 ± 3.6 | 17.11 ± 9.2 | ND | ND |
| | CXCL8 (IL-8) | 13.18 ± 1.9 | 16.30 ± 5.9 | 0.13 ± 0.1 | 105.5 ± 74.2 |
| | CXCLIO (IP-10) | 2.10 ± 0.9 | 6.34 ± 2.3 | 0 | 0.19 ± 0.1 |
| | CXCL11 (1- | 243.53 ± 63.5 | 302.01 ± 111.8 | 0 | 0.53 ± 1.0 |
| | TAC) | | | | |
| | CCL20 (MIP-3 | 119.65 ± 46.8 | 170.92 ± 36.8 | 0 | 0.74 ±0.9 |
| | alpha) | | | | |
| Basal | CCL25 (TECK) | 62.59 ± 10.3 | 117.91 ± 16.1 | ND | ND |
| | CXCL8 (IL-8) | 0.20 ± 0.0 | 1868.99 ± 198.7 | 1.59 ± 1.5 | 1835.44 ± 282.8 |
| | CXCLIO (IP-10) | 0.33 ± 0.2 | 502.04 ± 287.1 | 0 | 35.22 ± 20.1 |
| | CXCL11 (1- | 4.79 ± 1.9 | 26.00 ± 9.1 | 0 | 1.74 ± 1.4 |
| | TAC) | | | | |
| | CCL20 (MIP-3 | 0.78 ± 0.3 | 93.93 ± 18.3 | 0 | 3.51 ± 2.8 |
| | alpha) | | | | |

ND—not detectable.
n = 1, 4 chips/condition.

Summary of Evaluation of Microvascular Function in Ileum-Chip. We have demonstrated that Ileum-Chip responds to inflammatory stimuli (100 U/ml of TNF-α. 24 h) by: increased vascular expression of adhesion molecules (I-CAM1 and MadCAM1) and their redistribution into the endothelial cell surface; increased vascular release of inflammatory chemokines (IL-8, MIP-3 alpha, IP-10,1-TAC).

Notably, Ileum-Chip recapitulated the landscape of chemokines present in the native human intestine in homeostasis and inflammation state: constitutive secretion of lymphocyte homing molecule (TECK; CCL25) inflammation-induced vascular release of IL-8 (CXCL8), MIP-3 alpha (CCL20) and IP-10 (CXCL10) as well as polarized secretion of 1-TAC (CXCL11).

Assessment of the L-Cells Function in Ileum-Chip.

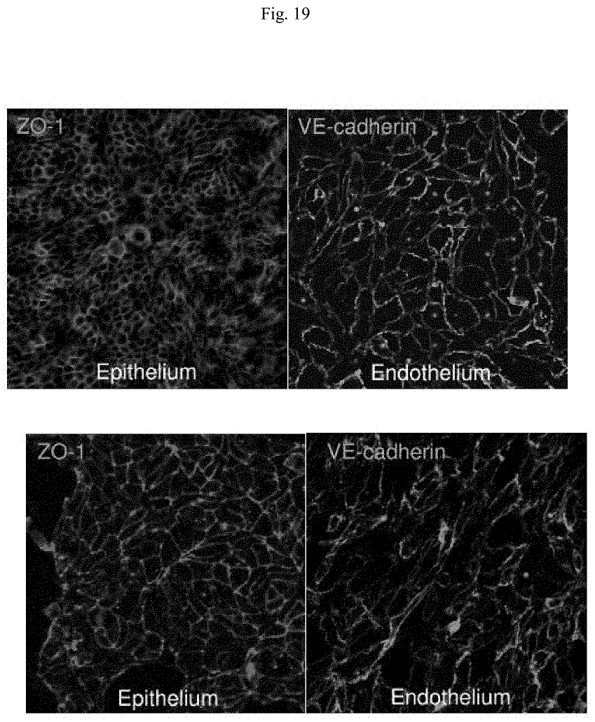

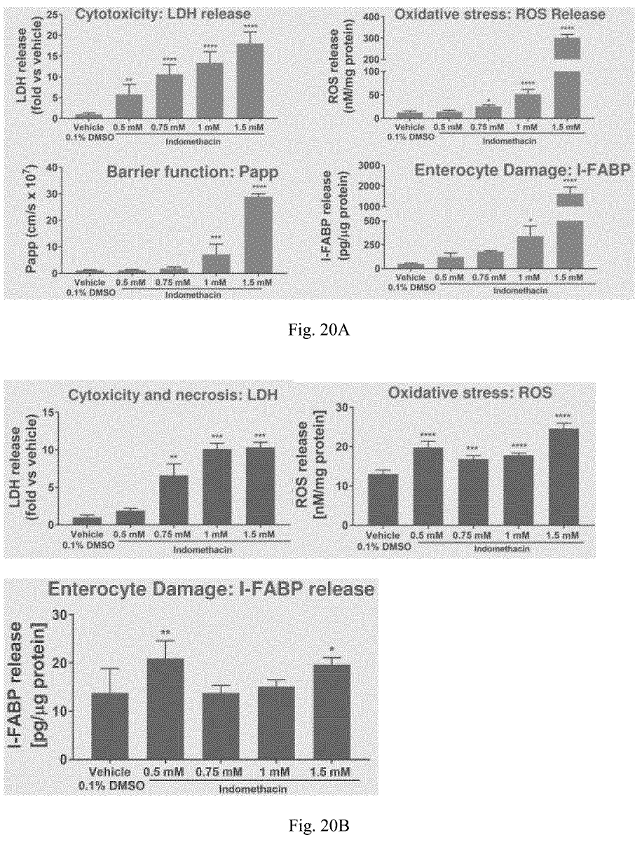

Evaluation of the Presence of L-cells in Ileum-Chip. FIG. 18. FIG. 19 Shows one embodiment of an exemplary timeline for providing L-cells in Ileum-Chip. Pre-Stimulation: Ileum-Chips is pre-exposed to 100 μM DPPIV inhibitor (diprotin A) before stimulation as well as during the course of stimulation and sampling (to prevent GLP-1 degradation).

Stimulation: Luminal exposure to SCFA mix (5 mM acetate, 1 mM propionate and 1 mM butyrate; 3 h of apical stimulation) in the presence and absence of cyclic stretch (10%, 0.15 Hz)

Read-outs: Luminal and vascular secretion as well as intracellular content of GLP-1 using GLP-1 ELISA (Meso Scale Discovery); V-PLEX Plus GLP-1 Total Kit (Dynamic range: 0.017-120 pM; Sensitivity: 0.18 pM).

L-cells Present in Intestine-Chips Are Biologically Active. FIG. 99 Shows an exemplary Predominantly basolateral release of GLP-1 was detected in Ileum-Chip. Luminal secretion and intracellular GLP-1 content remain constant, vascular release seemed to increase with stretch.

In some Ileum-Chip embodiments, the presence of highly specialized gut endocrine cells, designated L cells were detected. In some embodiments, observations indicate: L-cells are biologically active, e.g. they are able to release an intestinal hormone glucagon-like peptide (GLP-1); GLP-1 secretion is polarized—predominantly basolateral; GLP-1 release might be regulated by stretch.

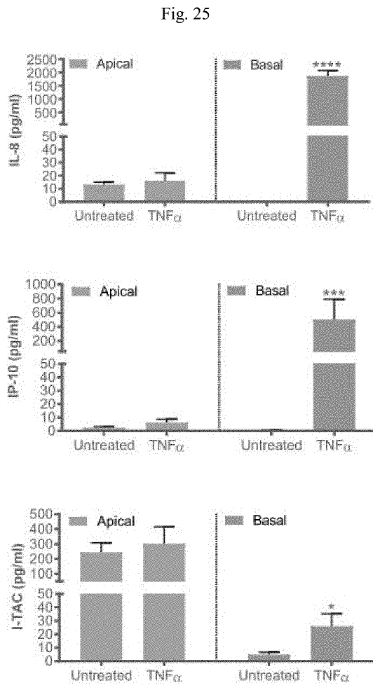

FIG. 25 Shows one embodiment of an exemplary Creation of Mini-BioBank of Ileal Enteroids: Timeline.

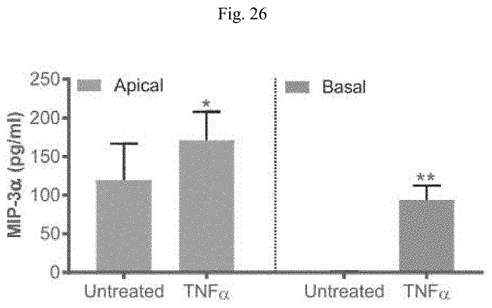

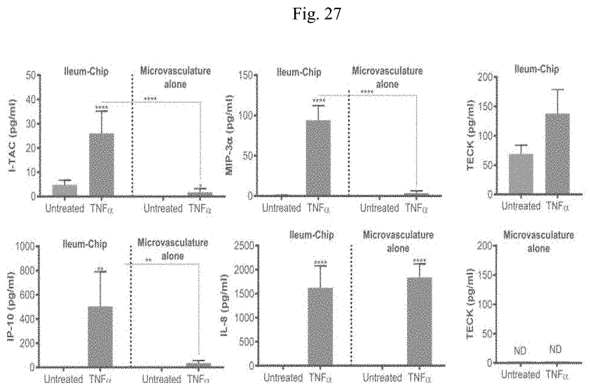

FIG. 26 Shows one embodiment of an exemplary Establishment of Intestine-Chip Using Ileal Enteroids,

FIG. 27 Shows one embodiment of an exemplary Experimental Timeline: Ileum.

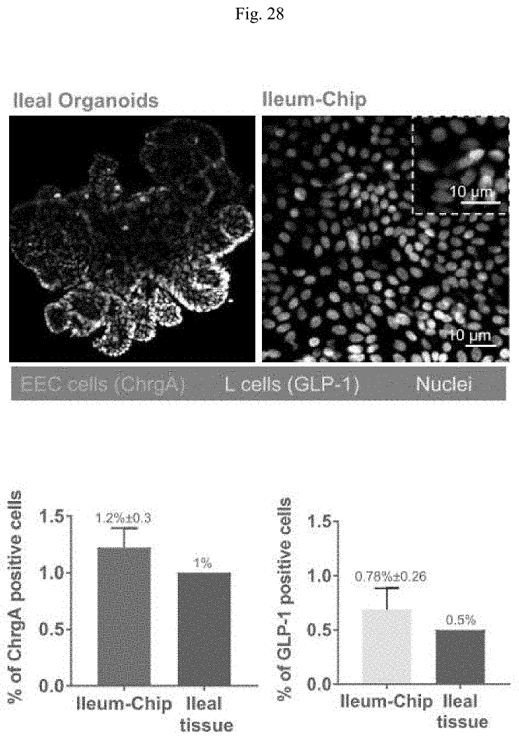

FIG. 28 Shows an exemplary 3D Tissue Architecture: Formation of Intestinal Villi-like Structures: Ileum. Morphogenesis of villi-like structures in Ileum-Chip is achieved across entire length of the epithelial channel. Representative images from day 8 of growth are shown.

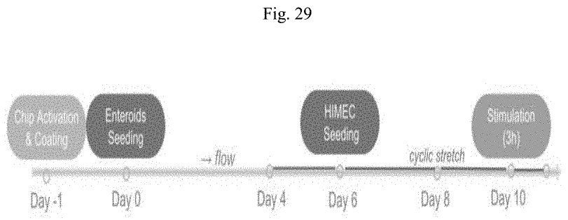

FIG. 29 Shows an exemplary Comparison Between Ileum-Chip and 3D Ileal Organoids. Heal enteroids and Ileum-Chip has been grown for up to 4, 8 and 12 days in the presence of Intesticult Media and compared using imaging and gene expression analysis.

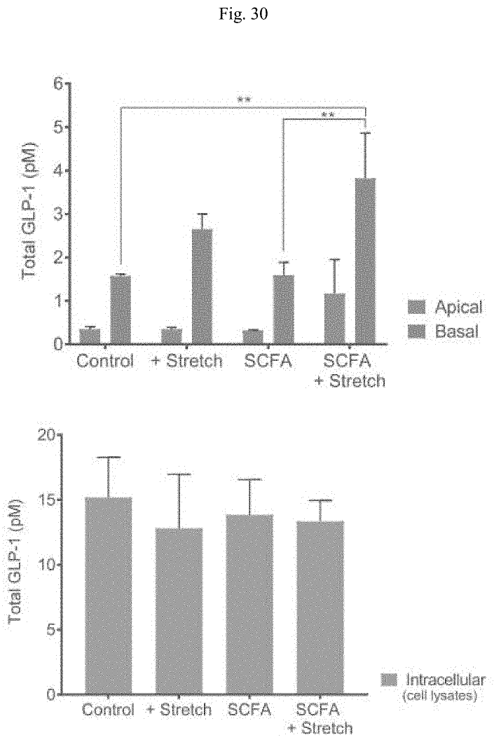

FIG. 30 Shows one embodiment of an exemplary Experimental Timeline: Ileum. Ileum-Chip and Heal enteroids were analyzed at day 4, 8 and 12 of post-seeding.

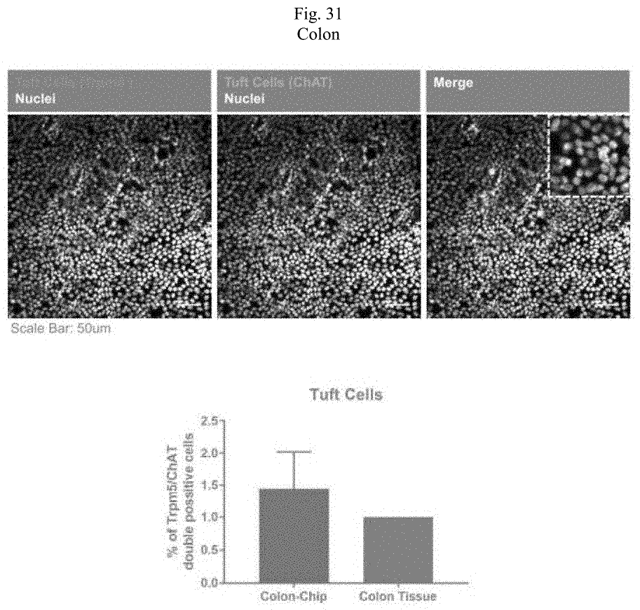

FIG. 31 Shows an exemplary Cell Types Detected in 3D Heal Organoids. Presence of all major intestinal cell types (except for tuft cells) has been detected in 3D ileal enteroids. Representative images from day 8 of growth are shown.

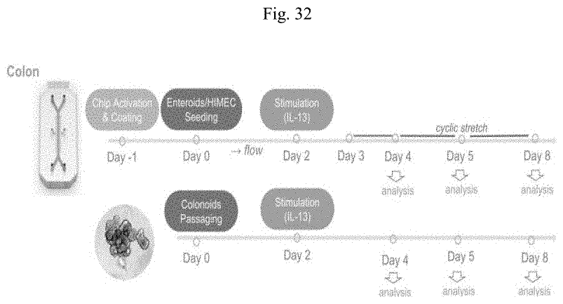

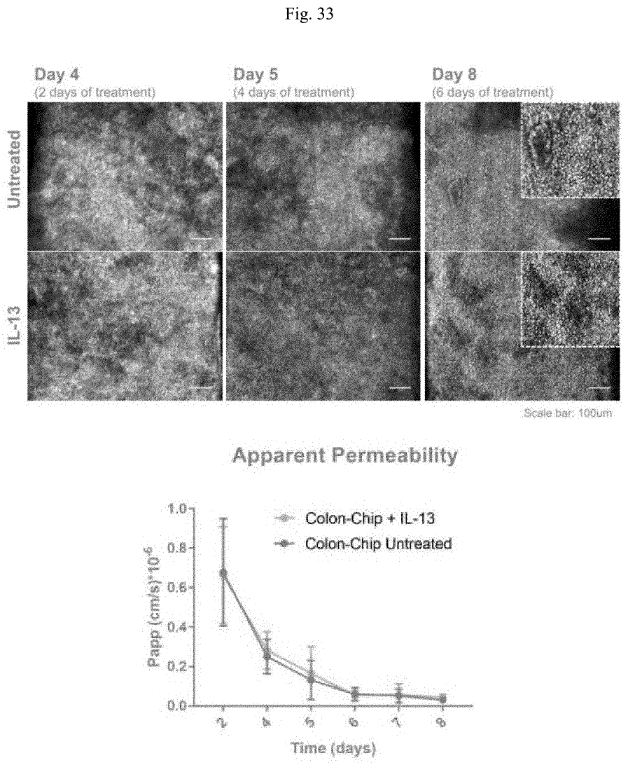

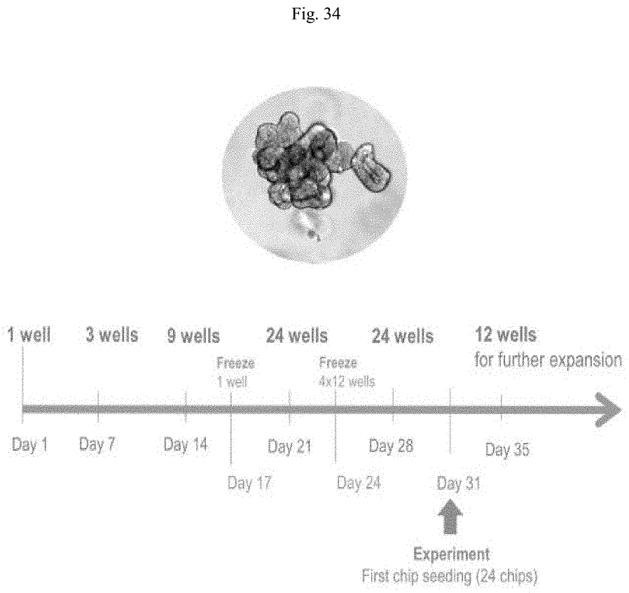

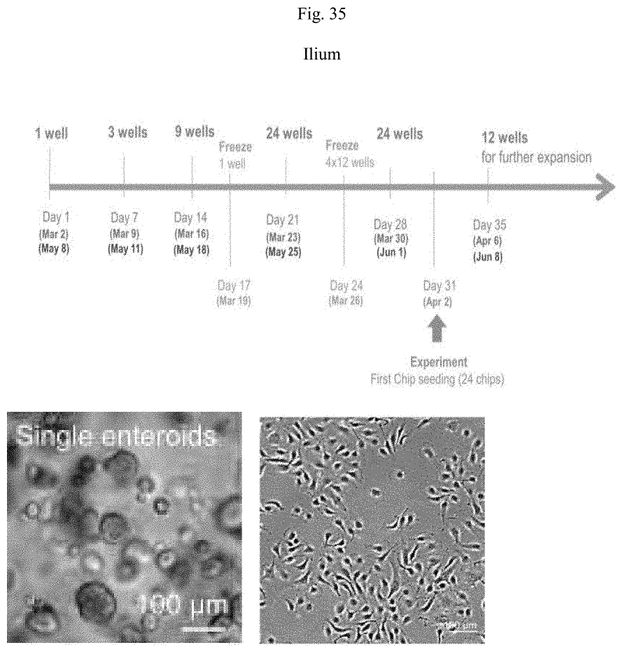

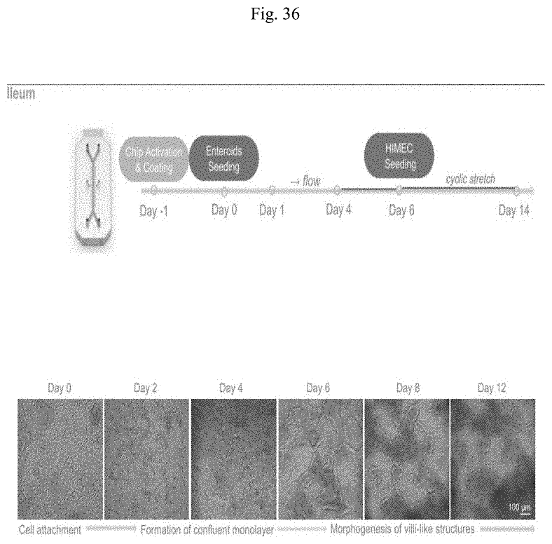

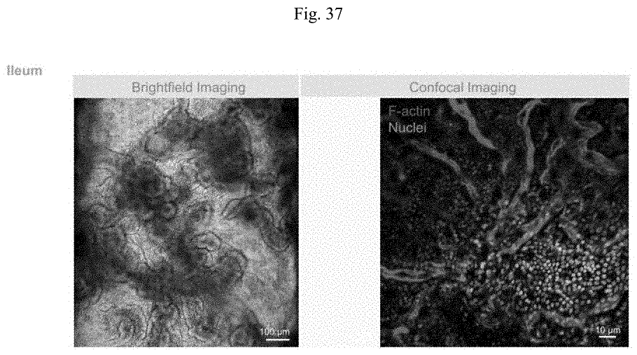

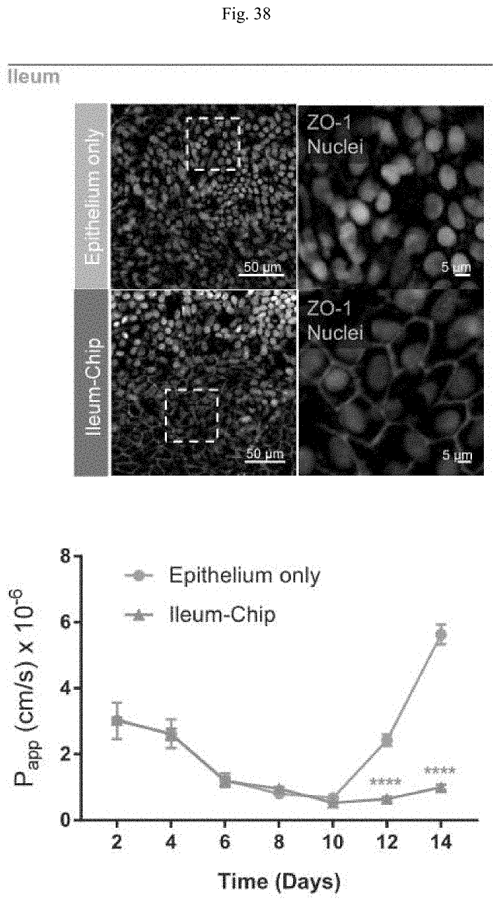

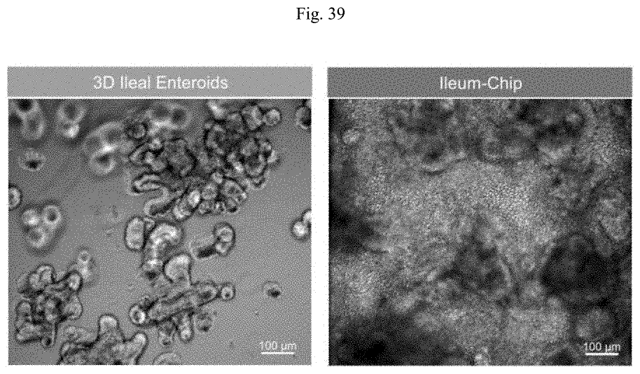

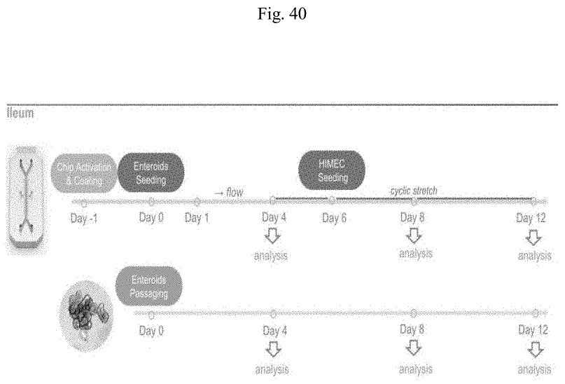

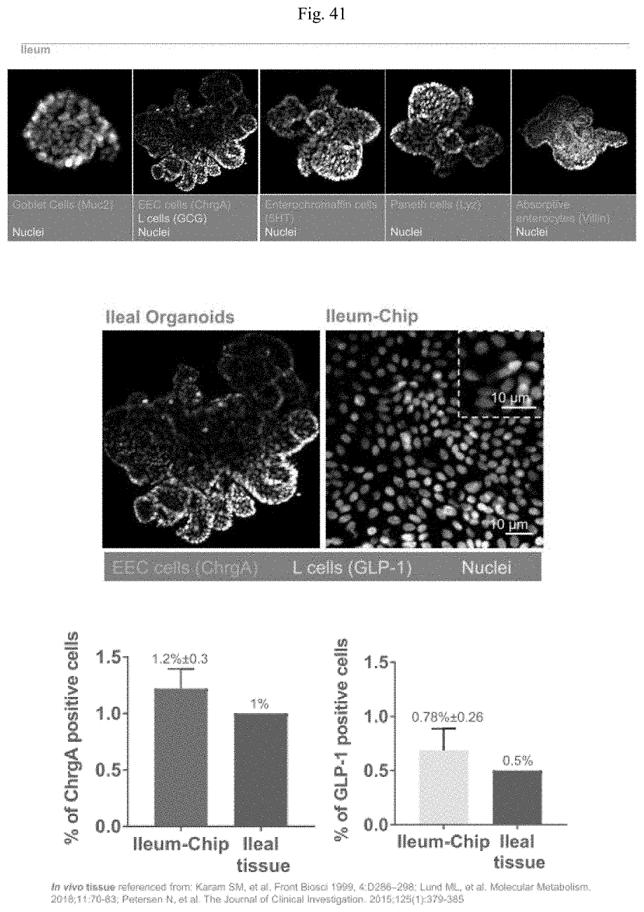

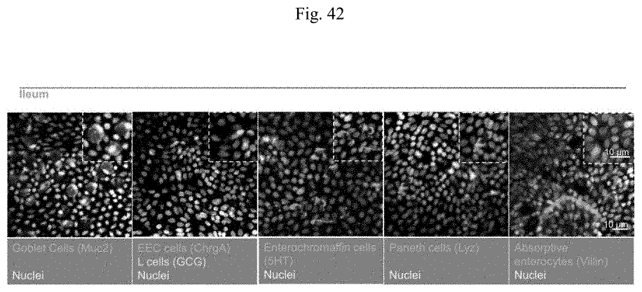

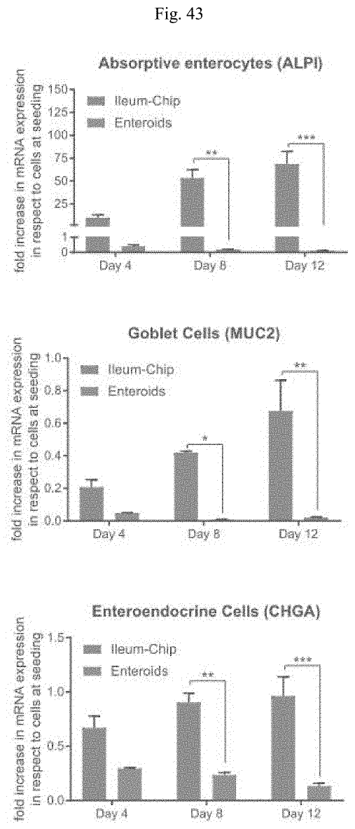

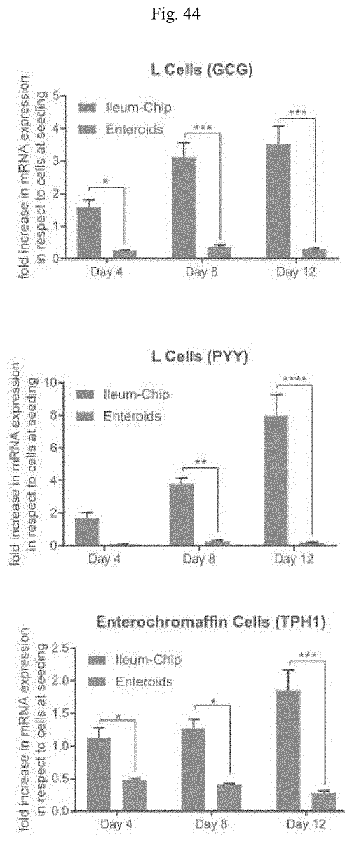

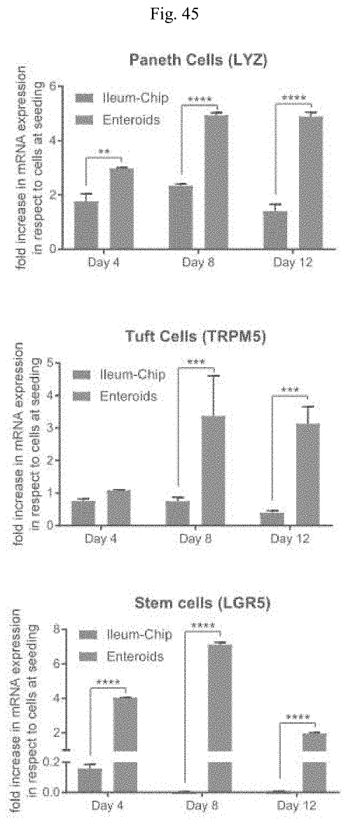

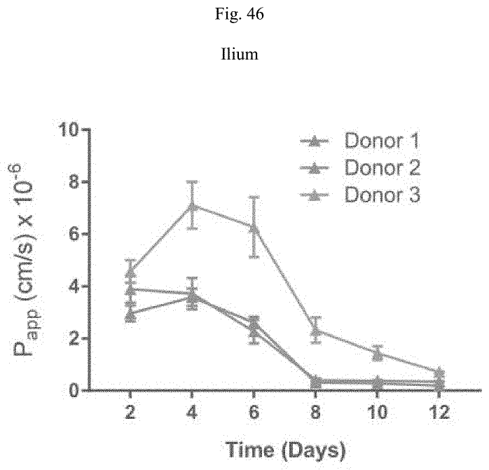

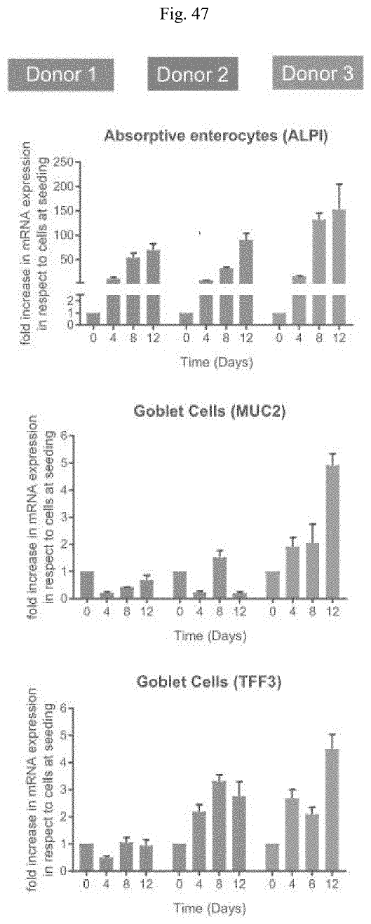

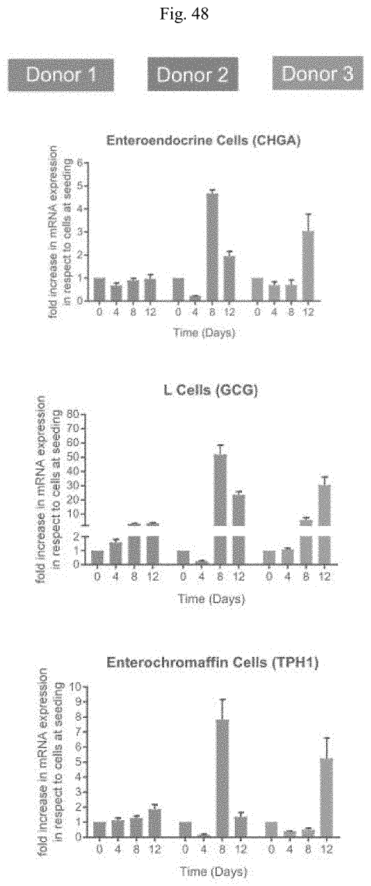

FIG. 32 Shows an exemplary Presence of Major Intestinal Cell Types Detected in Ileum-Chip.

Functional Validation of Ileum-Chip: GLP-1 Release.

Potential Stimulations: medium and long chain fatty acids (MCFAs and LCFAs) through the receptors GPR40 and 120; short chain fatty acids SCFA through GPR41 (small intestine) and GPR43 (large intestine); ethanolamides that signal through the receptor GPR119, which is expressed by L-cells; latrunculin mediated alteration in cytoskeleton; forskolin/IBMX via elevation of intracellular cAMP.

Potential Readouts: Meso Scale Discovery V-PLEX GLP-1 Total Kit (Dynamic range: 0.017-120 pM) has been detecting GLP-1 concentrations secreted from the Ileum-Chip at the LLOD (˜1 pM); Ultrasensitive Assays developed by PerkinElmer o Alternative solutions: pooling effluent samples, samples lyophilization Optimization of Experimental Protocol; Flow Rate; Time of stimulation; Site of stimulation (apical vs basal); Concentration of the stimulant and DPPIV inhibitor.

TABLE 5
References herein incorporated by reference in their entirety.

| | Model System | | | | | Assessment |
|---|---|---|---|---|---|---|
| | & Intestinal | | | | GLP-1 | Method (Range |
| Reference | Region | Stimulant | Time | | Concentration | of Detection) |
| Kasuma et al. | STC-1 Cells | 30 uM Lithocholic | 1 | hour | 4-fold increase | GLP-1 (7-36) |
| (doi: 10.1016/ | (Intestinal | Acid (LCA; Bile | | | | amide Enzyme |
| j.bbrc.2005.01.139) | Neuroendocrine | Acid) | | | | Immunoassay Kit |
| | Tumor Cells) | | | | | (Wako) (0.94-30 pM) |
| Simpson et al. | GLUTag Cells | 10 uM | 2 | hours | 11-fold increase | Linco GLP-1 |
| (doi: 10.l007/ | (Glucagon | Forskolin/10 uM | | | | active ELISA-kit |
| s00125 -007-0750-9) | producing | IBMX | | | | (N/A) |
| | enteroendocrine | | | | | |
| | cell tumor) | | | | | |
| Clara et al. | GLUTag Cells | 250 uM Oleic | 36 | minutes | 4-fold increase | Active GLP-1 |
| (DOI: 10.1016/ | | Acid | | | | (ver. 2) Assay Kit |
| j.metabol.2015.10.003) | | | | | | (Mesoscale |
| | | | | | | Discovery) |
| | | | | | | (0.12-1000 pg/ml) |
| Petersen et al. | Human | SCFA Mix (5 mM | 1 | hour | 9-fold increase | Multi-Species |
| (DOI: 10.2337/ | intestinal crypts | Acetate, 1 mM | | | GLP-1 release | GLP-1 total |
| db13-0991) | isolated from | Propionate, 1 mM | | | expressed as | ELISA (Millipore) |
| | jejunum, grown | Butyrate) | | | pg ug of | (4.1-1000 pM) and |
| | into organoids | | | | organoid | Human Total |
| | | | | | DNA/h) | GLP-1 multi-array |
| | | | | | | (Meso Scale |
| | | | | | | Diagnostics) |
| | | | | | | (0.98-1000 pg/ml) |
| Sun et al. | Endoscopic | 300 mM Glucose | 30 | min | 3-fold increase | Glucagon-Like |
| (DOI: 10.2337/ | duodenal | (Intraduodenal | | | | Peptide 1 (Active) |
| db 17-0058) | biopsies | Glucose Infusion) | | | | ELISA Kit |
| | | | | | | (Millipore) |
| | | | | | | (2-1000 pM) |
| Reimann et al. | Mixed primary | 10 mM Glucose | 2-4 | hours | 10-fold increase | Active GLP-1 |
| (DOI: 10.1016/ | cultures (upper | | | | (Colon), 2-fold | ELISA-kit |
| j.cemet.2008.11.002) | half of SI or | | | | increase (SI) | (Millipore) |
| | Colon) | | | | | (2-100 pM) |
| Parker et al. | Mixed primary | 3 uM GPBAR-A | 2 | hours | 2.5 fold increase | GLP-1 active |
| (DOI: 10.1111/ | murine intestinal | (Bile Acid | | | and further to | ELISA kit, |
| j.1476- | cultures | Receptor Agonist) | | | 3.5 fold increase | Millipore |
| 5381.2011.01561.x) | | | | | in the presence | (2-100 pM) |
| | | | | | of 10 mM | |
| | | | | | glucose | |
| Brighton et al. | Primary murine | 10 uM | 2 | hours | 40% increase in | Total GLP-1 assay |
| (DOI: 10.1210/ | intestinal | FSK/IBMX, | | | the presence of | (MesoScale |
| en.2015-1321) | cultures (SI and | | | | 10 mM Glucose | Discovery) |
| | Colonic) | | | | | (0.98-1000 pg/ml) |
| Zietek et al. | Small Intestinal | 30 uM DCA 10 uM | 2 | hours | 2.5-fold increase | Glucagon-Like |
| (doi: 10.1038/sre | Organoids | FSK/IBMX | | | 15-fold increase | Peptide 1 (Active) |

III. Intestine Colon-On-Chip.

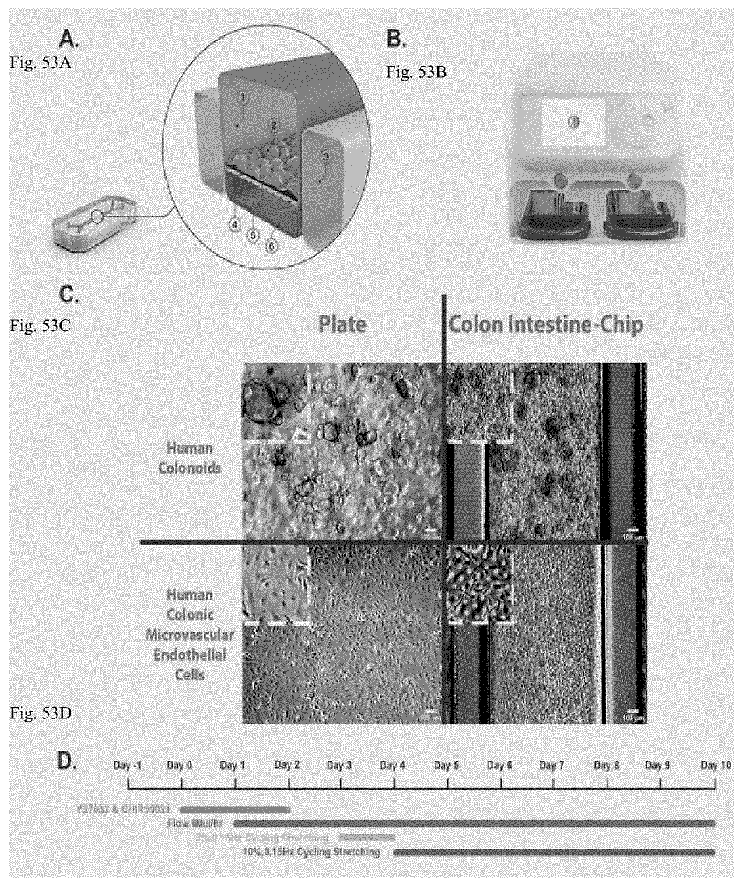

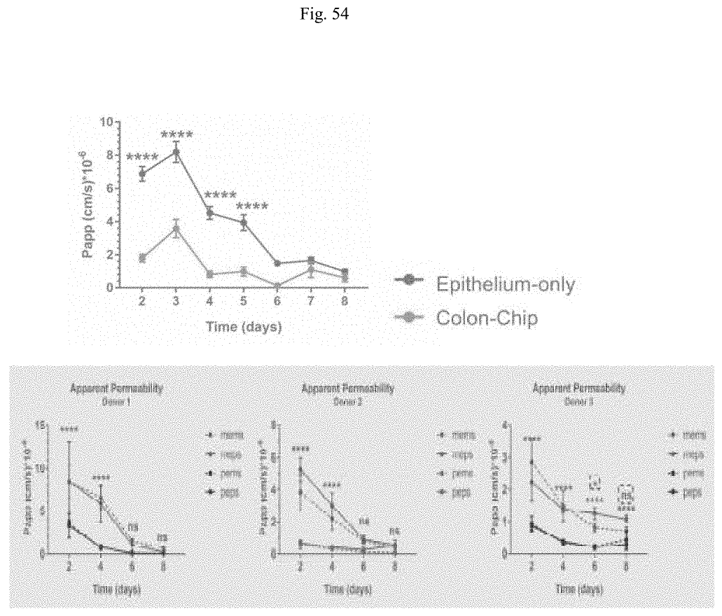

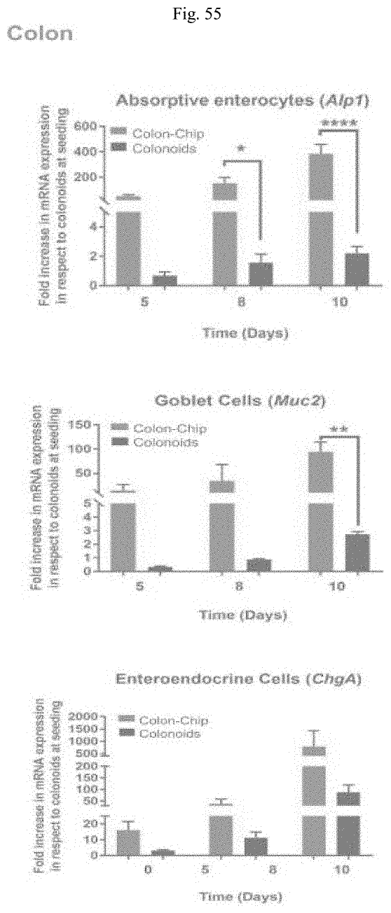

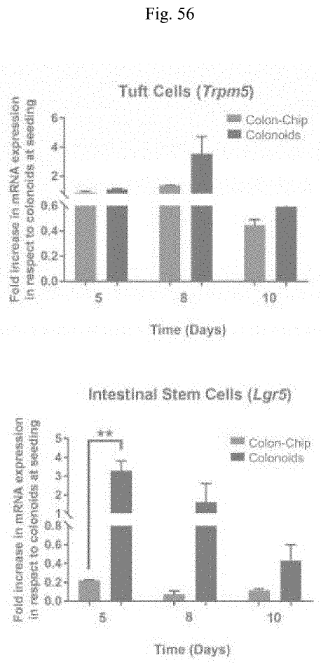

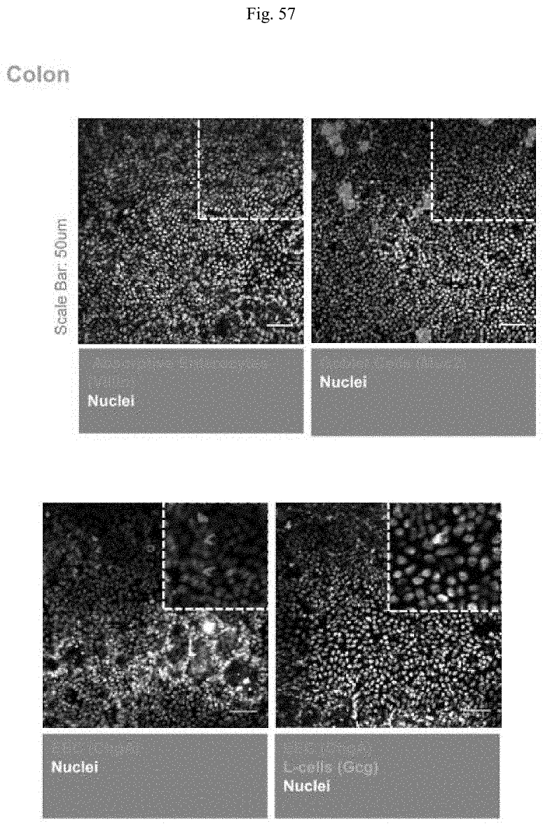

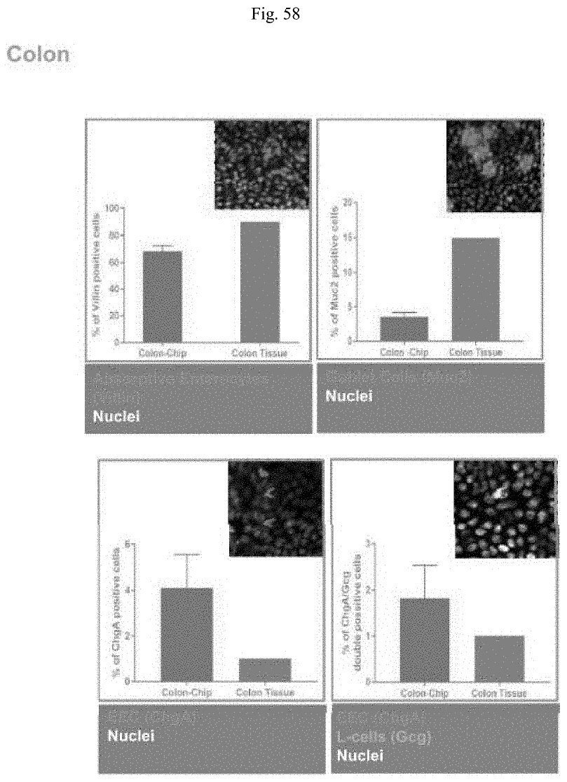

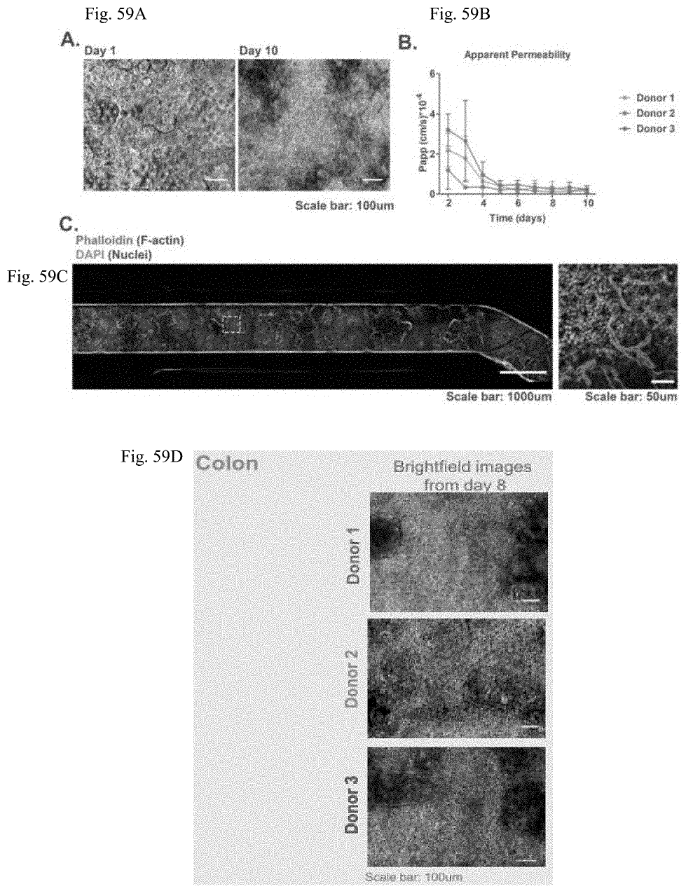

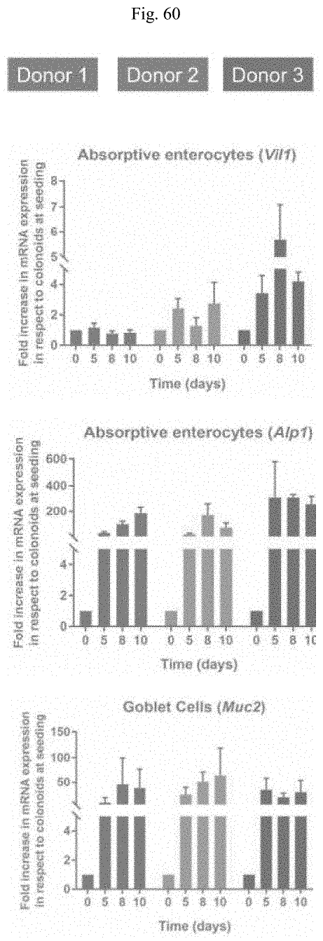

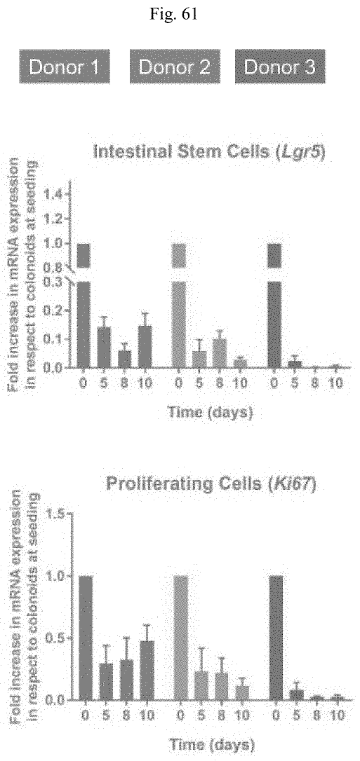

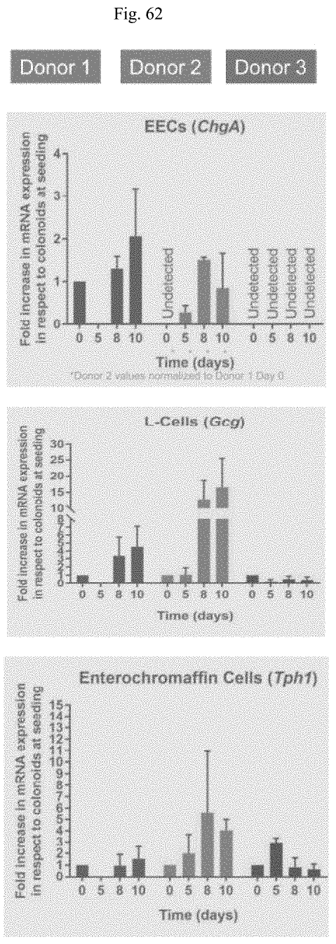

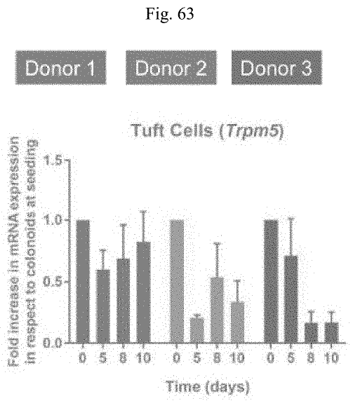

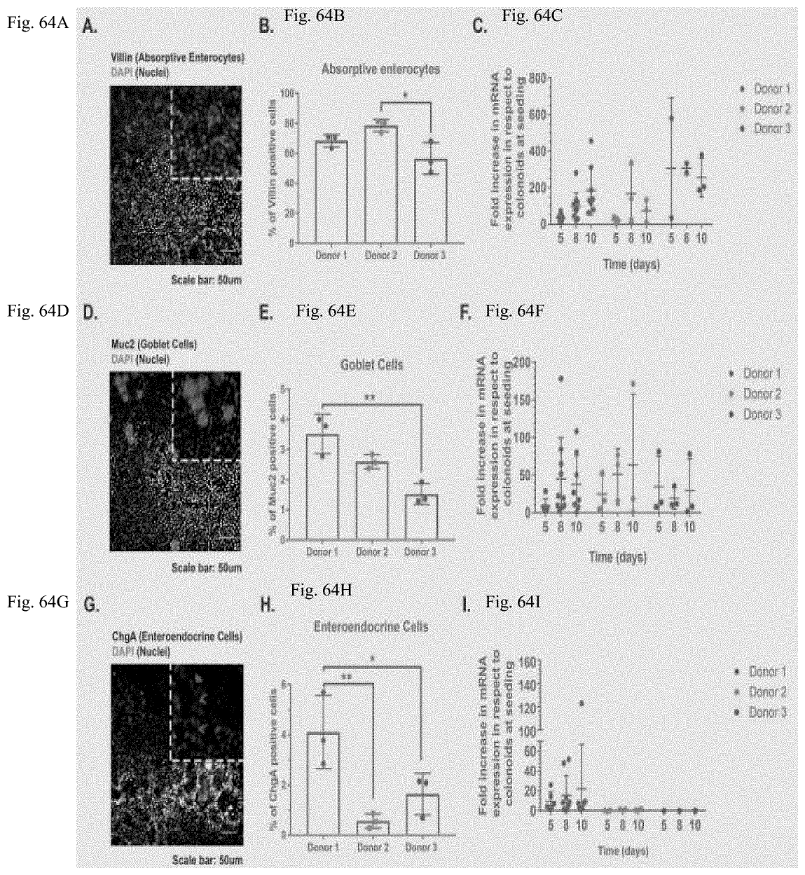

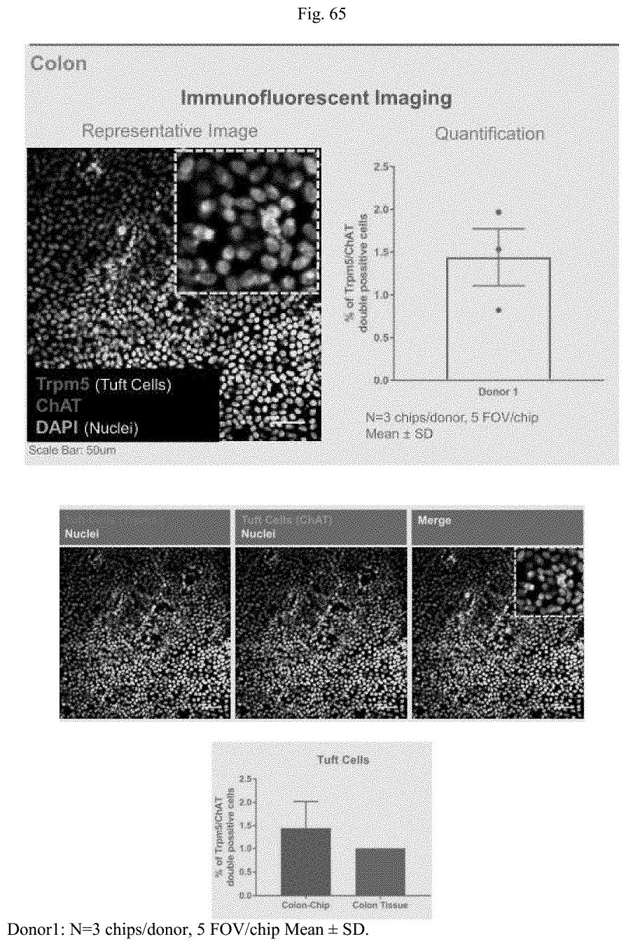

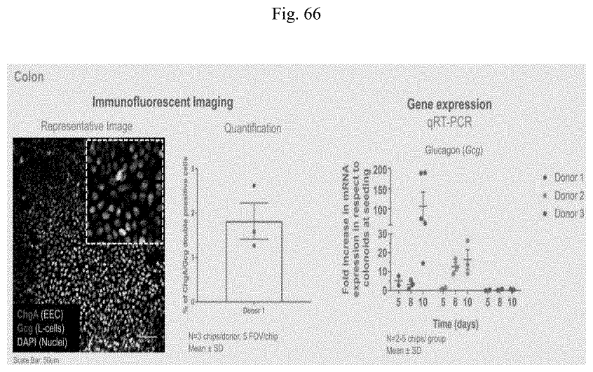

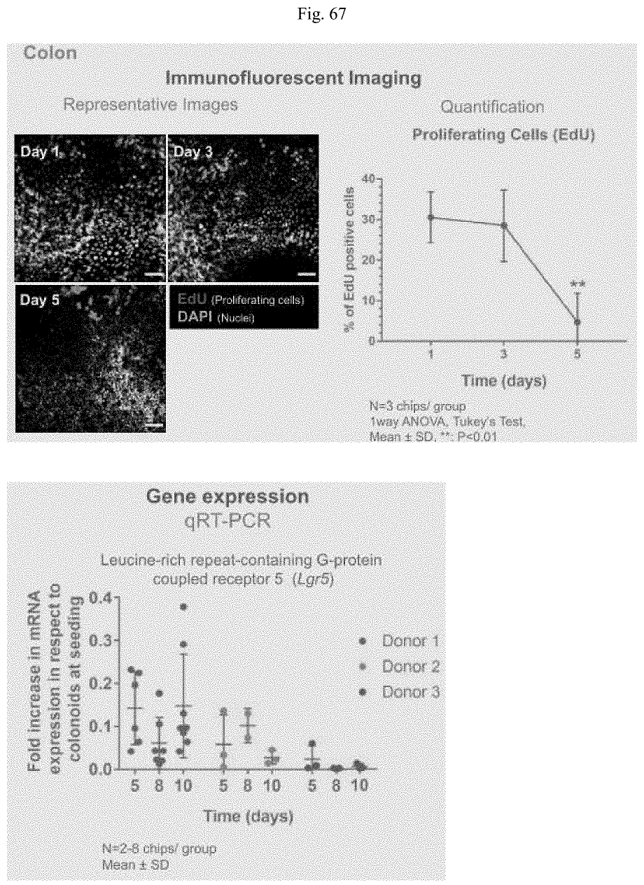

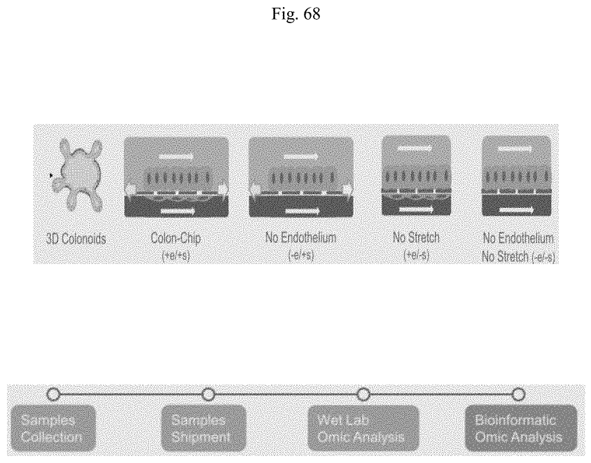

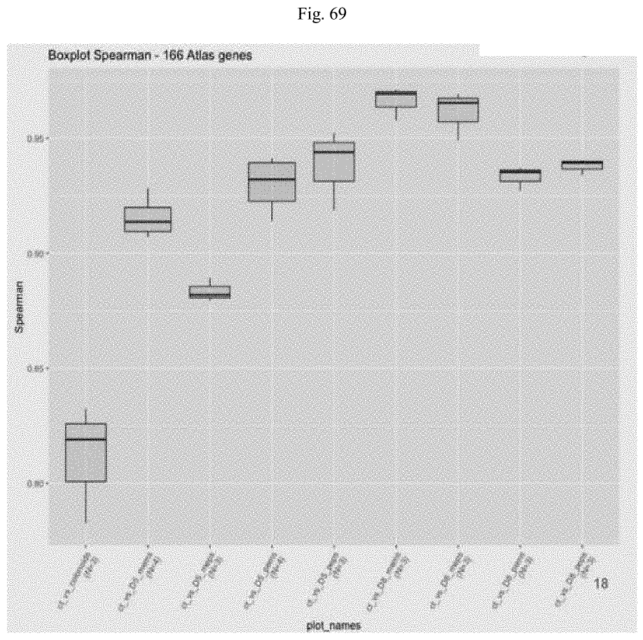

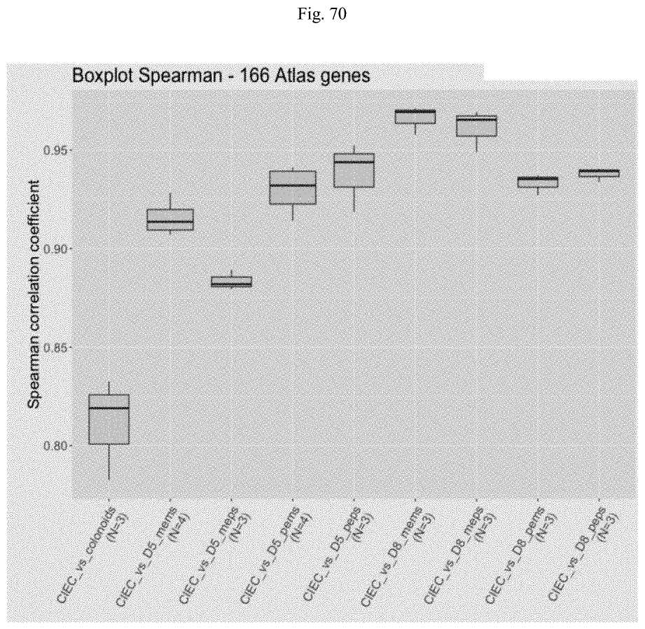

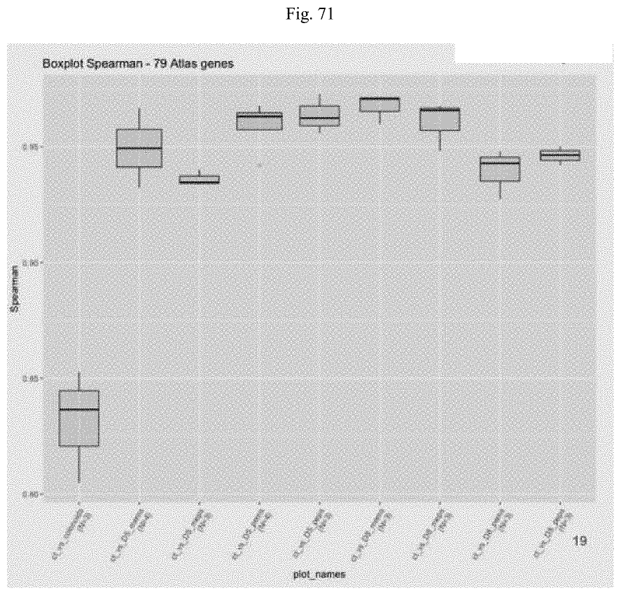

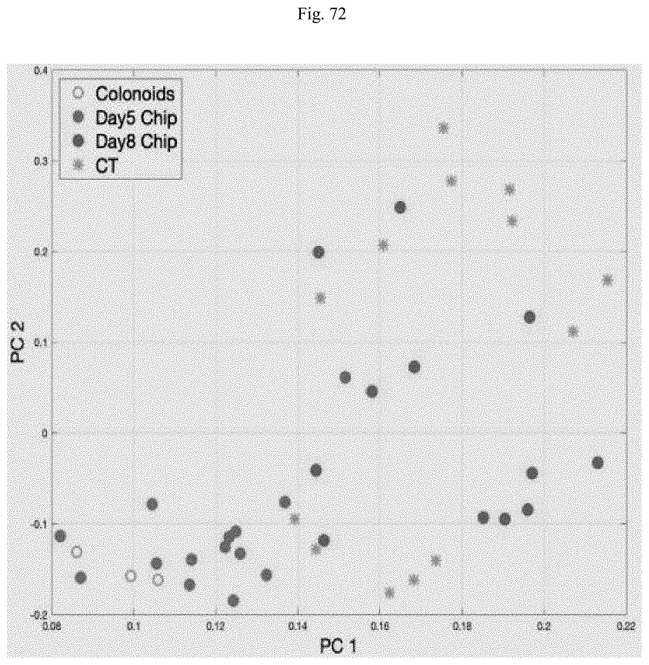

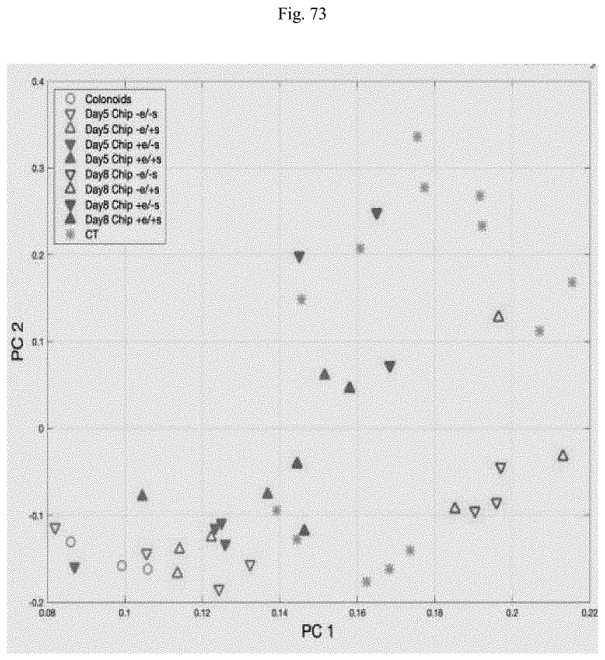

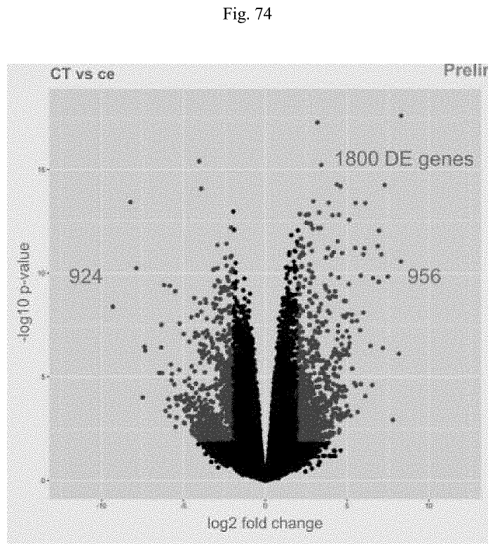

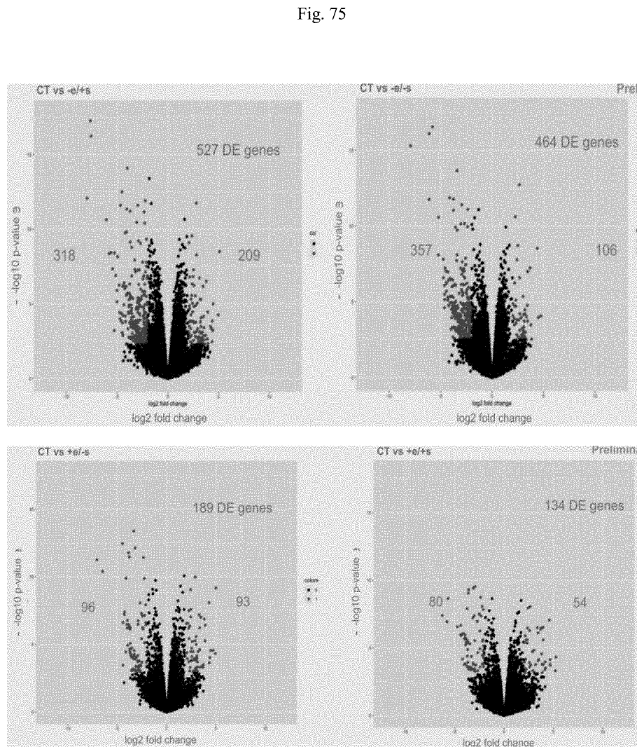

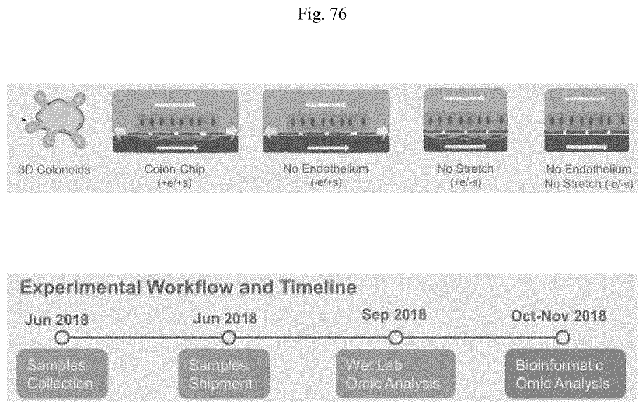

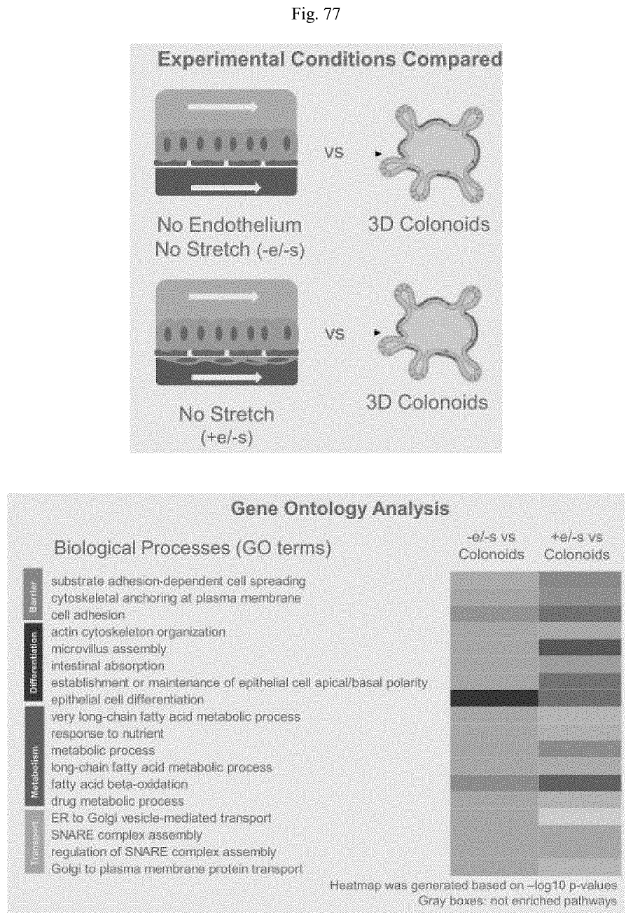

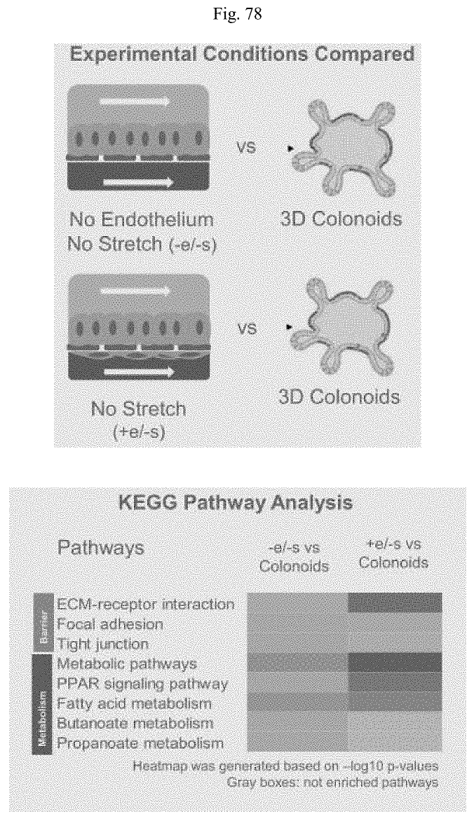

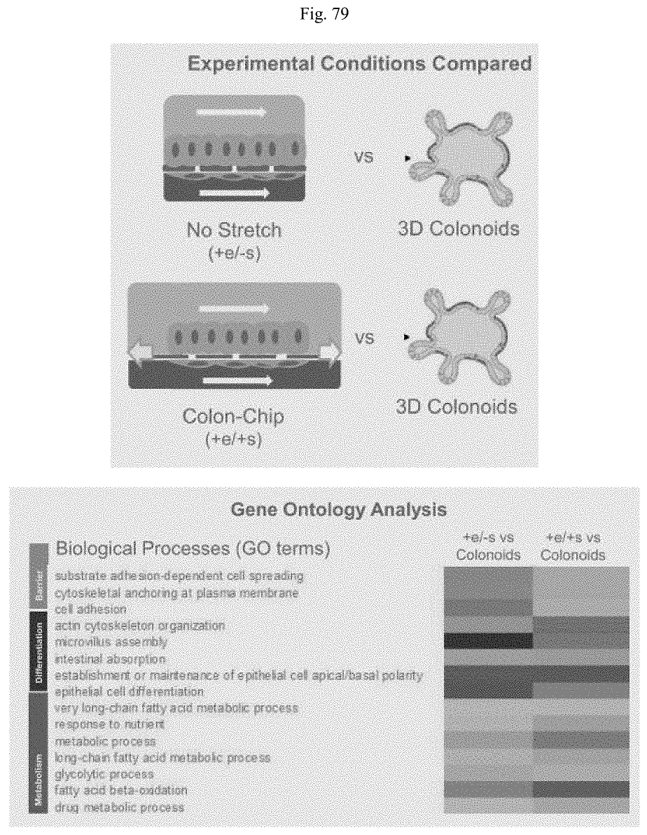

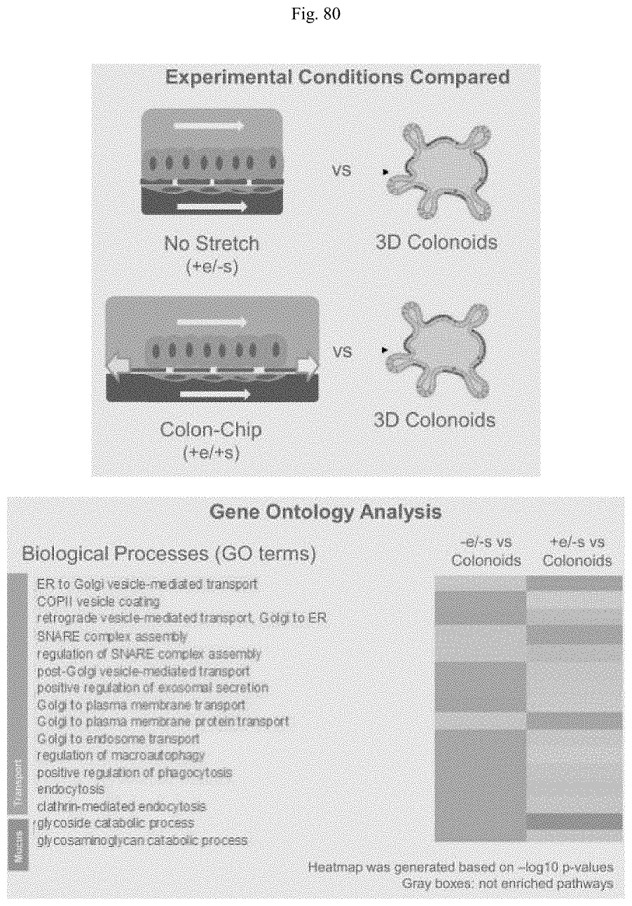

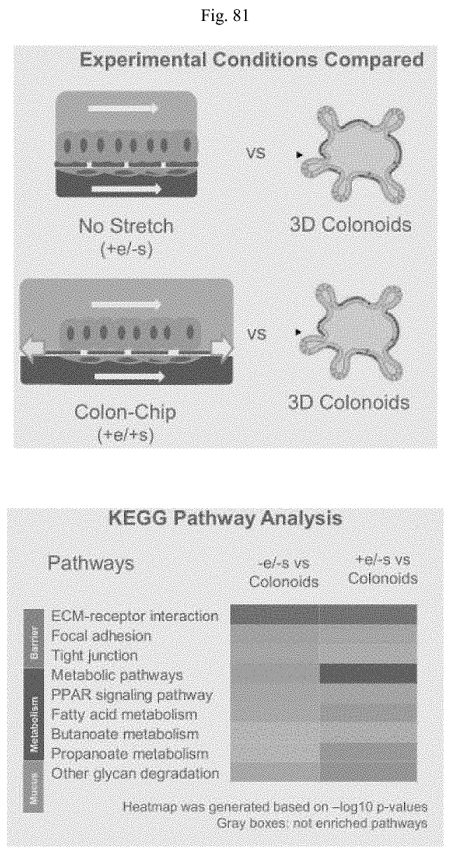

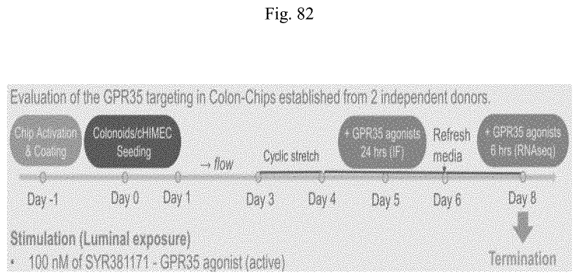

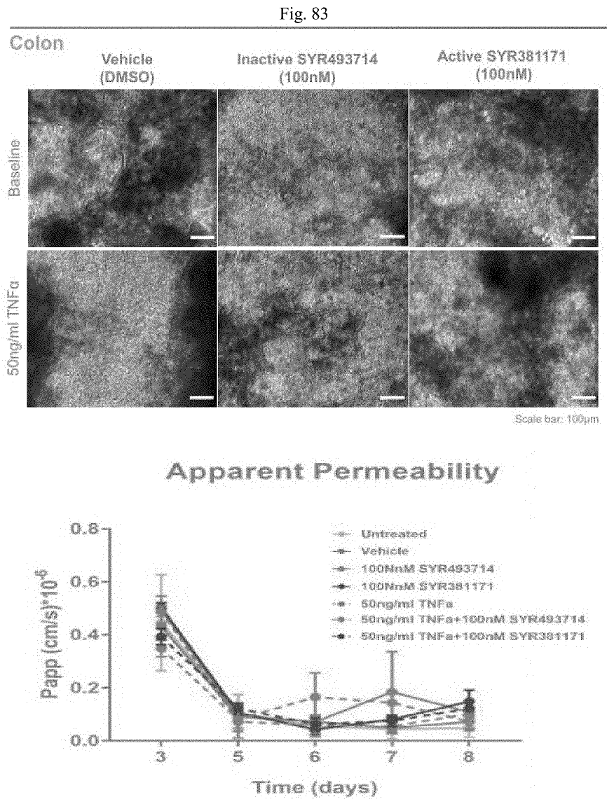

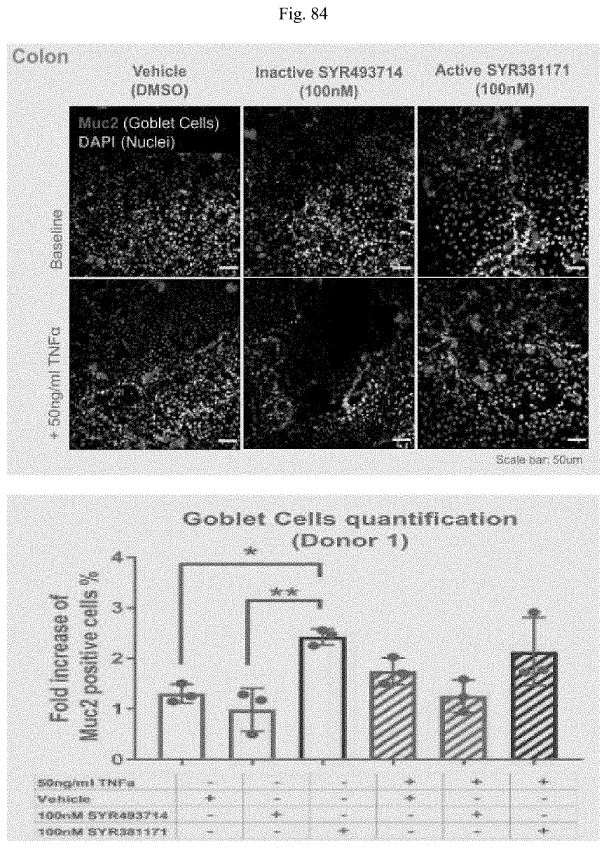

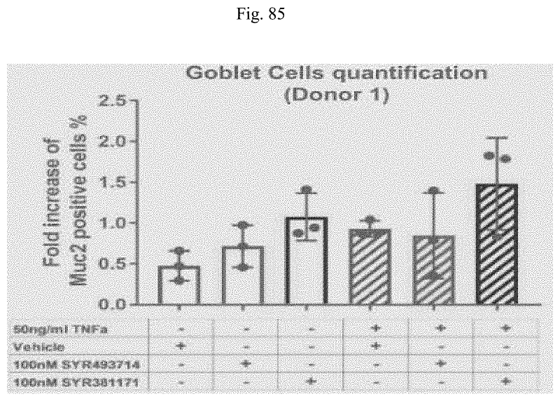

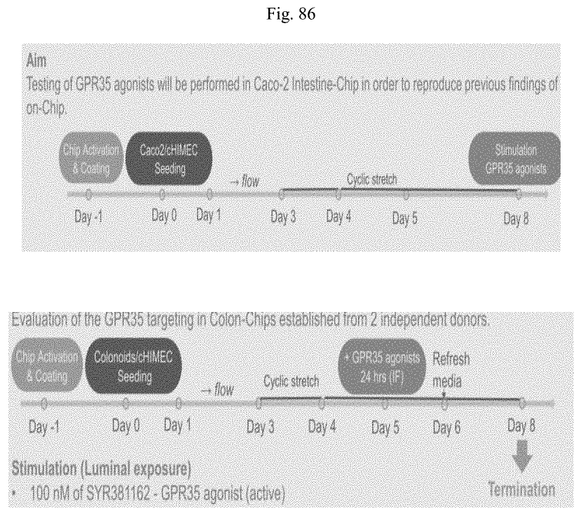

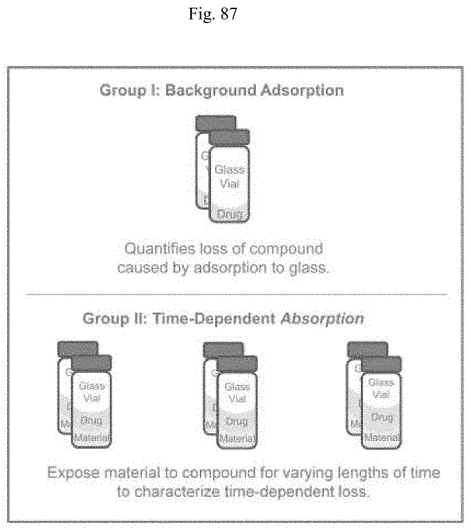

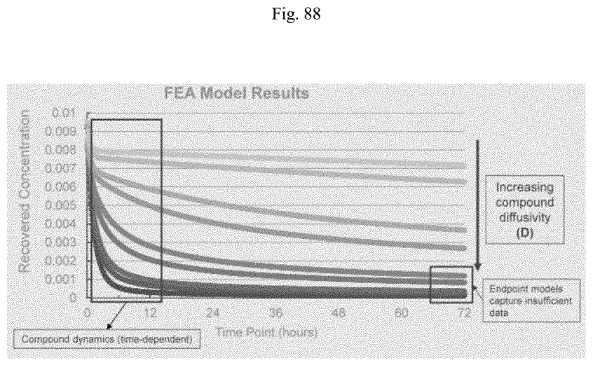

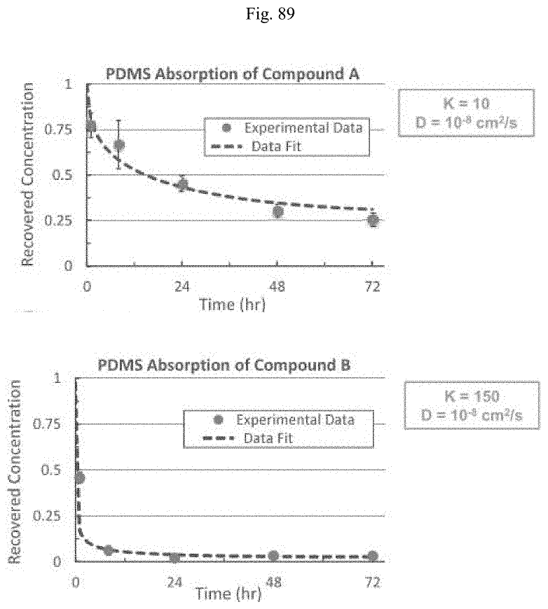

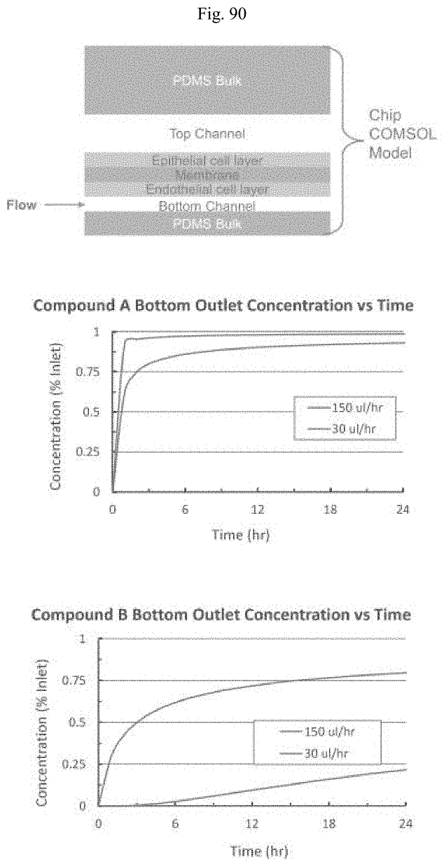

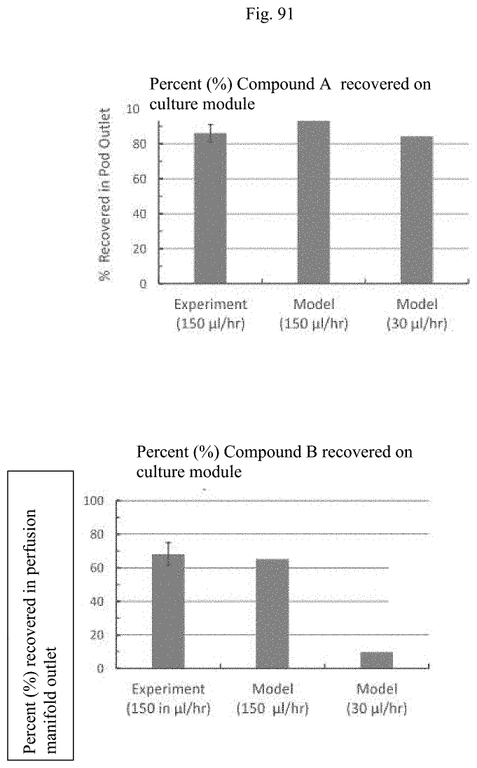

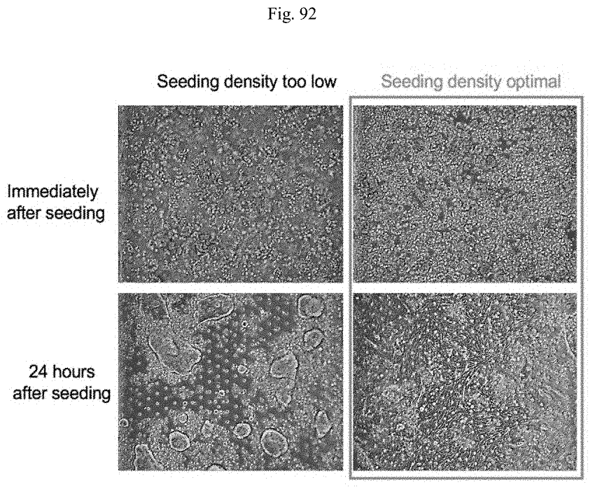

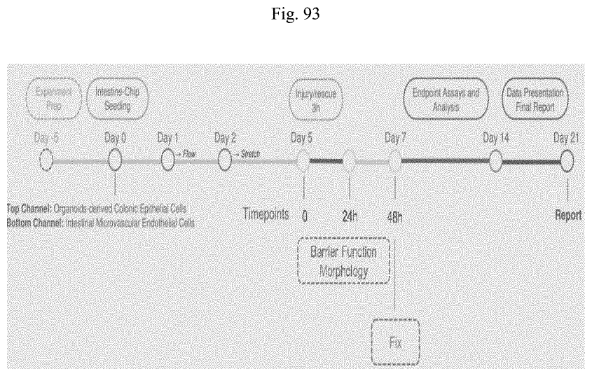

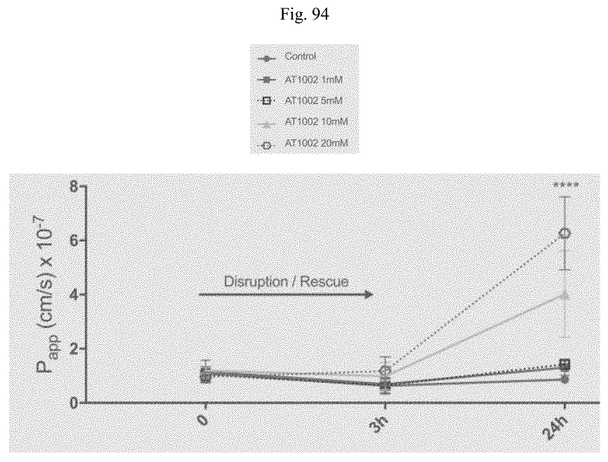

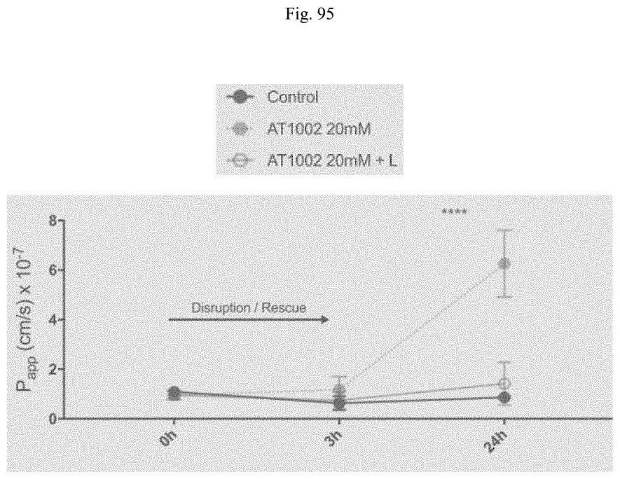

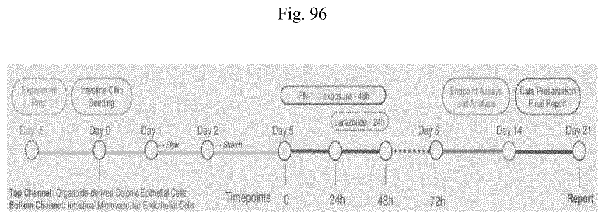

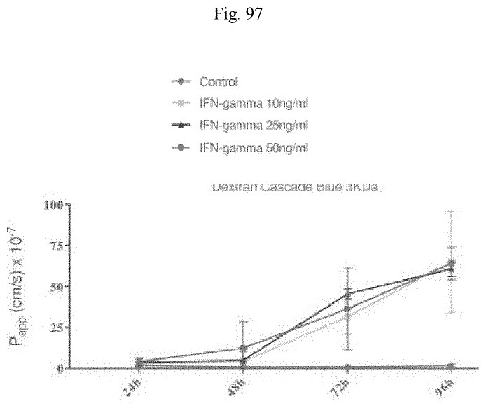

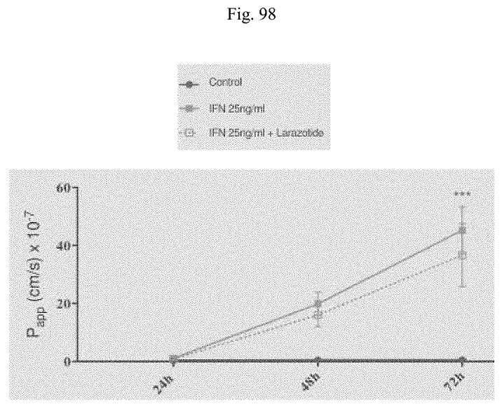

In some embodiments, an intestine chip is a colon chip (colon-on-chip). Exemplary time lines are provided showing when flow a cyclic stretch is applied to chips seeded on the same day with enteroids and endothelial cells. See, FIG. 52 and FIG. 53.

A. Stretching Vs Non-Stretching.

In some embodiments, enteroids are seeded without endothelial cells for evaluating contributions of endothelial cells to epithelial cell morphology and physiology. See, FIG. 54.

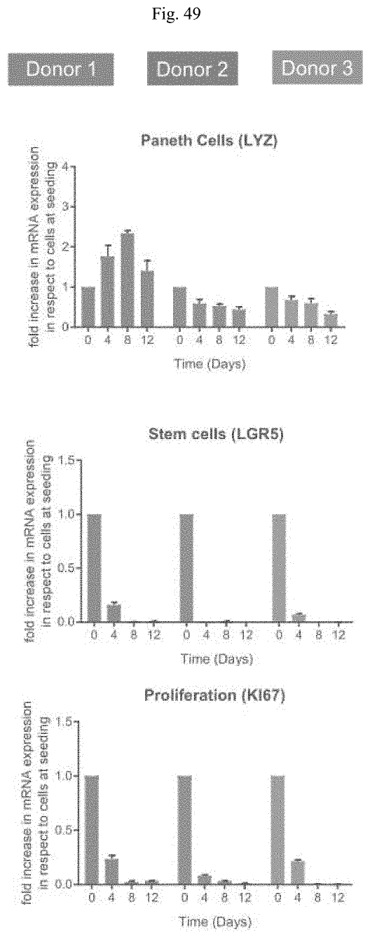

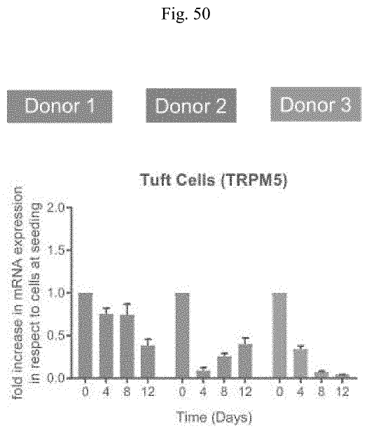

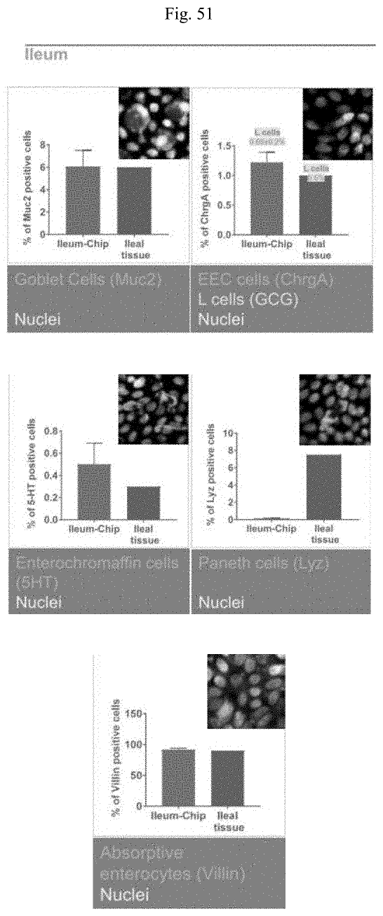

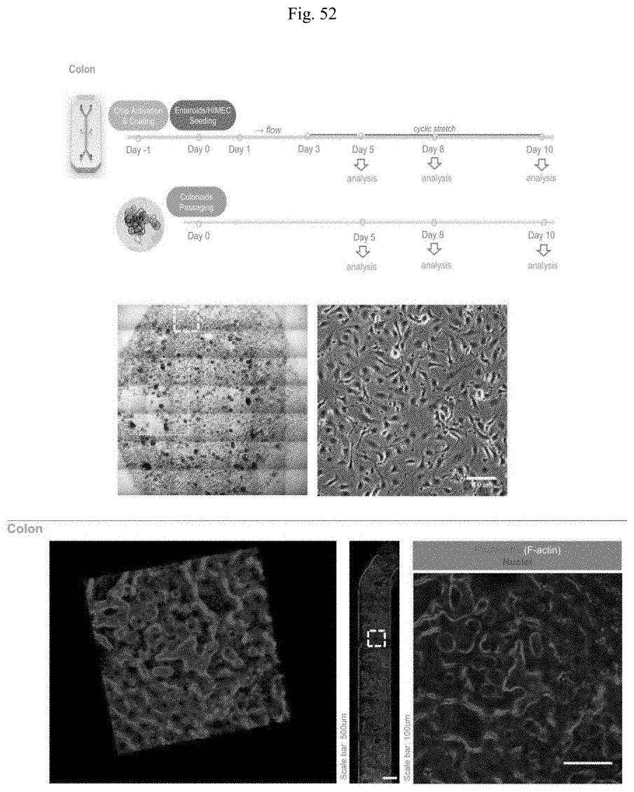

Establishment of Epithelial Barrier Across the Three Different Donors. Colon-Chips established from 3 donors reached similar levels of intestinal barrier function. FIG. 49.

B. Tuft Cells.

Tuft Cells refer to chemosensory cells located in epithelium having a microtubule bundle located at the cell apex. Tuft cells in the intestine are found both neighboring the ISC zone within the crypt and scattered upward along the crypt-villus axis. The majority of tuft cells within the intestinal epithelium express Dclkl. Tuft Cells may monitor luminal nutrients in addition to having immune regulatory functions.

Biomarkers of Tuft cells include acetylated-α-tubulin (acTub) antibody to detect the microtubule bundle; doublecortin-like kinase 1 (DCLK1) which may co-localize with acTub; and chemosensory markers, e.g., TRPM5.

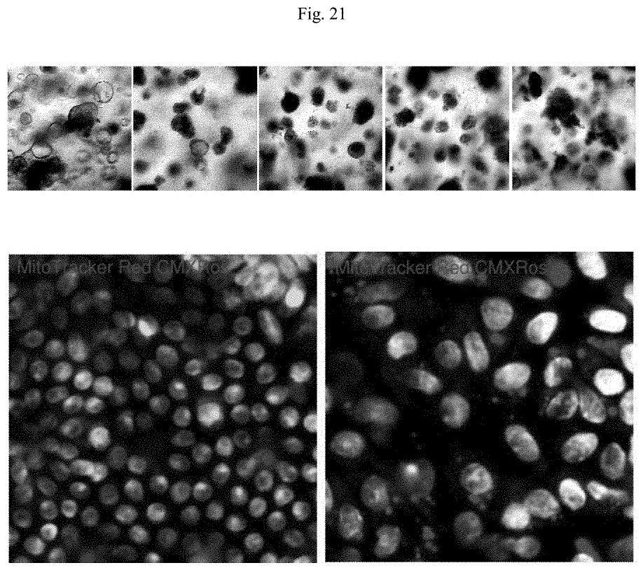

In some embodiments, tuft cells were present in Colon-Chips at physiologically relevant ratios under healthy conditions. FIG. 21.

FIG. 21 Shows an exemplary identification of tuft cells in one embodiment of a Colon-Chip System.

Tuft cell numbers expand, and may be considered hyperplasic under conditions of injury, e.g., during chronic inflammation and in preneoplastic tissues. Thus, in some embodiments, enrichment of tuft cells is desired in an intestine-chip.

C. IL-13 Mediated Enrichment of the Tuft Cells in Colon-Chip.

Stimulation: Exposure of Colon-Chips and ileal and colonic organoids (serve as a control) to 10 ng/ml of IL-13.

Read-outs: mRNA expression of genes specific for Tuft cells: Trpm5, ChAT, Dclkl and for goblet cells: Muc2, TFF3, etc., (to assess concomitant expansion of goblet cell populations) at day 2, 3 and 6 post-treatment corresponding to day 4, 5 and 8 of Colon-Chip culture.

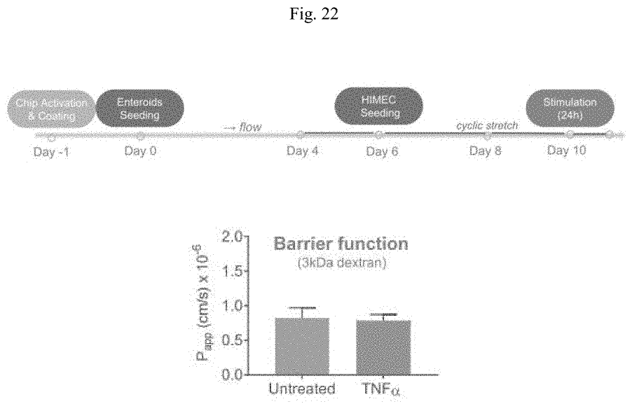

FIG. 22 Shows an exemplary Enrichment of Tuft Cell Population by IL-13 Treatment in one embodiment of a Colon-Chip.

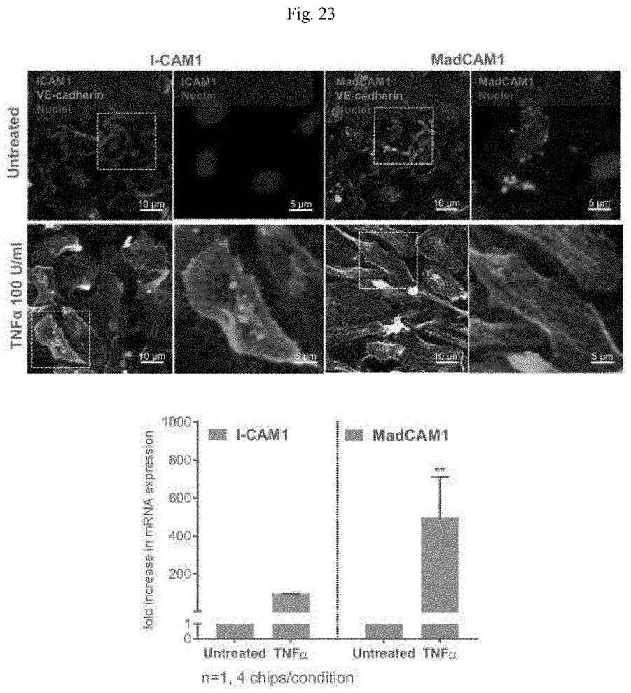

IL-13 treatment has no negative impact of Colon-Chip tissue morphology and barrier function. FIG. 23 shows exemplary results that IL-13 treatment does not affect establishment of barrier function one embodiment of a Colon-Chip.

Summary of IL-13 mediated enrichment of the tuft cells in Colon-Chip. We have demonstrated the presence of Tuft cells in Colon-Chip at the physiologically relevant ratios. We performed stimulation of Colon-Chips with IL-13 in order to drive tuft cell differentiation and enrich for the presence of these cells in Intestine-Chip platform. IL-13 showed no negative effect on Colon-Chip morphology and barrier function. In some embodiments, Gene expression levels of Tuft cell (Trpm5, ChAT) and goblet cell (Muc2, TFF3) specific markers are evaluated.

D. Summary of Colon-Chip Characterization:

Characterization of Colon-Chip (in the presence of microvasculature) revealed: Cross-talk between the epithelium and endothelium led to accelerated barrier formation; Confirmation of the differentiation of the major intestinal cell types and quantification of cell populations. Increased expression of genes specific for differentiated cell lineages in Colon-Chips in comparison to 3D colonic organoids; Increasing epithelial maturation is correlated with the decrease in sternness and proliferation; Differentiation of enteroendocrine specific cell subtypes; Confirmation of Tuft cell differentiation and quantification of tuft cell population similar to colonic tissue.

V. Disease Modeling, Biomarker Identification, and Drug Testing.

In the effort to increase efficacy and specificity on newly developed drugs for intestinal disorders, human-based systems provide a promising new alternative to the animal-based studies. The interface of epithelium with endothelium allows for nutrient exchange and oxygen diffusion, as well as a gateway for circulating immune cells in states of inflammation. Our knowledge, on the contribution of the microvasculature during intestinal mucosa development is still limited. We use Organ-Chips and our proprietary Human Emulation System to develop a platform where organoids are able to expand and differentiate to monolayers consisted of the three major intestinal epithelial cell types. Here, we report the development of a colonoids-based platform for disease modeling, biomarkers identification, and drug testing on a patient-specific approach.

A. Colon Intestine-Chip.

Methods: Human crypt-derived colonic organoids, derived from adult male individuals, are seeded in the Colon Intestine-Chip interfacing with colonic human intestinal microvascular endothelial cells (cHIMECs). Intestine-Chip is maintained in a perfusion manifold, an instrumentation for the culture of the Intestine-Chip. Colonic organoids cultured in the chip for 10 days in the presence of Wnt3a, Noggin, and Rspol, under physiologically-relevant mechanical and shear stress, were expanded to epithelial monolayers (IECs). Epithelial barrier function was assessed over time by the apparent permeability (Papp) of 3 kDa Dextran. The relative abundance of the main epithelial cell subtypes was assessed by qPCR and immunofluorescence staining.

In depth transcriptomic analysis was performed using bulk RNAseq. Specifically, colonoids either in suspension or expanded to monolayers on Intestine-Chip, in the presence or absence of endothelium (HIMECs) and/or cycling stretching, were harvested on days 5 and 8 of the fluidic culture and analyzed accordingly. Differential Gene Expression Analysis of these samples as compared to publicly available bulk RNAseq data from human colonic IECs (cIECs) (Kraiczy J, et al. Gut 2017; 0:1-13. doi:10.1136/gutjnl-2017-314817, Howell K J et al. Gastroenterology 2018; 154:585-598. doi:10.1053j.gastro.2017.10.007). In order to further delineate the closeness between Colon Intestine-Chip and epithelial cells isolated from colon tissue (cIECs) gene expression, we referred to the strict transcriptomic signature (166 genes) of the colon tissue as per the Human Protein Atlas project (https://www.proteinatlas.org/humanproteome/tissue/colon).

The establishment of a confluent monolayer on the Colon Intestine-Chip was confirmed by contrast phase microscopy whereas barrier function was assessed by the apparent permeability of 3 kDa Dextran, measured over the course of 10-days in fluidic culture. Development of an in vivo relevant cytoarchitecture, in the presence of applied physiologically relevant forces, was evident by F-actin staining.

We confirmed the multilineage differentiation capacity of the Colon Intestine-Chip, towards absorptive enterocytes, Goblet cells, and enteroendocrine cells, by qPCR and immunofluorescence against genes and specific protein markers for each of the above epithelial cell types, in three different donors.

The Differential Gene Expression Analysis, revealed significant decrease in the Differentially Expressed Genes (DEGs) on cells cultured on the Colon Intestine-Chip, in the presence of endothelium on day 5 of the fluidic culture. Stretching did not have any significant effect on gene expression as per RNAseq analysis.

Spearman correlation of the ranking of the average expression of the 166 genes-mentioned as the strict transcriptomic profile of the colon tissues in Atlas-between cIECs, Colonoids or cells in the Colon Intestine-Chip, from different culture conditions, demonstrated the closeness between the Colon Intestine-Chip and the colonic epithelium directly derived from the tissue (in vivo).

Comparison of the Transcriptomic Profile of the Colon Intestine-Chip and the Colon Tissue. [A] PCA analysis based on the 166 Atlas colon-specific genes in Colonoids, colonic epithelial cells cultured in the Colon Intestine-Chip under different conditions on day 5 and 8 of the fluidic culture, and hIECs. PC1: 93.84% of variance, PC2: 3.59% of variance [B] Spearman correlation of the ranking of the average expression of the 166 Atlas genes between the hIECs and the Colonoids or cells from the Colon Intestine-Chip. Colonic epithelial cells were cultured in the Colon Intestine-Chip in the presence (+e) or absence (−e) of cHIMECs and with (+s) or without (−s) cycling stretching.

B. Summary.

We report the development of a new microengineered Colon Intestine-Chip made of a transparent elastomeric polymer, poly-dimethylsiloxane (PDMS). Colon Intestine-Chip contains two parallel microchannels separated by a thin porous flexible membrane coated with tissue specific extracellular matrix, seeded with human crypts-derived colonic epithelial cells and human colonic microvascular endothelial cells respectively. The cells in the Colon Intestine-Chip are exposed to maintained under low shear stress, recreated by continuous flow at physiological rates, and mechanical forces recapitulating the in vivo intestinal peristalsis. Colon Intestine-Chip demonstrates a tight barrier over 10 days in culture, as shown across multiple donors. Colon Intestine-Chip allows for a multilineage epithelial differentiation in the presence of the sternness factors Wnt3a, Rspol and Noggin, as per published, standardized protocols. The Colon Intestine-Chip indicated the significance of endothelium and mechanical stretching for the functional maturation of human intestinal epithelial progenitor cells. The transcriptomic signature of the colonic epithelial cells in the Colon Intestine-Chip shows a great similarity to that of the human tissue, even in the hierarchical ranking of the colon-specific genes.

Human Colon Intestine-Chip model that recapitulates aspects of the human tissue biology including gene expression profiles. In ongoing studies, we incorporate additional relevant cell types, such as immune cells, as a necessary step towards development of gastrointestinal disease models. Our Colon Intestine-Chip platform provides a very much needed experimental system to be used to assess human efficacy and safety of novel drugs for gastrointestinal diseases, unmask disease mechanisms, and support new biomarkers identification.

Goal: To evaluate the role of intestinal microvasculature and stretching on the maturation and function of human colonic epithelium.

Shows an exemplary RNAseq Dataset. 42 samples. 31240 gene expressions. To remove the batch effect we used the ComBat function of the “Surrogate Variable Analysis” R package.

RNA-seq dataset

| Colonoids | Chips Day 5 | Chips Day 8 | Colon Tissue |
|---|---|---|---|
| 3 samples | 4 samples −e/−s | 3 samples −e/−s | 13 samples |
| | 3 samples −e/+s | 3 samples −e/+s | |
| | 4 samples +e/−s | 3 samples +e/−s | |
| | 3 samples +e/+s | 3 samples +e/+s | |

The Human Protein Atlas.

The Human tissue Atlas contains information regarding the expression profiles of human genes both on the mRNA and protein level. https://www.proteinatlas.org/humanproteome/tissue/colon

The mRNA expression data is derived from deep sequencing of RNA (RNA-seq) from 37 different normal tissue types (heart, liver, lung, colon, brain etc.). Transcriptome analysis reveals 166 genes that have significantly elevated expression in colon compared to other tissue types. 79 of these genes have at least five-fold higher mRNA levels in colon tissue as compared to average levels in all tissues.

Human Colon Tissue Atlas

These genes can be considered as a genomic signature of the colon tissue.

Spearman correlation of the ranking of the average expression of the 166 genes-mentioned as the strict transcriptomic profile of the colon tissues in Atlas-between cIECs, Colonoids or cells in the Colon.

Intestine-Chip, from different culture conditions, demonstrated the closeness between the Colon Intestine-Chip and the colonic epithelium directly derived from the tissue (in vivo).

Comparison of the Transcriptomic Profile of the Colon Intestine-Chip and the Colon Tissue [A] PCA analysis based on the 166 Atlas colon-specific genes in Colonoids, colonic epithelial cells cultured in the Colon Intestine-Chip under different conditions on day 5 and 8 of the fluidic culture, and hIECs. PC1: 93.84% of variance, PC2: 3.59% of variance [B] Spearman correlation of the ranking of the average expression of the 166 Atlas genes between the hIECs and the Colonoids or cells from the Colon Intestine-Chip. Colonic epithelial cells were cultured in the Colon Intestine-Chip in the presence (+e) or absence (−e) of cHIMECs and with (−s) or without (−s) cycling stretching.

Exemplary Spearman Correlation Analysis (based on 166 Atlas genes).
1. We calculate the average expressions of all genes across the 13 colon tissue (ct) samples.
2. We create a vector with the expressions of the 166 Atlas genes. We repeat the same procedure (steps 1-3 above) for each sample (colonoid or chip). Finally, for each sample we compute its Spearman correlation with the tissue vector produced in step 3 (tissue expressions of the 166 Atlas genes).
3. We sort this average ct gene expressions vector in descending order.

Spearman Correlation Analysis (based on 79 Atlas genes)
1. We calculate the average expressions of all genes across the 13 colon tissue (ct) samples. 2. We create a smaller vector with the expressions of the 79 Atlas genes. We repeat the same procedure (steps 1-3 above) for each sample (colonoid or chip). Finally, for each sample we compute its Spearman correlation with the tissue vector produced in step 3 (expressions of the 79 Atlasgenes).
2. We sort this average ct gene expressions vector in descending order.

PCA Analysis (1)

We perform PCA analysis using the expressions from the 166 Atlas genes.

We plot the samples using the information from the first 2 Principal Components:

The first 2 PCs explain the 97.43% of the total variance.
PC 1: explains 93.84% of the variance
PC 2: explains 3.59% of the variance
Chip samples are closer to tissue samples relative to colonoids.
   Day8 chip samples are closer to tissue samples than Day5 chip samples

PCA Analysis (2)

We perform PCA analysis using the expressions from the 166 Atlas genes.

We plot the samples using the information from the first 2 Principal Components:

The first 2 PCs explain the 97.43% of the total variance
PC 1: explains 93.84% of the variance
PC 2: explains 3.59% of the variance
+Endo Chip samples tend to have higher PC2 than—Endo of same Day
High PC2 tissue samples tend to be closer to +Endo Chip samples

Volcano Plots.

Differential Gene Expression Analysis. Using the batch corrected dataset, we applied

Differential Gene Expression Analyses between: a) colon tissue and colonoids and b) colon tissue and four Chip conditions.

Differential Gene Expression Analysis (CT vs Day 5-endo): Chips are found to be closer to tissue in comparison to colonoids. Stretch seems to increase the number of DE genes.

Differential Gene Expression Analysis (CT vs Day 5+endo). Chips are found to be closer to tissue in comparison to colonoids. Stretch seems to reduce the number of DE genes.

Proteomic Analysis of Colon-Chip

Experimental Conditions Tested

Goal: To evaluate the role of intestinal microvasculature and stretching on the maturation and function of human colonic epithelium. Conditions were tested in Donor 1 at one experimental timepoint (day 8).

Presence of Endothelial Cells Accelerates Barrier Formation in Colon-Chip. Colonic endothelial cells decrease the time required for epithelial barrier formation in Colon-Chips. Epithelium-only Colon-Chip.

On-Chip Culture Shows Increased Differentiation of Epithelial Cell Types: Similarly to ileum, Colon-chip culture has showed an increased level of differentiation of absorptive enterocytes, Goblet cells and enteroendocrine cells in comparison to colonoids.

Similar Levels of Tuft Cells Maturation in Colon-Chips and Colonoids. Similar levels of Trpm5 expression, marker specific for Tuft cells observed in Colon-Chip and colonoids.

Decreased levels of stem cell marker LGR5 on-Chip in comparison to 3D colonoids. For test conditions Chips are found to be closer to tissue relatively to colonoids (smaller number of differentiallyexpressed genes).

Condition Up-regulated Down-regulated Total DE Genes.

In the presence of Endos, stretching seems to reduce the number of DE genes

In the absence of Endos, stretching seems to increase the number of DE genes

The number of DE genes is only indicative of trends, pathway analysis is necessary to probe deeper.

TABLE 7
Differential Gene Expression Analysis Summary.

| Condition | Up-regulated | Down- regulated | Total DE Genes |
|---|---|---|---|
| ct vs colonoid | 956 | 924 | 1880 |
| ct vs D5 −e/+s | 209 | 318 | 527 |
| ct vs D5 −e/−s | 106 | 357 | 464 |
| ct vs D5 +e/−s | 93 | 96 | 189 |
| ct vs D5 +e/+s | 54 | 80 | 134 |
| ct vs D8 +e/−s | 654 | 506 | 1160 |
| ct vs D8 −e/+s | 207 | 761 | 968 |
| ct vs D8 +e/+s | 640 | 328 | 968 |
| ct vs D8 −e/−s | 151 | 586 | 737 |

Green - Italics.
Red BOLD.

Assessment of GPR35 Targeting in Intestine-Chip.

Testing of GPR35 agonists in Colon-Chip. Aim, Timeline, Readouts.

Stimulation (Luminal exposure): Evaluation of the GPR35 targeting in Colon-Chips established from 2 independent donors.

Stimulations will be performed in presence/absence of 50 ng/ml TNFα.

Colon-Chip was apically treated with 100 nM of SYR381171, SYR493714 or DMSO.
100 nM of SYR381162—GPR35 agonist (active)
100 nM of SYR381171—GPR35 agonist (active)
100 nM of SYR493714—GPR35 agonist (inactive)
Vehicle control—DMSO

In other embodiments, stimulations may be performed in presence/absence of basally applied 20 ng/ml TNF-α and 10 ng/ml IL-1b.

Exemplary Readouts: IF: against Muc2 and ChrgA to determine the abundance of goblet and enteroendocrine cells. RNAseq. LC-MS: chip effluents and dosing solutions containing GPR35 agonists will be assessed and compared. Barrier function and Brightfield imaging.

GPR35 Treatment Effect on Colon-Chip Morphology and Barrier Function.

Aim: Testing of GPR35 agonists will be performed in Caco-2 Intestine-Chip. Stimulation (Luminal exposure). Stimulations may be performed in presence/absence of 50 ng/ml TNFα.
100 nM of SYR381171—GPR35 agonist (active)
100 nM of SYR493714—GPR35 agonist (inactive)
Vehicle control—DMSO

Exemplary Readouts: RNAseq

GPR35 stimulation in Caco-2 Intestine-Chip Experimental Conditions. RNAseq analysis may be performed at 6 hours post-stimulation.

| | | | | Untreated | +TNF- |
|---|---|---|---|---|---|
| Compound | Timepoints | | Readouts | (Chips) | α(Chips) |
| TO Control | 0 | min | RNAseq | 3 | — |
| SYR381171 | 6 | hrs | RNAseq | 3 | 3 |
| (Active) | | | | | |
| SYR493714 | 6 | hrs | RNAseq | 3 | 3 |
| (Inactive) | | | | | |
| Vehicle | 6 | hrs | RNAseq | 3 | 3 |
| (DMSO) | | | | | |

| | | |
|---|---|---|
| Subtotal | 24 | 18 |

| | |
|---|---|
| Total | 42 |

Sampled Collected for RNAseq, LC-MS and Cytokine Analysis
The total of 58 samples (Colon-Chips lysed in TRIzol) and 21 samples (Caco2 Intestine-Chips lysed in TRIzol) was collected and shipped to Qsolutions (on 28 October and on 5 November) for the further assessment.
The total of 328 samples (Colon-Chip effluents) were collected for the evaluation of the cytokine profiles. The total of 210 samples (Colon-Chip effluents) were collected for the LC-MS assessment.

Testing of GPR35 agonists in Colon-Chip—Other embodiments, Experimental Repeat.

| | | | | | | +TNF-α/ | |
|---|---|---|---|---|---|---|---|
| | Treatment | Termination | Duration of | | Untreated | IL-1p | Biological |
| Compound | Day | Day | Treatment | Readouts | (Chips) | (Chips) | Replicates |
| TO Control | — | — | 0 min | Permeability and IF | 3 | — | Donor 1 |
| | | | | (Muc2, ChrgA) | | | |
| SYR381162 | Day 5 | Day 8 | 24 hrs exposure, | Permeability and IF | 3 | 3 | Donor 1 |
| (Active) | | | followed by 48 h | (Muc2, ChrgA) | | | |
| | | | incubation (fresh | | | | |
| | | | media) | | | | |
| SYR381171 | Day 5 | Day 8 | 24 hrs exposure, | Permeability and IF | — | 3 | Donor 1 |
| (Active) | | | followed by 48 h | (Muc2, ChrgA) | | | |
| | | | incubation (fresh | | | | |
| | | | media) | | | | |
| SYR493714 | Day 5 | Day 8 | 24 hrs exposure, | Permeability and IF | — | 3 | Donor 1 |
| (Inactive) | | | followed by 48 h | (Muc2, ChrgA) | | | |
| | | | incubation (fresh | | | | |
| | | | media) | | | | |
| Vehicle | Day 5 | Day 8 | 24 hrs exposure, | Permeability and IF | — | 3 | Donor 1 |
| (DMSO) | | | followed by 48 h | (Muc2, ChrgA) | | | |
| | | | incubation (fresh | | | | |
| | | | media) | | | | |
| | | | | Subtotal | 6 | 12 | |
| | | | | Total | 18 | 18 | |

Colon-Chip was basically stimulated with 20 ng/ml TNF-α and 10 ng/ml IL-1b. Colon-Chip was apically treated with 100 nM of SYR381162, SYR381171, SYR493714 or DMSO.

Functional Validation of Colon-Chip: Mucus Production and Release.

Potential Triggers/Additional Features of the System: short chain fatty acids SCFA; butyrate; cytokines e.g. IL-13, 11-25, IL-22; acetylcholine or other cholinergic agonists such as carbachol; immune modulator prostaglandin E2 (PGE2); culture at the Air-Liquid Interface (ALI).

Potential Readouts: Mucus composition: LC-MS, ELISA; Mucus thicknes; imaging-based assessment of the localization of fluorescent beads/bioparticules; Mucus penetrability/protective function; co-culture with a commensal (e.g. E. coli Nissle 1917) and pathogen bacterial strains (e.g. EHEC), subsequent; measurement of mucus thickness as well as apical and basolateral secretome profile of the IECs.

Exemplary TaqMan Assay Genes.

| Gene | Gene Name | TaqMan Assay ID |
|---|---|---|
| ALPI | Alkaline Phosphatase | Hs00357579_gl |
| BCRP/ABCG2 | ATP binding cassette subfamily G member 2 | Hs01053790_ml |
| CHGA | Chromogranin A | Hs00900375_ml |
| CYP3A4 | cytochrome P450 family 3 subfamily A member 4 | Hs00604506_ml |
| EpCAM | Epithelial Cell Adhesion Molecule | Hs00901885_ml |
| LYZ | Lysozyme | Hs00426232_ml |
| MDR1/ABCB1 | ATP binding cassette subfamily B member 1 | Hs00184500_ml |
| MRP2/ABCC2 | ATP binding cassette subfamily C member 2 | Hs00960489_ml |
| MRP3/ABCC3 | ABCC3 | Hs00978452_ml |
| MUC2 | Mucin 2 | Hs00894025_ml |
| OAT2B1/ | solute carrier organic anion transporter family | Hs01030343_ml |
| SLC02B1 | member 2B1 | |
| OCT1/SLC22A1 | solute carrier family 22 member 1 | Hs00427552_ml |
| PEPT1/SLC15A1 | solute carrier family 15 member 1 | Hs00192639_ml |
| PXR/NR1I2 | nuclear receptor subfamily 1 group I member 2 | Hs01114267_ml |
| SLC40A1 | solute carrier family 40 member 1 | Hs00205888_ml |
| VDR | vitamin D (1,25- dihydroxyvitamin D3) receptor | Hs01045843_ml |

The following references relate to colon function:

| | | CELLS | Demonstrated Effect of Constant |
|---|---|---|---|
| REFERENCE | MODEL SYSTEM | ORIGIN | ALI |
| Nichols, J. E. et al., | airway epithelium | human | Differentiation of basal cells to ciliated |
| doi: 10.1177/1535370214536679 | | | and secretory goblet cells |
| Schilders, K. A. A. et al., | airway epithelium | human | Differentiation of basal cells to ciliated |
| do [symbol] 2931 [symbol] 6-0358 [symbol] | | | and secretory goblet cells |
| Nossol, C. et al., | intestinal | porcine | Improved intestinal epithelial |
| doi: 10.1007/S00418-0110826-y | epithelial cell line | | cells differentiation and |
| | (IPEC-J2) | | function through optimized |
| | | | oxygenation |
| Viney, M. E. et al., | Rat-2, IEC 6, IPI- | human, | Extended epithelial culture in vitro |
| doi: 10.2217/rme.09.4 | 21, CRL· 2102* | rat | |
| Dimarco, R. L. et al., | C57BL/6J neonatal | mouse | Enhanced oxygen transport rates, |
| doi: 10.1039/c3ib40188j | intestinal | | higher equilibrium oxygen |
| | organoids* | | concentrations, decreased oxygen |
| | | | gradients |
| Ootani, A et al., | jejunal and colonic | mouse | Extended epithelial culture in vitro |
| doi: 10.1038 nm.1951 | organoids* | | |
| Klasvogt, S. et al., | intestinal epithelial | porcine | Enhanced oxidative |
| doi: 10.1038 cddiscovery 2017 [symbol] | cell line (IPEC-J2) | | phosphorylation and reduced |
| | | | glycolysis |
| Grivel, J. C. & Margolis, L., | rectosigmoidal | human | Extended epithelial culture in vitro |
| doi 10.1038 nprot 2008.245 | biopsies | | |

[symbol]

indicates data missing or illegible when filed

VI. Treatments for Improving Barrier Function: Rescuing Damaged Cells-Tissues-Organs.

There are few methods available for monitoring in vivo treatment, e.g. drug, responses on specific areas of cells, or tissues, or organs, e.g., including responses in both damaged areas of patients showing disease symptoms, e.g., demonstrating disease effects and adjacent nondamaged areas (e.g., including areas demonstrating lesser damage or no damage). Further, there are even fewer methods available for monitoring such cellular, tissue or organ drug responses in vivo. Therefore, in vitro methods are needed for monitoring responses mimicking in vivo-like responses. Thus, in some embodiments, a microfluidic device as described herein, is contemplated for use in assays for determining in vivo-like responses of damaged cells, tissues or organs to a test substance for rescuing functions of damaged tissue and preventing damage of adjacent areas. In preferred embodiments, a test rescue substance improves function of damaged cells, tissues or organs, e.g. improves barrier function of an epithelial cell layer, an endothelial cell layer, or both types of cell layers in a microfluidic device. In some embodiments, microfluidic device based evaluation of rescue substances is more robust than assays in transwell plates or other static platforms. As one example, the amount of damage induced by an injury substance to a cell layer in a microfluidic device, may be at least 2-fold, at least 3-fold, at least 4-fold, at least 5-fold, at least 10-fold, at least 20-fold, up to at least 50-fold or more. Moreover, the amount of rescue, i.e. recovery or prevention of injury, may be at least 50%, at least 70%, at least 80%, at least 85%, at least 90%, at least 95% or more up to 100%.

Exemplary epithelial cells include but are not limited to hepatocytes, liver cells, kidney tubule cells, skin cells, intestinal cells, etc., In some embodiments, epithelial cells are organoid-derived colonic epithelial cells. In some embodiments, endothelial cells are intestinal microvascular endothelial cells.

Exemplary endothelial cells include but are not limited to microvascular endothelial cells (MECs), intestine-derived endothelial cells, human intestinal microvascular endothelial cells (HIMEC); brain microvascular endothelial cells (BMECs), human brain microvascular endothelial cells (hBMECs), rat brain microvascular endothelial cells (RBMVEC), renal glomerular endothelial cells (GEC), and human umbilical vein endothelial cells (HUVECs).

In preferred embodiments, epithelial cells and endothelial cells form a cell layer on-chip. It is not intended to limit cell layers to epithelial cells and endothelial cells, such that other cell types may be present including but not limited to endocrine cells, sensory cells, etc.

In some embodiments, a test injury inducing substance is evaluated as described herein, for reversibility. Thus, reversible injury inducing substances identified as described herein, are further used for testing efficacy of test rescue substances. In some embodiments, substances may be identified that are nonreversible using known rescue substances. Thus, further testing is contemplated for identifying substances for counteracting this type of injury.

A. Improving Functions of Cells-Tissues-Organs, e.g. Increasing Barrier Functions.

Contemplated embodiments of microfluidic organ devices, such as described herein, include inducing a disease state for a specific period of time on-chip for use in evaluating responses of organ mimicking microfluidic chips to substances intended to improve, e.g. reverse, cell and/or tissue and or organ damage.

Thus, in some embodiments, a substance is evaluated for rescuing damaged barrier morphology and/or function. In some embodiments, a barrier refers to a tissue barrier, e.g. epithelial tissue barrier. In some embodiments, a barrier refers to a cellular barrier, e.g., an endothelial cell barrier. In some embodiments, a barrier refers to both an epithelial tissue barrier and an endothelial cell barrier. In some embodiments, evaluation of barrier rescue is observing recovery of a morphologically intact cellular layer; or an increase in barrier function, e.g., lowering amounts of small molecules crossing a damaged barrier. In some embodiments, barrier function is observed morphologically, e.g., tight junction immunohistochemistry, tight junction immunofluorescent evaluations, such as with ZO-1 staining. In some embodiments, barrier function is evaluated by measuring electrical barrier function, e.g. Papp.

1. Exemplary Evaluation for Reversibility of an Injury Inducing Substance.

In some embodiments, a microfluidic device such as described herein, is used for evaluating a test injury inducing substance, e.g. for increasing permeability of a cell layer compared to an untreated cell layer, wherein increased permeability is reversible. Such substances identified as injury inducing substances would be further tested for reversibility of effect in the presence of a known rescue substance or a rescue substance discovered using the methods described herein. Thus, in some embodiments, a microfluidic device such as described herein, is used for evaluating the reversibility of a test injury inducing substance. In some embodiments, a microfluidic device is used for testing the combitorial effects of more than one test injury inducing substance, sequentially or in a mixture, further tested for reversibility of effect in the presence of a known rescue substance.

In some embodiments, an injury (damage) substance continuously flows through a microfluidic device with or without the presence of a test substance for ‘rescuing’ injury/damage.

In some embodiments, cells are exposed to a test injury inducing substance for up to 3 hours. In some embodiments, cells are exposed to a test injury inducing substance for up to 12 hours, up to 24 hours, up to 48 hours up to 72 hours or more. In some embodiments, cells are exposed to a test injury inducing substance in combination with a known rescue substance. In some embodiments, cells are simultaneously exposed to a test injury inducing substance and a known rescue substance for up to 12 hours, up to 24 hours, up to 48 hours up to 72 hours or more.

Thus, in some embodiments, a known substance for ‘rescuing’ damage, e.g. barrier damage, is flowed into a microfluidic device up to at least 3 hours simultaneously with a first exposure to a test damaging substance. In some embodiments, a known substance for ‘rescuing’ barrier damage is flowed into a microfluidic device at least 3 hours after a first exposure to a test damaging substance. In some embodiments, a known substance for ‘rescuing’ barrier damage is flowed into a microfluidic device over a 12 hour time period. In some embodiments, a known substance for ‘rescuing’ barrier damage is flowed into a microfluidic device over a 24 hour time period. In some embodiments, a known substance for ‘rescuing’ barrier damage is flowed into a microfluidic device over a 48 hour time period. In some embodiments, a known substance for ‘rescuing’ barrier damage is flowed into a microfluidic device over a 72 hour time period.

One example of an injury inducing substance, e.g., a barrier disrupting substance is AT1002. AT1002 refers to a six-mer synthetic peptide H-FCIGRL-OH of a Zonula occludens toxin, with or without amidation at a C-terminal end, representing an active domain of Vibrio cholerae's second toxin, zonula occludens toxin (ZOT). It is known to reversibly open tight junctions (Ti) in endothelial and epithelial cells, e.g. causes the redistribution of ZO-1 away from cell junctions as observed by fluorescence microscopy. This synthetic peptide (H-FCIGRL-OH) retains Zot permeating effects on intercellular Ti (tight junctions) resulting in decreased transepithelial electrical resistance (TEER) and increased Ti permeability. Thus, AT-1002 caused a reversible reduction in transepithelial electrical resistance (TEER) and an increase in lucifer yellow permeability in Caco-2 cell monolayers. AT1002 also enhances the transport of molecular weight markers or agents with low bioavailability through barriers with no overt cytotoxicity. AT1002 has tight junction modulator activity with permeation-enhancing activity. Gopalakrishnan et al, 2009.

Another example of an injury inducing substance is a cytokine, e.g., IFN-gamma, TNF-alpha, etc.

2. Exemplary Evaluation of a Rescue Substance.

In some embodiments, a microfluidic device such as described herein, is used for evaluating a test rescue substance. Thus, in some embodiments, a microfluidic device such as described herein, is used for evaluating a test rescue substance after treatment with a known reversible injury inducing substance. In some embodiments, a known injury inducing substance or an injury inducing substance discovered using the methods described herein, is used for injuring a barrier in a microfluidic device for use in testing a rescue substance for reversing or counteracting the effects of a known injury inducing substance. In addition, agents are tested on cells of a barrier (e.g. intestinal barrier) that degrade function (e.g. increase permeability) and restore function (e.g. reduce permeability).

One example of a rescue substance, e.g., a barrier restoration substance, is larazotide acetate, referring to an eight amino acid synthetic peptide. Larazotide acetate is the first in a novel class of drug candidates for treating celiac disease (CeD), currently in Phase 3 trials (INN-202). INN; also known as AT-1001; is formulated as a salt with acetic acid, as larazotide acetate refers to a synthetic eight amino acid peptide that functions as a tight junction regulator and reverses leaky junctions to their normally closed state. It acts as tight junction regulator, helping to restore “leaky” barrier or open junctions, including barriers induced to leak using AT1002 in vitro. Gopalakrishnan et al, 2012, Khaleghi 2016 Khaleghi et al, 2016; Pearce et al, 2018.

One example of a barrier restoration assay in vitro is a method where AT-1002-induced TEER reduction and Ti opening in Caco-2 cells that was ‘rescued’ by larazotide acetate treatment. Larazotide acetate refers to a synthetic peptide derived from the zonula occludens toxin secreted by Vibrio cholera. Additionally, larazotide acetate inhibited the translocation of a gliadin 13-mer peptide, which was implicated in celiac disease, across Caco-2 cell monolayers. Further, apically applied larazotide acetate inhibited the increase in TJ permeability elicited by basolaterally applied cytokines. See, for one example, Gopalakrishnan et al, 2012. Examples of amounts used for clinical testing include (0.25, 1, 4, or 8 mg). It is not intended to limit a test rescue substance to one substance. Indeed, more than one substance may be tested, e.g., larazotide acetate in combination with obeticholic acid (OCA). Gopalakrishnan et al, 2012.

3. Exemplary Barrier Disruption/Rescue Assays.

In some embodiments, a microfluidic device as described herein, is used for an in vitro assay for evaluating a substance for its ability or efficacy of ‘rescuing’ barrier function. In some embodiments, a human intestine chip is used for an in vitro assay for evaluating a substance for ‘rescuing’ barrier function. In some embodiments, a human intestine chip is an organoids-derived colonic intestine chip. In some embodiments, Organoids-derived Colonic Epithelial Cells are seeded into one channel with Intestinal Microvascular Endothelial Cells seeded into an opposing channel separated by a membrane, as described herein. After seeding cells on Day 0, flow was initiated on Day 1 with stretch initiated on Day 2. A substance for inducing injury, e.g. AT1002 (e.g., 1 mM-20 mM), is flowed into a channel on Day 5 followed by treatment by flowing in a test substance, e.g. larazotide acetate, for evaluating rescue on Day 7 (or 12-48 hours after the first injury treatment).

a. Exemplary Rescue Using from an Injury Inducing Peptide Substance.

The following is an exemplary timeline for one embodiment of a Barrier Disruption/Rescue Assay, e.g. rescuing 3 hours of injury by a peptide substance, e.g. AT1002 (synthetic peptide H-FCIGRL-OH of ZO toxin) with AT1001, larzaride acetate. Day −5: experiment preparation (prep), as described herein; Day 0: Intestine chip seeding both cells types, epithelial and endothelial; Day 1 starting flow; Day 2 starting stretch, in some embodiments starting flow at lower levels then increasing up to 10% over 24 hours (1 day), over 2 days, or over 3 days; Day 5; starting Injury/Rescue Assay: using a known reversible injury inducing substance: Injure period starting at 0 hour continuing up to 3 hours, or more. Day 6 first readout time point (24 hours); Day 7, in some embodiments, Fix cells at 48 hours post injury; Days 7-21, in some embodiments, initiate at least one Endpoint Assay for analysis; and in some embodiments Day 21 is a report-out of assay. Exemplary check timepoints for readouts: 0, 24 and 48 hours after initiation of injury inducing substance. In some embodiments, Endpoint Assays and Analysis are done on Day 14. In some embodiments, Endpoint Assays and Analysis are done on Day 21. In some embodiments, a test substance for ‘rescuing’ damage may be evaluated within 14 days. In some embodiments, a test substance for ‘rescuing’ damage may be evaluated within 21 days from the beginning of cell culturing.

FIG. 93 shows an exemplary time line for embodiments of a rescue assay; first inducing barrier injury (e.g. reducing apparent permeability) then testing a substance for improving (e.g. increasing apparent permeability).

Embodiments of read-outs include using small molecules, e.g. 3 KDa Dextran Cascade Blue, and electrical measurements, e.g. TEER, for evaluating barrier function.

As one exemplary injury substance, AT1002 treatment shows injury effects by inducing “leaky gut”/barrier disruption in a concentration-dependent manner in one embodiment of an organoid-derived colon chip.

FIG. 94 shows an exemplary concentration dependent response of an organoid-derived colon chip to AT1002 measured by Apparent Permeability using 3 KDa Dextran Cascade Blue (P^{App} (cm/s)×10⁻⁷).

One exemplary rescue substance, larazotide Acetate, was tested for counteracting damage of AT1002. Thus, efficacy of Larazotide Acetate e.g. 20 mM, for rescuing barrier function was tested in one embodiment of an organoid-derived colon chip exposed to AT1002. Larazotide acetate (20 mM) is able to restore/rescue barrier integrity in Colon Intestine-Chip exposed to AT1002.

FIG. 95 shows exemplary efficacy of Larazotide Acetate to restore/rescue up to 100% of barrier integrity in Colon Intestine-Chip exposed to AT1002 after 24 hours. Larazotide acetate at 20 mM. N=5. Apparent Permeability was measured using 3 KDa Dextran Cascade Blue.

b. Rescuing Injury Induced by Cytokines.

Barrier Disruption by Cytokines, e.g., IFN-gamma. In some embodiments, one or a combination of at least two injury inducing cytokines are used for testing rescue from cytokine injury. Thus, in one embodiment, IFN-γ induced injury to barrier function is evaluated in a barrier disruption/rescue assay of a microfluidic device comprising: Top Channel: Organoids-derived Colonic Epithelial Cells and Bottom Channel: Intestinal Microvascular Endothelial Cells.

Day −5: experiment preparation (prep); Day 0: Intestine chip seeding both cells types (epithelial cells and endothelial cells); Day 1 flow; Day 2 stretch; Day 5; Injury/rescue: Time points 0, 24 and 48 hours; Injure 0 hour starting FN-gamma exposure—up to 48 hs; Day 6 first time point (24 hours) add Larazotide—24 h; Day 7 Fix at 48 hours; Day 8 up to 72 hour assay; Days 7-21 initiate at least one Endpoint Assay for analysis; Day 21 report out of assay. Test barrier function morphology at timepoints 0, 24, 48 and 72 hours.

FIG. 96 shows an exemplary time line for embodiments of a cytokine substance rescue assay; first inducing barrier injury using a cytokine (e.g. IFN-γ) then testing a substance for improving (e.g. increasing apparent permeability), e.g. Larazotide acetate (12.5 mM).

Permeability of Colon Intestine Chip increases post 48 hours independent of IFN-gamma concentrations tested, between 10 ng/ml—50 ng/ml. Apparent Permeability: 3 KDa Dextran Cascade Blue.

FIG. 97 shows an exemplary concentration independent response of an organoid-derived colon chip to a substance, e.g. cytokine, such as IFN-gamma, 10, 25 and 50 ng/ml. Apparent Permeability to 3 KDa Dextran Cascade Blue (P^{App} (cm/s)×10⁻⁷). N=5.

Restoration of barrier disruption through cytokines, e.g., Larazotide acetate. Efficacy of Larazotide Acetate, e.g. 12.5 mM, for rescuing barrier function was demonstrated in one embodiment of an organoid-derived colon chip. Larazotide acetate (12.5 mM) partially restored/rescued barrier integrity/function when injured by IFN-gamma, showing statistically significance when compare to a disrupted condition. IFN-gamma: 25 ng/ml. Top Channel: Organoids-derived Colonic Epithelial Cells. Bottom Channel: Intestinal Microvascular Endothelial Cells (HIMEC).

FIG. 98 shows an exemplary restoration by Larazotide acetate (12.5 mM) of barrier permeability disrupted by a cytokine (e.g. IFN-γ). Statistically significance between 0 and 24 hours after disruption (injury). Apparent Permeability: 3 KDa Dextran Texas Red (P^{App} (cm/s)×10⁻⁷). N=5.

VII. Detailed Description of Methods and Fluidic Chips.

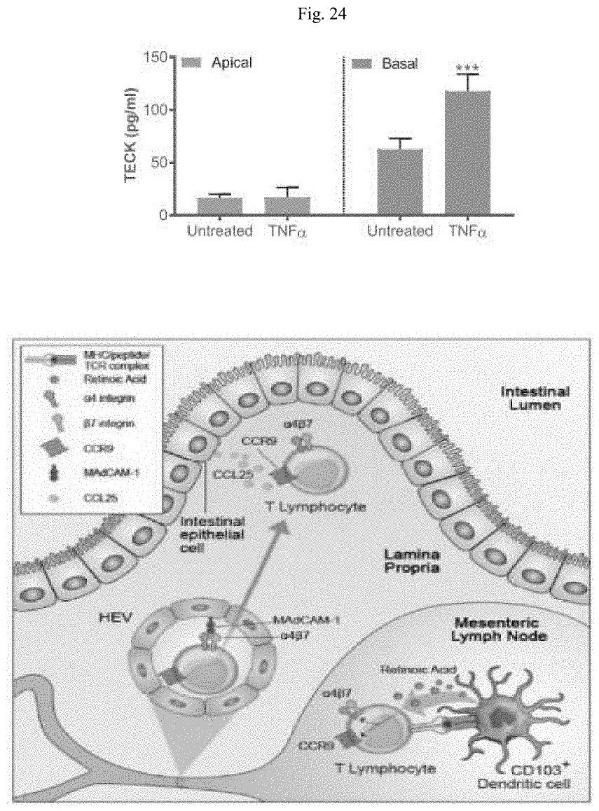

Propagation of Suspension Enteroids/Colonoids. FIG. 24 Shows an exemplary Propagation of Suspension Enteroids/Colonoids.

### Example A: Normal Human Organoids On-Chip

We assess the functionality of multiple cell types of organoids on Chip compared to suspension organoids in some instances. MDR1 fluorescent substrate rhodamine 123 will be used to confirm MDR1 transporter activity functionality. Rhodamine is expected to enter the cell and be blocked by the MDR1 blocker verapamil. In addition, we could test efflux activity by one of the drugs digoxin or erythromycin. Organoids loaded with the calcium indicator Fluo-4, demonstrating an increase in intracellular calcium upon stimulation with ATP and acetylcholine. Measurement of CYP3A4 enzyme activity. Stimulation of the release of GLP-1 by medium and long chain fatty acids (MCFAs and LCFAs) through the receptors GPR40 and 120 short chain fatty acids SCFA through GPR41 (small intestine) and GPR43 (large intestine) S Ethanolamides that signal through the receptor GPR119, which is expressed by L-cells Latrunculin mediated alteration in cytoskeleton.

CYP3A4 Enzyme Activity Across Multiple Donors in The Duodenum Intestine-Chip. Duodenum Intestine-Chip reflects clinically-relevant donor-to-donor variability and demonstrates in vivo relevant CY3A4 activity levels versus organoids that show lower drug metabolizing capacity. In vivo referenced from: Obach R. S. et all. Drug Metabolism and Disposition. 2001, 29 (3) 347-352 (human small intestinal microsomal preparations). See, FIG. 12, for example.

### Example B

Propagation of Suspension Enteroids/Colonoids includes but is not limited to: Passaging ratio—1:3 (duodenum, jejunum, ileum, colon); Passaging frequency—every 7 days; Fragmentation technique—digestion using TrypLE (1:1 vol/vol in DPBS) in the presence of ROCK inhibitor.

Exemplary Timeline: FIG. 24, includes but is not limited to: Freezing time: 3 days after passaging; Freezing media: Recovery™ Cell Culture Freezing Medium (Gibco); Enteroids/colonoids Stock Vials: 12 wells of enteroids/colonoids per vial.

Mini-Biobank of Enteroids/Colonoids.

Mini-biobank of normal human small intestine ileal derived organoids and normal human colonic derived colonoids has been successfully established. Established and optimized protocols for propagation and bio-banking of suspension ileal and colonic organoids samples from screening colonoscopy, includes but is not limited to: Donor 351 (53 y, female); Donor 461(50 y, male); Donor 601 (52 y, male).

Created a mini-biobank of at least 3 normal human small intestinal organoids (enteroids) and at least 4 normal human colonic organoids (colonoids). Mini-biobank of normal human small intestine ileal derived organoids and normal human colonic derived colonoids has been successfully established, samples from screening colonoscopy: Donor 351 (53 y, female); Donor 461 (50 y, male); Donor 601 (52 y, male).

Methods and Protocols: Establishment of Organoids Culture, includes but is not limited to: Thawing procedure; Embedding & Seeding protocol; Propagation of Suspension Organoids; Recovery from the Matrigel; Fragmentation procedure; Embedding & Seeding protocol; Culture maintenance; Bio-banking of Suspension;

Organoids; Recovery from the Matrigel; Organoids freezing protocol, etc.

TABLE 7
Bio-bank of Enteroids and Colonoids.

| | | Intestinal | | | Passage |
|---|---|---|---|---|---|
| Number | Donor | region | Age | Gender | received |
| 1 | 35I | Ileum | 53 | F | 26 |
| 2 | 46I | Ileum | 50 | M | 19 |
| 3 | 60I | Ileum | 52 | M | 6 |
| 4 | 70C | Colon | 66 | M | 15 |
| 5 | 73C | Colon | 56 | F | 24 |
| 6 | 75C | Colon | 58 | M | 23 |
| 7 | NA | Colon | TBD | M | 0 |

TABLE 8
Cell Types Characterization Summary.

| | | | Evidence | | Est. |
|---|---|---|---|---|---|
| Chip/Cell | Present/Absent | Based on | Immunofluorescence | qPCR | Frequency |
| Ileum-Chip | | | | | |
| Absorptive | Present | IF, qPCR | Villtn+ | Alpi+ | 91.2% ± 1.3^{b} |
| enterocytes | | | | | |
| Goblet cells | Present | IFqPCR | Muc2+ | Muc2+, | 6.1% ± 1.4^{b} |
| | | | | TFF3+ | |
| Enteroendocrine | Present | IFqPCR | ChrqA+ | ChrqA+ | 1.2% ± 0.3^{b} |
| cells | | | | | |
| Paneth cells | Present | IF, qPCR | Lyz+ | Lyz+ | 0.1% ± 0.2^{b} |
| Stem cells | Present | qPCR | nd | Lgr5+ | |
| L cells | Present | IFqPCR | Gcg+ | Gcg+ | 0.7% ± 0.2^{b} |
| Enterochromaffin | Present | IFqPCR | 5-HT+ | Tph1+ | 0.5% ± 0.2^{b} |
| cells | | | | | |
| Tuft cells | Inconclusive | IFqPCR | Delkl−, Trp5−, | Dclkl−, | nd |
| | | | Chat− | Trp5+, Chat− | |
| Colon-Chip | | | | | |
| Absorptive | Present | IFqPCR | Villin+ | Alpi+ | 68.3% ± 4.2^{C} |
| enterocytes | | | | | |
| Goblet cells | Present | IF, qPCR | Muc2+ | Muc2+, | 4.0% ± 0.5^{C} |
| | | | | TFF3+ | |
| Enteroendocrine | Present | IFqPCR | ChrgA+ | ChrgA+ | 4.0% ± 0.7^{C} |
| cells | | | | | |
| Stem cells | Present | qPCR | nd | Lgr5+ | nd |
| L cells | Present | IFqPCR | GLP-1+ | Gcg+ | 1.9% ± 0.1^{c} |

^{a}quantification performed on day 8 of fluidic culture and based on 10 FOV/chip, 3 Chips/donor, expressed as average across 3 different donors +/− SD.
^{b}quantification performed on day 8 of fluidic culture based on 5 FOV/chip, 1 Chips/donor, expressed as average across 3 different donors +/− SD.
^{c}quantification performed on day 8 of fluidic culture based on 5 FOV/chip, 3 Chips/donor, expressed as average across the Chips from 1 donor +/− SD (quantification for other 2 donors is on-going).

### Example C: Testing of Small Molecules in Intestine-Chip

GOALS: Directly Quantify Partitioning of Compound

Compound loss at equilibrium (kinetics); Directly quantify diffusion of compound; Time-dependent compound loss (dynamics). This is a method for quantifying the drug-specific extent of compound loss.

Single time point experiments are only capable of extracting kinetics, not dynamics. Time-dependent studies capture not only equilibrium endpoints (K), but also time-dependent changes/dynamics (D). 1(one)D computational models are used to fit experimental results of time-dependent studies.

Group I: Background Adsorption: Quantifies loss of compound caused by adsorption to glass.

Group II: Time-Dependent Absorption:

STEP 1: Preliminary evaluation of the PDMS absorption can be performed by the Modeling Team based on molecular weight (MW) and lipophilicity (log P) of the compound—poor prediction of the extend of the absorption.

STEP 2: Time-dependent static PDMS Absorption Test—to determine fundamental parameters (Diffusivity and Partition Coefficient).

STEP 3: Modeling of the compound loss in the chip for different flow rates—to make recommendation to minimize loss.

STEP 4: Comparison of the model and experimental results.

### Example D: Small Molecule Compound A and B

| | | | Dosing |
|---|---|---|---|
| | Molecular Weight | | Concentration |
| Compound | (g/mol) | logP | (MM) |
| Compound A | 446.9 | 3.75-4.02 | 1 |
| Compound B | 499.61 | 4.47-4.49 | 1 |

STEP 1: Compounds flagged as potentially absorbing onto fluidic components, e.g. Small molecule. High log P value of a compound, which is the logarithm of its partition coefficient between n-octanol and water log(c_{octanol}/c_{water}), is a well established measure of the compound's hydrophilicity.

STEP 2: Standard 72 hour absorption studies run for PDMS (glass vials). D and K determined in 1D COMSOL model by fitting experimental data.

STEP 3: 2D Chip model run to determine cellular exposure concentrations

Compound dosed in basal channel (which minimizes absorption) Flow modeled at: 30 μl/hr to imitate standard flow condition, 150 μl/hr to minimize PDMS exposure. Flow rate for Compound B exposure—150 μL/hr flow rate recommended for experiment.

STEP 4: Comparison of 2D Chip model and experimental results

Model predicted well the expected perfusion manifold outlet concentration collected in first 24 hrs. >80% recovery of dosing concentration for Compound A, regardless of flow rate. Approximately 70% recovery for Compound B at 150 μL/hr, <10% recovery at 304/hr.

CONCLUSION: Absorption Tests and Modeling Approach allowed to implement experimental modifications ensuring optimal exposure of highly absorbed compounds.

### Example E: Contemplated Example: Establish and Optimize GPR35 Stimulation Conditions in Intestine-Chip System, Including Compounds (GPR35 Agonists, Vedolizumab, TGR5 Agonists, SCFA)

Microfluidic chips will be treated luminally with below mentioned compounds and downstream MAPK activation, specifically pERK1/2 relative to total ERK1/2, will be examined at 0, 15 min post-stimulation. TNF-α treatment which also induces pERK1/2 would be used as control.

Functional validation of GPR35 stimulation will be assessed by following: pERK112 and pSTAT3 assessment. Functional impact of various GPR35 agonists examined by Q-PCR analyses of MAPK-dependent upregulated genes e.g. DUSP5/1/4, cyclinDI, COX2, PHRP, etc. Gene expression assessment will be performed 0, 6 hrs post-stimulation. As warranted by Q-PCR data, LC-MS and RNAseq profiling of treated Microfluidic chips will be implemented in future experiments for small molecules assessments.

| | | ELISA | | RNA seq | | Technical | | Intestine |
|---|---|---|---|---|---|---|---|---|
| Stimulant | Concentrations | Timepoints | | Timepoints | | replicates | ±TNF-α | Chips |
| SYR381162 | 100 nM | 0, 15 | mins | 0, 6 | hrs | 2 | ×2 | 16 |
| SYR493714 | 100 nM | 0, 15 | mins | 0, 6 | hrs | 2 | ×2 | 16 |
| (inactive) | | | | | | | | |
| Vehicle | DMSO | 15 | mins | 6 | hrs | 2 | NA | 4 |

| | |
|---|---|
| Total | 36 |

I. Microfluidic Chips, Devices and Systems.

In some embodiments, chips that may find use are described herein, with additional embodiments and descriptions in U.S. Pat. No. 8,647,861, “Organ mimic device with microchannels and methods of use and manufacturing thereof.” filed Jul. 16, 2009, and WO2017035484; U.S. Ser. No. 10/233,416 (U.S. patent application Ser. No. 15/248,690), as examples, both of which are herein incorporated by reference in their entirety. Some Embodiments for Organ-On-Chip cultures as described herein, are contemplated to show physiological and morphological changes in epithelial cell layers directly related to the source of or differentiation stage or functions status of derived cells. Accordingly, some embodiments described herein relate to devices for simulating a function of epithelial tissue (also referred to as “organ-on-a-chip device”). The organ-on-a-chip microfluidic devices described herein can be used to simulate at least one or more (e.g., 1, 2, 3, 4, 5 or more) phenotypes and or functions of a variety of tissues. Microfluidic devices includes both closed-top and open-top chips, in addition to any one or more of embodiments as described herein and in referenced patent documents.

II. Closed Top Chips.

The present disclosure relates to organ-on-chips, such as fluidic devices comprising one or more cells types for the simulation of one or more of the function of organ components. Accordingly, the present disclosure additionally describes closed-top liver-on-chips, kidney-on-chips, e.g. proximal tubule-kidney-on-chips, lung-on-chips, etc., see, e.g. schematic in FIG. 1C. The present disclosure also relates to lymph node-on-chips, and BBB (blood brain barrier)-on-chips, which may also use a fluidic device such as depicted schematically in FIGS. 1A-D. The present disclosure relates to organ-on-chips, such as fluidic devices comprising one or more cells types for the simulation one or more of the function of epithelial components. The present disclosure additionally relates to fluidic devices comprising cells described herein as part of closed-top devices.

FIGS. 1A-B illustrates a perspective view of the devices in accordance with some embodiments described herein.

For example, as shown in FIGS. 1A-1B, the device 200 can include a body 202 comprising a first structure 204 and a second structure 206 in accordance with an embodiment. The body 202 can be made of an elastomeric material, although the body can be alternatively made of a non-elastomeric material, or a combination of elastomeric and non-elastomeric materials. It should be noted that the microchannel design 203 is only exemplary and not limited to the configuration shown in FIGS. 1A-1B. While operating channels 252 (e.g., as a pneumatics means to actuate the membrane 208, see herein for information on membrane 208 and see, International Appl. No. PCT/US2009s050830, the content of which is herein incorporated by reference in its entirety, for further details of the operating channels, are shown in FIGS. 1A-1B, they are not required in all of the embodiments described herein. In some embodiments, the devices do not comprise operating channels on either side of the microchannel. In other embodiments, the devices described herein can be configured to provide other means to actuate the membrane, e.g., as described in the International Pat. Appl. No. PCT/US2014/071570, the content of which is herein incorporated by reference in its entirety.

In some embodiments, various organ chip devices described in the International Patent Application Nos. PCT/US2009/050830; PCT/US2012/026934; PCT/US2012/068725; PCT/US2012/068766; PCT/US2014/071611; and PCT/US2014/071570, the contents of each of which are herein incorporated by reference in their entireties, can be modified to form the devices described herein. For example, the organ chip devices described in those patent applications can be modified in accordance with the devices described herein.

The first structure 204 and/or second structure 206 can be fabricated from a rigid material, an elastomeric material, or a combination thereof.

As used herein, the term “rigid” refers to a material that is stiff and does not bend easily, or maintains very close to its original form after pressure has been applied to it.

The term “elastomeric” as used herein refers to a material or a composite material that is not rigid as defined herein. An elastomeric material is generally moldable and curable, and has an elastic property that enables the material to at least partially deform (e.g., stretching, expanding, contracting, retracting, compressing, twisting, and/or bending) when subjected to a mechanical force or pressure and partially or completely resume its original form or position in the absence of the mechanical force or pressure. In some embodiments, the term “elastomeric” can also refer to a material that is flexible/stretchable but does not resume its original form or position after pressure has been applied to it and removed thereafter. The terms “elastomeric” and “flexible” are interchangeably used herein.

In some embodiments, the material used to make the first structure and/or second structure or at least the portion of the first structure 204 and/or second structure 206 that is in contact with a gaseous and/or liquid fluid can comprise a biocompatible polymer or polymer blend, including but not limited to, polydimethylsiloxane (PDMS), polyurethane, polyimide, styrene-ethylene-butylene-styrene (SEBS), polypropylene, polycarbonate, cyclic polyolefin polymer/copolymer (COP/COC), or any combinations thereof.

As used herein, the term “biocompatible” refers to any material that does not deteriorate appreciably and does not induce a significant immune response or deleterious tissue reaction, e.g., toxic reaction or significant irritation, over time when implanted into or placed adjacent to the biological tissue of a subject, or induce blood clotting or coagulation when it comes in contact with blood.

Additionally or alternatively, at least a portion of the first structure 204 and/or second structure 206 can be made of non-flexible or rigid materials like glass, silicon, hard plastic, metal, or any combinations thereof.

In some embodiments, the first structure and/or second structure of the device and/or the membrane can comprise or is composed of an extracellular matrix polymer, gel, and/or scaffold. Any extracellular matrix can be used herein, including, but not limited to, silk, chitosan, elastin, collagen, proteoglycans, hyaluronic acid, collagen, fibrin, and any combinations thereof.

The membrane 208 can be made of the same material as the first structure 204 and/or second structure 206 or a material that is different from the first structure 204 and/or second structure 206 of the devices described herein. In some embodiments, the membrane 208 can be made of a rigid material. In some embodiments, the membrane is a thermoplastic rigid material. Examples of rigid materials that can be used for fabrication of the membrane include, but are not limited to, polyester, polycarbonate or a combination thereof. In some embodiments, the membrane 208 can comprise a flexible material, e.g., but not limited to PDMS. Additional information about the membrane is further described herein.

The device in FIG. 1A can comprise a plurality of access ports 205. In addition, the branched configuration 203 can comprise a tissue-tissue interface simulation region or regions (such as a region on the membrane 208 in FIG. 1B) where cell behavior and/or passage of gases, chemicals, molecules, particulates and cells are monitored.

FIG. 1B illustrates an exploded view of the device in accordance with an embodiment. In one embodiment, the body 202 of the device 200 comprises a first outer body portion (first structure) 204, a second outer body portion (second structure) 206 and an intermediary membrane 208 configured to be mounted between the first and second outer body portions 204 and 206 when the portions 204 and 206 are mounted onto one another to form the overall body.

The first outer body portion or first structure 204 can have a thickness of any dimension, depending, in part, on the height of the first chamber 204. In some embodiments, the thickness of the first outer body portion or first structure 204 can be about 1 mm to about 100 mm, or about 2 mm to about 75 mm, or about 3 mm to about 50 mm, or about 3 mm to about 25 mm. In some embodiments, the first outer body portion or first structure 204 can have a thickness that is more than the height of the first chamber by no more than 5 mm, no more than 4 mm, no more than 3 mm, no more than 2 mm, no more than 1 mm, no more than 500 microns, no more than 400 microns, no more than 300 microns, no more than 200 microns, no more than 100 microns, no more than 70 microns or less. In some embodiments, it is desirable to keep the first outer body portion or first structure 204 as thin as possible such that cells on the membrane can be visualized or detected by microscopic, spectroscopic, and/or electrical sensing methods.

The second outer body portion or second structure 206 can have a thickness of any dimension, depending, in part, on the height of the second chamber 206. In some embodiments, the thickness of the second outer body portion or second structure 206 can be about 50 μm to about 10 mm, or about 75 μm to about 8 mm, or about 100 μm to about 5 mm, or about 200 μm to about 2.5 mm. In one embodiment, the thickness of the second outer body portion or second structure 206 can be about 1 mm to about 1.5 mm. In one embodiment, the thickness of the second outer body portion or second structure 206 can be about 0.2 mm to about 0.5 mm. In some embodiments, the second outer first structure and/or second structure portion 206 can have a thickness that is more than the height of the second chamber by no more than 5 mm, no more than 4 mm, no more than 3 mm, no more than 2 mm, no more than 1 mm, no more than 500 microns, no more than 400 microns, no more than 300 microns, no more than 200 microns, no more than 100 microns, no more than 70 microns or less. In some embodiments, it is desirable to keep the second outer body portion or second structure 206 as thin as possible such that cells on the membrane can be visualized or detected by microscopic, spectroscopic, and/or electrical sensing methods.

In some embodiments, the first chamber and the second chamber can each independently comprise a channel. In some embodiments, the channel(s) may be microchannel(s). The channel(s) can be substantially linear or they can be non-linear. In some embodiments, the channels are not limited to straight or linear channels and can comprise curved, angled, or otherwise non-linear channels. It is to be further understood that a first portion of a channel can be straight, and a second portion of the same channel can be curved, angled, or otherwise non-linear. Without wishing to be bound by a theory, a non-linear channel can increase the ratio of culture area to device area, thereby providing a larger surface area for cells to grow. This can also allow for a higher amount or density of cells in the channel.

FIG. 1B illustrates an exploded view of the device in accordance with an embodiment. As shown in FIG. 1B, the first outer body portion or first structure 204 includes one or more inlet fluid ports 210 in communication with one or more corresponding inlet apertures 211 located on an outer surface of the first structure 204. The device 200 can be connected to a fluid source via the inlet aperture 211 in which fluid travels from the fluid source into the device 200 through the inlet fluid port 210.

In some embodiments, while not necessary, the first structure 204 can include one or more pressure inlet ports 214 and one or more pressure outlet ports 216 in which the inlet ports 214 are in communication with corresponding apertures 217 located on the outer surface of the device 200. Although the inlet and outlet apertures are shown on the top surface of the first structure 204, one or more of the apertures can alternatively be located on one or more lateral sides of the first structure and or second structure. In operation, one or more pressure tubes (not shown) connected to an external force source (e.g., pressure source) can provide positive or negative pressure to the device via the apertures 217. Additionally, pressure tubes (not shown) can be connected to the device 200 to remove the pressurized fluid from the outlet port 216 via the apertures 223. It should be noted that the device 200 can be set up such that the pressure port 214 is an outlet and pressure port 216 is an inlet. It should be noted that although the pressure apertures 217, 223 are shown on the top surface of the first structure 204, one or more of the pressure apertures 217, 223 can be located on one or more side surfaces of the first structure 204.

Referring to FIG. 1B, in some embodiments, the second structure 206 can include one or more inlet fluid ports 218 and one or more outlet fluid ports 220. As shown in FIG. 29B, the inlet fluid port 218 is in communication with aperture 219 and outlet fluid port 220 is in communication with aperture 221, whereby the apertures 219 and 221 are located on the outer surface of the second structure 206. Although the inlet and outlet apertures are shown on the surface of the second structure, one or more of the apertures can be alternatively located on one or more lateral sides of the second structure.

As with the first outer body portion or first structure 204 described above, one or more fluid tubes connected to a fluid source can be coupled to the aperture 219 to provide fluid to the device 200 via port 218. Additionally, fluid can exit the device 200 via the outlet port 220 and outlet aperture 221 to a fluid reservoir/collector or other component. It should be noted that the device 200 can be set up such that the fluid port 218 is an outlet and fluid port 220 is an inlet.

Additionally, the first outer body portion or first structure 204 can include one or more outlet fluid ports 212 in communication with one or more corresponding outlet apertures 215 on the outer surface of the first structure 204. In some embodiments, a fluid passing through the device 200 can exit the device to a fluid collector or other appropriate component via the corresponding outlet aperture 215. It should be noted that the device 200 can be set up such that the fluid port 210 is an outlet and fluid port 212 is an inlet. In some embodiments, as shown in FIG. 1B, the device 200 can comprise an inlet channel 225 connecting an inlet fluid port 210 to the first chamber 204. The inlet channels and inlet ports can be used to introduce cells, agents (e.g., but not limited to, stimulants, drug candidate, particulates), airflow, and/or cell culture media into the first chamber 204.

The device 200 can also comprise an outlet channel 227 connecting an outlet fluid port 212 to the first chamber 204. The outlet channels and outlet ports can also be used to introduce cells, agents (e.g., but not limited to, stimulants, drug candidate, particulates), airflow, and/or cell culture media into the first chamber 204.

Although the inlet and outlet apertures 211, 215 are shown on the top surface of the first structure 204 and are located perpendicular to the inlet and outlet channels 225, 227, one or more of the apertures 211, 215 can be located on one or more lateral surfaces of the first structure and/or second structure such that at least one of the inlet and outlet apertures 211, 215 can be in-plane with the inlet and/or outlet channels 225, 227, respectively, and/or be oriented at an angle from the plane of the inlet and/or outlet channels 225, 227.

In another embodiment, the fluid passing between the inlet and outlet fluid ports can be shared between the first chamber 204 and second chamber 206. In either embodiment, characteristics of the fluid flow, such as flow rate, fluid type and/or composition, and the like, passing through the first chamber 204 can be controllable independently of fluid flow characteristics through the second chamber 206 and vice versa.

In some embodiments, the second outer body portion and: or second structure 206 can include one or more pressure inlet ports 222 and one or more pressure outlet ports 224. In some embodiments, the pressure inlet ports 222 can be in communication with apertures 227 and pressure outlet ports 224 are in communication with apertures 229, whereby apertures 227 and 229 are located on the outer surface of the second structure 206. Although the inlet and outlet apertures are shown on the bottom surface of the second structure 206, one or more of the apertures can be alternatively located on one or more lateral sides of the second structure. Pressure tubes connected to an external force source (e.g., pressure source) can be engaged with ports 222 and 224 via corresponding apertures 227 and 229. It should be noted that the device 200 can be set up such that the pressure port 222 is an outlet and fluid port 224 is an inlet.

In some embodiments where the operating channels (e.g., 252 shown in FIG. 1A) are not mandatory, the first structure 204 does not require any pressure inlet port 214, pressure outlet port 216. Similarly, the second structure 206 does not require any pressure inlet port 222 or pressure outlet port 224.

In an embodiment, the membrane 208 is mounted between the first structure 204 and the second structure 206, whereby the membrane 208 is located within the first structure 204 and/or second structure 206 of the device 200. In an embodiment, the membrane 208 is a made of a material having a plurality of pores or apertures therethrough, whereby molecules, cells, fluid or any media is capable of passing through the membrane 208 via one or more pores in the membrane 208. As discussed in more detail below, the membrane 208 in one embodiment can be made of a material which allows the membrane 208 to undergo stress and/or strain in response to an external force (e.g., cyclic stretching or pressure). In one embodiment, the membrane 208 can be made of a material, which allows the membrane 208 to undergo stress and/or strain in response to pressure differentials present between the first chamber 204, the second chamber 206 and the operating channels 252. Alternatively, the membrane 208 is relatively inelastic or rigid in which the membrane 208 undergoes minimal or no movement.

In some embodiments where the device simulates a function of a tissue, such as a lymph node, the membrane can be rigid.

The first chamber 204 and/or the second chamber 206 can have a length suited to the need of an application (e.g., a physiological system to be modeled), desirable size of the device, and/or desirable size of the view of field. In some embodiments, the first chamber 204 and/or the second chamber 206 can have a length of about 0.5 cm to about 10 cm. In one embodiment, the first chamber 204 and/or the second chamber 206 can have a length of about 1 cm to about 3 cm. In one embodiment, the first chamber 204 and/or the second chamber 206 can have a length of about 2 cm.

The width of the first chamber and/or the second chamber can vary with desired cell growth surface area. The first chamber 204 and the second chamber 206 can each have a range of width dimension between 100 microns and 50 mm, or between 200 microns and 10 mm, or between 200 microns and 1500 microns, or between 400 microns and 1 mm, or between 50 microns and 2 mm, or between 100 microns and 5 mm. In some embodiments, the first chamber 204 and the second chamber 206 can each have a width of about 500 microns to about 2 mm. In some embodiments, the first chamber 204 and the second chamber 206 can each have a width of about 1 mm.

In some embodiments, the widths of the first chamber and the second chamber can be configured to be different, with the centers of the chambers aligned or not aligned. In some embodiments, the channel heights, widths, and/or cross sections can vary along the length of the devices described herein.

The heights of the first chamber and the second chamber can vary to suit the needs of desired applications (e.g., to provide a low shear stress, and/or to accommodate cell size). The first chamber and the second chamber of the devices described herein can have the same heights or different heights. In some embodiments, the height of the second chamber 206 can be substantially the same as the height of the first chamber 204. In some embodiments, the height of at least a length portion of the first chamber 204 (e.g., the length portion where the cells are designated to grow) can be substantially greater than the height of the second chamber 206 within the same length portion. For example, the height ratio of the first chamber to the second chamber can be greater than 1:1, including, for example, greater than 1.1:1, 1.5:1, 2:1, 2.5:1, 3:1, 3.5:1, 4:1, 4.5:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1. In some embodiments, the height ratio of the first chamber to the second chamber can range from 1.1:1 to about 50:1, or from about 2.5:1 to about 50:1, or from 2.5 to about 25:1, or from about 2.5:1 to about 15:1. In one embodiment, the height ratio of the first chamber to the second chamber ranges from about 1:1 to about 20:1. In one embodiment, the height ratio of the first chamber to the second chamber ranges from about 1:1 to about 15:1. In one embodiment, the height ratio of the first chamber to the second chamber can be about 10:1.

The height of the first chamber 204 can be of any dimension, e.g., sufficient to accommodate cell height and/or to permit a low shear flow. For example, in some embodiments, the height of the first chamber can range from about 100 μm to about 50 mm, about 200 μm to about 10 mm, about 500 μm to about 5 mm, or about 750 urn to about 2 mm. In one embodiment, the height of the first chamber can be about 150 urn. In one embodiment, the height of the first chamber can be about 1 mm.

The height of the second chamber 206 can be of any dimension provided that the flow rate and/or shear stress of a medium flowing in the second chamber can be maintained within a physiological range, or does not cause any adverse effect to the cells. In some embodiments, the height of the second chamber can range from 20 μm to about 1 mm, or about 50 μm to about 500 μm, or about 75 μm to about 400 μm, or about 100 μm to about 300 μm. In one embodiment, the height of the second chamber can be about 150 μm. In one embodiment, the height of the second chamber can be about 100 μm. The first chamber and/or the second chamber can have a uniform height along the length of the first chamber and/or the second chamber, respectively. Alternatively, the first chamber and/or the second chamber can each independently have a varying height along the length of the first chamber and/or the second chamber, respectively. For example, a length portion of the first chamber can be substantially taller than the same length portion of the second chamber, while the rest of the first chamber can have a height comparable to or even smaller than the height of the second chamber.

In some embodiments, the first structure and/or second structure of the devices described herein can be further adapted to provide mechanical modulation of the membrane. Mechanical modulation of the membrane can include any movement of the membrane that is parallel to and/or perpendicular to the force/pressure applied to the membrane, including, but are not limited to, stretching, bending, compressing, vibrating, contracting, waving, or any combinations thereof. Different designs and/or approaches to provide mechanical modulation of the membrane between two chambers have been described, e.g., in the International Patent App. Nos. PCT/US2009/050830, and PCT/US2014/071570, the contents of which are incorporated herein by reference in their entireties, and can be adapted herein to modulate the membrane in the devices described herein.

In some embodiments, the devices described herein can be placed in or secured to a cartridge. In accordance with some embodiments of some aspects described herein, the device can be integrated into a cartridge and form a monolithic part. Some examples of a cartridge are described in the International Patent App. No. PCT/US2014/047694, the content of which is incorporated herein by reference in its entirety. The cartridge can be placed into and removed from a cartridge holder that can establish fluidic connections upon or after placement and optionally seal the fluidic connections upon removal. In some embodiments, the cartridge can be incorporated or integrated with at least one sensor, which can be placed in direct or indirect contact with a fluid flowing through a specific portion of the cartridge during operation. In some embodiments, the cartridge can be incorporated or integrated with at least one electric or electronic circuit, for example, in the form of a printed circuit board or flexible circuit. In accordance with some embodiments of some aspects described herein, the cartridge can comprise a gasketing embossment to provide fluidic routing.

In some embodiments, the cartridge and/or the device described herein can comprise a barcode. The barcode can be unique to types and/or status of the cells present on the membrane. Thus, the barcode can be used as an identifier of each device adapted to mimic function of at least a portion of a specific tissue and/or a specific tissue-specific condition. Prior to operation, the barcode of the cartridge can be read by an instrument so that the cartridge can be placed and/or aligned in a cartridge holder for proper fluidic connections and/or proper association of the data obtained during operation of each device. In some embodiments, data obtained from each device include, but are not limited to, cell response, immune cell recruitment, intracellular protein expression, gene expression, cytokine/chemokine expression, cell morphology, functional data such as effectiveness of an endothelium as a barrier, concentration change of an agent that is introduced into the device, or any combinations thereof

In some embodiments, the device can be connected to the cartridge by an interconnect adapter that connects some or all of the inlet and outlet ports of the device to microfluidic channels or ports on the cartridge. Some examples interconnect adapters are disclosed in U.S. Provisional Application No. 61/839,702, filed on Jun. 26, 2013, and the International Patent Application No. PCT/US2014/044417 filed Jun. 26, 2014, the contents of each of which are hereby incorporated by reference in their entirety. The interconnect adapter can include one or more nozzles having fluidic channels that can be received by ports of the device described herein. The interconnect adapter can also include nozzles having fluidic channels that can be received by ports of the cartridge.

In some embodiments, the interconnect adaptor can comprise a septum interconnector that can permit the ports of the device to establish transient fluidic connection during operation, and provide a sealing of the fluidic connections when not in use, thus minimizing contamination of the cells and the device. Some examples of a septum interconnector are described in U.S. Provisional Application No. 61/810,944 filed Apr. 11, 2013, the content of which is incorporated herein by reference in its entirety.

A Membrane Located in Between the First Structure and Second Structure.

In one embodiment, the membrane 208 is oriented along a plane 208P parallel to the x-y plane between the first chamber 204 and the second chamber 206. It should be noted that although one membrane 208 is shown, more than one membrane 208 can be configured in devices which comprise more than two chambers.

The membrane separating the first chamber and the second chamber in the devices described herein can be porous (e.g., permeable or selectively permeable), non-porous (e.g., non-permeable), rigid, flexible, elastic or any combinations thereof. Accordingly, the membrane 208 can have a porosity of about 0% to about 99%. As used herein, the term “porosity” is a measure of total void space (e.g., through-holes, openings, interstitial spaces, and/or hollow conduits) in a material, and is a fraction of volume of total voids over the total volume, as a percentage between 0 and 100% (or between 0 and 1). A membrane with substantially zero porosity is non-porous or non-permeable.

As used interchangeably herein, the terms “non-porous” and “non-permeable” refer to a material that does not allow any molecule or substance to pass through.

In some embodiments, the membrane can be porous and thus allow molecules, cells, particulates, chemicals and/or media to migrate or transfer between the first chamber 204 and the second chamber 206 via the membrane 208 from the first chamber 204 to the second chamber 206 or vice versa.

As used herein, the term “porous” generally refers to a material that is permeable or selectively permeable. The term “permeable” as used herein means a material that permits passage of a fluid (e.g., liquid or gas), a molecule, a whole living cell and/or at least a portion of a whole living cell, e.g., for formation of cell-cell contacts. The term “selectively permeable” as used herein refers to a material that permits passage of one or more target group or species, but act as a barrier to non-target groups or species. For example, a selectively-permeable membrane can allow passage of a fluid (e.g., liquid and/or gas), nutrients, wastes, cytokines, and/or chemokines from one side of the membrane to another side of the membrane, but does not allow whole living cells to pass through. In some embodiments, a selectively-permeable membrane can allow certain cell types to pass through but not other cell types.

The permeability of the membrane to individual matter/species can be determined based on a number of factors, including, e.g., material property of the membrane (e.g., pore size, and/or porosity), interaction and/or affinity between the membrane material and individual species/matter, individual species size, concentration gradient of individual species between both sides of the membrane, elasticity of individual species, and/or any combinations thereof.

A porous membrane can have through-holes or pore apertures extending vertically and/or laterally between two surfaces 208A and 208B of the membrane (FIG. 1B), and/or a connected network of pores or void spaces (which can, for example, be openings, interstitial spaces or hollow conduits) throughout its volume. The porous nature of the membrane can be contributed by an inherent physical property of the selected membrane material, and/or introduction of conduits, apertures and/or holes into the membrane material.

In some embodiments, a membrane can be a porous scaffold or a mesh. In some embodiments, the porous scaffold or mesh can be made from at least one extracellular matrix polymer (e.g., but not limited to collagen, alginate, gelatin, fibrin, laminin, hydroxyapatite, hyaluronic acid, fibroin, and/or chitosan), and/or a biopolymer or biocompatible material (e.g., but not limited to, polydimethylsiloxane (PDMS), polyurethane, styrene-ethylene-butylene-styrene (SEBS), poly(hydroxyethylmethacrylate) (pHEMA), polyethylene glycol, polyvinyl alcohol and/or any biocompatible material described herein for fabrication of the device first structure and/or second structure) by any methods known in the art, including, e.g., but not limited to, electrospinning, cryogelation, evaporative casting, and/or 3D printing. See, e.g., Sun et al. (2012) “Direct-Write Assembly of 3D Silk/Hydroxyapatite Scaffolds for Bone Co-Cultures.” Advanced Healthcare Materials, no. 1: 729-735; Shepherd et al. (2011) “3D Microperiodic Hydrogel Scaffolds for Robust Neuronal Cultures.” Advanced Functional Materials 21: 47-54; and Barry III et al. (2009) “Direct-Write Assembly of 3D Hydrogel Scaffolds for Guided Cell Growth.” Advanced Materials 21: 1-4, for examples of a 3D biopolymer scaffold or mesh that can be used as a membrane in the device described herein.

In some embodiments, a membrane can comprise an elastomeric portion fabricated from a styrenic block copolymer-comprising composition, e.g., as described in the International Pat. App. No. PCT/US2014/071611, can be adopted in the devices described herein, the contents of each of which are incorporated herein by reference in its entirety. In some embodiments, the styrenic block copolymer-comprising composition can comprise SEBS and polypropylene.

In some embodiments, a membrane can be a hydrogel or a gel comprising an extracellular matrix polymer, and/or a biopolymer or biocompatible material. In some embodiments, the hydrogel or gel can be embedded with a conduit network, e.g., to promote fluid and/or molecule transport. See, e.g., Wu et al. (2011) “Omnidirectional Printing of 3D Microvascular Networks.” Advanced Materials 23: H178-1-1183; and Wu et al. (2010) “Direct-write assembly of biomimetic microvascular networks for efficient fluid transport.” Soft Matter 6: 739-742, for example methods of introducing a conduit network into a gel material.

In some embodiments, a porous membrane can be a solid biocompatible material or polymer that is inherently permeable to at least one matter/species (e.g., gas molecules) and/or permits formation of cell-cell contacts. In some embodiments, through-holes or apertures can be introduced into the solid biocompatible material or polymer, e.g., to enhance fluid/molecule transport and/or cell migration. In one embodiment, through-holes or apertures can be cut or etched through the solid biocompatible material such that the through-holes or apertures extend vertically and/or laterally between the two surfaces of the membrane 208A and 208B. It should also be noted that the pores can additionally or alternatively incorporate slits or other shaped apertures along at least a portion of the membrane 208 which allow cells, particulates, chemicals and/or fluids to pass through the membrane 208 from one section of the central channel to the other.

The pores of the membrane (including pore apertures extending through the membrane 208 from the top 208A to bottom 208B surfaces thereof and/or a connected network of void space within the membrane 208) can have a cross-section of any size and/or shape. For example, the pores can have a pentagonal, circular, hexagonal, square, elliptical, oval, diamond, and/or triangular shape.

The cross-section of the pores can have any width dimension provided that they permit desired molecules and/or cells to pass through the membrane. In some embodiments, the pore size of the membrane should be big enough to provide the cells sufficient access to nutrients present in a fluid medium flowing through the first chamber and/or the second chamber. In some embodiments, the pore size can be selected to permit passage of cells (e.g., immune cells) from one side of the membrane to the other. In some embodiments, the pore size can be selected to permit passage of nutrient molecules. In some embodiments, the width dimension of the pores can be selected to permit molecules, particulates and/or fluids to pass through the membrane 208 but prevent cells from passing through the membrane 208. In some embodiments, the width dimension of the pores can be selected to permit cells, molecules, particulates and/or fluids to pass through the membrane 208. Thus, the width dimension of the pores can be selected, in part, based on the sizes of the cells, molecules, and/or particulates of interest. In some embodiments, the width dimension of the pores (e.g., diameter of circular pores) can be in the range of 0.01 microns and 20 microns, or in one embodiment, approximately 0.1-15 microns, or approximately 1-10 microns. In one embodiment, the pores have a width of about 7 microns.

In an embodiment, the porous membrane 208 can be designed or surface patterned to include micro and/or nanoscopic patterns therein such as grooves and ridges, whereby any parameter or characteristic of the patterns can be designed to desired sizes, shapes, thicknesses, filling materials, and the like.

The membrane 208 can have any thickness to suit the needs of a target application. In some embodiments, the membrane can be configured to deform in a manner (e.g., stretching, retracting, compressing, twisting and/or waving) that simulates a physiological strain experienced by the cells in its native microenvironment. In these embodiments, a thinner membrane can provide more flexibility. In some embodiments, the membrane can be configured to provide a supporting structure to permit growth of a defined layer of cells thereon. Thus, in some embodiments, a thicker membrane can provide a greater mechanical support. In some embodiments, the thickness of the membrane 208 can range between 70 nanometers and 100 μm, or between 1 μm and 100 μm, or between 10 and 100 μm. In one embodiment, the thickness of the membrane 208 can range between 10 μm and 80 μm. In one embodiment, the thickness of the membrane 208 can range between 30 μm and 80 μm. In one embodiment, the thickness of the membrane 208 can be about 50 μm.

While the membrane 208 generally have a uniform thickness across the entire length or width, in some embodiments, the membrane 208 can be designed to include regions which have lesser or greater thicknesses than other regions in the membrane 208. The decreased thickness area(s) can run along the entire length or width of the membrane 208 or can alternatively be located at only certain locations of the membrane 208. The decreased thickness area can be present along the bottom surface of the membrane 208 (i.e. facing second chamber 206), or additionally/alternatively be on the opposing surface of the membrane 208 (i.e. facing second chamber 204). It should also be noted that at least portions of the membrane 208 can have one or more larger thickness areas relative to the rest of the membrane, and capable of having the same alternatives as the decreased thickness areas described above.

In some embodiments, the membrane can be coated with substances such as various cell adhesion promoting substances or ECM proteins, such as fibronectin, laminin, various collagen types, glycoproteins, vitronectin, elastins, fibrin, proteoglycans, heparin sulfate, chondroitin sulfate, keratin sulfate, hyaluronic acid, fibroin, chitosan, or any combinations thereof. In some embodiments, one or more cell adhesion molecules can be coated on one surface of the membrane 208 whereas another cell adhesion molecule can be applied to the opposing surface of the membrane 208, or both surfaces can be coated with the same cell adhesion molecules. In some embodiments, the ECMs, which can be ECMs produced by cells, such as primary cells or embryonic stem cells, and other compositions of matter are produced in a serum-free environment.

In an embodiment, one can coat the membrane with a cell adhesion factor and/or a positively-charged molecule that are bound to the membrane to improve cell attachment and stabilize cell growth. The positively charged molecule can be selected from the group consisting of polylysine, chitosan, poly(ethyleneimine) or acrylics polymerized from acrylamide or methacrylamide and incorporating positively-charged groups in the form of primary, secondary or tertiary amines, or quaternary salts. The cell adhesion factor can be added to the membrane and is fibronectin, laminin, various collagen types, glycoproteins, vitronectin, elastins, fibrin, proteoglycans, heparin sulfate, chondroitin sulfate, keratin sulfate, hyaluronic acid, tenascin, antibodies, aptamers, or fragments or analogs having a cell-binding domain thereof. The positively-charged molecule and/or the cell adhesion factor can be covalently bound to the membrane. In another embodiment, the positively-charged molecule and/or the cell adhesion factor are covalently bound to one another and either the positively-charged molecule or the cell adhesion factor is covalently bound to the membrane. Also, the positively-charged molecule or the cell adhesion factor or both can be provided in the form of a stable coating non-covalently bound to the membrane.

In some embodiments, cells are cultured on and/or under the membrane under flow conditions. In some embodiments, there is a steady-state perfusion of the cells. In other embodiments described herein, the devices can comprise a flowing culture medium in the first chamber and/or the second chamber, wherein the flowing culture medium generates a shear stress. Based on the viscosity of the culture medium and/or dimensions of the chambers, one of skill in the art can determine appropriate flow rates of culture medium through the chambers to achieve desired shear stress. In some embodiments, the flow rate of the culture medium through the first chamber can range from about 5 μL/hr to about 50 μL/hr. In some embodiments, the flow rate of the culture medium through the second chamber can range from about 15 μL/hr to about 150μL/hr. Thus, in one embodiment, fluidic shear forces are generated.

An exemplary schematic of one embodiment of a closed top chip is shown in FIG. 1C-D. FIG. 1C shows cells in relation to device parts, e.g. upper and lower channels and optional vacuum chamber: 1. Epithelial channel (upper); 2. Epithelial cells; 3. Air chambers; 4. Membrane; 5. simulated capillaries (e.g. endothelial cells); and 6. Vascular channel (lower).

A. Closed Top Microfluidic Chips without Gels.

In one embodiment, closed top organ-on-chips do not contain gels, either as a bulk gel or a gel layer. Thus, in one embodiment, the device generally comprises (i) a first structure defining a first chamber; (ii) a second structure defining a second chamber; and (iii) a membrane located at an interface region between the first chamber and the second chamber to separate the first chamber from the second chamber, the membrane including a first side facing toward the first chamber and a second side facing toward the second chamber, wherein the first and second chambers are enclosed. The first side of the membrane may have an extracellular matrix composition disposed thereon, wherein the extracellular matrix (ECM) composition comprises an ECM coating layer. In some embodiments, an ECM gel layer e.g. ECM overlay, is located over the ECM coating layer.

Additional embodiments are described herein that may be incorporated into closed top chips without gels.

B. Closed Top Microfluidic Chips with Gels.

In one embodiment, closed top organ-on-chips do contain gels, such as a gel layer, including but not limited to a gel matrix, hydrogel, bulk gels, etc. Thus, in one embodiment, the device generally comprises (i) a first structure defining a first chamber; (ii) a second structure defining a second chamber; and (iii) a membrane located at an interface region between the first chamber and the second chamber to separate the first chamber from the second chamber, the membrane including a first side facing toward the first chamber and a second side facing toward the second chamber, wherein the first and second chambers are enclosed. In some embodiments, the device further comprises a gel. In some embodiments, the gel is a continuous layer. In some embodiments, the gel is a layer of approximately the same thickness across the layer. In some embodiments, the gel is a discontinuous layer. In some embodiments, the gel has different thicknesses across the layer. In some embodiments, the first side of the membrane may have a gel layer. In some embodiments, a gel is added to the first side of the membrane without an ECM layer. The first side of the membrane may have an extracellular matrix composition disposed thereon, wherein the extracellular matrix (ECM) composition comprises an ECM coating layer. In some embodiments, an ECM gel layer e.g. ECM overlay, is located over the ECM coating layer. In some embodiments, the gel layer is above the ECM coating layer. In some embodiments, the ECM coating layer may have a gel layer on the bottom, i.e. the side facing the membrane. In some embodiments, the gel overlays the ECM gel layer.

Additional embodiments are described herein that may be incorporated into closed top chips with gels.

C. Closed Top Microfluidic Chips with Simulated Lumens.

A closed top organ-on-chip comprising a gel-lined simulated lumen may be used for generating a more physiological relevant model of epithelial tissue. In some embodiments, closed top organ-on-chips further comprise a gel simulated three-dimensional (3-D) lumen. In other words, a 3-D lumen may be formed using gels (e.g. viscous fingers) and/or mimicking tissue folds. In a preferred embodiment, the gel forms a lumen, i.e. by viscous fingering patterning.

Using viscous fingering techniques, e.g. viscous fingering patterning, a simulated lumen may be formed. As one example, viscous fingers may be formed and used to mimic epithelial projections in the respiratory system.

Methods to create three-dimensional (3-D) lumen structures in permeable matrices are known in the art. One example of a 3-D structure forming at least one lumen is referred to as “viscous fingering”. One example of viscous fingering methods that may be used to for form lumens, e.g. patterning lumens, is described by Bischel, et al. “A Practical Method for Patterning Lumens through ECM Hydrogels via Viscous Finger Patterning.” J Lab Autom. 2012 April; 17(2): 96-103. Author manuscript; available in PMC 2012 Jul. 16, herein incorporated by reference in its entirety. In one example of a viscous finger patterning method for use with microfluidic organ-on-chips, lumen structures are patterned with an ECM hydrogel.

“Viscous” generally refers to a substance in between a liquid and a solid, i.e. having a thick consistency. A “viscosity” of a fluid refers to a measure of its resistance to gradual deformation by shear stress or tensile stress. For liquids, it corresponds to an informal concept of “thickness”; for example, honey has a much higher viscosity than water.

“Viscous fingering” refers in general to the formation of patterns in “a morphologically unstable interface between two fluids in a porous medium.

A “viscous finger” generally refers to the extension of one fluid into another fluid. Merely as an example, a flowable gel or partially solidified gel may be forced, by viscous fingering techniques, into another fluid, into another viscous fluid in order to form a viscous finger, i.e. simulated lumens.

In some embodiments, the lumen can be formed by a process comprising (i) providing the first chamber filled with a viscous solution of the first matrix molecules; (ii) flowing at least one or more pressure-driven fluid(s) with low viscosity through the viscous solution to create one or more lumens each extending through the viscous solution; and (iii) gelling, polymerizing, and/or cross linking the viscous solution. Thus, one or a plurality of lumens each extending through the first permeable matrix can be created.

In another embodiment, gel is added to a channel for making a lumen.

In some embodiments as described herein, the first and second permeable matrices can each independently comprise a hydrogel, an extracellular matrix gel, a polymer matrix, a monomer gel that can polymerize, a peptide gel, or a combination of two or more thereof. In one embodiment, the first permeable matrix can comprise an extracellular matrix gel, (e.g. collagen). In one embodiment, the second permeable matrix can comprise an extracellular matrix gel and/or protein mixture gel representing an extracellular miroenvironment, (e.g. MATRIGEL®. In some embodiments, the first and second permeable matrixes can each independently comprise a polymer matrix. Methods to create a permeable polymer matrix are known in the art, including, e.g. but not limited to, particle leaching from suspensions in a polymer solution, solvent evaporation from a polymer solution, sold-liquid phase separation, liquid—liquid phase separation, etching of specific “block domains” in block co-polymers, phase separation to block-co-polymers, chemically cross-linked polymer networks with defined permabilities, and a combination of two or more thereof.

Another example for making branched structures using fluids with differing viscosities is described in “Method And System For Integrating Branched Structures In Materials” to Katrycz, Publication number US20160243738, herein incorporated by reference in its entirety.

Regardless of the type of lumen formed by a gel and/or structure, cells can be attached to theses structures either to lumen side of the gel and/or within the gel and/or on the side of the gel opposite the lumen. Thus, three-dimensional (3-D) lumen gel structures may be used in several types of embodiments for closed top microfluidic chips, e.g. epithelial cells can be attached to outside of the gel, or within the gel. In some embodiments, LPDCs may be added within the gel, or below the gel, on the opposite side of the lumen. In some embodiments, stoma cells are added within the gel. In some embodiments, stomal cells are attached to the side of the gel opposite from the lumen. In some embodiments, endothelial cells are located below the gel on the side opposite the lumen. In some embodiments, endothelial cells may be present within the gel.

Additional embodiments are described herein that may be incorporated into closed top chips with simulated 3D lumens containing a gel.

Optional Vacuum Channels.

Fluidic channels in devices of the present inventions are optionally flanked by two vacuum channels that allow the pneumatically actuated stretching forces mimicking peristalsis, for a non-limiting example, bronchial spasms. In some embodiments, stretching forces are for stretching an epithelial layer. In one embodiment, mechanical forces are generated.

Exemplary Devices for Simulating a Function of a Tissue.

Some embodiments described herein relate to devices for simulating a function of a tissue, in particular an epithelial tissue. In one embodiment, the device generally comprises (i) a first structure defining a first chamber; (ii) a second structure defining a second chamber; and (iii) a membrane located at an interface region between the first chamber and the second chamber to separate the first chamber from the second chamber, the membrane including a first side facing toward the first chamber and a second side facing toward the second chamber. The first side of the membrane may have an extracellular matrix composition disposed thereon, wherein the extracellular matrix (ECM) composition comprises an ECM coating layer. In some embodiments, an ECM gel layer e.g. ECM overlay, is located over the ECM coating layer.

ECM Coating.

To determine optimum conditions for cell attachment, the surface-treated material (e.g., APTES-treated or plasma-treated PDMS) can be coated with an ECM coating of different extracellular matrix molecules at varying concentrations (based on the resulting cell morphology and attachment).

ECM Overlay.

The ECM overlay is typically a “molecular coating,” meaning that it is done at a concentration that does not create a bulk gel. In some embodiments, an ECM overlay is used. In some embodiments, an ECM overlay is left in place throughout the co-culturing. In some embodiments, an ECM overlay is removed, e.g. when before seeding additional cells into a microfluidic device. In some embodiments, the ECM layer is provided by the cells seeded into the microfluidic device.

Although cells described for use in an organ-on-chip make their own ECM, it is contemplated that ECM in predisease and diseased states may contribute to inflammatory states. Further, the protein microenvironment provided by ECM also affects cells. Thus it is contemplated that tissue-derived ECM may carry over a disease state. Therefore, in addition to the ECM described herein, ECM used in microfluidic devises of the present inventions may be tissue-derived (native) ECM. In one embodiment, a device comprising tissue-derived ECM may be used as described herein, to identity contributions to healthy or disease states affected by native ECM. For example, ECM may be isolated from biopsies of healthy, non-disease and disease areas as tissue-derived ECM. Isolates for use may include cells within or attached or further processed to remove embedded cells for use in the absence of the cells. Additional examples of ECM materials include but are not limited to Matrigel®, Cultrex®, ECM harvested from humans, etc.

Matrigel® is a trade name for a solubilized basement membrane preparation extracted from the Engelbreth-Holm-Swarm (EHS) mouse sarcoma, a tumor rich in such ECM proteins as laminin (a primary component), collagen IV, heparin sulfate proteoglycans, entactin/nidogen, and a number of growth factors as produced and marketed by Corning Life Sciences. Matrigel® gels to form a reconstituted basement membrane. Versions of Matrigel® include BD Matrigel® (Basement Membrane) Matrix, offered as Standard, Growth Factor Reduced, Growth Factor Reduced-High Concentration (HC) and Growth Factor Reduced-Phenol Red-Free formulations, BD Matrigel® hESC-qualified Matrix, by BD Biosciences.

Trevigen, Inc. markets other ECM versions of BME harvested as a soluble form of basement membrane purified from Engelbreth-Holm-Swarm (EHS) tumor cells under the trade name Cultrex® Basement Membrane Extract (BME).

III. Open Top Microfluidic Chips.

The present disclosure relates to embodiments of organ-on-chips, such as fluidic devices comprising one or more cells types for the simulation one or more of the function of organ components. The present disclosure relates to embodiments of gut-on-chips, such as fluidic devices comprising one or more cells types for the simulation one or more of the function of gastrointestinal tract components. Many of the problems associated with earlier systems can be solved by providing an open-top style microfluidic device that allows topical access to one or more parts of the device or cells that it comprises. For example, the microfluidic device can include a removable cover, that when removed, provides access to the cells of interest in the microfluidic device. In some aspects, the microfluidic devices include systems that constrain fluids, cells, or biological components to desired area(s). The improved systems provide for more versatile experimentation when using microfluidic devices, including improved application of treatments being tested, improved seeding of additional cells, and/or improved aerosol delivery for select tissue types.

It is also desirable in some aspects to provide access to regions of a cell-culture device. For example, it can be desirable to provide topical access to cells to (i) apply topical treatments with liquid, gaseous, solid, semi-solid, or aerosolized reagents, (ii) obtain samples and biopsies, or (iii) add additional cells or biological/chemical components

Therefore, the present disclosure relates to fluidic systems that include a fluidic device, such as a microfluidic device with an opening that provides direct access to device regions or components (e.g. access to the gel region, access to one or more cellular components, etc.). Although the present disclosure provides an embodiment wherein the opening is at the top of the device (referred to herein with the term “open top”), the present invention contemplates other embodiments where the opening is in another position on the device. For example, in one embodiment, the opening is on the bottom of the device. In another embodiment, the opening is on one or more of the sides of the device. In another embodiment, there is a combination of openings (e.g. top and sides, top and bottom, bottom and side, etc.).

While detailed discussion of the “open top” embodiment is provided herein, those of ordinary skill in the art will appreciate that many aspects of the “open top” embodiment apply similarly to open bottom embodiments, as well as open side embodiments or embodiments with openings in any other regions or directions, or combinations thereof. Similarly, the device need not remain “open” throughout its use; rather, as several embodiments described herein illustrate, the device may further comprise a cover or seal, which may be affixed reversibly or irreversibly. For example, removal of a removable cover creates an opening, while placement of the cover back on the device closes the device. The opening, and in particular the opening at the top, provides a number of advantages, for example, allowing (i) the creation of one or more gel layers for simulating the application of topical treatments on the cells, tissues, or organs, or (ii) the addition of chemical or biological components such as the seeding of additional cell types for simulated tissue and organ systems. The present disclosure further relates to improvement in fluidic system(s) that improve the delivery of aerosols to simulated tissue and organ systems, such as simulated gastrointestinal tissues.

The present invention contemplates a variety of uses for these open top microfluidic devices and methods described herein. In one embodiment, the present invention contemplates a method of topically testing an agent (whether a drug, food, gas, or other substance) comprising 1) providing a) an agent and b) microfluidic device comprising i) a chamber, said chamber comprising a lumen and projections into the lumen, said lumen comprising ii) a gel matrix anchored by said projections and comprising cell in, on or under said gel matrix, said gel matrix positioned above iii) a porous membrane and under iv) a removable cover, said membrane in contact with v) fluidic channels; 2) removing said removable cover; and 3) topically contacting said cells in, on or under said gel matrix with said agent. In one embodiment, said agent is in an aerosol. In one embodiment, agent is in a liquid, gas, gel, semi-solid, solid, or particulate form. These uses may apply to the open top microfluidic chips described below and herein.

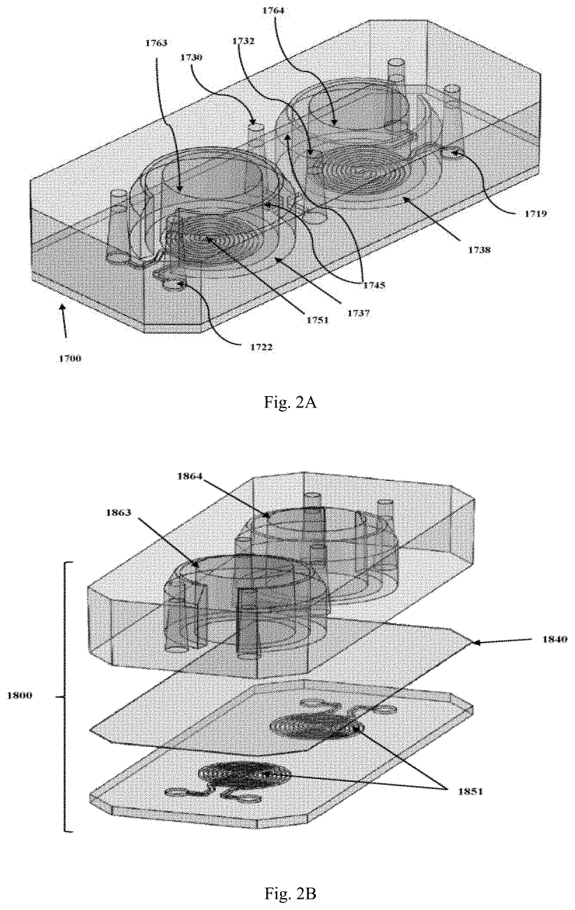

Accordingly, the present disclosure additionally describes open-top organ-on-chips, gut-on-chips, etc., see, e.g. schematic in FIGS. 2A-B. Although a device having two sets of chambers is depicted, in some embodiments, an open top chip has one upper and one lower channel.

FIG. 2A shows, one embodiment, an open-top chip device 1700 comprising: i) a first chamber 1763 and a second chamber 1764, wherein each chamber is surrounded by a deformable surface 1745; and ii) at least one spiral microchannel 1751 located on the bottom surface of the chamber, wherein each of the microchannels are in fluidic communication with an inlet port 1719 and an outlet port 1722 and are respectively configured with a first vacuum port 1730 or a second vacuum port 1732, such that each vacuum port is respectively connected to a first vacuum chamber 1737 or a second vacuum chamber 1738.

FIG. 2A shows optional vacuum chambers 4. Open top microfluidic chips include but are not limited to chips having removable covers, such as removable plastic covers.

FIG. 2B shows an exemplary exploded view of one embodiment of an open-top chip device 1800, wherein a membrane 1840 resides between the bottom surface of the first chamber 1863 and the second chamber 1864 and the at leastone spiral microchannel 1851, also shown in FIG. 2A.

II. Instrumentation.

WO2017035484. In one embodiment (as shown in FIGS. 1A, 1B and 1C), the perfusion manifold assembly (10) comprises i) a cover or lid (11) configured to serve as to top of ii) one or more fluid reservoirs (12), iii) a capping layer (13) under said fluid reservoir(s), iv) a fluidic backplane (14) under, and in fluidic communication with, said fluid reservoir(s), said fluidic backplane comprising a fluidic resistor, and v) a projecting member or skirt (15) for engaging the microfluidic device (16) or chip which is preferably positioned in a carrier (17), the chip having one or more microchannels (1) and in fluidic communication with one or more ports (2). The assembly can be used with or without the lid or cover. Other embodiments (discussed below) lack a skirt or projecting member. In one embodiment, the carrier (17) has a tab or other gripping platform (18), a retention mechanism such as a clip (19), and a visualization cutout (20) for imaging the chip. The cutout (20) can enable placing a carrier (e.g. a carrier engaged with the perfusion manifold assembly or “pod” or not so engaged) onto a microscope or other inspection device, allowing the chips to be observed without having to remove the chip from the carrier. In one embodiment, the fluidic resistor comprises a series of switchbacks or serpentine fluid channels.

A perfusion manifold assembly (10) comprises i) a cover or lid (11) configured to serve as to top of ii) one or more fluid reservoirs (12), iii) a capping layer (13) under said fluid reservoir(s), iv) a fluidic backplane (14) under, and in fluidic communication with, said fluid reservoir(s), said fluidic backplane comprising a fluidic resistor, and v) a projecting member or skirt (15) for engaging the microfluidic device (16) or chip which is preferably positioned in a carrier (17), the chip having one or more microchannels (1) and in fluidic communication with one or more ports (2). The assembly can be used with or without the lid or cover. Other embodiments (discussed below) lack a skirt or projecting member. In one embodiment, the carrier (17) has a tab or other gripping platform (18), a retention mechanism such as a clip (19), and a visualization cutout (20) for imaging the chip. The cutout (20) can enable placing a carrier (e.g. a carrier engaged with the perfusion manifold assembly or “pod” or not so engaged) onto a microscope or other inspection device, allowing the chips to be observed without having to remove the chip from the carrier. In one embodiment, the fluidic resistor comprises a series of switchbacks or serpentine fluid channels.

An exploded view of one embodiment of the perfusion manifold assembly (also called the perfusion disposable or perfusion manifold or “pod”) showing the cover (or cover assembly) off of the reservoirs (the reservoir body can be made of acrylic, for example), the reservoirs positioned above the backplane, the backplane in fluidic communication with the reservoirs, the skirt with a side track for engaging a representative microfluidic device or “chip” (which can be fabricated out of plastic, such as PDMS, for example) having one or more inlet, outlet and (optional) vacuum ports, and one or more microchannels, the chip shown next to (but not in) one embodiment of a chip carrier (which can be fabricated out of a thermoplastic polymer, such as acrylonitrile butadiene styrene (ABS), for example), the carrier being configured to support and carrier the chip, e.g. dimensioned so that the chip fits within a cavity.

The same embodiment of the perfusion manifold assembly with the cover on and over the reservoirs, and the chip inside the chip carrier fully linked to the skirt of the perfusion manifold assembly, and thereby in fluidic communication with the reservoirs. In one embodiment, each chip has two inputs, two outputs and (optionally) two connections for the vacuum stretch. In one embodiment, putting the chip in fluidic communication connects all six in one action, rather than connecting them one at a time.

An exploded view of one embodiment of the perfusion manifold assembly (before the components have been assembled) comprising reservoirs positioned over a fluidic backplane (comprising a fluid resistor), that is fluidically sealed with a capping layer and is positioned over a skirt, with each piece dimensioned to fit over the next. In one embodiment, the skirt comprises structure (e.g. made of polymer) that borders or defines two open spaces, one of the spaces configured to receive the carrier with the chip inside. In one embodiment, the skirt has structure that completely surrounds one open space and two “arms” that extend outwardly that define a second open space for receiving the carrier. In one embodiment, the two arms have sidetracks for slideably engaging the carrier edges.

An exploded view of one embodiment of the cover assembly (11) comprising a pressure cover or pressure lid. In the illustrated embodiment, the pressure lid comprises a port (5) that allows pneumatic (e.g. vacuum) control of (optional) chip stretching to be communicated through the lid and a plurality of ports (36) (e.g. through-hole ports) (e.g. through-hole ports) associated with filters (38) (e.g. a 0.2 um filter) and corresponding holes (39) in a gasket (37) positioned underneath the cover. In one embodiment, the cover or lid is made of polycarbonate. The illustrated design of the holes in the gasket is intended to permit the gasket to aid in retaining the illustrated filters in position. In alternative embodiments, gasket openings may employ a shape different from openings in the lid. For example, the gasket can be shaped to follow the contour of one or more reservoirs with which it is intended to form a fluidic or pressure seal. In some embodiments, a plurality of gaskets may be employed. FIG. 2B shows the same embodiment of the cover assembly illustrated in FIG. 2A with the filters and gasket positioned within (and under) the cover.

One embodiment of the microfluidic device or chip (16), showing two channels (1), each with an inlet (2) and outlet port, as well as (optional) vacuum ports. FIG. 3B is a topside schematic of an alternative embodiment of the perfusion disposable or “pod” (10) featuring the transparent (or translucent) cover (11) over the reservoirs, with the chip (16) inserted. The chip (16) can be seeded with cells and then placed in a carrier (17) for insertion into the perfusion disposable.

A side view of one embodiment of a chip carrier (17) (with the chip inside) approaching (but not yet engaging) a side track (25) of a skirt of one embodiment of the perfusion manifold assembly (10), the carrier aligned at an angle matching an angled front end portion of the side track, angled slide (27) which provides a larger opening for easier initial positioning, followed by a linear or essentially linear portion (28), the carrier comprising a retention mechanism (19) configured as a upwardly protecting clip. Without being bound by theory, a suitably large angle permits chip engagement without smearing or premature engagement of liquid droplets present on the chip and/or the perfusion manifold assembly during the insertion and alignment processes. FIG. 4B shows a side view of one embodiment of a chip carrier (with the chip (16) inside) engaging a side track of a skirt of one embodiment of (but not yet linked to) the perfusion manifold assembly. A side view of one embodiment of a chip carrier (with the chip inside) fully engaging a side track of a skirt of one embodiment of (but not yet linked to) the perfusion manifold assembly (with an arrow showing the necessary direction of movement to get a snap fit whereby the retention mechanism will engage to prevent movement). FIG. 4D shows a side view of one embodiment of a chip carrier (with the chip inside) detachably linked to the perfusion manifold assembly, where the retention mechanism is engaged to prevent movement.

A schematic of one embodiment of a work flow (with arrows showing each progressive step), where the chip (16) is linked (e.g. snapped in) to a disposable perfusion manifold assembly (“perfusion disposable”) (10), which in turn is positioned with other assemblies on a culture module (30), which is placed in an incubator (31).

A schematic of another embodiment of the culture module (30) showing the tray (or rack) (32) and sub-tray or nest (47) for transporting and inserting the perfusion disposables (10) into the culture module (30), which has two openings (48, 49) in the housing to receive the trays, and a user interface (46) to control the process of engaging the perfusion disposables and applying pressure. A typical incubator (not shown) can hold up to six modules (30).

A schematic of the interior of one embodiment of the module (i.e. the housing has been removed), showing the pressure manifold (50) is in an open position, positioning of the tray (or rack) (32), sub-tray (or nest) (47), perfusion disposables (PDs) (10) under a pressure manifold (50) (but not engaging it, so the clearance is sufficient to remove them), with the actuation assembly (51) (including the pneumatic cylinder) (52) above. Three microfluidic devices or perfusion disposables are shown to illustrate, although more (e.g. 6, 9 or 12) are typically used at once.

A schematic of the interior of one embodiment of the module (i.e. the housing has been removed), showing the pressure manifold (50) in a closed position, with the positioning of the tray or rack (32), sub-tray or nest (47), perfusion disposables (10) under the pressure manifold (50) and engaging it, with the actuation assembly (51) including the pneumatic cylinder (52) above. The pressure manifold (50) simultaneously engages all of the perfusion disposables (10) while media perfusion is required or needed. Independent control of the flow rate in the top and bottom channels of the chip (16) can be achieved. The pressure manifold (50) can disengage (without complicated fluid disconnects) as desired to allow removal of the trays (32) or nests (47) for imaging or other tasks. In one embodiment, the pressure manifold (50) can simultaneously disengage from a plurality of perfusion manifold assemblies. In one embodiment, the perfusion disposables (10) are not rigidly fixed inside the nests (47), allowing them to locate relative to the pressure manifold (50) as it closes. In a preferred embodiment, integrated alignment features in the pressure manifold (50) provide guidance for each perfusion disposable (10). Again, three microfluidic devices or perfusion disposables are shown to illustrate, although more (e.g. 6, 9 or 12) are typically used at once.

A schematic of one embodiment of a connection scheme comprising a tube connecting manifold (82) permitting four culture modules (30) (three are shown) to be connected inside a single incubator (31) using one or more hub modules (the two circles provide magnified views of a first end (83) and second end (84) of the connections).

III. Chip Activation.

A. Chip Activation Compounds.

In one embodiment, bifunctional crosslinkers are used to attach one or more extracellular matrix (ECM) proteins. A variety of such crosslinkers are available commercially, including (but not limited to) the following compounds:

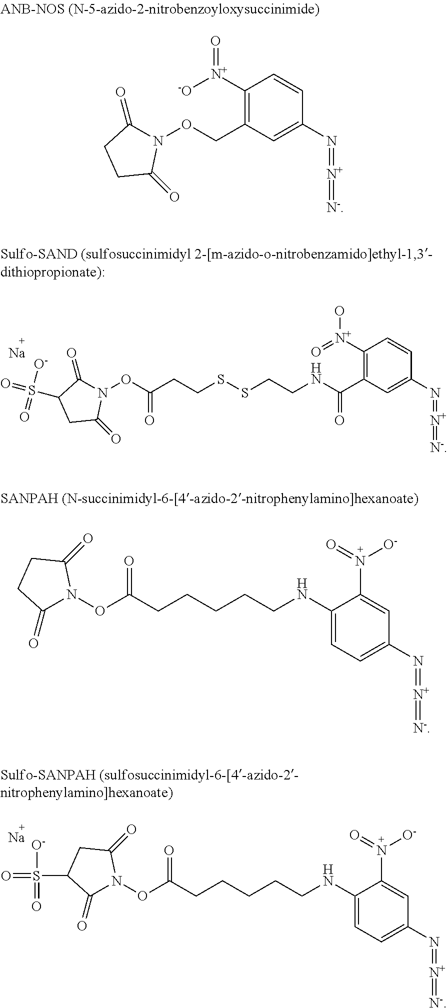

By way of example, sulfosuccinimidyl 6-(4′-azido-2′-nitrophenyl-amino) hexanoate or “Sulfo-SANPAH” (commercially available from Pierce) is a long-arm (18.2 angstrom) crosslinker that contains an amine-reactive N-hydroxysuccinimide (NHS) ester and a photoactivatable nitrophenyl azide. NHS esters react efficiently with primary amino groups (−NH₂) in pH 7-9 buffers to form stable amide bonds. The reaction results in the release of N-hydroxy-succinimide. When exposed to UV light, nitrophenyl azides form a nitrene group that can initiate addition reactions with double bonds, insertion into C—H and N—H sites, or subsequent ring expansion to react with a nucleophile (e.g., primary amines). The latter reaction path dominates when primary amines are present.

Sulfo-SANPAH should be used with non-amine-containing buffers at pH 7-9 such as 20 mM sodium phosphate, 0.15M NaCl; 20 mM HEPES; 100 mM carbonate/bicarbonate; or 50 mM borate. Tris, glycine or sulfhydryl-containing buffers should not be used. Tris and glycine will compete with the intended reaction and thiols can reduce the azido group.

For photolysis, one should use a UV lamp that irradiates at 300-460 nm. High wattage lamps are more effective and require shorter exposure times than low wattage lamps. UV lamps that emit light at 254 nm should be avoided; this wavelength causes proteins to photodestruct. Filters that remove light at wavelengths below 300 nm are ideal. Using a second filter that removes wavelengths above 370 nm could be beneficial but is not essential.

B. Exemplary Methods of Chip Activation.

1. Prepare and sanitize hood working space

2. S-1 Chip Handling—Use aseptic technique, hold Chip using Carrier
a. Use 70% ethanol spray and wipe the exterior of Chip package prior to bringing into hood
b. Open package inside hood
c. Remove Chip and place in sterile Petri dish (6 Chips/Dish)
d. Label Chips and Dish with respective condition and Lot #

3. Surface Activation with Chip Activation Compound (light and time sensitive)
a. Turn off light in biosafety hood
b. Allow vial of Chip Activation Compound powder to fully equilibrate to ambient temperature (to prevent condensation inside the storage container, as reagent is moisture sensitive)
c. Reconstitute the Chip Activation Compound powder with ER-2 solution
   i. Add 10 ml Buffer, such as HEPES, into a 15 ml conical covered with foil
   ii. Take 1 ml Buffer from above conical and add to chip Activation Compound (5 mg) bottle, pipette up and down to mix thoroughly and transfer to same conical
   iii. Repeat 3-5 times until chip Activation Compound is fully mixed
   iv. NOTE: Chip Activation Compound is single use only, discard immediately after finishing Chip activation, solution cannot be reused
d. Wash channels
   i. Inject 200 ul of 70% ethanol into each channel and aspirate to remove all fluid from both channels
   ii. Inject 200 ul of Cell Culture Grade Water into each channel and aspirate to remove all fluid from both channels
   iii. Inject 200 ul of Buffer into each channel and aspirate to remove fluid from both channels
e. Inject Chip Activation Compound Solution (in buffer) in both channels
   i. Use a P200 and pipette 200 ul to inject Chip Activation Compound/Buffer into each channel of each chip (200 ul should fill about 3 Chips (Both Channels))
   ii. Inspect channels by eye to be sure no bubbles are present. If bubbles are present, flush channel with Chip Activation Compound/Buffer until bubbles have been removed
f. UV light activation of Chip Activation Compound Place Chips into UV light box
   i. UV light treat Chips for 20 min
   ii. While the Chips are being treated, prepare ECM Solution.
   iii. After UV treatment, gently aspirate Chip Activation Compound/Buffer from channels via same ports until channels are free of solution
   iv. Carefully wash with 200 ul of Buffer solution through both channels and aspirate to remove all fluid from both channels
   v. Carefully wash with 200 ul of sterile DABS through both channels
   vi. Carefully aspirate PBS from channels and move on to: ECM-to-Chip

VI. ECM-to-Chip

A. Calculate total volume of ECM solution needed to coat Chips.
1. Volume required per Chip=50 ul/Channel
2. ECM diluent: PBS, prepared on ice
3. Stock Concentrations for ECM coating:
   a. Collagen IV: 1 mg/ml (200 ul aliquots in −20 C)
   b. Fibronectin: 1 mg/ml (50 ul aliquots in 4 C)
   c. Matrigel: 10 mg/ml (200 ul aliquots in −20 C)
4. Working Concentrations for ECM coating:
   a. Collagen IV: 200 ug/ml
   b. Fibronectin: 30 ug/ml

5. Top Channel Coating: 50 ul Collagen IV (200 ug/ml) and Matrigel (100 ug/ml)

6. Bottom Channel Coating: 50 ul Collagen IV (200 ug/ml) and Fibronectin (30 ug/ml)

B. Load Channels with ECM solution.
1. Place Chips in hood
2. Pipette 50 μl of Top Channel Coating into Top Channel keep the pipette plunger depressed until you see fluid come out opposite end of the channel, then take another pipette tip (200 μl tip) to close the outlet port. Once closed off, carefully remove the pipette tip, leaving the tip in the inlet port.
3. Aspirate excess fluid from the surface of Chip (avoid direct contact with the port)
4. Repeat 2b-2c, but with Bottom Channel Coating into Bottom Channel
5. Incubate at 37 C for a minimum of 2 hours up to overnight

C. Exemplary Matrigel Coating.
1. Thaw Matrigel on ice and keep chilled to prevent solidification.
1. Prepare Matrigel
   a. Matrigel Stock Concentration: 10 mg/ml
   a. Matrigel Final Concentration: 250 μg/ml
   a. Determine the volume of Matrigel needed to coat 50 μl of each Top Channel and resuspend accordingly in cell culture media
      a. Transfer the seeded Chips into the hood
      b. Wash both channels of each chip twice with 200 ul media
   a. Before inserting the tips, add a drop of media to prevent formation of bubbles
   b. Leave 50 ul media in bottom channel (Tips inserted)
   c. Add 50 ul 250 ug/ml Matrigel™ to top channel (Tips inserted)
   d. Incubate at 37 C overnight

V. Cells-to-Chip—Chip Preparation
1. Transfer the ECM coated Chips into the hood
   a. Gently wash Chips after ECM coating
2. Pipette 200 μl of DPBS into bottom channel inlet port—keep the pipette plunger depressed until you see fluid come out opposite end of channel and aspirate outflow
3. Repeat the same procedure to wash top channel
4. Pipette 200 μl of DPBS into top channel inlet port—keep the pipette plunger depressed until you see fluid come out opposite end of the channel, then take another pipette tip (200 μl) to close the outlet port. Once closed off, carefully remove the pipette tip, leaving the tip in the inlet port
5. Repeat the same with the bottom channel. Place back in incubator until cells are ready.

### EXAMPLES

The following examples are offered to illustrate various embodiments of the invention, but should not be viewed as limiting the scope of the invention.

### Example A: Normal Human Organoids On-Chip

We assess the functionality of multiple cell types of organoids on Chip compared to suspension organoids in some instances. MDR1 fluorescent substrate rhodamine 123 will be used to confirm MDR1 transporter activity functionality. Rhodamine is expected to enter the cell and be blocked by the MDR1 blocker verapamil. In addition, we could test efflux activity by one of the drugs digoxin or erythromycin. Organoids loaded with the calcium indicator Fluo-4, demonstrating an increase in intracellular calcium upon stimulation with ATP and acetylcholine. Measurement of CYP3A4 enzyme activity. Stimulation of the release of GLP-1 by medium and long chain fatty acids (MCFAs and LCFAs) through the receptors GPR40 and 120 short chain fatty acids SCFA through GPR41 (small intestine) and GPR43 (large intestine) S Ethanolamides that signal through the receptor GPR119, which is expressed by L-cells Latrunculin mediated alteration in cytoskeleton.

### Example B

Propagation of Suspension Enteroids/Colonoids Includes but is not Limited to: Passaging ratio—1:3 (duodenum, jejunum, ileum, colon); Passaging frequency—every 7 days; Fragmentation technique—digestion using TrypLE (1:1 vol/vol in DPBS) in the presence of ROCK inhibitor.

Exemplary Timeline: FIG. 24, includes but is not limited to: Freezing time: 3 days after passaging; Freezing media: Recovery™ Cell Culture Freezing Medium (Gibco); Enteroids/colonoids Stock Vials: 12 wells of enteroids/colonoids per vial.

Mini-Biobank of Enteroids/Colonoids.

Mini-biobank of normal human small intestine ileal derived organoids and normal human colonic derived colonoids has been successfully established. Established and optimized protocols for propagation and bio-banking of suspension ileal and colonic organoids samples from screening colonoscopy, includes but is not limited to: Donor 351 (53 y, female); Donor 461 (50 y, male); Donor 601 (52 y, male).

Created a mini-biobank of at least 3 normal human small intestinal organoids (enteroids) and at least 4 normal human colonic organoids (colonoids). Mini-biobank of normal human small intestine ileal derived organoids and normal human colonic derived colonoids has been successfully established, samples from screening colonoscopy: Donor 351 (53 y, female); Donor 461 (50 y, male); Donor 601 (52 y, male).

Methods and Protocols: Establishment of Organoids Culture, includes but is not limited to: Thawing procedure; Embedding & Seeding protocol; Propagation of Suspension Organoids; Recovery from the Matrigel; Fragmentation procedure; Embedding & Seeding protocol; Culture maintenance; Bio-banking of Suspension; Organoids; Recovery from the Matrigel; Organoids freezing protocol, etc.

### Example C: Testing of Small Molecules in Intestine-Chip

In some embodiments, a compound's availability to cells within a microfluidic system is determined.

STEP 1: Preliminary evaluation of the PDMS absorption can be performed by the Modeling Team based on molecular weight (MW) and lipophilicity (log P) of the compound—poor prediction of the extend of the absorption.

STEP 2: Time-dependent static PDMS Absorption Test—to determine fundamental parameters (Diffusivity and Partition Coefficient).

To estimate the amount of compounds needed to run a PDMS absorption test we need to know:

Final concentration: What is the final concentration (or the range of concentrations) that we are going to use?

Stock preparation: What is the initial concentration of the Stock solution? Is the DMSO used for reconstituting the compounds?

Molecular characteristics of the compound: 1. log P. 2. Molecular weight. 3. Is the compound light sensitive?

Quantification of PDMS Absorption. This is a method for quantifying the drug-specific extent of compound loss.

GOALS:

Directly quantify partitioning of a compound.

Compound loss at equilibrium (kinetics);

Directly quantify diffusion of compound; Time-dependent compound loss (dynamics). This is a method for quantifying the drug-specific extent of compound loss.

Single time point experiments are only capable of extracting kinetics, not dynamics. Time-dependent studies capture both equilibrium endpoints (K), and time-dependent changes/dynamics (D). 1D computational models are used to fit experimental results of time-dependent studies.

Group I: Background Adsorption: Quantifies loss of compound caused by adsorption to glass.

Group II: Time-Dependent Absorption:

STEP 1: Preliminary evaluation of the PDMS absorption can be performed by the Modeling Team based on molecular weight (MW) and lipophilicity (log P) of the compound—poor prediction of the extend of the absorption.

STEP 2: Time-dependent static PDMS Absorption Test—to determine fundamental parameters (Diffusivity and Partition Coefficient).

STEP 3: Modeling of the compound loss in the chip for different flow rates—to make recommendation to minimize loss.

STEP 4: Comparison of the model and experimental results.

### Example D: Small Molecule Compound A and B

| | | | Dosing |
|---|---|---|---|
| | Molecular Weight | | Concentration |
| Compound | (g/mol) | logP | (MM) |
| Compound A | 446.9 | 3.75-4.02 | 1 |
| Compound B | 499.61 | 4.47-4.49 | 1 |

STEP 1: Compounds flagged as potentially absorbing onto fluidic components, e.g. Small molecule. High log P value of a compound, which is the logarithm of its partition coefficient between n-octanol and water log(c_{octanol}/c_{water}), is a well established measure of the compound's hydrophilicity.

STEP 2: Standard 72 hour absorption studies run for PDMS (glass vials). D and K determined in 1 D COMSOL model by fitting experimental data.

STEP 3: 2D Chip model run to determine cellular exposure concentrations

Compound dosed in basal channel (which minimizes absorption)

Flow modeled at: 30 μl/hr to imitate standard flow condition, 150 μl/hr to minimize PDMS exposure. Flow rate for Compound B exposure—150 μL/hr flow rate recommended for experiment.

STEP 4: Comparison of 2D Chip model and experimental results

Model predicted well the expected perfusion manifold outlet concentration collected in first 24 hrs. >80% recovery of dosing concentration for Compound A, regardless of flow rate. Approximately 70% recovery for Compound B at 150 μL/hr, <10% recovery at 30 μL/hr.

CONCLUSION: Absorption Tests and Modeling Approach allowed to implement experimental modifications ensuring optimal exposure of highly absorbed compounds.

### Example E: Contemplated Example: Establish and Optimize GPR35 Stimulation Conditions in Microfluidic Intestine-Chip System, Including Compounds (GPR35 Agonists, Vedolizumab, TGR5 Agonists, SCFA)

Microfluidic chips will be treated luminally with below mentioned compounds and downstream MAPK activation, specifically pERK1/2 relative to total ERK1/2, will be examined at 0, 15 min post-stimulation. TNF-α treatment which also induces pERK1/2 would be used as control.

Functional validation of GPR35 stimulation will be assessed by following: pERK1/2 and pSTAT3 assessment. Functional impact of various GPR35 agonists examined by Q-PCR analyses of MAPK-dependent upregulated genes e.g. DUSP5/1/4, cyclinDI, COX2, PHRP, etc. Gene expression assessment will be performed 0, 6 hrs post-stimulation. As warranted by Q-PCR data, LC-MS and RNAseq profiling of treated Microfluidic chips will be implemented in future experiments for small molecules assessments.

All publications and patents mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in medicine, molecular biology, cell biology, genetics, statistics, engineering, physics, or related fields are intended to be within the scope of the following claims.

1. A method for treating a cell barrier, comprising,
a) providing;
i) a microfluidic device comprising one or more layers of cells comprising a barrier having a first level of permeability;
ii) a first substance capable of increasing the permeability of said barrier to a second level, and
iii) a second substance capable of decreasing the permeability of said barrier;
b) contacting said barrier with said first substance so as to create a treated barrier; and
c) contacting said treated barrier with said second substance.

2. The method of claim 1, further comprising, prior to step c), determining whether said first substance increases said permeability of said barrier.

3. The method of claim 1, further comprising, after step c), determining whether said second substance decreases said permeability of said treated barrier.

4. The method of claim 1, wherein said second level is up to 50-fold greater than said first level.

5. The method of claim 1, wherein said second substance counteracts up to 100% of said increase in said permeability caused by said first substance.

6. The method of claim 1, wherein said microfluidic device comprises an inlet and an outlet that are fluidically connected by a microchannel, wherein said microchannel comprises said one or more layers of cells perfused by fluid.

7. The method of claim 6, wherein said contacting of step b) is performed by introducing said first substance into said inlet.

8. The method of claim 1, wherein said second substance is a zonulin receptor antagonist.

9. The method of claim 1, wherein said second substance blocks zonulin receptors.

10. The method of claim 1, wherein said second substance is N-(2-bromophenyl)-9-methyl-9-azabicyclo[3.3.1] nonan-3-amine (AT1001/larazotide acetate).

11. The method of claim 1, wherein said second substance is larazotide acetate.

12. The method of claim 1, wherein said cell layer is selected from the group consisting of epithelial cells and endothelial cells.

13. The method of claim 1, wherein said cells are selected from the group consisting of primary cells, biopsy derived cells, induced pluripotent (iPS) cells, organoid-derived cells and cell lines.

14. The method of claim 1, wherein said cells are selected from the group consisting of healthy cells, disease cells, cells derived from patients having a disease, cells derived from a patient suspected of developing a disease, and cells derived from a patient identified as having a disease susceptibility.

15. The method of claim 1, wherein said cells are selected from the group consisting of cells derived from a disease affected tissue of a patient and cells derived from an area of tissue next to a disease affected tissue of a patient.

16. The method of claim 1, wherein said cells are selected from the group consisting of cells having at least one known gene sequence, cells having at least one known gene mutation, cells having at least one known genetic allele, and cells having at least one gene that is genetically engineered.

17. The method of claim 1, wherein said cells are derived from patients having at least one disease symptom selected from the group consisting of an inflammatory bowel disease (IBD), celiac disease, Crohn's disease (CD), and ulcerative colitis (UC).

18. The method of claim 1, wherein said cells are derived from patients having at least one disease symptom selected from the group consisting of neurodegenerative disorders, neuro-inflammatory disorders, and X-linked adrenoleukodystrophy (X-ALD).

19. The method of claim 1, wherein said cells are derived from patients having at least one disease symptom selected from the group consisting of diabetes and chronic kidney disease (CKD).

20. The method of claim 1, wherein said cells are derived from patients having at least one disease symptom selected from the group consisting of alcoholic liver disease (ALD) and non-alcoholic fatty liver disease (NAFLD).

21. The method of claim 1, wherein said microfluidic device comprises at least a first cell layer and a second cell layer.

22. The method of claim 21, wherein said first cell layer comprises epithelial cells and said second cell layer comprises endothelial cells.

23. The method of claim 22, wherein said epithelial cells are selected from the group consisting of organoid-derived epithelial cells.

24. The method of claim 23, wherein said organoid-derived epithelial cells are human.

25. The method of claim 22, wherein said epithelial cells are selected from the group consisting of intestine-derived cells, organoid-derived intestine epithelial cells, organoid-derived duodenal epithelial cells, organoid-derived ileal epithelial cells, and organoid-derived colonic epithelial cells.

26. The method of claim 25, wherein said epithelial cells are human organoid-derived colonic epithelial cells.

27. The method of claim 22, wherein said endothelial cells are selected from the group consisting of microvascular endothelial cells (MECs) and umbilical vein endothelial cells (HUVECs).

28. The method of claim 22, wherein said endothelial cells are selected from the group consisting of intestine-derived endothelial cells and intestinal microvascular endothelial cells (IMEC).

29. The method of claim 22, wherein said endothelial cells are selected from the group consisting of brain microvascular endothelial cells (BMECs).

30. The method of claim 22, wherein said endothelial cells are selected from the group consisting of renal glomerular endothelial cells (GEC).

31. The method of claim 22, wherein said endothelial cells are human.

32. The method of claim 22, wherein said endothelial cells are human organoid-derived endothelial cells.

33. The method of claim 22, wherein said endothelial cells are rat.

34. The method of claim 22, wherein said endothelial cells are human biopsy derived endothelial cells.

35. The method of claim 22, wherein said endothelial cells are a human cell line.

36. The method of claim 1, wherein said first substance comprises a cytokine.

37. The method of claim 1, wherein said first substance is selected from the group consisting of a live microbe; a live bacterium; a bacterial substance; Lipopolysaccharides (LPS); and endotoxins.

38. The method of claim 1, wherein said first substance is selected from the group consisting of IFN-γ, TNF-α, IL-1β, IL-4, IL-6, IL-12, IL-17, IL-22, IL-23, and IL-26.

39. The method of claim 1, wherein said first substance is IFN-γ.

40. The method of claim 1, wherein said first substance is a population of white blood cells comprising neutrophils (PMNs).

41. The method of claim 1, wherein said barrier of step a) i) has tight junctions.

42. The method of claim 41, wherein said first substance opens at least a portion of said tight junctions.

43. The method of claim 41, wherein first substance is a six-mer synthetic peptide H-FCIGRL-OH of a Zonula occludens toxin (AT1002).

44. The method of claim 1, wherein said level of permeability comprises molecule permeability, dye permeability, transepithelial electrical resistance, transendothelial electrical resistance, expression of permeability related proteins and visual observation of permeability related proteins.

45. The method of claim 1, wherein said level of permeability is measured by a molecule diffusion assay.

46. The method of claim 1, wherein said level of permeability is measured by a dye diffusion assay.

47. The method of claim 1, wherein said level of permeability is electrically measured.

48. The method of claim 1, wherein said level of permeability is visually observed.

49. The method of claim 1, wherein said barrier is on a membrane.

50. The method of claim 1, wherein said barrier is stretched.
